(19) United States
(12) Patent Application Publication
(10) Pub. No.: US 2015/0291597 A1
(43) Pub. Date: Oct. 15, 2015

(54) NOVEL ORALLY BIOAVAILABLE BREATHING CONTROL MODULATING COMPOUNDS, AND METHODS OF USING SAME

(71) Applicant: Galleon Pharmaceuticals, Inc., Horsham, PA (US)

(72) Inventors: James C. Mannion, Plainsboro, NJ (US); Scott L. Dax, Landenberg, PA (US); Francis John Golder, Philadelphia, PA (US); Duncan Euan MacIntyre, Scotch Plains, NJ (US); James McLeod, Hatboro, PA (US); Vita Ozola, Adazu nov. (LV); Edgars Suna, Riga (LV); Kirill Shubin, Riga (LV); James Joseph Mencel, North Wales, PA (US); Sean X. Peng, Ambler, PA (US)

(21) Appl. No.: 14/442,777

(22) PCT Filed: Nov. 14, 2013
(86) PCT No.: PCT/US2013/070160

§ 371 Date: May 14, 2015

Related U.S. Application Data
(60) Provisional application No. 61/783,451, filed on Mar. 14, 2013.
(60) Provisional application No. 61/726,823, filed on Nov. 15, 2012.

(51) Int. Cl. C07D 487/04; A61K 45/06; A61K 31/519; C07D 251/54; A61K 31/53

(52) U.S. Cl. CPC C07D 487/04; C07D 251/54; A61K 31/53; A61K 31/519; A61K 45/06

(57) ABSTRACT

The present invention includes compositions that are useful in the prevention and/or treatment of breathing control diseases or disorders in a subject in need thereof. The present invention also includes a method of preventing and/or treating a respiratory disease or disorder in a subject in need thereof, comprising administering to the subject a therapeutically effective amount of a composition of the invention. The present invention further includes a method of preventing destabilization or stabilizing breathing rhythm in a subject in need thereof, comprising administering to the subject a therapeutically effective amount of a composition of the invention.

### CROSS-REFERENCE TO RELATED APPLICATIONS

The present application claims priority to U.S. Provisional Applications No. 61/726,823, filed Nov. 15, 2012, and No. 61/783,451, filed Mar. 14, 2013, all of which applications are hereby incorporated by reference in their entireties herein.

### BACKGROUND OF THE INVENTION

Normal control of breathing is a complex process that involves, in part, the body's interpretation and response to chemical stimuli such as carbon dioxide, pH and oxygen levels in blood, tissues and the brain. Normal breathing control is also affected by other factors such as wakefulness (i.e., whether the patient is awake or sleeping), emotion, posture and vocalization. Within the brain medulla, there are respiratory control centers that interpret various feed-forward and feed-back signals that affect respiration by issuing commands to the muscles that perform the work of breathing. Key muscle groups are located in the abdomen, diaphragm, pharynx and thorax. Sensors located centrally and peripherally then provide input to the brain's central respiration control areas that enables response to changing metabolic requirements.

For example, ventilation sufficient to meet the body's metabolic needs is maintained primarily by the body's rapid response to changes in carbon dioxide (CO₂) levels. Increased CO₂ levels (hypercapnia) signal the body to increase breathing rate and depth, resulting in higher blood oxygen levels and subsequent lower blood CO₂ levels. Conversely, low CO₂ levels (hypocapnia) can result in periods of hypopnea (decreased breathing) or, in the extreme case, apnea (no breathing) since the stimulation to breathe is diminished.

There are many diseases in which loss of normal breathing control is a primary or secondary feature of the disease. Examples of diseases with a primary loss of breathing control are sleep apneas (central, mixed or obstructive; where the breathing repeatedly stops for 10 to 60 seconds) and congenital central hypoventilation syndrome. Secondary loss of breathing control may be due to chronic cardio-pulmonary diseases (e.g., heart failure, chronic bronchitis, emphysema, and impending respiratory failure), excessive weight (e.g., obesity-hypoventilation syndrome), certain drugs (e.g., anesthetics, sedatives, sleeping aids, anxiolytics, hypnotics, alcohol, and narcotic analgesics and/or factors that affect the neurological system (e.g., stroke, tumor, trauma, radiation damage, and ALS). In chronic obstructive pulmonary diseases where the body is exposed to chronically high levels of carbon dioxide, the body adapts to the respiratory acidosis (lower pH) by a kidney mediated retention of bicarbonate, which has the effect of partially neutralizing the CO₂/pH respiratory stimulation. Thus, the patient is unable to mount a normal ventilatory response to changes in metabolic demand.

Sleep disordered breathing is an example of where abnormalities in the control of breathing lead to a serious and prevalent disease in humans. Sleep apnea is characterized by frequent periods of no or partial breathing. Key factors that contribute to these apneas include anatomical factors (e.g., obesity), decreased hypercapnic and hypoxic ventilatory responses (e.g., decreased response to high carbon dioxide and low oxygen levels, respectively) and loss of “wakefulness” (respiratory drive to pharyngeal dilator muscles during sleep). Apneic events result in intermittent hypoxia (and the associated oxidative stress) and eventually severe cardiovascular consequences (high blood pressure, stroke, heart attack).

Estimates for U.S. individuals afflicted with conditions wherein there is compromised respiratory control include sleep apneas (15-20 millions); obesity-hypoventilation syndrome (3-5 millions); chronic heart disease (5 millions); chronic obstructive pulmonary disease (COPD)/chronic bronchitis (10 millions); drug-induced hypoventilation (2-10 millions); and mechanical ventilation weaning (0.5 million).

Drugs are most often eliminated by biotransformation and/or excretion into urine, feces or bile. The liver is the major organ for xenobiotic biotransformation, and is thereby important in characterizing the metabolic stability, toxicology, and drug-drug interaction properties of drugs. Drug metabolism is achieved via two major liver-located enzyme reactions: Phase I and Phase II reactions. Phase I enzymes include the cytochrome P450 (CYP450) family of enzymes, which are located in the smooth endoplasmic reticulum. The basic processes in Phase I reactions are oxidation, reduction and/or hydrolysis, many of which are catalyzed by the CYP450 system and require NADPH as a cofactor. Phase II enzymes are located in the cytoplasm and endoplasmic reticulum, and perform conjugation reactions including glucuronic acid, glutathione, sulfate, and glutamine conjugations. Phase II reactions generally inactivate the drug if it is not already therapeutically inactive following Phase I metabolism, and also make the drug more water soluble to facilitate its elimination. Some drugs are metabolized by Phase I or Phase II enzymes alone, whereas others are metabolized by both Phase I and Phase II enzymes (Baranczewski et al., 2006, Pharmacol. Rep. 58:453-472). Microsomes are subcellular liver tissue fractions (membrane vesicles of the smooth endoplasmic reticulum) and contain the Phase I CYP450 family of enzymes. Compounds undergo only Phase I metabolism in liver microsomes in the presence of NADPH cofactors. Significant parent-drug disappearance in the presence of liver microsomes thus indicates that the drug will be significantly modified by the CYP450 enzymes in the body (Rodrigues, 1994, Biochem, Pharm. 48(12):2147).

The purpose of a pharmacokinetic (PK) study is to use drug concentration-time profiles and associated pharmacokinetic parameters to understand how the drug is processed, modified, distributed and/or eliminated upon administration to an animal. In drug discovery, a pharmacokinetic study is performed to (1) guide dosage regimen design for animal efficacy and toxicity studies, (2) understand and interpret pharmacology and toxicology study results, and (3) select the drug candidates with desired pharmacokinetic properties for the disease indication intended. The PK data from the animal studies can be extrapolated to predict PK profiles in humans so as to select and optimize dosage regimens for a drug candidate in human clinical trials.

There is a need in the art for novel compounds useful for restoring all or part of the body's normal breathing control system in response to changes in CO₂ and/or oxygen levels, with minimal side effects. Further, there is a need in the art for novel compounds that are useful for restoring all or part of the body's normal breathing control system and possess suitable metabolic stability and suitable pharmacokinetic properties, such as oral bioavailability. Further, there is a need in the art for novel compounds that are useful for restoring all or part of the body's normal breathing control system and may be administered orally and used in a chronic or acute manner. The present invention addresses and meets these needs.

### BRIEF SUMMARY OF THE INVENTION

The invention includes a compound of formula (I) or a salt thereof:

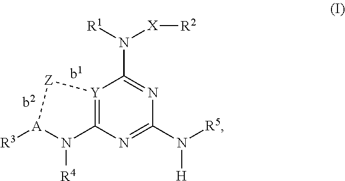

wherein R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl; R³ is H, alkyl, substituted alkyl, alkynyl or substituted alkynyl; R⁴ is H, alkyl, or substituted alkyl; R⁵ is alkyl, propargylic, substituted propargylic, homopropargylic, or substituted homopropargylic, wherein at least one substituent selected from the group consisting of R¹, R², R³ and R⁵ is alkynyl or substituted alkynyl; R⁶ is H, alkyl, substituted alkyl or alkenyl; X is a bond, O or NR⁴; and, Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C.

In one embodiment, R³ is H, alkyl or substituted alkyl, and R⁵ is propargylic, substituted propargylic, homopropargylic, or substituted homopropargylic. In another embodiment, R³ is H or alkynyl, and R⁵ is alkyl, propargylic, substituted propargylic, homopropargylic, or substituted homopropargylic.

In one embodiment, the compound is at least one selected from the group consisting of: (i) Y is N, bond b¹ is nil, Z is H, bond b² is a single bond, A is CH, and the at least one compound is a compound of formula (II-a):

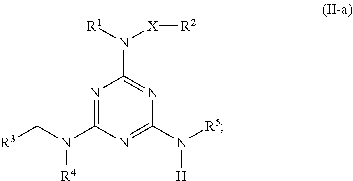

(ii) Y is N, bond b¹ is nil, Z is nil, bond b² is nil, and A is a bond, and the compound of the invention is a 1,3,5-triazine of formula (II-b):

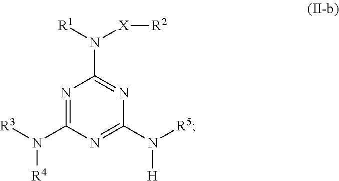

(iii) Y is CR⁶, bond b¹ is nil, Z is H, bond b² is a single bond, A is C, and the at least one compound is a compound of formula (III-a):

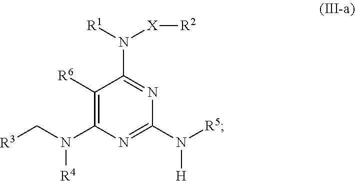

(iv) Y is CR⁶, bond b¹ is nil, Z is nil, bond b² is nil, and A is a bond, and the compound of the invention is a pyrimidine of formula (III-b):

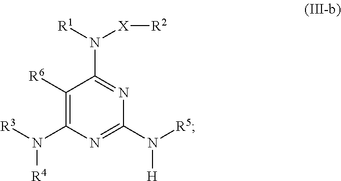

(v) Y is C, bond b¹ is a single bond, Z is CH₂, bond b² is a single bond, A is CH, and the at least one compound is a compound of formula (IV):

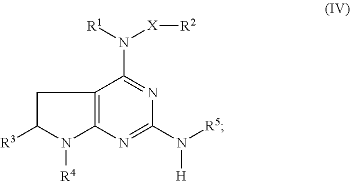

and,
(vi) Y is C, bond b¹ is a single bond, Z is CH, bond b² is a double bond, A is C, and the at least one compound is a compound of formula (V):

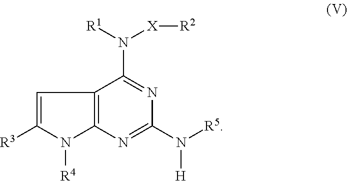

In one embodiment, the at least one compound is selected from the group consisting of O,N-Dimethyl-N-[4(-n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; N-(4-Fluorobenzyl)-O-methyl-N-[4-(n-propylamino)-6-(prop-2-ynylamino)-[1,3,5]triazin-2-yl]-hydroxylamine; N-(4-Fluorobenzyl)-N′-n-propyl-N″-prop-2-ynyl-[1,3,5]triazine-2,4,6-triamine; N-[4-(4-Fluorobenzylamino)-6-(prop-2-ynylamino)-[1,3,5]triazin-2-yl]-O,N-dimethyl-hydroxylamine; N-(4-Fluoro-benzyl)-N-[4-(4-fluorobenzylamino)-6-(prop-2-ynylamino)-[1,3,5]triazin-2-yl]-O-methyl-hydroxylamine; N,N′-Bis-(4-fluorobenzyl)-N″-prop-2-ynyl-[1,3,5]triazine-2,4,6-triamine; N-(4,6-Bis-prop-2-ynylamino-[1,3,5]triazin-2-yl)-O,N-dimethyl-hydroxylamine; N-Methyl-N′,N″-di-prop-2-ynyl-[1,3,5]triazine-2,4,6-triamine; N,N′-Bis-(4-fluoro-benzyl)-N″-n-propyl-[1,3,5]triazine-2,4,6-triamine; O-(4-Fluorophenyl)-N-(4-n-propylamino-6-prop-2-ynylamino-[1,3,5]triazin-2-yl)-hydroxylamine; N-[4-(1,1-Dimethyl-prop-2-ynylamino)-6-n-propylamino-[1,3,5]triazin-2-yl]-O,N-dimethyl-hydroxylamine; O,N-Dimethyl-N-(4-n-propylamino-6-but-2-ynylamino-[1,3,5]triazin-2-yl)-hydroxylamine; O,N-Dimethyl-N-(6-n-propylamino-2-prop-2-ynylamino-pyrimidin-4-yl)-hydroxylamine; O,N-Dimethyl-N-(2-n-propylamino-6-prop-2-ynylamino-pyrimidin-4-yl)-hydroxylamine; O,N-Dimethyl-N-(4-methylamino-6-prop-2-ynylamino-[1,3,5]triazin-2-yl)-hydroxylamine; O,N-Dimethyl-N-(4-ethylamino-6-prop-2-ynylamino-[1,3,5]triazin-2-yl)-hydroxylamine; O,N-Dimethyl-N-(4-isopropylamino-6-prop-2-ynylamino-[1,3,5]triazin-2-yl)-hydroxylamine; O,N-Dimethyl-N-(4-cyclopropylamino-6-prop-2-ynylamino-[1,3,5]triazin-2-yl)-ydroxylamine; O,N-Dimethyl-N-(4-n-butylamino-6-prop-2-ynylamino-[1,3,5]triazin-2-yl)-hydroxylamine; O,N-Dimethyl-N-(4-cyclobutylamino-6-prop-2-ynylamino-[1,3,5]triazin-2-yl)-hydroxylamine; O,N-Dimethyl-N-(4-cyclopropylmethylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-benzylamino-6-prop-2-ynylamino-[1,3,5]triazin-2-yl)-hydroxylamine; O,N-Dimethyl-N-[4-(1-methyl-prop-2-ynylamino)-6-n-propylamino-[1,3,5]triazin-2-yl]-hydroxylamine; O,N-Dimethyl-N-(4-but-3-ynylamino-6-n-propylamino-[1,3,5]triazin-2-yl)-hydroxylamine; N-But-3-ynyl-N′-methyl-N″-n-propyl-[1,3,5]triazine-2,4,6-triamine; O-tert-Butyl-N-(4-n-propylamino-6-prop-2-ynylamino-[1,3,5]triazin-2-yl)-hydroxylamine; O-Ethyl-N-methyl-N-(4-n-propylamino-6-prop-2-ynylamino-[1,3,5]triazin-2-yl)-hydroxylamine; O-Ethyl-N-(4-n-propylamino-6-prop-2-ynylamino-[1,3,5]triazin-2-yl)-hydroxylamine; O-Methyl-N-(4-n-propylamino-6-prop-2-ynylamino-[1,3,5]triazin-2-yl)-hydroxylamine; N-Methyl-N-(4-n-propylamino-6-prop-2-ynylamino-[1,3,5]triazin-2-yl)-hydroxylamine; N-(4-n-Propylamino-6-prop-2-ynylamino-[1,3,5]triazin-2-yl)-hydroxylamine; O-(2-Methoxy-ethyl)-N-methyl-N-(4-n-propylamino-6-prop-2-ynylamino-[1,3,5]triazin-2-yl)-hydroxylamine; N-Methyl-O-(4,4,5,5,5-pentafluoropentyl)-N-(4-n-propylamino-6-prop-2-ynylamino-[1,3,5]triazin-2-yl)-hydroxylamine; N-(4-Fluorophenyl)-N′-propyl-N″-prop-2-ynyl-[1,3,5]triazine-2,4,6-triamine; N-(3-Chloro-2-methyl-benzyl)-N′-n-propyl-N″-prop-2-ynyl-[1,3,5]triazine-2,4,6-triamine; N-(3,4-Dichlorobenzyl)-N′-n-propyl-N″-prop-2-ynyl-[1,3,5]triazine-2,4,6-triamine; O,N-Dimethyl-N-(2-prop-2-ynylamino-7H-pyrrolo[2,3-d]pyrimidin-4-yl)-hydroxylamine; N-(4,6-Bis-n-propylamino-[1,3,5]triazin-2-yl)-O-methyl-N-prop-2-ynyl-hydroxylamine; O-Methyl-N-(4-n-propylamino-6-prop-2-ynylamino-[1,3,5]triazin-2-yl)-N-prop-2-ynyl-hydroxylamine; N-(4,6-Bis-n-propylamino-[1,3,5]triazin-2-yl)-N-methyl-O-prop-2-ynyl-hydroxylamine; N-(4,6-Bis-n-propylamino-[1,3,5]triazin-2-yl)-O-prop-2-ynyl-hydroxylamine; N-Methyl-N-(4-n-propylamino-6-prop-2-ynylamino-[1,3,5]triazin-2-yl)-O-prop-2-ynyl-hydroxylamine; N-(4-n-Propylamino-6-prop-2-ynylamino-[1,3,5]triazin-2-yl)-O-prop-2-ynyl-hydroxylamine; N-(4-Allylamino-6-prop-2-ynylamino-[1,3,5]triazin-2-yl)-O,N-dimethyl-hydroxylamine; 1-[4-(N-Methoxy-N-methyl-amino)-6-prop-2-ynylamino-[1,3,5]triazin-2-ylamino]-propan-2-ol; 3-[4-(N-Methoxy-N-methyl-amino)-6-prop-2-ynylamino-[1,3,5]triazin-2-ylamino]-propan-1-ol; N-(4-Amino-6-prop-2-ynylamino-[1,3,5]triazin-2-yl)-O,N-dimethyl-hydroxylamine; 3-[4-(N-Methoxy-N-methylamino)-6-prop-2-ynylamino-[1,3,5]triazin-2-ylamino]-propionaldehyde; 3-[4-(N-Methoxy-N-methylamino)-6-prop-2-ynylamino-[1,3,5]triazin-2-ylamino]-propionic acid ethyl ester hydrochloride; N-Propyl-N′-prop-2-ynyl-[1,3,5]triazine-2,4,6-triamine; N-[4-(N′-Methoxy-N′-methyl-amino)-6-prop-2-ynylamino-[1,3,5]triazin-2-yl]-N-propyl acetamide; N-[4-(N′-Methoxy-N′-methyl-amino)-6-prop-2-ynylamino-[1,3,5]triazin-2-yl]-N-propyl adamantylamide; N-Ethyl-N′-methyl-N″-prop-2-ynyl-[1,3,5]triazine-2,4,6-triamine; N-Cyclopropyl-N′-methyl-N″-prop-2-ynyl-[1,3,5]triazine-2,4,6-triamine; N-Butyl-N′-methyl-N″-prop-2-ynyl-[1,3,5]triazine-2,4,6-triamine; N-Cyclopropylmethyl-N′-methyl-N″-prop-2-ynyl-[1,3,5]triazine-2,4,6-triamine; N-Methyl-N′-prop-2-ynyl-N″-(3,3,3-trifluoro-propyl)-[1,3,5]triazine-2,4,6-triamine; N-Methyl-N′-(2,2,3,3,3-pentafluoro-propyl)-N″-prop-2-ynyl-[1,3,5]triazine-2,4,6-triamine; N-(1-Ethyl-propyl)-N′-methyl-N″-prop-2-ynyl-[1,3,5]triazine-2,4,6-triamine; N,N-Dimethyl-N′-propyl-N″-prop-2-ynyl-[1,3,5]triazine-2,4,6-triamine; N,N-Ethyl-methyl-N′-propyl-N″-prop-2-ynyl-[1,3,5]triazine-2,4,6-triamine; N-Ethyl-N′-propyl-N″-prop-2-ynyl-[1,3,5]triazine-2,4,6-triamine; N-Propyl-N′-propyl-N″-prop-2-ynyl-[1,3,5]triazine-2,4,6-triamine; N-Cyclopropyl-N′-propyl-N″-prop-2-ynyl-[1,3,5]triazine-2,4,6-triamine; N-Isopropyl-N′-propyl-N″-prop-2-ynyl-[1,3,5]triazine-2,4,6-triamine; N-Butyl-N′-propyl-N″-prop-2-ynyl-[1,3,5]triazine-2,4,6-triamine; N-Cyclopropylmethyl-N′-propyl-N″-prop-2-ynyl-[1,3,5]triazine-2,4,6-triamine; a salt thereof, and any combinations thereof.

In one embodiment, the compound is selected from the group consisting of O,N-dimethyl-N-[4-(n-propylamino)-6-(prop-2-ynylamino)-[1,3,5]triazin-2-yl]-hydroxylamine; N-methyl-N′-n-propyl-N″-prop-2-ynyl-[1,3,5]triazine-2,4,6-triamine; a salt thereof; and any combinations thereof.

In one embodiment, the salt comprises an acid addition salt, and the acid is at least one selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, maleic, glucuronic, fumaric, pyruvic, aspartic, glutamic, benzoic, anthranilic, mandelic, pamoic, 4-hydroxybenzoic, phenylacetic, methanesulfonic, ethanesulfonic, alginic, benzenesulfonic, pantothenic, sulfanilic, stearic, trifluoromethanesulfonic, 2-hydroxyethanesulfonic, p-toluenesulfonic, cyclohexylaminosulfonic, β-hydroxybutyric, salicylic, galactaric and galacturonic, and any combinations thereof.

The invention further includes a pharmaceutical composition comprising a compound of the invention and at least one pharmaceutically acceptable carrier.

In one embodiment, the composition further comprises at least one agent selected from the group consisting of doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists and ampakines. In another embodiment, the compound and the agent are physically mixed in the composition. In yet another embodiment, the compound and the agent are physically separated in the composition.

In one embodiment, the composition further comprises at least one additional agent that causes changes in breathing control. In another embodiment, the additional agent is at least one selected from the group consisting of opioid narcotics, benzodiazepines, sedatives, sleeping aids, hypnotics, propofol, and any combinations thereof. In yet another embodiment, the compound and the additional agent are physically mixed in the composition. In yet another embodiment, the compound and the additional agent are physically separated in the composition.

In one embodiment, the composition allows for modified delivery of the compound following oral administration to a subject. In another embodiment, the composition minimizes delivery of the compound to the stomach of the subject and maximizes delivery of the compound to the intestine of the subject. In yet another embodiment, the composition includes an enteric coating. In yet another embodiment, the compound is contained in a pharmaceutically suitable capsule. In yet another embodiment, the capsule contains granules or powder of the compound, or an admixture of the compound with an excipient. In yet another embodiment, the excipient comprises a binder, disintegrant, diluent, buffer, lubricant, glidant, antioxidant, antimicrobial preservative, colorant, or flavorant. In yet another embodiment, the capsule is enterically coated but the granules or powders of the compound are not enterically coated. In yet another embodiment, the granules or powders of the compound are coated with an enteric coating before being placed into the capsule. In yet another embodiment, the granules or powders of the compound are coated with a multiplicity of enteric coatings, as to provide delivery of drug to different regions of the intestine of the subject. In yet another embodiment, at least a portion of the granules or powders of the compound are enterically coated. In yet another embodiment, the capsule is coated with an enteric coating that is different from the enteric coating that coats the granules or powders of the compound. In yet another embodiment, the compound is coated onto a base particle, whereby a core comprising the drug as a coating over the base particle is formed. In yet another embodiment, the base particle is not enterically coated and the composition is contained in a pharmaceutically acceptable capsule that is enterically coated. In yet another embodiment, the core is coated with an enteric coating, thereby forming an enterically coated bead.

In one embodiment, the enterically coated bead is contained in a pharmaceutically acceptable capsule. In another embodiment, the capsule contains beads coated with a multiplicity of enteric coatings, so that the capsule provides delivery of the compound to different regions of the intestine of the subject. In yet another embodiment, the contents of the capsule are dissolved or suspended in a pharmaceutically acceptable liquid as to provide a liquid-filled capsule. In yet another embodiment, the capsule is enterically coated but the liquid formulation contained within does not comprise an enteric coating. In yet another embodiment, the granules or powders of the compound are enterically coated. In yet another embodiment, the granules or powders of the compound are coated with a multiplicity of enteric coatings, as to provide delivery of drug to different regions of the intestine of the subject. In yet another embodiment, the enteric coating applied to the capsule differs from the enteric coating applied to any of the granules or powders of the compound. In yet another embodiment, the compound is coated onto a base particle to form a core comprising the compound as a coating over the base particle, wherein the core is suspended in a pharmaceutically acceptable liquid, and wherein the suspended core is placed in a capsule. In yet another embodiment, the capsule is enterically coated but the core is not enterically coated. In yet another embodiment, the capsule and the core are enterically coated.

The invention further includes a method of preventing or treating a breathing control disorder or disease in a subject in need thereof. The method comprising administering to the subject an effective amount of a pharmaceutical composition comprising at least one pharmaceutically acceptable carrier and at least one compound of formula (I) or a salt thereof:

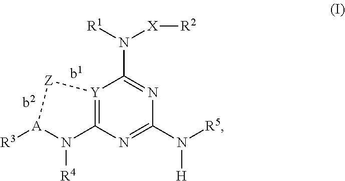

wherein R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl; R³ is H, alkyl, substituted alkyl, alkynyl or substituted alkynyl; R⁴ is H, alkyl, or substituted alkyl; R⁵ is alkyl, propargylic, substituted propargylic, homopropargylic, or substituted homopropargylic, wherein at least one substituent selected from the group consisting of R¹, R², R³ and R⁵ is alkynyl or substituted alkynyl; R⁶ is H, alkyl, substituted alkyl or alkenyl; X is a bond, O or NR⁴; and, Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C.

In one embodiment, the breathing control disorder or disease is at least one selected from the group consisting of respiratory depression, sleep apnea, apnea of prematurity, obesity-hypoventilation syndrome, primary alveolar hypoventilation syndrome, dyspnea, altitude sickness, hypoxia, hypercapnia, chronic obstructive pulmonary disease (COPD), sudden infant death syndrome (SIDS), congenital central hypoventilation syndrome, Alzheimer's disease, Parkinson's disease, stroke, Duchenne muscular dystrophy, and brain and spinal cord traumatic injury. In another embodiment, the respiratory depression is caused by an anesthetic, a sedative, a sleeping aid, an anxiolytic agent, a hypnotic agent, alcohol or a narcotic.

In one embodiment, the subject is further administered at least one agent useful for treating the breathing disorder or disease. In another embodiment, the agent is at least one selected from the group consisting of doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists and ampakines. In yet another embodiment, the compound and the agent are separately administered to the subject. In yet another embodiment, the compound and the agent are co-administered to the subject, further wherein the compound and the agent are physically mixed or physically separated when administered to the subject.

In one embodiment, the subject is further administered at least one additional therapeutic agent that changes normal breathing control in the subject. In another embodiment, at least one additional agent is selected from the group consisting of opioid narcotics, benzodiazepines, sedatives, sleeping aids, hypnotics, propofol, and any combinations thereof.

In one embodiment, the composition is administered in conjunction with the use of a mechanical ventilation device or positive airway pressure device on the subject. In another embodiment, the subject is a mammal or bird. In yet another embodiment, the mammal is a human. In yet another embodiment, the composition is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes. In yet another embodiment, the composition is orally administered to the subject.

In one embodiment, the at least one compound is selected from the group consisting of: O,N-Dimethyl-N-[4(-n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; N-(4-Fluorobenzyl)-O-methyl-N-[4-(n-propylamino)-6-(prop-2-ynylamino)-[1,3,5]triazin-2-yl]-hydroxylamine; N-(4-Fluorobenzyl)-N′-n-propyl-N″-prop-2-ynyl-[1,3,5]triazine-2,4,6-triamine; N-[4-(4-Fluorobenzylamino)-6-(prop-2-ynylamino)-[1,3,5]triazin-2-yl]-O,N-dimethyl-hydroxylamine; N-(4-Fluoro-benzyl)-N-[4-(4-fluorobenzylamino)-6-(prop-2-ynylamino)-[1,3,5]triazin-2-yl]-O-methyl-hydroxylamine; N,N′-Bis-(4-fluorobenzyl)-N″-prop-2-ynyl-[1,3,5]triazine-2,4,6-triamine; N-(4,6-Bis-prop-2-ynylamino-[1,3,5]triazin-2-yl)-O,N-dimethyl-hydroxylamine; N-Methyl-N′,N″-di-prop-2-ynyl-[1,3,5]triazine-2,4,6-triamine; N,N′-Bis-(4-fluoro-benzyl)-N″-n-propyl-[1,3,5]triazine-2,4,6-triamine; O-(4-Fluorophenyl)-N-(4-n-propylamino-6-prop-2-ynylamino-[1,3,5]triazin-2-yl)-hydroxylamine; N-[4-(1,1-Dimethyl-prop-2-ynylamino)-6-n-propylamino-[1,3,5]triazin-2-yl]-O,N-dimethyl-hydroxylamine; O,N-Dimethyl-N-(4-n-propylamino-6-but-2-ynylamino-[1,3,5]triazin-2-yl)-hydroxylamine; O,N-Dimethyl-N-(6-n-propylamino-2-prop-2-ynylamino-pyrimidin-4-yl)-hydroxylamine; O,N-Dimethyl-N-(2-n-propylamino-6-prop-2-ynylamino-pyrimidin-4-yl)-hydroxylamine; O,N-Dimethyl-N-(4-methyl amino-6-prop-2-ynylamino-[1,3,5]triazin-2-yl)-hydroxylamine; O,N-Dimethyl-N-(4-ethylamino-6-prop-2-ynylamino-[1,3,5]triazin-2-yl)-hydroxylamine; O,N-Dimethyl-N-(4-isopropylamino-6-prop-2-ynylamino-[1,3,5]triazin-2-yl)-hydroxylamine; O,N-Dimethyl-N-(4-cyclopropylamino-6-prop-2-ynylamino-[1,3,5]triazin-2-yl)-hydroxylamine; O,N-Dimethyl-N-(4-n-butylamino-6-prop-2-ynylamino-[1,3,5]triazin-2-yl)-hydroxylamine; O,N-Dimethyl-N-(4-cyclobutylamino-6-prop-2-ynylamino-[1,3,5]triazin-2-yl)-hydroxylamine; O,N-Dimethyl-N-(4-cyclopropylmethylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-benzylamino-6-prop-2-ynylamino-[1,3,5]triazin-2-yl)-hydroxylamine; O,N-Dimethyl-N-[4-(1-methyl-prop-2-ynylamino)-6-n-propylamino-[1,3,5]triazin-2-yl]-hydroxylamine; O,N-Dimethyl-N-(4-but-3-ynylamino-6-n-propylamino-[1,3,5]triazin-2-yl)-hydroxylamine; N-But-3-ynyl-N′-methyl-N″-n-propyl-[1,3,5]triazine-2,4,6-triamine; O-tert-Butyl-N-(4-n-propylamino-6-prop-2-ynylamino-[1,3,5]triazin-2-yl)-hydroxylamine; O-Ethyl-N-methyl-N-(4-n-propylamino-6-prop-2-ynylamino-[1,3,5]triazin-2-yl)-hydroxylamine; O-Ethyl-N-(4-n-propylamino-6-prop-2-ynylamino-[1,3,5]triazin-2-yl)-hydroxylamine; O-Methyl-N-(4-n-propylamino-6-prop-2-ynylamino-[1,3,5]triazin-2-yl)-hydroxylamine; N-Methyl-N-(4-n-propylamino-6-prop-2-ynylamino-[1,3,5]triazin-2-yl)-hydroxylamine; N-(4-n-Propylamino-6-prop-2-ynylamino-[1,3,5]triazin-2-yl)-hydroxylamine; O-(2-Methoxy-ethyl)-N-methyl-N-(4-n-propylamino-6-prop-2-ynylamino-[1,3,5]triazin-2-yl)-hydroxylamine; N-Methyl-O-(4,4,5,5,5-pentafluoropentyl)-N-(4-n-propylamino-6-prop-2-ynylamino-[1,3,5]triazin-2-yl)-hydroxylamine; N-(4-Fluorophenyl)-N′-propyl-N″-prop-2-ynyl-[1,3,5]triazine-2,4,6-triamine; N-(3-Chloro-2-methyl-benzyl)-N′-n-propyl-N″-prop-2-ynyl-[1,3,5]triazine-2,4,6-triamine; N-(3,4-Dichlorobenzyl)-N′-n-propyl-N″-prop-2-ynyl-[1,3,5]triazine-2,4,6-triamine; O,N-Dimethyl-N-(2-prop-2-ynylamino-7H-pyrrolo[2,3-d]pyrimidin-4-yl)-hydroxylamine; N-(4,6-Bis-n-propylamino-[1,3,5]triazin-2-yl)-O-methyl-N-prop-2-ynyl-hydroxylamine; O-Methyl-N-(4-n-propylamino-6-prop-2-ynylamino-[1,3,5]triazin-2-yl)-N-prop-2-ynyl-hydroxylamine; N-(4,6-Bis-n-propylamino-[1,3,5]triazin-2-yl)-N-methyl-O-prop-2-ynyl-hydroxylamine; N-(4,6-Bis-n-propylamino-[1,3,5]triazin-2-yl)-O-prop-2-ynyl-hydroxylamine; N-Methyl-N-(4-n-propylamino-6-prop-2-ynylamino-[1,3,5]triazin-2-yl)-O-prop-2-ynyl-hydroxylamine; N-(4-n-Propylamino-6-prop-2-ynylamino-[1,3,5]triazin-2-yl)-O-prop-2-ynyl-hydroxylamine; N-(4-Allylamino-6-prop-2-ynylamino-[1,3,5]triazin-2-yl)-O,N-dimethyl-hydroxylamine; 1-[4-(N-Methoxy-N-methyl-amino)-6-prop-2-ynylamino-[1,3,5]triazin-2-ylamino]-propan-2-ol; 3-[4-(N-Methoxy-N-methyl-amino)-6-prop-2-ynylamino-[1,3,5]triazin-2-ylamino]-propan-1-ol; N-(4-Amino-6-prop-2-ynylamino-[1,3,5]triazin-2-yl)-O,N-dimethyl-hydroxylamine; 3-[4-(N-Methoxy-N-methylamino)-6-prop-2-ynylamino-[1,3,5]triazin-2-ylamino]-propionaldehyde; 3-[4-(N-Methoxy-N-methylamino)-6-prop-2-ynylamino-[1,3,5]triazin-2-ylamino]-propionic acid ethyl ester hydrochloride; N-Propyl-N′-prop-2-ynyl-[1,3,5]triazine-2,4,6-triamine; N-[4-(N′-Methoxy-N′-methyl-amino)-6-prop-2-ynylamino-[1,3,5]triazin-2-yl]-N-propyl acetamide; N-[4-(N′-Methoxy-N′-methyl-amino)-6-prop-2-ynylamino-[1,3,5]triazin-2-yl]-N-propyl adamantylamide; N-Ethyl-N′-methyl-N″-prop-2-ynyl-[1,3,5]triazine-2,4,6-triamine; N-Cyclopropyl-N′-methyl-N″-prop-2-ynyl-[1,3,5]triazine-2,4,6-triamine; N-Butyl-N′-methyl-N″-prop-2-ynyl-[1,3,5]triazine-2,4,6-triamine; N-Cyclopropylmethyl-N′-methyl-N″-prop-2-ynyl-[1,3,5]triazine-2,4,6-triamine; N-Methyl-N′-prop-2-ynyl-N″-(3,3,3-trifluoro-propyl)-[1,3,5]triazine-2,4,6-triamine; N-Methyl-N′-(2,2,3,3,3-pentafluoro-propyl)-N″-prop-2-ynyl-[1,3,5]triazine-2,4,6-triamine; N-(1-Ethyl-propyl)-N′-methyl-N″-prop-2-ynyl-[1,3,5]triazine-2,4,6-triamine; N,N-Dimethyl-N′-propyl-N″-prop-2-ynyl-[1,3,5]triazine-2,4,6-triamine; N,N-Ethyl-methyl-N′-propyl-N″-prop-2-ynyl-[1,3,5]triazine-2,4,6-triamine; N-Ethyl-N′-propyl-N″-prop-2-ynyl-[1,3,5]triazine-2,4,6-triamine; N-Propyl-N′-propyl-N″-prop-2-ynyl-[1,3,5]triazine-2,4,6-triamine; N-Cyclopropyl-N′-propyl-N″-prop-2-ynyl-[1,3,5]triazine-2,4,6-triamine; N-Isopropyl-N′-propyl-N″-prop-2-ynyl-[1,3,5]triazine-2,4,6-triamine; N-Butyl-N′-propyl-N″-prop-2-ynyl-[1,3,5]triazine-2,4,6-triamine; N-Cyclopropylmethyl-N′-propyl-N″-prop-2-ynyl-[1,3,5]triazine-2,4,6-triamine; a salt thereof, and any combinations thereof.

In one embodiment, the compound of formula (I) is selected from the group consisting of O,N-dimethyl-N-[4-(n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; a salt thereof; and any combinations thereof.

In one embodiment, the salt comprises an acid addition salt, and the acid is at least one selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, pamoic, methanesulfonic, ethanesulfonic, benzenesulfonic, pantothenic, sulfanilic, stearic, alginic, trifluoromethanesulfonic, 2-hydroxyethanesulfonic, p-toluenesulfonic, cyclohexylaminosulfonic, β-hydroxybutyric, salicylic, galactaric and galacturonic, and any combinations thereof.

The invention further includes a method of preventing destabilization or stabilizing breathing rhythm in a subject in need thereof. The method comprises administering to the subject an effective amount of a pharmaceutical composition comprising at least one pharmaceutically acceptable carrier and at least one compound of formula (I) or a salt thereof:

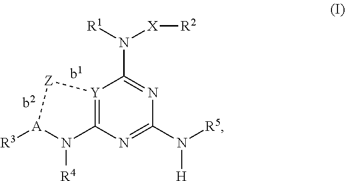

wherein: R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl; R³ is H, alkyl, substituted alkyl, alkynyl or substituted alkynyl; R⁴ is H, alkyl, or substituted alkyl; R⁵ is alkyl, propargylic, substituted propargylic, homopropargylic, or substituted homopropargylic, wherein at least one substituent selected from the group consisting of R¹, R², R³ and R⁵ is alkynyl or substituted alkynyl; R⁶ is H, alkyl, substituted alkyl or alkenyl; X is a bond, O or NR⁴; and, Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C.

In one embodiment, the destabilization is associated with a breathing control disorder or disease selected from the group consisting of respiratory depression, sleep apnea, apnea of prematurity, obesity-hypoventilation syndrome, primary alveolar hypoventilation syndrome, dyspnea, altitude sickness, hypoxia, hypercapnia, chronic obstructive pulmonary disease (COPD), sudden infant death syndrome (SIDS), congenital central hypoventilation syndrome, Alzheimer's disease, Parkinson's disease, stroke, Duchenne muscular dystrophy, and brain and spinal cord traumatic injury. In another embodiment, the respiratory depression is caused by an anesthetic, a sedative, a sleeping aid, an anxiolytic agent, a hypnotic agent, alcohol or a narcotic.

In one embodiment, the subject is further administered at least one agent useful for treating the breathing disorder or disease. In another embodiment, the agent is selected from the group consisting of doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists and ampakines. In yet another embodiment, the compound and the agent are separately administered to the subject. In yet another embodiment, the compound and the agent are co-administered to the subject, further wherein the compound and the agent are physically mixed or physically separated when administered to the subject.

In one embodiment, the subject is further administered at least one additional therapeutic agent that changes normal breathing control in the subject. In another embodiment, the additional agent is at least one selected from the group consisting of opioid narcotics, benzodiazepines, sedatives, sleeping aids, hypnotics, propofol, and any combinations thereof.

In one embodiment, the composition is administered in conjunction with the use of a mechanical ventilation device or positive airway pressure device on the subject. In yet another embodiment, the subject is a mammal or bird. In yet another embodiment, the subject is a mammal. In yet another embodiment, the composition is administered to the subject by at least one route selected from the group consisting of a nasal, inhalational, topical, oral, buccal, rectal, pleural, peritoneal, vaginal, intramuscular, subcutaneous, transdermal, epidural, intrathecal and intravenous routes.

In one embodiment, the at least one compound is selected from the group consisting of: O,N-Dimethyl-N-[4(-n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; N-(4-Fluorobenzyl)-O-methyl-N-[4-(n-propylamino)-6-(prop-2-ynylamino)-[1,3,5]triazin-2-yl]-hydroxylamine; N-(4-Fluorobenzyl)-N′-n-propyl-N″-prop-2-ynyl-[1,3,5]triazine-2,4,6-triamine; N-[4-(4-Fluorobenzylamino)-6-(prop-2-ynylamino)-[1,3,5]triazin-2-yl]-O,N-dimethyl-hydroxylamine; N-(4-Fluoro-benzyl)-N-[4-(4-fluorobenzylamino)-6-(prop-2-ynylamino)-[1,3,5]triazin-2-yl]-O-methyl-hydroxylamine; N,N′-Bis-(4-fluorobenzyl)-N″-prop-2-ynyl-[1,3,5]triazine-2,4,6-triamine; N-(4,6-Bis-prop-2-ynylamino-[1,3,5]triazin-2-yl)-O,N-dimethyl-hydroxylamine; N-Methyl-N′,N″-di-prop-2-ynyl-[1,3,5]triazine-2,4,6-triamine; N,N′-Bis-(4-fluoro-benzyl)-N″-n-propyl-[1,3,5]triazine-2,4,6-triamine; O-(4-Fluorophenyl)-N-(4-n-propylamino-6-prop-2-ynylamino-[1,3,5]triazin-2-yl)-hydroxylamine; N-[4-(1,1-Dimethyl-prop-2-ynylamino)-6-n-propylamino-[1,3,5]triazin-2-yl]-O,N-dimethyl-hydroxylamine; O,N-Dimethyl-N-(4-n-propylamino-6-but-2-ynylamino-[1,3,5]triazin-2-yl)-hydroxylamine; O,N-Dimethyl-N-(6-n-propylamino-2-prop-2-ynylamino-pyrimidin-4-yl)-hydroxylamine; O,N-Dimethyl-N-(2-n-propylamino-6-prop-2-ynylamino-pyrimidin-4-yl)-hydroxylamine; O,N-Dimethyl-N-(4-methyl amino-6-prop-2-ynylamino-[1,3,5]triazin-2-yl)-hydroxylamine; O,N-Dimethyl-N-(4-ethylamino-6-prop-2-ynylamino-[1,3,5]triazin-2-yl)-hydroxylamine; O,N-Dimethyl-N-(4-isopropylamino-6-prop-2-ynylamino-[1,3,5]triazin-2-yl)-hydroxylamine; O,N-Dimethyl-N-(4-cyclopropylamino-6-prop-2-ynylamino-[1,3,5]triazin-2-yl)-hydroxylamine; O,N-Dimethyl-N-(4-n-butylamino-6-prop-2-ynylamino-[1,3,5]triazin-2-yl)-hydroxylamine; O,N-Dimethyl-N-(4-cyclobutylamino-6-prop-2-ynylamino-[1,3,5]triazin-2-yl)-hydroxylamine; O,N-Dimethyl-N-(4-cyclopropylmethylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-benzylamino-6-prop-2-ynylamino-[1,3,5]triazin-2-yl)-hydroxylamine; O,N-Dimethyl-N-[4-(1-methyl-prop-2-ynylamino)-6-n-propylamino-[1,3,5]triazin-2-yl]-hydroxylamine; O,N-Dimethyl-N-(4-but-3-ynylamino-6-n-propylamino-[1,3,5]triazin-2-yl)-hydroxylamine; N-But-3-ynyl-N′-methyl-N″-n-propyl-[1,3,5]triazine-2,4,6-triamine; O-tert-Butyl-N-(4-n-propylamino-6-prop-2-ynylamino-[1,3,5]triazin-2-yl)-hydroxylamine; O-Ethyl-N-methyl-N-(4-n-propylamino-6-prop-2-ynylamino-[1,3,5]triazin-2-yl)-hydroxylamine; O-Ethyl-N-(4-n-propylamino-6-prop-2-ynylamino-[1,3,5]triazin-2-yl)-hydroxylamine; O-Methyl-N-(4-n-propylamino-6-prop-2-ynylamino-[1,3,5]triazin-2-yl)-hydroxylamine; N-Methyl-N-(4-n-propylamino-6-prop-2-ynylamino-[1,3,5]triazin-2-yl)-hydroxylamine; N-(4-n-Propylamino-6-prop-2-ynylamino-[1,3,5]triazin-2-yl)-hydroxylamine; O-(2-Methoxy-ethyl)-N-methyl-N-(4-n-propylamino-6-prop-2-ynylamino-[1,3,5]triazin-2-yl)-hydroxylamine; N-Methyl-O-(4,4,5,5,5-pentafluoropentyl)-N-(4-n-propylamino-6-prop-2-ynylamino-[1,3,5]triazin-2-yl)-hydroxylamine; N-(4-Fluorophenyl)-N′-propyl-N″-prop-2-ynyl-[1,3,5]triazine-2,4,6-triamine; N-(3-Chloro-2-methyl-benzyl)-N′-n-propyl-N″-prop-2-ynyl-[1,3,5]triazine-2,4,6-triamine; N-(3,4-Dichlorobenzyl)-N′-n-propyl-N″-prop-2-ynyl-[1,3,5]triazine-2,4,6-triamine; O,N-Dimethyl-N-(2-prop-2-ynylamino-7H-pyrrolo[2,3-d]pyrimidin-4-yl)-hydroxylamine; N-(4,6-Bis-n-propylamino-[1,3,5]triazin-2-yl)-O-methyl-N-prop-2-ynyl-hydroxylamine; O-Methyl-N-(4-n-propylamino-6-prop-2-ynylamino-[1,3,5]triazin-2-yl)-N-prop-2-ynyl-hydroxylamine; N-(4,6-Bis-n-propylamino-[1,3,5]triazin-2-yl)-N-methyl-O-prop-2-ynyl-hydroxylamine; N-(4,6-Bis-n-propylamino-[1,3,5]triazin-2-yl)-O-prop-2-ynyl-hydroxylamine; N-Methyl-N-(4-n-propylamino-6-prop-2-ynylamino-[1,3,5]triazin-2-yl)-O-prop-2-ynyl-hydroxylamine; N-(4-n-Propylamino-6-prop-2-ynylamino-[1,3,5]triazin-2-yl)-O-prop-2-ynyl-hydroxylamine; N-(4-Allylamino-6-prop-2-ynylamino-[1,3,5]triazin-2-yl)-O,N-dimethyl-hydroxylamine; 1-[4-(N-Methoxy-N-methyl-amino)-6-prop-2-ynylamino-[1,3,5]triazin-2-ylamino]-propan-2-ol; 3-[4-(N-Methoxy-N-methyl-amino)-6-prop-2-ynylamino-[1,3,5]triazin-2-ylamino]-propan-1-ol; N-(4-Amino-6-prop-2-ynylamino-[1,3,5]triazin-2-yl)-O,N-dimethyl-hydroxylamine; 3-[4-(N-Methoxy-N-methylamino)-6-prop-2-ynylamino-[1,3,5]triazin-2-ylamino]-propionaldehyde; 3-[4-(N-Methoxy-N-methylamino)-6-prop-2-ynylamino-[1,3,5]triazin-2-ylamino]-propionic acid ethyl ester hydrochloride; N-Propyl-N′-prop-2-ynyl-[1,3,5]triazine-2,4,6-triamine; N-[4-(N′-Methoxy-N′-methyl-amino)-6-prop-2-ynylamino-[1,3,5]triazin-2-yl]-N-propyl acetamide; N-[4-(N′-Methoxy-N′-methyl-amino)-6-prop-2-ynylamino-[1,3,5]triazin-2-yl]-N-propyl adamantylamide; N-Ethyl-N′-methyl-N″-prop-2-ynyl-[1,3,5]triazine-2,4,6-triamine; N-Cyclopropyl-N′-methyl-N″-prop-2-ynyl-[1,3,5]triazine-2,4,6-triamine; N-Butyl-N′-methyl-N″-prop-2-ynyl-[1,3,5]triazine-2,4,6-triamine; N-Cyclopropylmethyl-N′-methyl-N″-prop-2-ynyl-[1,3,5]triazine-2,4,6-triamine; N-Methyl-N′-prop-2-ynyl-N″-(3,3,3-trifluoro-propyl)-[1,3,5]triazine-2,4,6-triamine; N-Methyl-N′-(2,2,3,3,3-pentafluoro-propyl)-N″-prop-2-ynyl-[1,3,5]triazine-2,4,6-triamine; N-(1-Ethyl-propyl)-N′-methyl-N″-prop-2-ynyl-[1,3,5]triazine-2,4,6-triamine; N,N-Dimethyl-N′-propyl-N″-prop-2-ynyl-[1,3,5]triazine-2,4,6-triamine; N,N-Ethyl-methyl-N′-propyl-N″-prop-2-ynyl-[1,3,5]triazine-2,4,6-triamine; N-Ethyl-N′-propyl-N″-prop-2-ynyl-[1,3,5]triazine-2,4,6-triamine; N-Propyl-N′-propyl-N″-prop-2-ynyl-[1,3,5]triazine-2,4,6-triamine; N-Cyclopropyl-N′-propyl-N″-prop-2-ynyl-[1,3,5]triazine-2,4,6-triamine; N-Isopropyl-N′-propyl-N″-prop-2-ynyl-[1,3,5]triazine-2,4,6-triamine; N-Butyl-N′-propyl-N″-prop-2-ynyl-[1,3,5]triazine-2,4,6-triamine; N-Cyclopropylmethyl-N′-propyl-N″-prop-2-ynyl-[1,3,5]triazine-2,4,6-triamine; a salt thereof, and any combinations thereof.

In one embodiment, the compound of formula (I) is selected from the group consisting of O,N-dimethyl-N-[4-(n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; a salt thereof; and any combinations thereof.

In one embodiment, the salt comprises an acid addition salt, and the acid is at least one selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, pamoic, methanesulfonic, ethanesulfonic, benzenesulfonic, pantothenic, sulfanilic, stearic, alginic, trifluoromethanesulfonic, 2-hydroxyethanesulfonic, p-toluenesulfonic, cyclohexylaminosulfonic, β-hydroxybutyric, salicylic, galactaric and galacturonic, and any combinations thereof.

The invention further includes a method of preparing O,N-dimethyl-N-[4-(n-propylamino)-6-(prop-2-ynylamino)-[1,3,5]triazin-2-yl]-hydroxylamine or a salt thereof. The method comprising the steps of: (a) contacting cyanuric chloride with n-propyl amine in a solvent in the presence of a base; (b) adding propargyl amine and a base to the mixture of step (a) and heating the resulting mixture; (c) isolating from the mixture of step (b) solid 6-chloro-N-propyl-N′-prop-2-ynyl-[1,3,5]triazine-2,4-diamine; (d) contacting the product of step (c) with O,N-dimethylhydroxylamine in a solvent at a temperature; (e) isolating from the mixture of step (d) solid O,N-dimethyl-N-[4-(n-propylamino)-6-(prop-2-ynylamino)-[1,3,5]triazin-2-yl]-hydroxylamine; and, (f) optionally contacting the product of step (e) with an acid, thereby forming an acid addition salt of O,N-dimethyl-N-[4-(n-propylamino)-6-(prop-2-ynylamino-[1,3,5]triazin-2-yl]-hydroxylamine.

In one embodiment, the acid addition salt formed in step (f) is at least one selected from the group consisting of: a sulfuric acid addition salt with an XRPD spectrum as illustrated in FIG. 22, 23, 24 or 25; an L(+)-tartaric acid addition salt with an XRPD spectrum as illustrated in FIG. 27; a maleic acid addition salt with an XRPD spectrum as illustrated in FIG. 29; a DL-mandelic acid addition salt with an XRPD spectrum as illustrated in FIG. 31; a malonic acid addition salt with an XRPD spectrum as illustrated in FIG. 33; a fumaric acid addition salt with an XRPD spectrum as illustrated in FIG. 35; and, a saccharin addition salt with an XRPD spectrum as illustrated in FIG. 37.

In one embodiment, the solid O,N-dimethyl-N-[4-(n-propylamino)-6-(prop-2-ynylamino)-[1,3,5]triazin-2-yl]-hydroxylamine has an XRPD spectrum as illustrated in FIG. 18 or 19. In another embodiment, the product of step (f) is contacted with a base in a solvent, thereby yielding O,N-dimethyl-N-[4-(n-propylamino)-6-(prop-2-ynylamino)-[1,3,5]triazin-2-yl]-hydroxylamine free base. In yet another embodiment, the O,N-dimethyl-N-[4-(n-propylamino)-6-(prop-2-ynylamino-[1,3,5]triazin-2-yl]-hydroxylamine free base is contacted with an additional acid that is distinct from the acid in step (f), thereby yielding the additional acid addition salt of O,N-dimethyl-N-[4-(n-propylamino)-6-(prop-2-ynylamino)-[1,3,5]triazin-2-yl]-hydroxylamine. In yet another embodiment, formation of 6-chloro-N,N-propyl-[1,3,5]triazine-2,4-diamine in step (a) is minimized. In yet another embodiment, the propargyl amine used in step (b) comprises less than 0.01 weight % of 2-chloroallyl amine. In yet another embodiment, the propargyl amine used in step (b) comprises a 2:1 propargyl amine-sulfuric acid addition salt. In yet another embodiment, the isolated compound in step (c) contains less than 0.5% 6-chloro-N,N′-propyl-[1,3,5]triazine-2,4-diamine.

In one embodiment, step (e) comprises the steps of: cooling the mixture of step (d) below 60° C.; diluting the resulting mixture with 2 volumes of water with vigorous stirring over about 2-3 h; seeding the resulting system with a crystal of O,N-dimethyl-N-[4-(n-propylamino)-6-(prop-2-ynylamino)-[1,3,5]triazin-2-yl]-hydroxylamine; stirring the resulting system for 10-20 h, whereby crystallization of O,N-dimethyl-N-[4-(n-propylamino)-6-(prop-2-ynylamino)-[1,3,5]triazin-2-yl]-hydroxylamine takes place.

In one embodiment, the solid O,N-dimethyl-N-[4-(n-propylamino)-6-(prop-2-ynylamino)-[1,3,5]triazin-2-yl]-hydroxylamine contains less that 0.01 weight % of N,O-dimethyl-N-(4-n-propylamino-6-(2-chloro-prop-2-enylamino)-[1,3,5]triazin-2-yl)-hydroxylamine.

The invention further includes a method of preparing the compound O,N-dimethyl-N-[4-(n-propylamino)-6-(prop-2-ynylamino)-[1,3,5]triazin-2-yl]-hydroxylamine or a salt thereof, wherein the compound is substantially free of N,O-Dimethyl-N-[4-n-propylamino-6-(2-chloro-prop-2-enylamino)-[1,3,5]triazin-2-yl]-hydroxylamine. The method comprises the steps of: (a) contacting cyanuric chloride with n-propyl amine in a solvent in the presence of a base; (b) adding N,O-dimethylhydroxylamine, optionally along with a base, to the mixture of step (a) and heating the resulting mixture; (c) isolating from the mixture of step (b) the compound 6-chloro-N-n-propyl-N′-prop-2-ynyl-[1,3,5]triazine-2,4-diamine; (d) contacting the compound isolated in step (c) with trialkyl amine in a solvent at a temperature, and isolating the compound 4-(N-methoxy-N-methyl-amino)-6-n-propylamino-[1,3,5]triazin-2-yl]-trimethyl-ammonium chloride; (e) contacting the compound isolated in step (d) with a salt of tetrafluoroboric acid in a solvent at a temperature, and isolating the compound 4-(N-methoxy-N-methyl-amino)-6-n-propylamino-[1,3,5]triazin-2-yl]-trimethyl-ammonium tetrafluoroborate; (f) contacting the compound isolated in step (e) with propargyl amine at a temperature, and isolating the compound N,O-dimethyl-N-(4-n-propylamino-6-prop-2-ynylamino-[1,3,5]triazin-2-yl)-hydroxylamine; (g) optionally crystallizing the compound isolated in step (f) thus yielding crystalline N,O-dimethyl-N-(4-n-propylamino-6-prop-2-ynylamino-[1,3,5]triazin-2-yl)-hydroxylamine; (h) optionally contacting the product isolated in step (f) or (g) with about one molar equivalent of maleic acid, and isolating the hydrogen maleinate salt of N,O-dimethyl-N-(4-n-propylamino-6-prop-2-ynylamino-[1,3,5]triazin-2-yl)-hydroxylamine; (i) optionally contacting the product of step (h) with a base in a solvent, and isolating N,O-dimethyl-N-(4-n-propylamino-6-prop-2-ynylamino-[1,3,5]triazin-2-yl)-hydroxylamine free base; and, (j) optionally contacting the compound isolated in step (g) or (i) about one molar equivalent of L(+)-tartartic acid in a solvent, and isolating the L(+)-hydrogen tartrate salt of N,O-dimethyl-N-(4-n-propylamino-6-prop-2-ynylamino-[1,3,5]triazin-2-yl)-hydroxylamine.

In one embodiment, the compound O,N-dimethyl-N-[4-(n-propylamino)-6-(prop-2-ynylamino)-[1,3,5]triazin-2-yl]-hydroxylamine or a salt thereof contains <0.002 weight % N,O-dimethyl-N-(4-n-propylamino-6-(2-chloro-prop-2-enyl)amino-[1,3,5]triazin-2-yl)-hydroxylamine.

The invention further includes a composition comprising O,N-dimethyl-N-[4-(n-propylamino)-6-(prop-2-ynylamino)-[1,3,5]triazin-2-yl]-hydroxylamine or a salt thereof selected from the group consisting of: (a) a crystalline form of O,N-dimethyl-N-(4-n-propylamino-6-prop-2-ynylamino-[1,3,5]triazin-2-yl)-hydroxylamine with a XRPD spectrum as illustrated in FIG. 18 or 19; (b) a crystalline form of O,N-dimethyl-N-(4-n-propylamino-6-prop-2-ynylamino-[1,3,5]triazin-2-yl)-hydroxylamine/sulfuric acid (1:1) addition salt with a XRPD spectrum as illustrated in FIG. 22; (c) a crystalline form of O,N-dimethyl-N-(4-n-propylamino-6-prop-2-ynylamino-[1,3,5]triazin-2-yl)-hydroxylamine/sulfuric acid (2:1) addition salt with a XRPD spectrum as illustrated in FIG. 23; (d) a crystalline form of O,N-dimethyl-N-(4-n-propylamino-6-prop-2-ynylamino-[1,3,5]triazin-2-yl)-hydroxylamine/sulfuric acid (1:2) addition salt with a XRPD spectrum as illustrated in FIG. 24; (e) an amorphous form of O,N-dimethyl-N-(4-n-propylamino-6-prop-2-ynylamino-[1,3,5]triazin-2-yl)-hydroxylamine/sulfuric acid (4:3) addition salt with a XRPD spectrum as illustrated in FIG. 25; (f) a crystalline form of O,N-dimethyl-N-(4-n-propylamino-6-prop-2-ynylamino-[1,3,5]triazin-2-yl)-hydroxylamine/L(+)-tartaric acid (1:1) addition salt with a XRPD spectrum as illustrated in FIG. 27; (g) a crystalline form of O,N-dimethyl-N-(4-n-propylamino-6-prop-2-ynylamino-[1,3,5]triazin-2-yl)-hydroxylamine/maleic acid (1:1) addition salt with a XRPD spectrum as illustrated in FIG. 29; (h) a crystalline form of O,N-dimethyl-N-(4-n-propylamino-6-prop-2-ynylamino-[1,3,5]triazin-2-yl)-hydroxylamine/DL-mandelic acid (1:1) addition salt with a XRPD spectrum as illustrated in FIG. 31; (i) a crystalline form of O,N-dimethyl-N-(4-n-propylamino-6-prop-2-ynylamino-[1,3,5]triazin-2-yl)-hydroxylamine/malonic acid (1:1) addition salt with a XRPD spectrum as illustrated in FIG. 33; (j) a crystalline form of O,N-dimethyl-N-(4-n-propylamino-6-prop-2-ynylamino-[1,3,5]triazin-2-yl)-hydroxylamine/fumaric acid (1:1) addition salt with a XRPD spectrum as illustrated in FIG. 35; (k) a crystalline form of O,N-dimethyl-N-(4-n-propylamino-6-prop-2-ynylamino-[1,3,5]triazin-2-yl)-hydroxylamine/saccharin (1:1) addition salt with a XRPD spectrum as illustrated in FIG. 37; and any combinations thereof.

The invention further includes a composition comprising [4-(N-methoxy-N-methyl-amino)-6-n-propylamino-[1,3,5]triazin-2-yl]-trimethyl-ammonium tetrafluoroborate.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a table illustrating reagent volumes used in a microsomal stability assay.

FIG. 2, comprising FIGS. 2A-2F, is a set of tables illustrating ventilatory stimulation parameters peak increase in minute volume (V_{E}) and increase in minute volume (MV) area under the curve (AUC) versus reference compounds and microsomal half-life values for exemplary compounds of the invention.

FIG. 3 is a table illustrating plasma concentrations measured upon dosing of Compound 5b to the rat.

FIG. 4 is a table illustrating pharmacokinetic parameters of Compound 5b in the rat.

FIG. 5 is a graph illustrating plasma concentrations of Compound 5b when dosed IV in individual rats.

FIG. 6 is a graph illustrating plasma concentrations of Compound 5b when dosed PO in individual rats.

FIG. 7 is a graph illustrating time-course plasma concentrations of Compound 5b when dosed IV and PO in the rat.

FIG. 8 is a set of graphs illustrating the effect of Compound 5a on respiratory rate and tidal volume when dosed IV in the rat.

FIG. 9 is a graph illustrating the effect of Compound 5a on minute volume when dosed IV in the rat.

FIG. 10 is a set of graphs illustrating the effect of Compound 7a on respiratory rate and tidal volume when dosed IV in the rat.

FIG. 11 is a graph illustrating the effect of Compound 7a on minute volume when dosed IV in the rat.

FIG. 12 is a set of graphs illustrating the effect of Compound 9a on respiratory rate and tidal volume when dosed IV in the rat.

FIG. 13 is a graph illustrating the effect of Compound 9a on minute volume when dosed IV in the rat.

FIG. 14 is a graph illustrating the effect of Compound 5b when dosed PO on minute volume in the rat.

FIG. 15 is a graph illustrating the effect of Compound 5b when dosed PO on mean blood pressure in the rat.

FIG. 16 illustrates the ¹H NMR spectrum of O,N-dimethyl-N-[4-(n-propylamino)-6-(prop-2-ynylamino)-[1,3,5]triazin-2-yl]-hydroxylamine in CDCl₃ (Example 2C).

FIG. 17 illustrates the ¹³C NMR spectrum of O,N-dimethyl-N-[4-(n-propylamino)-6-(prop-2-ynylamino)-[1,3,5]triazin-2-yl]-hydroxylamine in CDCl₃ (Example 2C).

FIG. 18 illustrates the XRPD spectrum of O,N-dimethyl-N-[4-(n-propylamino)-6-(prop-2-ynylamino)-[1,3,5]triazin-2-yl]-hydroxylamine free base (C₁₁H₁₈N₆O) from a mixture of dimethylacetamide and water (Example 2C).

FIG. 19 illustrates the XRPD spectrum of O,N-dimethyl-N-[4-(n-propylamino)-6-(prop-2-ynylamino)-[1,3,5]triazin-2-yl]-hydroxylamine free base (C₁₁H₁₈N₆O) from a mixture of petroleum ether-40 and toluene (Example 2D).

FIG. 20 illustrates the ¹H NMR spectrum of O,N-dimethyl-N-[4-(n-propylamino)-6-(prop-2-ynylamino)-[1,3,5]triazin-2-yl]-hydroxylamine/sulfuric acid addition salt in a 1:1 molar ratio (C₁₁H₁₈N₆O*H₂SO₄) in CDCl₃ (Example 3C).

FIG. 21 illustrates the ¹³C NMR spectrum of O,N-dimethyl-N-[4-(n-propylamino)-6-(prop-2-ynylamino)-[1,3,5]triazin-2-yl]-hydroxylamine/sulfuric acid addition salt in a 1:1 molar ratio (C₁₁H₁₈N₆O*H₂SO₄) in CDCl₃ (Example 3C).

FIG. 22 illustrates the XRPD spectrum of O,N-dimethyl-N-[4-(n-propylamino)-6-(prop-2-ynylamino)-[1,3,5]triazin-2-yl]-hydroxylamine hydrogen sulfate salt (C₁₁H₁₈N₆O*H₂SO₄) obtained from methyl ethyl ketone (Example 3C).

FIG. 23 illustrates the XRPD spectrum of O,N-dimethyl-N-[4-(n-propylamino)-6-(prop-2-ynylamino)-[1,3,5]triazin-2-yl]-hydroxylamine/sulfuric acid addition salt in a 2:1 molar ratio (C₁₁H₁₈N₆O*0.5 H₂SO₄) (Example 3E-1).

FIG. 24 illustrates the XRPD spectrum of O,N-dimethyl-N-[4-(n-propylamino)-6-(prop-2-ynylamino)-[1,3,5]triazin-2-yl]-hydroxylamine/sulfuric acid addition salt in a 1:2 molar ratio (C₁₁H₁₈N₆O*2 H₂SO₄) (Example 3E-2).

FIG. 25 illustrates the XRPD spectrum of O,N-dimethyl-N-[4-(n-propylamino)-6-(prop-2-ynylamino)-[1,3,5]triazin-2-yl]-hydroxylamine/sulfuric acid addition salt in a 4:3 molar ratio (4 C₁₁H₁₈N₆O*3 H₂SO₄) (Example 3E-3).

FIG. 26 illustrates the ¹H NMR spectrum of O,N-dimethyl-N-[4-(n-propylamino)-6-(prop-2-ynylamino)-[1,3,5]triazin-2-yl]-hydroxylamine/L(+)-tartaric acid addition salt in a 1:1 molar ratio (Example 3F, Method 1).

FIG. 27 illustrates the XRPD spectrum of O,N-dimethyl-N-[4-(n-propylamino)-6-(prop-2-ynylamino)-[1,3,5]triazin-2-yl]-hydroxylamine/L(+)-tartaric acid addition salt in a 1:1 molar ratio as obtained from isopropanol (Example 3F, Method 1).

FIG. 28 illustrates the ¹H NMR spectrum of O,N-dimethyl-N-[4-(n-propylamino)-6-(prop-2-ynylamino)-[1,3,5]triazin-2-yl]-hydroxylamine/maleic acid addition salt in a 1:1 molar ratio (Example 3G, Method 1).

FIG. 29 illustrates the XRPD spectrum of O,N-dimethyl-N-[4-(n-propylamino)-6-(prop-2-ynylamino)-[1,3,5]triazin-2-yl]-hydroxylamine/maleic acid addition salt in a 1:1 molar ratio as obtained from methyl ethyl ketone (Example 3G, Method 1).

FIG. 30 illustrates the ¹H NMR spectrum of O,N-dimethyl-N-[4-(n-propylamino)-6-(prop-2-ynylamino)-[1,3,5]triazin-2-yl]-hydroxylamine/DL-mandelic addition salt in a 1:1 molar ratio (Example 3H, Method 1).

FIG. 31 illustrates the XRPD spectrum of O,N-dimethyl-N-[4-(n-propylamino)-6-(prop-2-ynylamino)-[1,3,5]triazin-2-yl]-hydroxylamine/DL-mandelic addition salt in a 1:1 molar ration as obtained from acetonitrile (Example 3H, Method 1).

FIG. 32 illustrates the ¹H NMR spectrum of O,N-dimethyl-N-[4-(n-propylamino)-6-(prop-2-ynylamino)-[1,3,5]triazin-2-yl]-hydroxylamine/malonic acid addition salt in a 1:1 molar ratio in CDCl₃.

FIG. 33 illustrates the XRPD spectra of O,N-dimethyl-N-[4-(n-propylamino)-6-(prop-2-ynylamino)-[1,3,5]triazin-2-yl]-hydroxylamine/malonic acid addition salt in a 1:1 molar ratio as obtained from ethanol admixed with diethyl ether (Example 3I, Method 1).

FIG. 34 illustrates the ¹H NMR spectrum of O,N-dimethyl-N-[4-(n-propylamino)-6-(prop-2-ynylamino)-[1,3,5]triazin-2-yl]-hydroxylamine/fumaric acid addition salt in a 1:1 molar ratio.

FIG. 35 illustrates the XRPD spectrum of O,N-dimethyl-N-[4-(n-propylamino)-6-(prop-2-ynylamino)-[1,3,5]triazin-2-yl]-hydroxylamine/fumaric acid addition salt in a 1:1 molar ratio as obtained from ethyl acetate admixed with ethanol (Example 3J, Method 1).

FIG. 36 illustrates the ¹H NMR spectrum for O,N-dimethyl-N-[4-(n-propylamino)-6-(prop-2-ynylamino)-[1,3,5]triazin-2-yl]-hydroxylamine/saccharin addition salt in a 1:1 molar ratio (Example 3L, Method 2).

FIG. 37 illustrates the XRPD spectrum of O,N-dimethyl-N-[4-(n-propylamino)-6-(prop-2-ynylamino)-[1,3,5]triazin-2-yl]-hydroxylamine/saccharin addition salt in a 1:1 molar ratio as obtained from isopropanol.

FIG. 38 is a graph that illustrates the time-course effect of Compound 5b on the frequency of central apneas during NREM sleep in rats that had been chronically treated with morphine, versus the vehicle-treated group. Compound 5b decreased apnea frequency at 120 min and 150 min post-gavage compared to vehicle. * different to vehicle p<0.05. Values are means±SEM.

FIG. 39 is a graph that illustrates the time-course effect of Compound 5b (lower trace) on central apneas during NREM sleep in rats, expressed as percent change from baseline (pre-treatment) versus the vehicle-treated group (upper trace). After the 60-min post-dose, the percent change (decrease) in apnea frequency was present in rats receiving Compound 5b compared to those receiving vehicle. The initial decrease in apnea frequency between 0 and 60 min post-dose was due to the arousal effect of oral gavage (seen in both vehicle and Compound 5b treated rats. * different to vehicle p<0.05. Values are means±SEM.

FIG. 40 is a bar graph that illustrates the effect of Compound 5b on central apnea frequency during NREM sleep in rats that had been chronically treated with morphine. Compound 5b decreased apnea frequency during NREM sleep compared to vehicle and the pre-treatment (baseline) values. The initial 60-min period post-dose was not included as this had a gavage artifact in both groups. * different to vehicle; # different to baseline; p<0.05. Values are means±SEM.

FIG. 41 is a graph illustrating the time-course effect of Compound 5b on percent time spent in NREM sleep in rats that had been chronically treated with morphine, versus vehicle-treated group. No differences were observed in time spent in NREM sleep between groups. The gavage artifact (arousal) is evident between 0 and 60 min post-dose. Values are means±SEM.

FIG. 42 is a bar graph illustrating the effect of Compound 5b on percent time spent in NREM sleep in rats that had been chronically treated with morphine. Compound 5b had no visible effect on time spent in NREM sleep compared to vehicle or the pre-treatment (baseline) values. The initial 60-min period post-dose was not included as this had a gavage artifact seen in both groups. Values are means±SEM.

FIG. 43 is a graph illustrating the time-course effect of Compound 5b on NREM minute volume (V_{E}) in rats that had been chronically treated with morphine, versus the vehicle-treated group. Compound 5b had no statistically significant effects on minute volume. There was a trend for an initial increase in minute volume between 0 to 60 min post-dose. Values are means±SEM.

FIG. 44 is a bar graph illustrating the effect of Compound 5b on NREM minute volume in rats that had been chronically treated with morphine. Compound 5b had no discernible effect on minute volume during NREM sleep compared to vehicle or the pre-treatment (baseline) values. The initial 60-min period post-dose was not included to be consistent with the prior bar graphs presented in this series. Values are means±SEM.

FIG. 45 is a graph that illustrates the time-course effect of Compound 5b on the frequency of central apneas during REM sleep in rats that had been chronically treated with morphine, versus the vehicle-treated group. Compound 5b did not visibly alter apnea frequency during REM sleep compared to vehicle. Values are means±SEM.

FIG. 46 is a bar graph illustrating the effect of Compound 5b on central apnea frequency during REM sleep in rats that had been chronically treated with morphine. Compound 5b had no visible effect on apnea frequency compared to vehicle or the pretreatment (baseline) values. The initial 60-min period post-dose was not included as this had a gavage artifact in both groups. Values are means±SEM.

FIG. 47 is a bar graph illustrating the effect of Compound 5b on REM minute volume in rats that had been chronically treated with morphine. Compound 5b had no visible effects on minute volume compared to vehicle or pre-treatment (baseline) values. The initial 60-min period post-dose was not included to be consistent with prior bar graphs presented in this series. Values are means±SEM.

FIG. 48 is a bar graph illustrating the effect of Compound 5b on percent time spent in REM sleep in rats that had been chronically treated with morphine. Compound 5b had no visible effect on time spent in REM sleep compared to vehicle or the pre-treatment (baseline) values. The initial 60-min period post-dose was not included as this had a gavage artifact seen in both groups. Values are means±SEM.

FIG. 49 is a bar graph illustrating the change in minute volume from baseline, before and after carotid sinus nerve transaction. Rats received saline or Compound 5b at one of two doses. Minute volume was determined prior to, and after transaction.

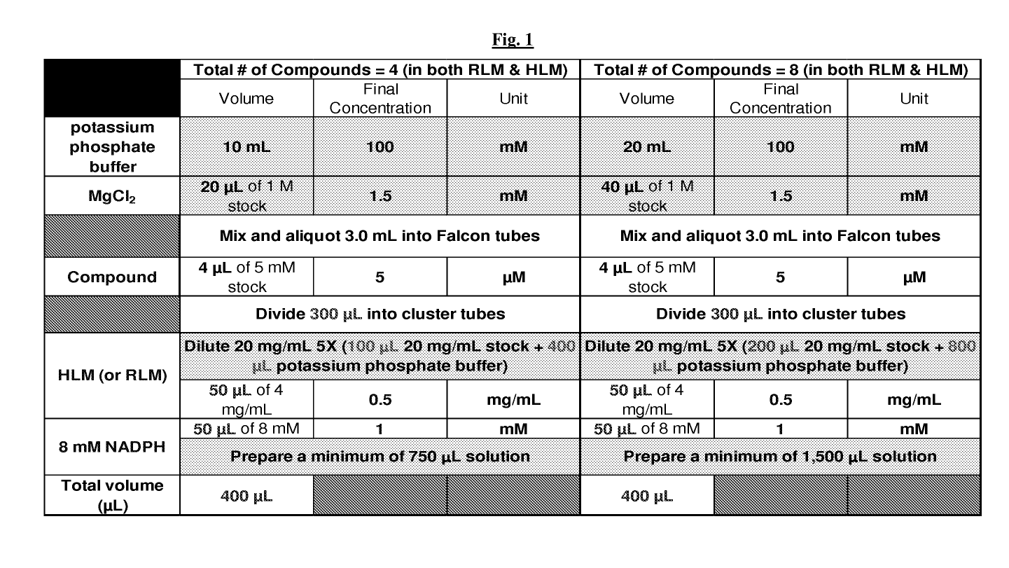

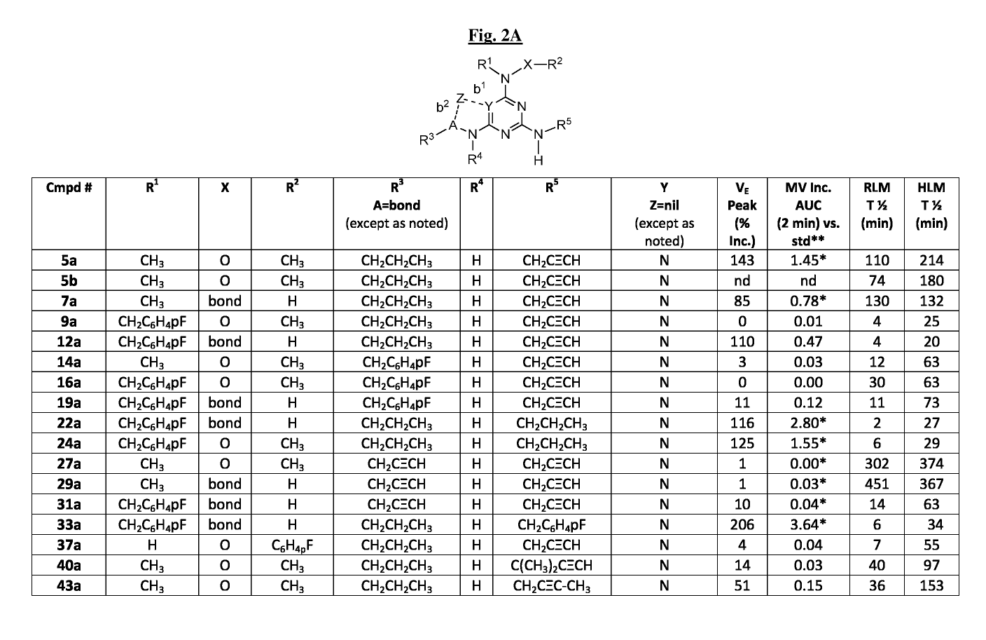

### DETAILED DESCRIPTION OF THE INVENTION

The present invention relates to the unexpected discovery that the compounds of the invention are orally bioavailable breathing control modulators and useful in the prevention or treatment of breathing control disorders or diseases. Further, the compounds of the invention are orally bioavailable breathing control modulators suitable for chronic use in the prevention or treatment of breathing control disorders or diseases. Further, the compounds of the invention are breathing control modulators and useful in the prevention or treatment of breathing control disorders or diseases upon oral administration.

In one aspect, the compounds of the invention prevent changes to the body's normal breathing control system, as a result of disorders and diseases and in response to changes in CO₂ and/or oxygen levels, with minimal side effects. In another aspect, the compounds of the invention decrease the incidence and severity of breathing control disturbances, such as apneas. In yet another aspect, the compounds of the invention decrease the incidence of apneic events and/or decrease the duration of apneic events. In yet another aspect, the compounds of the invention have good metabolic stability and oral bioavailability. In yet another aspect, the compounds of the invention do not interfere with the effectiveness of therapies that may cause changes to breathing control, such as opioid analgesia. Such breathing control-altering therapies benefit from administration of agents that support or restore normal breathing function.

In one aspect, the compounds of the invention are an improvement over previously reported breathing control modulating compounds, such as the compounds disclosed in U.S. application Ser. No. 13/306,349. In another, the compounds of the invention have improved microsomal stability and metabolic stability over the compounds of the prior art. In another aspect, the compounds of the invention have improved oral bioavailability over the compounds taught in the prior art. In yet another aspect, the compounds of the invention have improved pharmacological activities over the compounds taught in the prior art. In yet another aspect, the compounds of the invention display a developable cytochrome CYP450 profile (metabolism) and low activity at cardiac channels such as, but not limited to, hERG.

In one embodiment, the breathing control disorder or disease is selected from the group consisting of respiratory depression, sleep apnea, apnea of prematurity, obesity-hypoventilation syndrome, primary alveolar hypoventilation syndrome, dyspnea, altitude sickness, hypoxia, hypercapnia, chronic obstructive pulmonary disease (COPD) and sudden infant death syndrome (SIDS). In another embodiment, the respiratory depression is caused by an anesthetic, a sedative, a sleeping aid, an anxiolytic agent, a hypnotic agent, alcohol or a narcotic. In yet another embodiment, the respiratory depression is caused by genetic factors as manifested in congenital central hypoventilation syndrome. In yet another embodiment, the respiratory depression is caused by neurological conditions such as, but not limited to, Alzheimer's disease, Parkinson's disease, stroke, Duchenne muscular dystrophy, and brain and spinal cord traumatic injury.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, a “subject” may be a human or non-human mammal or a bird. Non-human mammals include, for example, livestock and pets, such as ovine, bovine, porcine, canine, feline and murine mammals. Preferably, the subject is human.

In a non-limiting embodiment, the following terminology used to report blood gas measurements is well known to those skilled in the art and may be defined as such: minute ventilation (MV) is a measure of breathing volume per unit time and is given herein as mL/min; pCO₂ is partial pressure of carbon dioxide (gas) in (arterial) blood measured in mm Hg (millimeters of Hg); pO₂ is partial pressure of oxygen (gas) in (arterial) blood measured in mmHg (millimeters of Hg); SaO₂ is the percentage of oxyhemoglobin saturation (oxygen gas bound to hemoglobin) that correlates to the percentage of hemoglobin binding sites in the bloodstream occupied by oxygen; end-tidal CO₂ is the measurement of exhaled carbon dioxide gas as detected using calorimetry, capnometry, or capnography techniques.

As used herein, the term ED₅₀ refers to the effective dose of a formulation that produces 50% of the maximal effect in subjects that are administered that formulation.

As used herein, the term “CYP450” as applied to enzymes refers to cytochrome P450 family of enzymes.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, an “effective amount,” “therapeutically effective amount” or “pharmaceutically effective amount” of a compound is that amount of compound that is sufficient to provide a beneficial effect to the subject to which the compound is administered.

The term “treat,” “treating” or “treatment,” as used herein, means reducing the frequency or severity with which symptoms of a disease or condition are experienced by a subject by virtue of administering an agent or compound to the subject.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “composition” or “pharmaceutical composition” refers to a mixture of at least one compound useful within the invention with a pharmaceutically acceptable carrier. The pharmaceutical composition facilitates administration of the compound to a subject.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

In one aspect, the terms “co-administered” and “co-administration” as relating to a subject refer to administering to the subject a compound of the invention or salt thereof along with a compound that may also treat breathing control disorders and/or with a compound that is useful in treating other medical conditions but which in themselves may alter breathing control. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

By the term “specifically bind” or “specifically binds,” as used herein, is meant that a first molecule preferentially binds to a second molecule (e.g., a particular receptor or enzyme), but does not necessarily bind only to that second molecule.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁-6 alkyl, —OH, C₁-6 alkoxy, halo, amino, acetamido and nitro. As used herein, where a substituent is an alkyl or alkoxy group, the carbon chain may be branched, straight or cyclic, with straight being preferred.

### THE FOLLOWING ABBREVIATIONS ARE USED HEREIN

ABG arterial blood gas;
AcOH acetic acid;
ASV adaptive servo ventilation;
AUC area under (the) curve;
BiPAP bi-level positive airway pressure;
nBuOH n-butanol;
C carbon atom or elemental carbon;
¹³C NMR carbon-13 nuclear magnetic resonance;
CHCl₃ chloroform;
CDCl₃ chloroform-d;
CH₂Cl₂ dichloromethane or methylene dichloride;
CPAP continous positive airway pressure;
DIPEA N,N-diisopropylethylamine;
DMAc N,N-dimethylacetamide;
DMSO dimethylsulfoxide;
EPAP expiratory positive airway pressure;
EtOAc ethyl acetate;
EtOH ethanol;
Et₂O (di)ethyl ether;
f frequency (of respiration);
F (%) bioavailability (percent);
FID flame ionization detector;
H hydrogen atom;
¹H NMR proton or hydrogen-1 nuclear magnetic resonance;
HCl hydrochloric acid or a hydrochloride salt;
HDPE high-density polyethylene;
hERG human Ether-a-go-go Related Gene (Kv11.1 ion channel);
H₂SO₄ sulfuric acid;
HLM human liver microsomes;
HPLC high pressure liquid chromatography;
ICU intensive care unit;
IPA isopropanol (or 2-propanol);
IPAP inspiratory positive airway pressure;
kPa kilopascal;
LCMS liquid chromatography-mass spectrometry;
LOQ limit of quantification;
m multiplet;
mbar millibar (0.001 bar);
MBP mean blood pressure;
MTBE methyl tert-butyl ether;
MeCN or CH₃CN acetonitrile;
MEK methyl ethyl ketone;
MeOH or CH₃OH methanol;
min minute;
mL (or ml) milliliter;
mpk mg/kg;
MV minute volume;
MS mass spectrometry;
N nitrogen atom;
NaCl sodium chloride;
NaHCO₃ sodium bicarbonate;
NaOH sodium hydroxide;
Na₂SO₄ sodium sulfate;
NAVA neurally adjusted ventilatory assist;
NIPPV non-invasive positive pressure ventilation;
NMR nuclear magnetic resonance;
PA propargylamine (propargylic amine);
PAV proportional assist ventilation;
PE or pet ether petroleum ether;
PEG polyethylene glycol;
ppm part per million;
RLM rat liver microsomes;
RR respiratory rate;
rt room (ambient) temperature;
s singlet;
std standard;
t triplet;
THF tetrahydrofuran;
TV tidal volume;
UPLC ultra performance liquid chromatography;
V_{E} minute (expired) volume;
XRPD x-ray powder diffraction (spectrum).
δ (delta) delta (ppm);
μL (μl) microliter;

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

### Compounds and Compositions of the Invention

The invention includes a compound of formula (I) or a salt thereof:

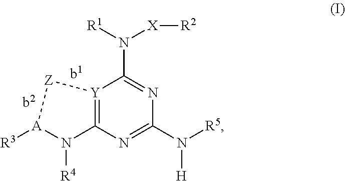

R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl; R³ is H, alkyl, substituted alkyl, alkynyl or substituted alkynyl; R⁴ is H, alkyl, or substituted alkyl; R⁵ is alkyl, propargylic, substituted propargylic, homopropargylic, or substituted homopropargylic, further wherein at least one substituent selected from the group consisting of R¹, R², R³ and R⁵ is alkynyl or substituted alkynyl; R⁶ is H, alkyl, substituted alkyl or alkenyl; X is a bond, O or NR⁴; and, Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C.

In one embodiment, R³ is H, alkyl or substituted alkyl, and R⁵ is propargylic, substituted propargylic, homopropargylic, or substituted homopropargylic. In another embodiment, R³ is H or alkynyl, and R⁵ is alkyl, propargylic, substituted propargylic, homopropargylic, or substituted homopropargylic. In yet another embodiment, R³ is propargylic, substituted propargylic, homopropargylic, or substituted homopropargylic.

In one embodiment, Y is N, bond b¹ is nil, Z is H, bond b² is a single bond, A is CH, and the compound of the invention is a 1,3,5-triazine of formula (II-a) or a salt thereof:

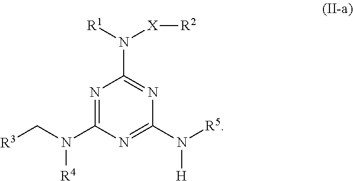

In one embodiment, Y is N, bond b¹ is nil, Z is nil, bond b² is nil, and A is a bond, and the compound of the invention is a 1,3,5-triazine of formula (II-b) or a salt thereof:

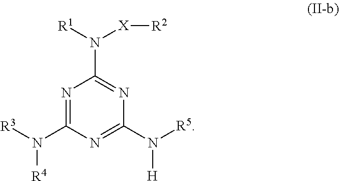

In one embodiment, Y is CR⁶, bond b¹ is nil, Z is H, bond b² is a single bond, A is CH, and the compound of the invention is a pyrimidine of formula (III-a) or a salt thereof:

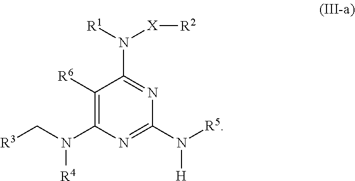

In one embodiment, Y is CR⁶, bond b¹ is nil, Z is nil, bond b² is nil, and A is a bond, and the compound of the invention is a pyrimidine of formula (III-b) or a salt thereof:

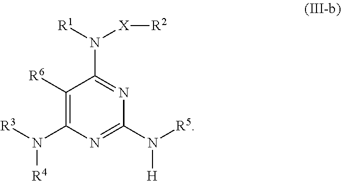

In one embodiment, Y is C, bond b¹ is a single bond, Z is CH₂, bond b² is a single bond, A is C, and the compound of the invention is a pyrrolidinopyrimidine of formula (IV) or a salt thereof:

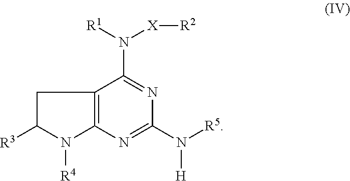

In one embodiment, Y is C, bond b¹ is a single bond, Z is CH, bond b² is a double bond, A is C, and the compound of the invention is a pyrrolopyrimidine of formula (V) or a salt thereof:

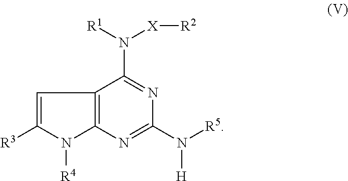

In one embodiment, the compound of formula (I) is selected from the group consisting of:
O,N-Dimethyl-N-[4(-n-propylamino)-6-(prop-2-ynylamino)-[1,3,5]triazin-2-yl]-hydroxylamine;
N-Methyl-N′-n-propyl-N″-prop-2-ynyl-[1,3,5]triazine-2,4,6-triamine;
N-(4-Fluorobenzyl)-O-methyl-N-[4-(n-propylamino)-6-(prop-2-ynylamino)-[1,3,5]triazin-2-yl]-hydroxylamine;
N-(4-Fluorobenzyl)-N′-n-propyl-N″-prop-2-ynyl-[1,3,5]triazine-2,4,6-triamine;
N-[4-(4-Fluorobenzylamino)-6-(prop-2-ynylamino)-[1,3,5]triazin-2-yl]-O,N-dimethyl-hydroxylamine;
N-(4-Fluoro-benzyl)-N-[4-(4-fluorobenzylamino)-6-(prop-2-ynylamino)-[1,3,5]triazin-2-yl]-O-methyl-hydroxylamine;
N,N′-Bis-(4-fluorobenzyl)-N″-prop-2-ynyl-[1,3,5]triazine-2,4,6-triamine;
N-(4,6-Bis-prop-2-ynylamino-[1,3,5]triazin-2-yl)-O,N-dimethyl-hydroxylamine;
N-Methyl-N′,N″-di-prop-2-ynyl-[1,3,5]triazine-2,4,6-triamine;
N-(4-Fluoro-benzyl)-N′,N″-di-prop-2-ynyl-[1,3,5]triazine-2,4,6-triamine; O-(4-Fluorophenyl)-N-(4-n-propylamino-6-prop-2-ynylamino-[1,3,5]triazin-2-yl)-hydroxylamine;
N-[4-(1,1-Dimethyl-prop-2-ynylamino)-6-n-propylamino-[1,3,5]triazin-2-yl]-O,N-dimethyl-hydroxylamine;
O,N-Dimethyl-N-(4-n-propylamino-6-but-2-ynylamino-[1,3,5]triazin-2-yl)-hydroxylamine;
O,N-Dimethyl-N-(6-n-propylamino-2-prop-2-ynylamino-pyrimidin-4-yl)-hydroxylamine;
O,N-Dimethyl-N-(2-n-propylamino-6-prop-2-ynylamino-pyrimidin-4-yl)-hydroxylamine;
O,N-Dimethyl-N-(4-methylamino-6-prop-2-ynylamino-[1,3,5]triazin-2-yl)-hydroxylamine;
O,N-Dimethyl-N-(4-ethylamino-6-prop-2-ynylamino-[1,3,5]triazin-2-yl)-hydroxylamine;
O,N-Dimethyl-N-(4-isopropylamino-6-prop-2-ynylamino-[1,3,5]triazin-2-yl)-hydroxylamine;
O,N-Dimethyl-N-(4-cyclopropylamino-6-prop-2-ynylamino-[1,3,5]triazin-2-yl)-hydroxylamine;
O,N-Dimethyl-N-(4-n-butylamino-6-prop-2-ynylamino-[1,3,5]triazin-2-yl)-hydroxylamine;
O,N-Dimethyl-N-(4-cyclobutylamino-6-prop-2-ynylamino-[1,3,5]triazin-2-yl)-hydroxylamine;
O,N-Dimethyl-N-(4-cyclopropylmethylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-benzylamino-6-prop-2-ynylamino-[1,3,5]triazin-2-yl)-hydroxylamine;
O,N-Dimethyl-N-[4-(1-methyl-prop-2-ynylamino)-6-n-propylamino-[1,3,5]triazin-2-yl]-hydroxylamine;
O,N-Dimethyl-N-(4-but-3-ynylamino-6-n-propylamino-[1,3,5]triazin-2-yl)-hydroxylamine;
N-But-3-ynyl-N′-methyl-N″-propyl-[1,3,5]triazine-2,4,6-triamine;
O-tert-Butyl-N-(4-n-propylamino-6-prop-2-ynylamino-[1,3,5]triazin-2-yl)-hydroxylamine;
O-Ethyl-N-methyl-N-(4-n-propylamino-6-prop-2-ynylamino-[1,3,5]triazin-2-yl)-hydroxylamine;
O-Ethyl-N-(4-n-propylamino-6-prop-2-ynylamino-[1,3,5]triazin-2-yl)-hydroxylamine;
O-Methyl-N-(4-n-propylamino-6-prop-2-ynylamino-[1,3,5]triazin-2-yl)-hydroxylamine;
N-Methyl-N-(4-n-propylamino-6-prop-2-ynylamino-[1,3,5]triazin-2-yl)-hydroxylamine;
N-(4-n-Propylamino-6-prop-2-ynylamino-[1,3,5]triazin-2-yl)-hydroxylamine;
O-(2-Methoxy-ethyl)-N-methyl-N-(4-n-propylamino-6-prop-2-ynylamino-[1,3,5]triazin-2-yl)-hydroxylamine;
N-Methyl-O-(4,4,5,5,5-pentafluoro-pentyl)-N-(4-n-propylamino-6-prop-2-ynylamino-[1,3,5]triazin-2-yl)-hydroxylamine;
N-(4-Fluorophenyl)-N′-propyl-N″-prop-2-ynyl-[1,3,5]triazine-2,4,6-triamine;
N-(3-Chloro-2-methyl-benzyl)-N′-n-propyl-N″-prop-2-ynyl-[1,3,5]triazine-2,4,6-triamine;
N-(3,4-Dichlorobenzyl)-N′-n-propyl-N″-prop-2-ynyl-[1,3,5]triazine-2,4,6-triamine;
O,N-Dimethyl-N-(2-prop-2-ynylamino-7H-pyrrolo[2,3-d]pyrimidin-4-yl)-hydroxylamine;
N-(4,6-Bis-n-propylamino-[1,3,5]triazin-2-yl)-O-methyl-N-prop-2-ynyl-hydroxylamine;
O-Methyl-N-(4-n-propylamino-6-prop-2-ynylamino-[1,3,5]triazin-2-yl)-N-prop-2-ynyl-hydroxylamine;
N-(4,6-Bis-n-propylamino-[1,3,5]triazin-2-yl)-N-methyl-O-prop-2-ynyl-hydroxylamine;
N-(4,6-Bis-n-propylamino-[1,3,5]triazin-2-yl)-O-prop-2-ynyl-hydroxylamine;
N-Methyl-N-(4-n-propylamino-6-prop-2-ynylamino-[1,3,5]triazin-2-yl)-O-prop-2-ynyl-hydroxylamine;
N-(4-n-Propylamino-6-prop-2-ynylamino-[1,3,5]triazin-2-yl)-O-prop-2-ynyl-hydroxylamine;
N-(4-Allylamino-6-prop-2-ynylamino-[1,3,5]triazin-2-yl)-O,N-dimethyl-hydroxylamine;
1-[4-(N-methoxy-N-methyl-amino)-6-prop-2-ynylamino-[1,3,5]triazin-2-ylamino]-propan-2-ol;
3-[4-(N-methoxy-N-methyl-amino)-6-prop-2-ynylamino-[1,3,5]triazin-2-ylamino]-propan-1-ol;
N-(4-Amino-6-prop-2-ynylamino-[1,3,5]triazin-2-yl)-O,N-dimethyl-hydroxylamine;
3-[4-(N-Methoxy-N-methylamino)-6-prop-2-ynylamino-[1,3,5]triazin-2-ylamino]-propionaldehyde;
3-[4-(N-Methoxy-N-methylamino)-6-prop-2-ynylamino-[1,3,5]triazin-2-ylamino]-propionic acid ethyl ester;
N-Propyl-N′-prop-2-ynyl-[1,3,5]triazine-2,4,6-triamine;
N-[4-(N′-Methoxy-N′-methyl-amino)-6-prop-2-ynylamino-[1,3,5]triazin-2-yl]-N-propyl acetamide;
N-[4-(N′-Methoxy-N′-methyl-amino)-6-prop-2-ynylamino-[1,3,5]triazin-2-yl]-N-propyl adamantylamide;
N-Ethyl-N′-methyl-N″-prop-2-ynyl-[1,3,5]triazine-2,4,6-triamine;
N-Cyclopropyl-N′-methyl-N″-prop-2-ynyl-[1,3,5]triazine-2,4,6-triamine;
N-Butyl-N′-methyl-N″-prop-2-ynyl-[1,3,5]triazine-2,4,6-triamine;
N-Cyclopropylmethyl-N′-methyl-N″-prop-2-ynyl-[1,3,5]triazine-2,4,6-triamine;
N-Methyl-N′-prop-2-ynyl-N″-(3,3,3-trifluoro-propyl)-[1,3,5]triazine-2,4,6-triamine;
N-Methyl-N′-(2,2,3,3,3-pentafluoro-propyl)-N″-prop-2-ynyl-[1,3,5]triazine-2,4,6-triamine;
N-(1-Ethyl-propyl)-N′-methyl-N″-prop-2-ynyl-[1,3,5]triazine-2,4,6-triamine;
N,N-Dimethyl-N′-propyl-N″-prop-2-ynyl-[1,3,5]triazine-2,4,6-triamine;
N,N-Ethyl-methyl-N′-propyl-N″-prop-2-ynyl-[1,3,5]triazine-2,4,6-triamine;
N-Ethyl-N′-propyl-N″-prop-2-ynyl-[1,3,5]triazine-2,4,6-triamine;
N-Propyl-N′-propyl-N″-prop-2-ynyl-[1,3,5]triazine-2,4,6-triamine;
N-Cyclopropyl-N′-propyl-N″-prop-2-ynyl-[1,3,5]triazine-2,4,6-triamine;
N-Isopropyl-N′-propyl-N″-prop-2-ynyl-[1,3,5]triazine-2,4,6-triamine;
N-Butyl-N′-propyl-N″-prop-2-ynyl-[1,3,5]triazine-2,4,6-triamine;
N-Cyclopropylmethyl-N′-propyl-N″-prop-2-ynyl-[1,3,5]triazine-2,4,6-triamine;
   a salt thereof; and any combinations thereof.

In a preferred embodiment, the compound of formula (I) is selected from the group consisting of O,N-Dimethyl-N-[4-(n-propylamino)-6-(prop-2-ynylamino-[1,3,5]triazin-2-yl]-hydroxylamine; N-Methyl-N′-n-propyl-N″-prop-2-ynyl-[1,3,5]triazine-2,4,6-triamine; a salt thereof; and any combinations thereof.

In one embodiment, the salt comprises an acid that is at least one selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, pamoic, methanesulfonic, ethanesulfonic, benzenesulfonic, pantothenic, sulfanilic, stearic, alginic, trifluoromethanesulfonic, 2-hydroxyethanesulfonic, p-toluenesulfonic, cyclohexylaminosulfonic, β-hydroxybutyric, salicylic, galactaric and galacturonic, and any combinations thereof.

In one embodiment, the at least one compound of formula (I) is a component of a pharmaceutical composition further including at least one pharmaceutically acceptable carrier.

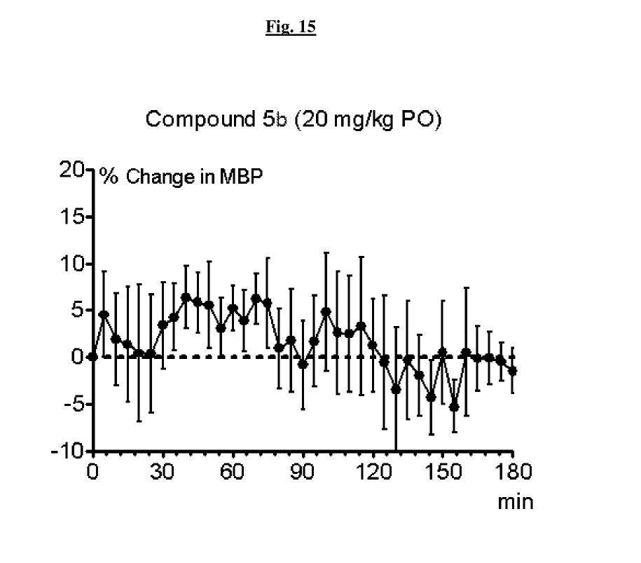

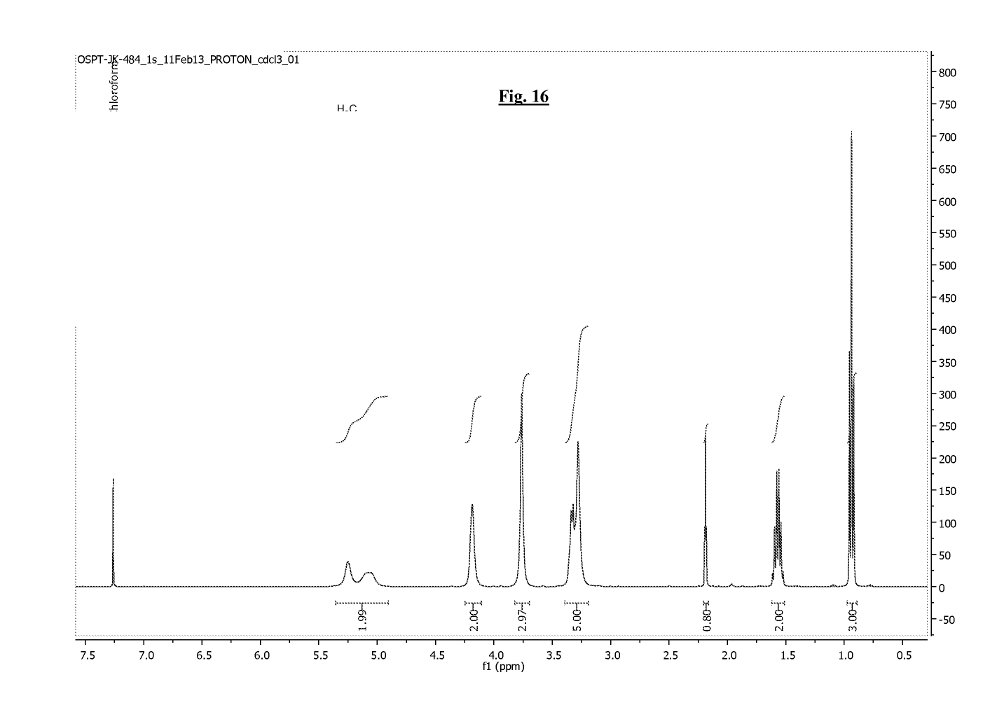

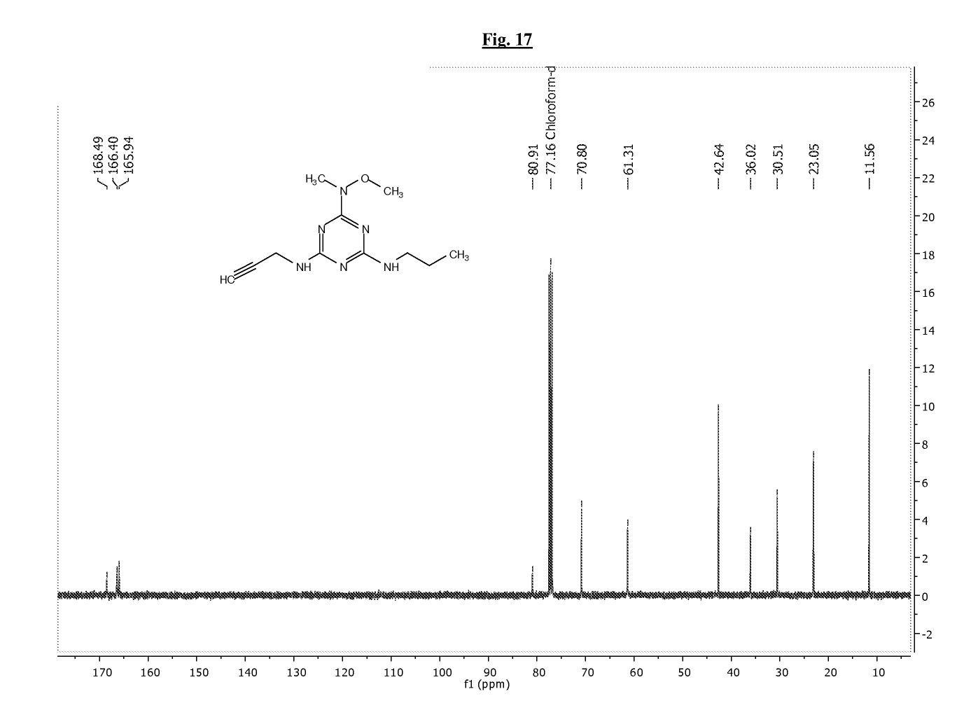

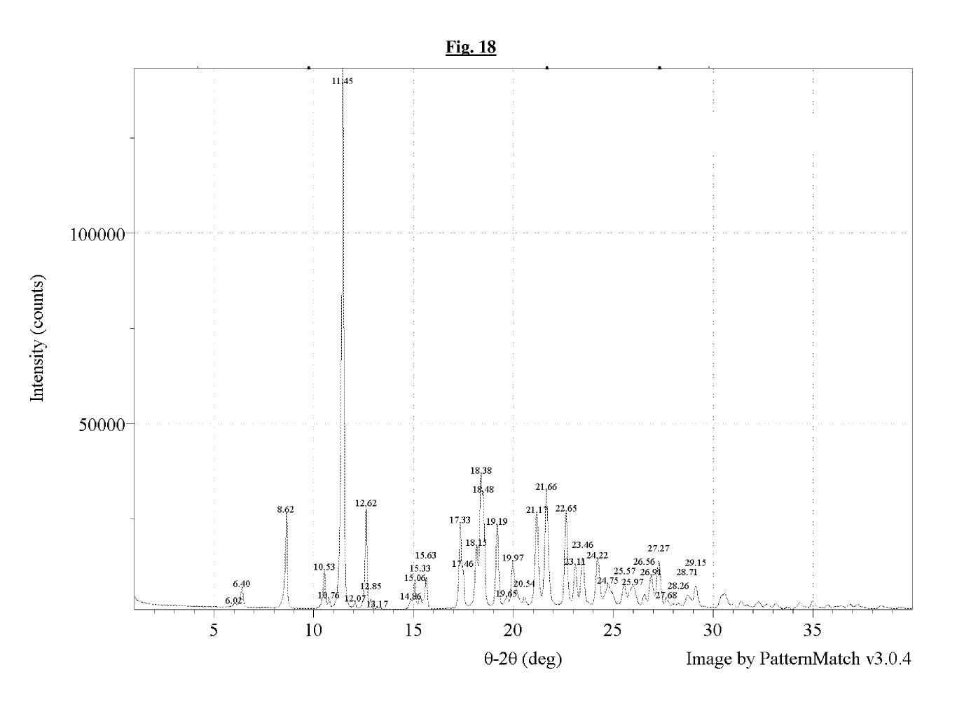

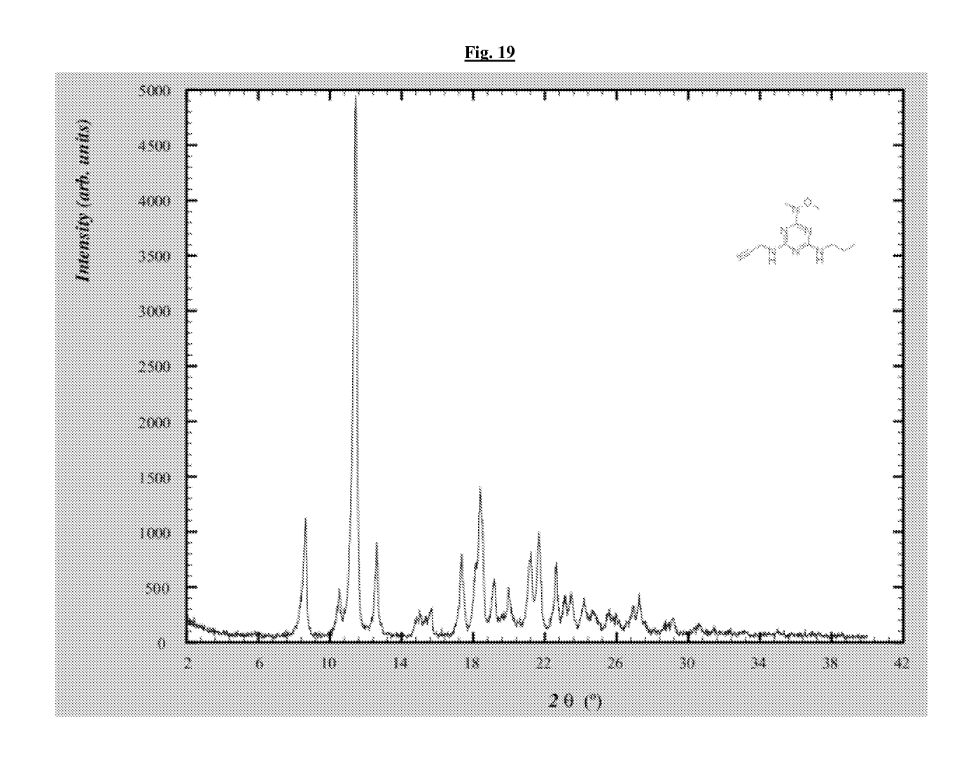

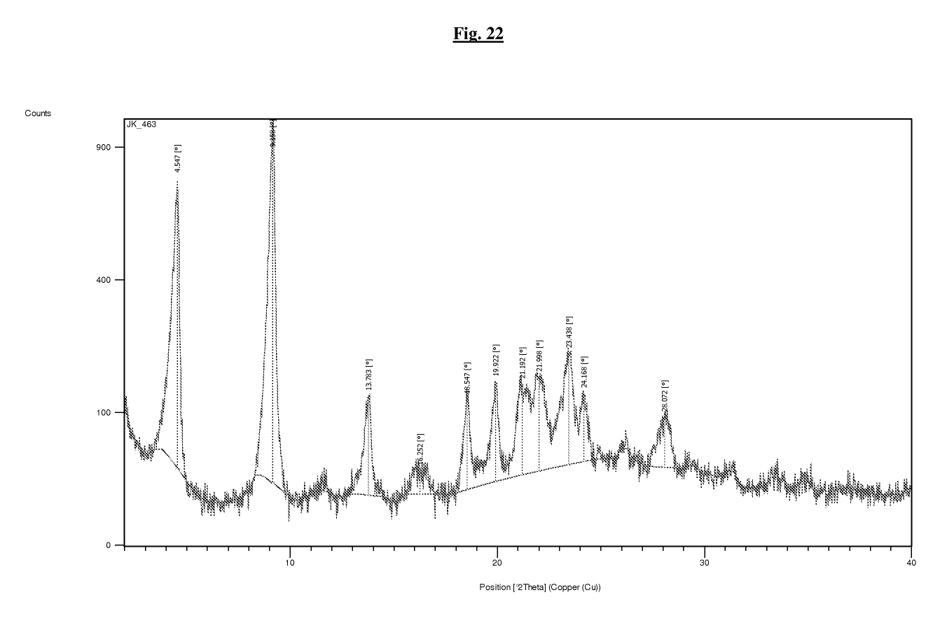

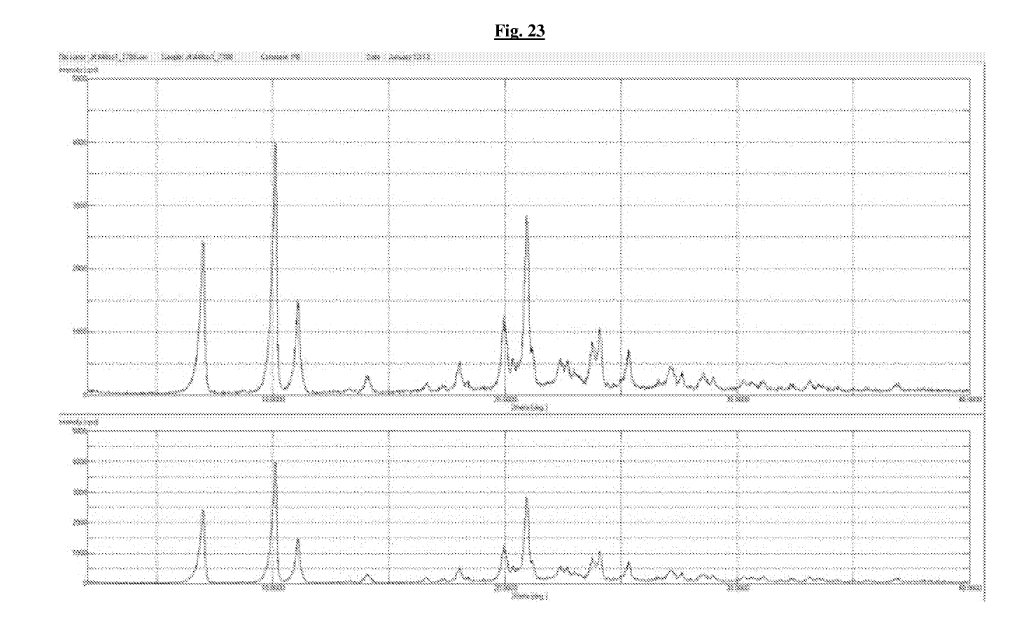

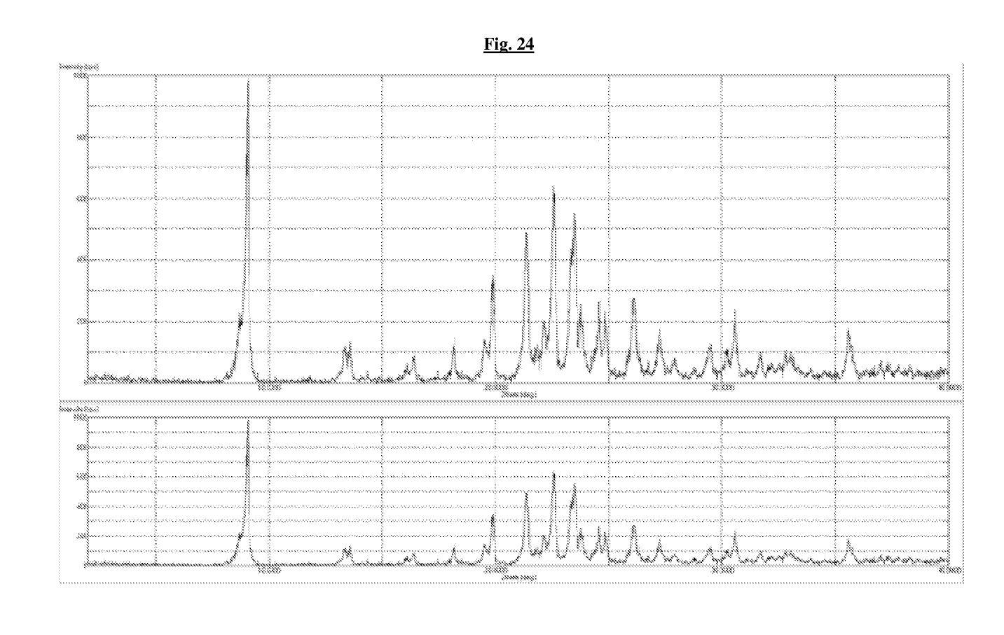

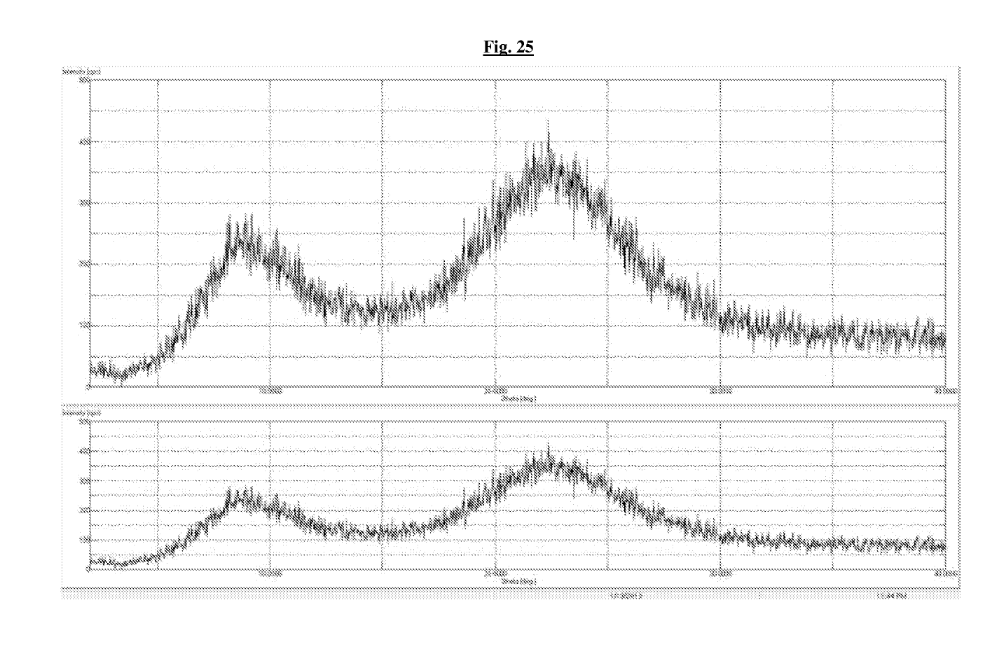

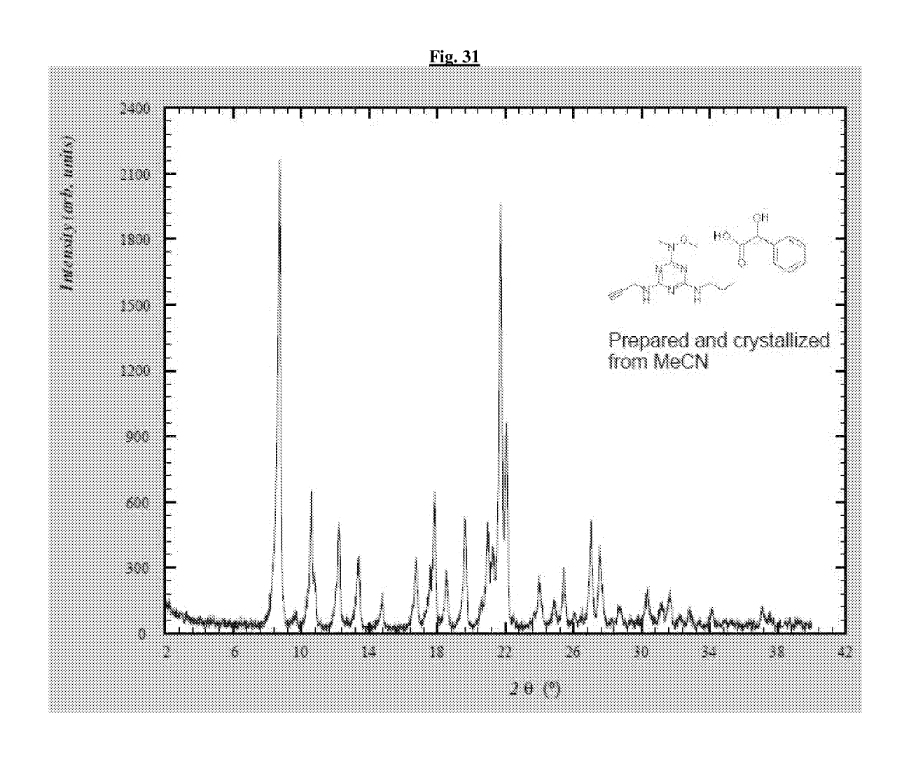

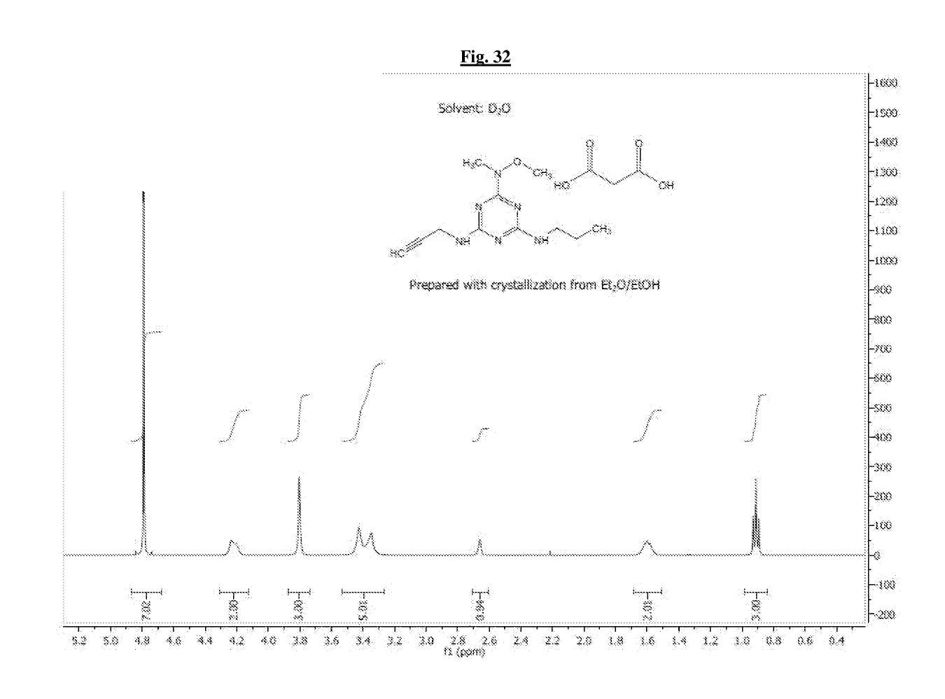

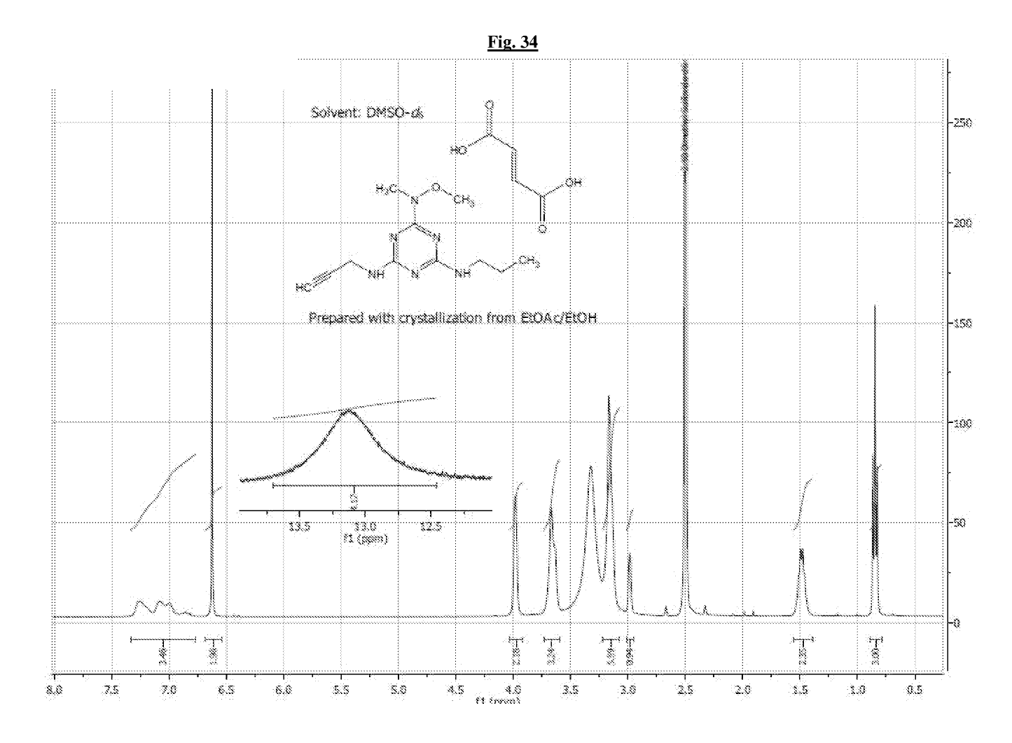

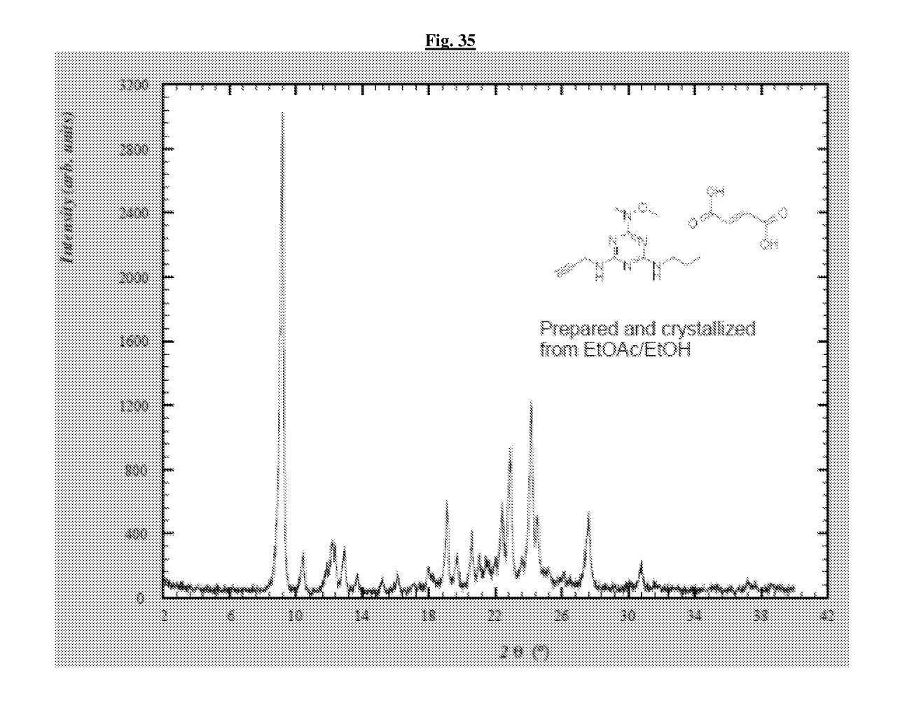

The invention also includes a composition comprising O,N-dimethyl-N-[4-(n-propylamino)-6-(prop-2-ynylamino)-[1,3,5]triazin-2-yl]-hydroxylamine or a salt thereof selected from the group consisting of:
(a) a crystalline form of O,N-dimethyl-N-(4-n-propylamino-6-prop-2-ynylamino-[1,3,5]triazin-2-yl)-hydroxylamine with an XRPD spectrum as illustrated in FIG. 18;
(b) a crystalline form of O,N-dimethyl-N-(4-n-propylamino-6-prop-2-ynylamino-[1,3,5]-hydroylamine with an XRPD spectrum as illustrated in FIG. 19;
(c) a crystalline form of O,N-dimethyl-N-(4-n-propylamino-6-prop-2-ynylamino-[1,3,5]triazin-2-yl)-hydroxylamine hydrogen sulfate with an XRPD spectrum as illustrated in FIG. 22;
(d) a crystalline form of O,N-dimethyl-N-(4-n-propylamino-6-prop-2-ynylamino-[1,3,5]triazin-2-yl)-hydroxylamine hemisulfate (2 moles Compound 4: 1 mole sulfuric acid) with an XRPD spectrum as illustrated in FIG. 23;
(e) a crystalline form of O,N-dimethyl-N-(4-n-propylamino-6-prop-2-ynylamino-[1,3,5]triazin-2-yl)-hydroxylamine dihydrogen sulfate (1 mole Compound 4: 2 moles sulfuric acid) with an XRPD spectrum as illustrated in FIG. 24;
(f) a crystalline form of O,N-dimethyl-N-(4-n-propylamino-6-prop-2-ynylamino-[1,3,5]triazin-2-yl)-hydroxylamine L(+) hydrogen tartrate with an XRPD spectrum as illustrated in FIG. 27;
(g) a crystalline form of O,N-dimethyl-N-(4-n-propylamino-6-prop-2-ynylamino-[1,3,5]triazin-2-yl)-hydroxylamine hydrogen maleinate with an XRPD spectrum as illustrated in FIG. 29;
(h) a crystalline form of O,N-dimethyl-N-(4-n-propylamino-6-prop-2-ynylamino-[1,3,5]triazin-2-yl)-hydroxylamine DL-mandelate with an XRPD spectrum as illustrated in FIG. 31;
(i) a crystalline form of O,N-dimethyl-N-(4-n-propylamino-6-prop-2-ynylamino-[1,3,5]triazin-2-yl)-hydroxylamine hydrogene malonate with an XRPD spectrum as illustrated in FIG. 33;
(j) a crystalline form of O,N-dimethyl-N-(4-n-propylamino-6-prop-2-ynylamino-[1,3,5]triazin-2-yl)-hydroxylamine hydrogen fumarate with an XRPD spectrum as illustrated in FIG. 35;
(k) a crystalline form of O,N-dimethyl-N-(4-n-propylamino-6-prop-2-ynylamino-[1,3,5]triazin-2-yl)-hydroxylamine saccharinate with an XRPD spectrum as illustrated in FIG. 37;
(l) an amorphous sulfuric acid addition salt of O,N-dimethyl-N-(4-n-propylamino-6-prop-2-ynylamino-[1,3,5]triazin-2-yl)-hydroxylamine, wherein the salt comprise a 4:3 molar ratio of Compound 4 to sulfuric acid and has an XRPD spectrum as illustrated in FIG. 25;
   and any combinations thereof.

The compounds of the invention may possess one or more stereocenters, and each stereocenter may exist independently in either the (R) or (S) configuration. In one embodiment, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention exist as tautomers. All tautomers are included within the scope of the compounds recited herein.

In one embodiment, compounds described herein are prepared as prodrugs. A “prodrug” is an agent converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

### Synthesis

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxy benzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

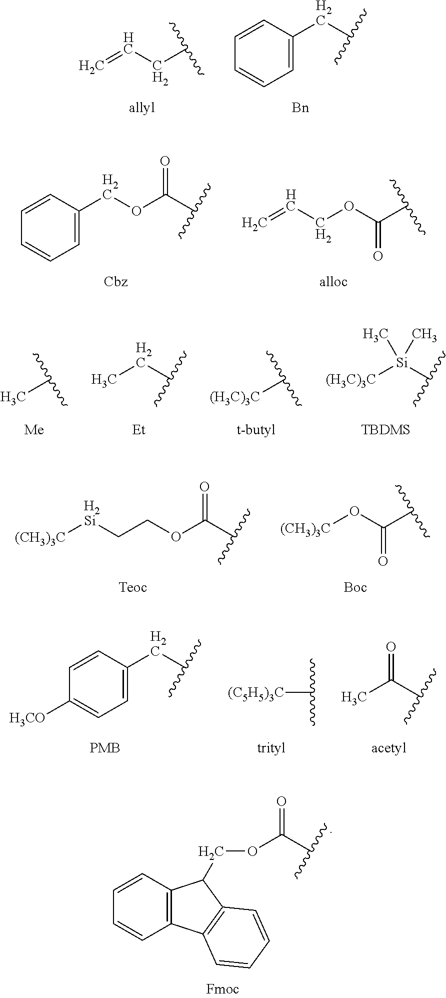

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

The compounds of the invention may be prepared according to the general methodology illustrated in the synthetic schemes described below. The reagents and conditions described herein may be modified to allow the preparation of the compounds of the invention, and such modifications are known to those skilled in the art. The scheme included herein are intended to illustrate but not limit the chemistry and methodologies that one skilled in the art may use to make compounds of the invention.

In one aspect, compounds of formula (I) may be prepared by the successive additions of (i) a primary, propargylic or homopropargylic amine, (ii) a N-alkoxy-N-alkylamine or (iii) an appropriately substituted hydrazine (H₂N—NHR² or R¹HN—NHR²) to suitably chlorinated intermediate (VI), as illustrated below in Scheme 1.

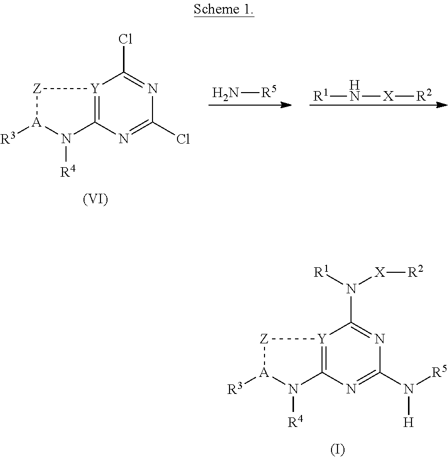

In another aspect, a compound of formula (IV) or (V) may be prepared by reductive alkylation of a suitably chlorinated amino-pyrrolidino-pyrimidine or amino-pyrrolo-pyrimidine, respectively (Scheme 2).

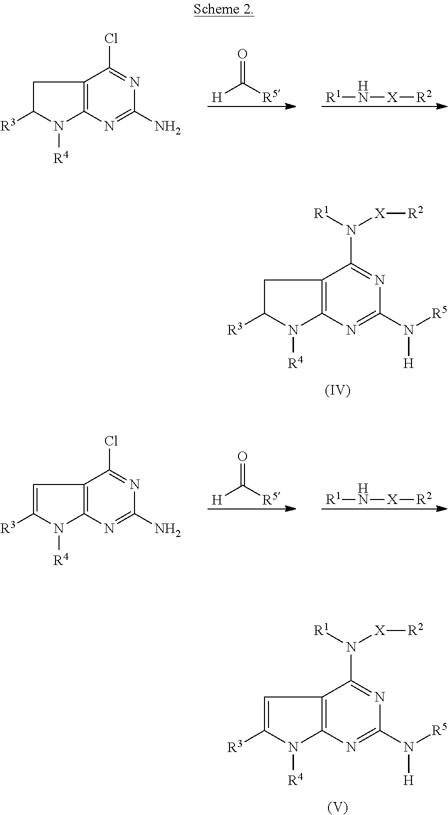

In yet another aspect, a triazine compound of formula (II) may be prepared by the successive additions of a primary, propargylic or homopropargylic amine, and (i) a N-alkoxy-N-alkylamine, (ii) a hydrazine H₂N—NHR², or (iii) a hydrazine R¹HN—NHR² to a suitably chlorinated triazine. Under appropriate conditions, the reaction may allow the addition of either one or two amine substituents to the triazine ring. Alternatively, first the N-alkoxy-N-alkylamine, the hydrazine H₂N—NHR², or the hydrazine R¹HN—NHR² may be added to the triazine, followed by the addition of the primary, propargylic or homopropargylic amine.

In a non-limiting example, to a solution of 2,4,6-trichlorotriazine in an appropriate aprotic or protic solvent containing an inorganic or organic base, is added a solution of a primary, propargylic or homopropargylic amine (VII) and the reaction is allowed to proceed at −20° C. to 10° C., ambient temperature, or heated, to isolate mono-amine adduct (VIII) or bis-amine adduct (IX).

In a subsequent reaction, mono-amine adduct (VIII) is reacted with another primary, secondary, propargylic or homopropargylic amine (X) to yield the unsymmetrical monochloro-bis-amino-triazine adduct (XI). In a subsequent reaction, monochloro-bis-amino-triazine adduct (XI) is reacted with (i) a N-alkoxy-N-alkylamine, (ii) a hydrazine H₂N—NHR² or (iii) a hydrazine R¹HN—NHR² in an appropriate aprotic or protic solvent containing an inorganic or organic base to produce desired compounds of formula (II) (Scheme 3).

Alternatively, in a subsequent reaction, bis-amine adduct (IX) is reacted with (i) a N-alkoxy-N-alkylamine, (ii) a hydrazine H₂N—NHR² or (iii) a hydrazine R¹HN—NHR² in an appropriate aprotic or protic solvent containing an inorganic or organic base to produce desired compounds of formula (II), wherein R³CH₂ is R⁵ (Scheme 4).

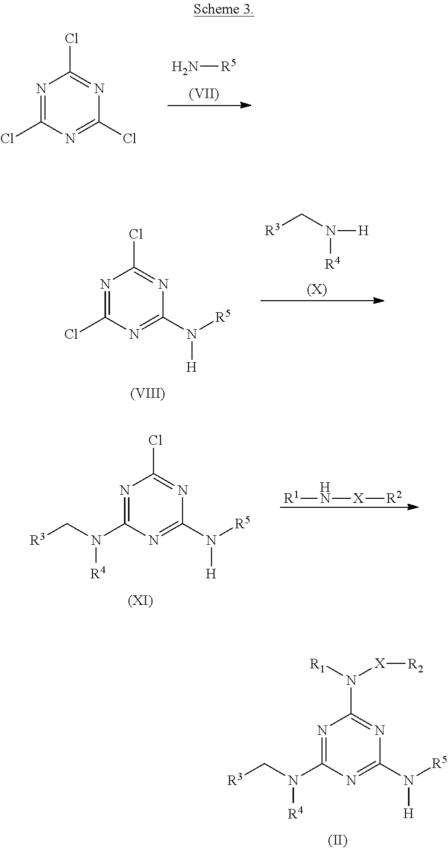

In yet another aspect, the pyrimidine compound of the formula (III) may be prepared by the successive additions of primary amines and (i) a N-alkoxy-N-alkylamine, (ii) a hydrazine H₂N—NHR² or (iii) a hydrazine R¹HN—NHR² to a suitably chlorinated pyrimidine.

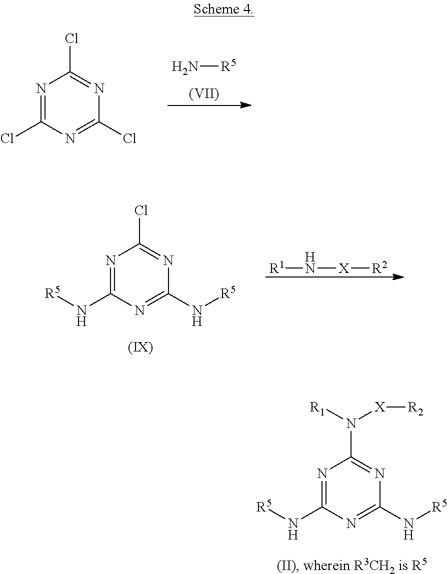

In a non-limiting example, to a solution of 2,4,6-trichloropyrimidine (XII) in an appropriate aprotic or protic solvent containing an inorganic or organic base is added a solution of a primary propargylic or homopropargylic amine (VII) and the reaction is allowed to proceed at ambient temperature or heated, yielding bis-amine adduct (XIII). In a subsequent reaction, bis-amine adduct (XIII) is reacted with (i) a N-alkoxy-N-alkylamine, (ii) a hydrazine H₂N—NHR², or (iii) a hydrazine R¹HN—NHR² in an appropriate aprotic or protic solvent containing an inorganic or organic base to produce desired compounds of formula (III) (Scheme 5).

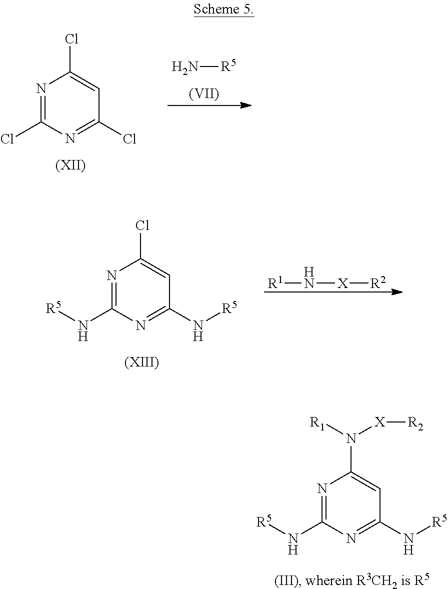

In yet another aspect, a pyrrolidino-pyrimidine of formula (IV) or a pyrrolo-pyrimidine compounds of formula (V) may be prepared from an appropriately chlorinated aminopyrrolidinopyrimidine or aminopyrrolopyrimidine intermediate, respectively.

In a non-limiting example, 2-chloroacetaldehyde may be added to a solution of 2,6-diamino-4-hydroxy-1,3-pyrimidine (XIV) in a polar protic solvent, at ambient temperature or under heating, to yield cyclized adduct (XV). Subsequent treatment with a chlorinating agent, such as, but not limited to, phosphorous oxychloride produces the chloro intermediate (XVI). Intermediate (XVI) may be submitted to reductive alkylation with an aldehyde in the presence of a reducing agent, such as a borohydride (in a non-limiting example, cyanoborohydride) in a protic solvent, at ambient temperature or elevated temperature, to produce the amino substituted adduct (XVII). In a subsequent reaction, amino substituted adduct (XVII) is reacted with (i) a N-alkoxy-N-alkylamine, (ii) a hydrazine H₂N—NHR², or (iii) a hydrazine R¹HN—NHR² in an appropriate aprotic or protic solvent containing an inorganic or organic base to produce desired compounds of formula (V), wherein R³ and R⁴ are H (Scheme 6).

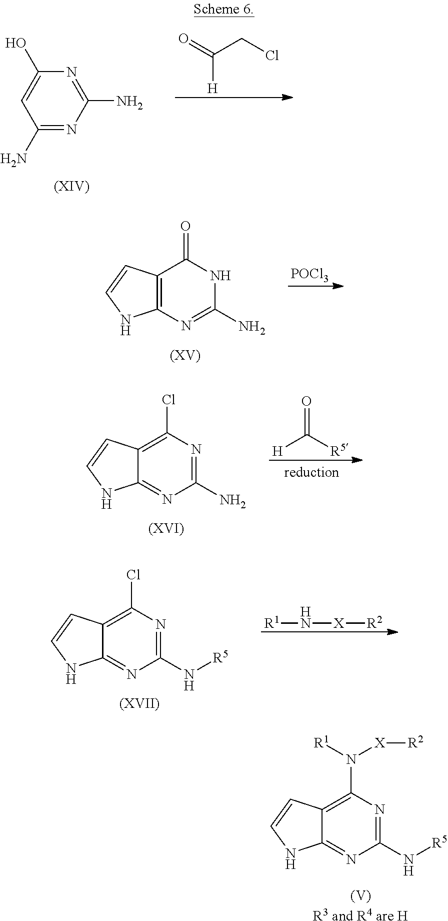

In a non-limiting example, a pyrrolidinopyrimidine compound of the formula (IV) may be prepared from the corresponding pyrrolopyrimidine analog via reduction (Scheme 7).

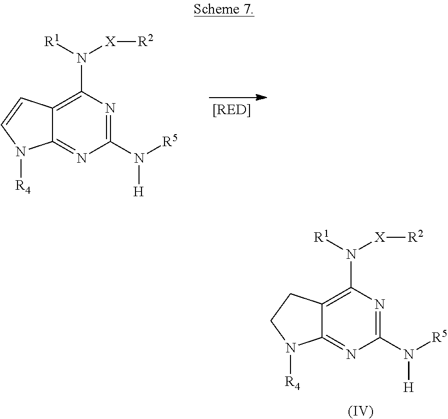

The invention includes a method of preparing O,N-dimethyl-N-[4-(n-propylamino)-6-(prop-2-ynylamino)-[1,3,5]triazin-2-yl]-hydroxylamine or a salt thereof. The method comprises the steps of: (a) contacting cyanuric chloride with n-propyl amine in a solvent in the presence of a base; (b) adding propargyl amine and a base to the mixture of step (a) and heating the resulting mixture; (c) isolating from the mixture of step (b) solid 6-chloro-N-n-propyl-N′-prop-2-ynyl-[1,3,5]triazine-2,4-diamine; (d) contacting the product of step (c) with O,N-dimethylhydroxylamine, or a salt thereof, with a suitable amount of a base in a solvent at a given temperature; and (e) isolating from the mixture of step (d) solid O,N-dimethyl-N-[4-(n-propylamino)-6-(prop-2-ynylamino)-[1,3,5]triazin-2-yl]-hydroxylamine.

In one embodiment, the method further comprises: (f) contacting the product of step (e) with sulfuric acid, as to form a hydrogen sulfate salt of O,N-dimethyl-N-[4-(n-propylamino)-6-(prop-2-ynylamino)-[1,3,5]triazin-2-yl]-hydroxylamine. In another embodiment, the hydrogen sulfate salt formed in step (f) is isolated as a solid and has the XRPD spectrum illustrated in FIG. 22.

In one embodiment, the method further comprises: (f) contacting the product of step (e) with L(+)-tartaric acid in a solvent, as to form a L(+) hydrogen tartrate salt of O,N-dimethyl-N-[4-(n-propylamino)-6-(prop-2-ynylamino)-[1,3,5]triazin-2-yl]-hydroxylamine. In another embodiment, the L(+) hydrogen tartrate salt formed in step (f) is isolated as a solid and has the XRPD spectrum in FIG. 27.

In one embodiment, the method further comprises: (f) contacting the product of step (e) with maleic acid in a solvent, as to form a hydrogen maleinate salt of O,N-dimethyl-N-[4-(n-propylamino)-6-(prop-2-ynylamino)-[1,3,5]-triazin-2-yl]-hydroxylamine. In another embodiment, the hydrogen maleinate salt formed in step (f) is isolated as a solid and has the XRPD spectrum in FIG. 29.

In one embodiment, the method further comprises: (f) contacting the product of step (e) with DL-mandelic acid in a solvent, as to form a DL-mandelate salt of O,N-dimethyl-N-[4-(n-propylamino)-6-(prop-2-ynylamino)-[1,3,5]-triazin-2-yl]-hydroxylamine. In another embodiment, the DL-mandelate salt formed in step (f) is isolated as a solid and has the XRPD spectrum in FIG. 31.

In one embodiment, the method further comprises: (f) contacting the product of step (e) with malonic acid in a solvent, as to form a hydrogen malonate salt of O,N-dimethyl-N-[4-(n-propylamino)-6-(prop-2-ynylamino)-[1,3,5]-triazin-2-yl]-hydroxylamine. In another embodiment, the hydrogen malonate salt formed in step (f) is isolated as a solid and has the XRPD spectrum in FIG. 33.

In one embodiment, the method further comprises: (f) contacting the product of step (e) with fumaric acid in a solvent, as to form a hydrogen fumarate salt of O,N-dimethyl-N-[4-(n-propylamino)-6-(prop-2-ynylamino)-[1,3,5]-triazin-2-yl]-hydroxylamine. In another embodiment, the hydrogen fumarate salt formed in step (f) is isolated as a solid and has the XRPD spectrum in FIG. 35.

In one embodiment, the method further comprises: (f) contacting the product of step (e) with saccharin in a solvent, as to form a saccharinate salt of O,N-dimethyl-N-[4-(n-propylamino)-6-(prop-2-ynylamino)-[1,3,5]-triazin-2-yl]-hydroxylamine. In another embodiment, the saccharinate salt formed in step (f) is isolated as a solid and has the XRPD spectrum in FIG. 37.

In one embodiment, the solvent in step (a) comprises isopropanol. In another embodiment, the base in step (a) comprises diisopropylethylamine, in an amount that is one molar equivalent relative to the cyanuric chloride. In yet another embodiment, formation of 6-chloro-N,N′-propyl-[1,3,5]triazine-2,4-diamine in step (a) is minimized by using a molar deficit of n-propyl amine to cyanuric chloride and conducting the reaction at a reduced temperature. In yet another embodiment, a 5-20% molar deficit of n-propyl amine relative to cyanuric chloride is used in step (a). In yet another embodiment, 0.95 molar equivalents of n-propylamine relative to cyanuric chloride is used in step (a). In yet another embodiment, 0.9 molar equivalents of n-propylamine relative to cyanuric chloride is used in step (a). In yet another embodiment, in step (a) the mixture of cyanuric chloride and solvent is cooled to −20° to 10° C., and a mixture of n-propyl amine and base are added over a 2-6 hour period while maintaining the batch at about 0° C. In yet another embodiment, the reaction is run at −2° C. to 0° C. In yet another embodiment, the product of step (a) is not isolated.

In one embodiment, step (b) further comprises contacting the mixture with one additional molar equivalent of base relative to the cyanuric chloride at room temperature for about 1 hour, whereby unreacted cyanuric chloride is consumed by reaction with the solvent.

In one embodiment, the isolated compound in step (c) contains less than 0.5% 6-chloro-N,N′-propyl-[1,3,5]triazine-2,4-diamine.

In one embodiment, in step (b) at least two molar equivalents of N,N-diisopropylethylamine are added to the mixture of step (a) and propargyl amine as a sulfate salt (two moles of propargyl amine per mole of sulfuric acid) is used in place of propargyl amine free base.

In one embodiment, the solvent in step (d) comprises dimethyl acetamide. In another embodiment, in step (d) a salt of O,N-methylhydroxylamine, and sufficient base to generate free O,N-methylhydroxylamine in solution, are used. In yet another embodiment, O,N-dimethyhydroxylamine free base is used. In yet another embodiment, the reaction of step (d) is run at 60-80° C.

In one embodiment, step (e) comprises the steps of: cooling the mixture of step (d) below 60° C.; diluting the resulting mixture with 2 volumes of water with vigorous stirring over about 2-3 h; seeding the resulting system with a crystal of O,N-dimethyl-N-[4-(n-propylamino)-6-(prop-2-ynylamino)-[1,3,5]triazin-2-yl]-hydroxylamine and stirring the resulting system for 10-20 h, whereby crystallization of the product takes place.

In one embodiment, the reaction mixture generated in step (d) is diluted with water, and product is extracted with toluene. In another embodiment, the toluene extract is washed with water to remove dimethylacetamide, and water content of the toluene extract is minimized by azeotropic distillation. In yet another embodiment, heptane is added to the mixture, and the crystalline product is collected by filtration.

In one embodiment, in step (e), before solid O,N-dimethyl-N-[4-(n-propylamino)-6-(prop-2-ynylamino)-[1,3,5]triazin-2-yl]-hydroxylamine is contacted with an acid to form a salt, a solution of the compound in methyl ethyl ketone is filtered at 50° C. to remove 6-hydroxy-N-propyl-N′-prop-2-ynyl-[1,3,5]triazine-2,4-diamine byproduct.

In one embodiment, step (f) comprises treating solid O,N-dimethyl-N-[4-(n-propylamino)-6-(prop-2-ynylamino)-[1,3,5]triazin-2-yl]-hydroxylamine in a solvent with about 1 molar equivalent of at least one selected from the group consisting of concentrated sulfuric acid, L(+)-tartaric acid, maleic acid, DL-mandelic acid, malonic acid, fumaric acid and saccharin, at either ambient temperature or with heating, followed by cooling and stirring at room temperature, thereby providing O,N-dimethyl-N-(4-n-propylamino-6-prop-2-ynylamino-[1,3,5]triazin-2-yl)-hydroxylamine as one of several solid salts with XRPD spectra as noted elsewhere herein.

Without wishing to be limited by any theory, the propargylamine reagent used within the methods of the invention may contain 2-chloroallylamine as an impurity, and this impurity may react similarly to propargyl amine with chlorinated aryl triazines and other suitably substituted aromatic heterocycles (Scheme 8), yielding a 2-chloroallylamine impurity. In Scheme 8, R¹, R², R³, R⁴, R⁵, A, X, Y, Z, b¹ and b² are defined as described above for Compound I.

Those skilled in the art recognize that the competing reaction with 2-chloroallylamine may occur at any point at which propargyl amine is reacted with an intermediate during the synthesis of a compound of the invention. In one embodiment, a 2-chloroallyl containing impurity is formed during synthesis of a compound of the invention during a reaction that does not involve 2-chloroallylamine.

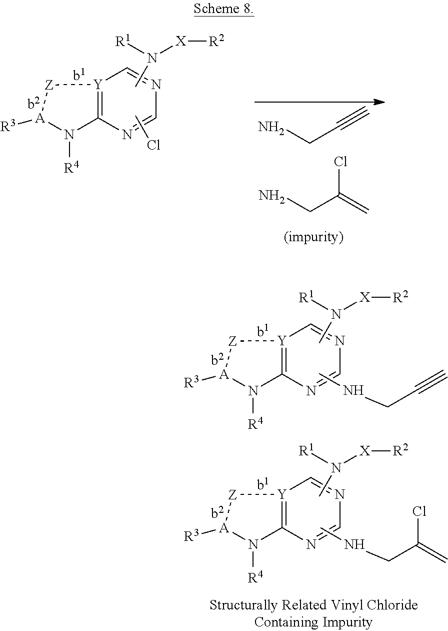

In one aspect of the invention, the compound of the invention is substantially free of a 2-chloroallylamine impurity.

In one embodiment, the propargyl amine is purified, whereby 2-chloroallylamine is removed before the propargyl amine is used within the synthetic methods of the invention. In a non-limiting example, propargylamine with greater than about 0.01 weight % to about 1 weight % of 2-chloroallylamine may be converted to its hemisulfate salt (2:1 propargyl amine-sulfuric acid), which upon isolation contains less than 0.01 weight and preferably less than 0.003 weight % of 2-chloroallylamine. Salt formation may comprise contacting propagylamine with one half of a molar equivalent of sulfuric acid in a solvent, after which point the solid propargylamine sulfate precipitates from the mixture. Suitable solvents include but are not limited to methanol and ethanol. In one embodiment, the reaction is run in ethanol. Suitable temperatures for the formation and aging of the salt range from 0° C. to the boiling point of the solvent used. Preferentially, the salt is formed and aged at a temperature ranging from 10° C. and 70° C. More preferentially, the salt is formed and aged at a temperature ranging about 20° C. to about 65° C. and isolated at room temperature. In one embodiment, propargylamine purified through sulfate salt formation affords a compound of the invention as a free base with about 0.015 weight % of the structurally related vinyl chloride impurity.

In one embodiment, the 2-chloroallylamine impurity is removed by forming and isolating a solid salt of a compound of the invention or an intermediate thereof. In a non-limiting example, a compound of the invention, or an intermediate thereof, as its free base may be purified of the structurally related 2-chloroallylamine impurity by the preparation of a salt. Preferred salts include the L(+)-hydrogen tartrate and hydrogen maleinate salts. Suitable solvents for salt formation include, but are not limited to, methyl ethyl ketone, methyl isobutyl ketone, acetone, isopropyl acetate, ethyl acetate, methyl-tert-butyl ether, isopropanol, n-propanol, isoamyl alcohol, 2-butanol, n-butanol or acetonitrile. Preferred solvents include isopropanol and methyl ethyl ketone. Methods for preparing a salt for the purpose of removing the 2-chloroallylamine impurity include contacting a compound of the invention as its free base with about one molar equivalent of a suitable acid in a suitable solvent at temperatures ranging from about 0° C. to the boiling point of the suitable solvent, and allowing the resulting mixture to age, with or without cooling, to yield the salt as a solid. Optionally, seed crystals may be added to the mixture to promote solid formation, wherein the formation of a specific polymorphic crystalline form may occur. The 2-chloroallylamine impurity impurity may be removed in the mother liquors of the salt formation and any solvent rinses of the isolated solid product. In one embodiment, the salt preparation purification method provides compounds of the invention with less than 0.03 weight %, less that 0.012 weight %, less than 0.01 weight %, less than 0.005 weight %, less than 0.004 weight %, less than 0.003 weight %, less than 0.0003 weight %, and substantially free of the contaminating 2-chloroallylamine impurity.

Without wishing to be limited by theory, a vinyl chloride impurity may be formed when a propargylamine group is attached to a chloroaromatic heterocycle. For example, when propargyl amine is attached to a chloroaromatic heterocycle through chloride displacement in the presence of a base, hydrochlorination of the propargyl triple bond may take place. Further, if the propargylamine is already attached to a chloroaromatic heterocycle, the introduction of another nucleophilc appendage by displacement of a chloro moiety in the presence of a base may result in hydrochlorination of the propargyl amine triple bond. In a non-limiting manner, such hydrochlorination may be prevented by avoiding the use of chloro as the leaving group during nucleophilic substitution of the aromatic heterocycle, and by minimizing the presence of chloride ion in any reaction mixture while or after propargyl amine is being or has been attached to the compound. In one embodiment, a purified propargyl amine containing 0.01 weight % or less of 2-chloroallyl amine may be used for a final substitution upon the aromatic heterocycle without using chloro as the leaving group.

In one embodiment, the chloro group is first displaced by a tertiary amine, forming a quaternary amino substituent with chloride as the counterion. In another embodiment, the quaternary amine heteroaryl chloride salt precipitates from solution, allowing for removal of impurities carried forward from earlier stages in the overall synthesis of a compound of the invention. Suitable tertiary amines include trimethyl amine, quinuclidine, N-methyl pyrrolidine, and 1,4-diazabicyclo[2.2.2]octane (DABCO). A preferred tertiary amine is trimethylamine. The quaternary amine chloride salt substituent may itself serve as a leaving group for a displacement reaction with propargylamine. In one embodiment, the chloride counterion is exchanged for tetrafluoroborate by contacting the quaternary amine heteroaryl chloride salt in water with an alkaline earth metal salt of tetrafluoroboric acid. A preferred alkaline earth metal salt is sodium tetrafluoroborate. In one embodiment, the quaternary amine heteroaryl tetrafluoroborate salt precipitates as a solid from water in high chemical purity. The isolated solid tetrafluoroborate salt may contain <1 ppm chlorine ion. This process may allow for minimizing or substantially eliminating the chloride group from the reaction mixture. The quaternary amine tetrafluoroborate salt of the aromatic heterocycle may then be contacted with propargylamine in a suitable solvent to displace the quaternary amine salt and attach propargyl amine on the aromatic heterocycle. For purposes of this transformation, the purified propargylamine may be used as a neat liquid, or as the sulfate salt (2:1 propargyl amine-H₂SO₄), in the presence of a base. Suitable solvents include, but are not limited to, polar solvents such as N-methyl pyrrolidinone, dimethyformamide, dimethyl acetamide, isopropanol, n-propanol, tetrahydrofuran, and dimethyl sulfoxide. In one embodiment, the solvent comprises dimethyl sulfoxide. In another embodiment, the solvent comprises neat purified propargyl amine. Both organic and inorganic bases may be used. In one embodiment, the base comprises potassium dihydrogen phosphate. In one embodiment, the organic base comprises N,N-diisopropylethyl amine. The reaction may be performed at a temperature ranging from about 20° C. to about 80° C. A temperature of about 45° C. is preferred. The propargyl amine-substituted heteroaryl product may be formed with about 10% of a dialkylamino impurity derived by mono dealkylation of the quaternary amine by propargyl amine. In one embodiment, the use of neat propargylamine as solvent unexpectedly yields a compound as a crude free base with as low as 3% of the dialkyl amino impurity relative to the desired product. Crystallization of the crude product yields the desired compound as its free base with less than or equal to 0.3% of the dimethyl amine impurity and less than or equal to 0.0003 weight % of their structurally-related vinyl chloride impurities. Suitable solvents for use in recrystallizing the compound include, but are not limited to toluene, light petroleum ether, heptane and admixtures thereof.

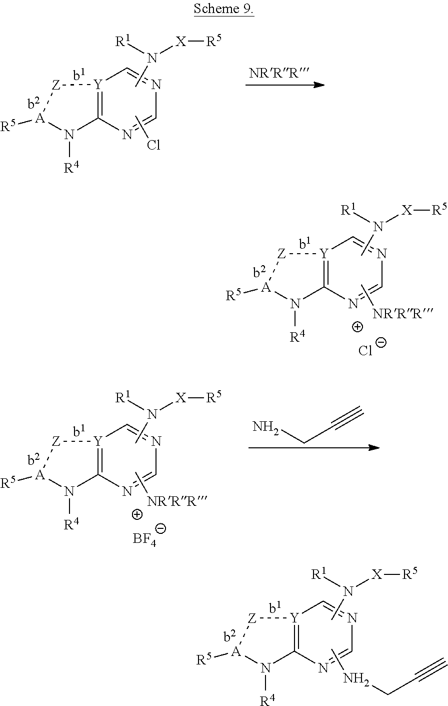

### Salts

The compounds described herein may form salts with acids, and such salts are included in the present invention. In one embodiment, the salts are pharmaceutically acceptable salts. The term “salts” embraces addition salts of free acids that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

### Combination and Concurrent Therapies

In one embodiment, the compounds of the invention are useful in the methods of present invention when used concurrently with at least one additional compound useful for preventing and/or treating breathing control disorders.

In one embodiment, the compounds of the invention are useful in the methods of present invention in combination with at least one additional compound useful for preventing and/or treating breathing control disorders.

These additional compounds may comprise compounds of the present invention or other compounds, such as commercially available compounds, known to treat, prevent, or reduce the symptoms of breathing disorders. In one embodiment, the combination of at least one compound of the invention or a salt thereof and at least one additional compound useful for preventing and/or treating breathing disorders has additive, complementary or synergistic effects in the prevention and/or treatment of disordered breathing, and in the prevention and/or treatment of sleep-related breathing disorders.

In a non-limiting example, the compounds of the invention or a salt thereof may be used concurrently or in combination with one or more of the following drugs: doxapram, enantiomers of doxapram, acetazolamide, almitrine, theophylline, caffeine, methylprogesterone and related compounds, sedatives that decrease arousal threshold in sleep disordered breathing patients (such as eszopiclone and zolpidem), sodium oxybate, benzodiazepine receptor agonists (e.g., zolpidem, zaleplon, eszopiclone, estazolam, flurazepam, quazepam, temazepam, triazolam), orexin antagonists (e.g., suvorexant), tricyclic antidepressants (e.g., doxepin), serotonergic modulators, adenosine and adenosine receptor and nucleoside transporter modulators, cannabinoids (such as, but not limited to, dronabinol), orexins, melatonin agonists (such as ramelteon) and compounds known as ampakines.

Non-limiting examples of ampakines are the pyrrolidine derivative racetam drugs such as piracetam and aniracetam; the “CX-” series of drugs which encompass a range of benzoylpiperidine and benzoylpyrrolidine structures, such as CX-516 (6-(piperidin-1-yl-carbonyl)quinoxaline), CX-546 (2,3-dihydro-1,4-benzodioxin-7-yl-(1-piperidyl)-methanone), CX-614 (2H,3H,6aH-pyrrolidino(2,1-3′,2′)-1,3-oxazino-(6′,5′-5,4)benzo(e)1,4-dioxan-10-one), CX-691 (2,1,3-benzoxadiazol-6-yl-piperidin-1-yl-methanone), CX-717, CX-701, CX-1739, CX-1763, and CX-1837; benzothiazide derivatives such as cyclothiazide and IDRA-21 (7-chloro-3-methyl-3,4-dihydro-2H-1,2,4-benzothiadiazine 1,1-dioxide); biarylpropylsulfonamides such as LY-392,098, LY-404,187 (N-[2-(4′-cyanobiphenyl-4-yl)propyl]propane-2-sulfonamide), LY-451,646 and LY-503,430 (4′-{(1S)-1-fluoro-2-[(isopropylsulfonyl)amino]-1-methylethyl}-N-methylbiphenyl-4-carboxamide).

In one embodiment, the invention includes a composition comprising a compound of formula (I) and at least one agent selected from the group consisting of doxapram, enantiomers of doxapram, enantiomers of doxapram, acetazolamide, almitrine, theophylline, caffeine, methylprogesterone and related compounds, sedatives that decrease arousal threshold in sleep disordered breathing patients (such as eszopiclone or zolpidem), sodium oxybate, benzodiazepine receptor agonists (such as zolpidem, zaleplon, eszopiclone, estazolam, flurazepam, quazepam, temazepam, or triazolam), orexin antagonists (e.g. suvorexant), tricyclic antidepressants (such as doxepin), serotonergic modulators, adenosine and adenosine receptor and nucleoside transporter modulators, cannabinoids (such as but not limited to dronabinol), orexins, melatonin agonists (such as ramelteon) and compounds known as ampakines.

In another non-limiting example, the compounds of the invention or a salt thereof may be used concurrently or in combination with one or more of the following drugs and drug classes known to cause changes in breathing control: opioid narcotics (such as morphine, fentanyl, codeine, hydromorphone, hydrocodone, oxymorphone, oxycodone, meperidine, butorphanol, carfentanil, buprenorphine, methadone, nalbuphine, propoxyphene, pentazocine, remifentanil, alfentanil, sufentanil and tapentadol); benzodiazepines (such as midazolam); and sedatives (such as zolipidem and eszopiclone); sodium oxybate and propofol. In one embodiment, the invention includes a composition comprising a compound of formula (I) and at least one agent known to cause changes in breathing control. In one embodiment, the at least one agent is selected from the group consisting of opioid narcotics, benzodiazepines, sedatives, sleeping aids and propofol.

In another non-limiting example, the compounds of the invention or a salt thereof may be used concurrently or in combination with one or more of the following drugs and drug classes known to either aid the onset of sleep, maintain sleep and/or alter arousal threshold: zolipidem, zaleplon, eszopiclone, ramelteon, estazolam, temazepam, doxepin, sodium oxybate, phenobarbital and other barbiturates, diphenhydramine, doxylamine and related compounds, for example. The combination of a sleep promoting/stabilizing drug and the compounds of the invention may act additively or synergistically to improve indices of sleep disordered breathing. In one embodiment, the compounds of the invention stabilize respiratory pattern (i.e., decrease variation in respiratory rate and tidal volume on a breath-by-breath basis) and respiratory drive (i.e., decrease fluctuations in the neural control of the respiratory muscles), thereby decreasing the incidence of central and obstructive apneas whilst the sleep promoting/stabilizing drug prevents patient arousal from sleep if residual apneas persist. Blood gas derangements associated with a residual apnea may elicit chemoreceptor stimulation, which in turn elicits generalized central nervous system arousal. Patients with a low arousal threshold from sleep wake early and often (i.e., experience sleep fragmentation) and these patients experience a ventilatory overshoot due to the sudden awakening in excess of the level of chemoreceptor stimulation. Sleep promoting/stabilizing drugs delay cortical arousal and permit a more appropriate ventilatory response to apnea-induced chemoreceptor stimulation. The patient benefits from delayed arousal from sleep because sleep fragmentation decreases and hyperventilation-driven central apneas decrease.

As used herein, combination of two or more compounds may refer to a composition wherein the individual compounds are physically mixed or wherein the individual compounds are physically separated. A combination therapy encompasses administering the components separately to produce the desired additive, complementary or synergistic effects.

In one embodiment, the compound and the agent are physically mixed in the composition. In another embodiment, the compound and the agent are physically separated in the composition.

In one embodiment, the compound of the invention is co-administered with a compound that is used to treat another disorders but causes loss of breathing control. In this aspect, the compound of the invention blocks or otherwise reduces depressive effects on normal breathing control caused by the compound with which they are co-administered. Such compound that treats another disorder but depresses breathing control includes but is not limited to anesthetics, sedatives, sleeping aids, anxiolytics, hypnotics, alcohol, and narcotic analgesics. The co-administered compound may be administered individually, or a combined composition as a mixture of solids and/or liquids in a solid, gel or liquid formulation or as a solution, according to methods known to those familiar with the art.

In one embodiment, a compound of the present invention is co-administered with at least one additional compound useful for treating breathing control disorders and with at least one compound that is used to treat other disorder but causes a loss of breathing control. In this aspect, the compound of the invention works in an additive, complementary or synergistic manner with the co-administered breathing control agent to block or otherwise reduce depressive effects on normal breathing control caused by other compounds with which they are combined. A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In one embodiment, a compound of the present invention may be packaged with at least one additional compound useful for treating breathing control disorders. In another embodiment, a compound of the present invention may be packaged with a therapeutic agent known to cause changes in breathing control, such as, but not limited to, anesthetics, sedatives, anxiolytics, hypnotics, alcohol, and narcotic analgesics. A co-package may be based upon, but not limited to, dosage units.

### Methods

In one aspect, the present invention includes a method of preventing or treating a breathing control disorder or disease in a subject in need thereof. The method includes administering to the subject an effective amount of a pharmaceutical formulation comprising at least a pharmaceutically acceptable carrier and at least one compound of formula (I) or a salt thereof:

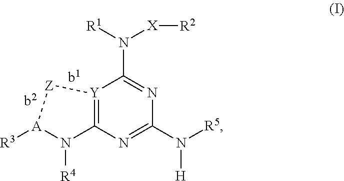

wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl; R³ is H, alkyl, substituted alkyl, alkynyl or substituted alkynyl; R⁴ is H, alkyl, or substituted alkyl; R⁵ is alkyl, propargylic, substituted propargylic, homopropargylic, or substituted homopropargylic, further wherein at least one substituent selected from the group consisting of R¹, R², R³ and R⁵ is alkynyl or substituted alkynyl; R⁶ is H, alkyl, substituted alkyl or alkenyl; X is a bond, O or NR⁴; and, Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C.

In another aspect, the present invention includes a method of preventing destabilization of or stabilizing breathing rhythm in a subject in need thereof. The method includes administering to the subject an effective amount of a pharmaceutical formulation comprising at least a pharmaceutically acceptable carrier and at least one compound of formula (I) or a salt thereof.

In one embodiment, administering the formulation of the invention stabilizes the breathing rhythm of the subject. In another embodiment, administering the formulation of the invention increases minute ventilation in the subject.

In one embodiment, the destabilization is associated with a breathing control disorder or disease.

In one embodiment, the breathing disorder or disease is selected from the group consisting of narcotic-induced respiratory depression, anesthetic-induced respiratory depression, sedative-induced respiratory depression, sleeping aid-induced respiratory depression, anxiolytic-induced respiratory depression, hypnotic-induced respiratory depression, alcohol-induced respiratory depression, analgesic-induced respiratory depression, sleep apnea (includes but not limited to mixed central, obstructive, anatomical), apnea of prematurity, obesity-hypoventilation syndrome, primary alveolar hypoventilation syndrome, dyspnea, altitude sickness, hypoxia, hypercapnia, chronic obstructive pulmonary disease (COPD), sudden infant death syndrome (SIDS), Alzheimer's disease, Parkinson's disease, stroke, Duchenne muscular dystrophy, and brain and spinal cord traumatic injury. In another embodiment, the respiratory depression is caused by an anesthetic, a sedative, an anxiolytic agent, a hypnotic agent, alcohol or a narcotic. In yet another embodiment, the compounds of the invention or a salt thereof may be used concurrently or in combination with one or more of the following drugs and drug classes known to either aid the onset of sleep, maintain sleep and/or alter arousal threshold: zolipidem, zaleplon, eszopiclone, ramelteon, estazolam, temazepam, sodium oxybate, doxepin, phenobarbital and other barbiturates, diphenhydramine, doxylamine and related compounds for example.

In one embodiment, the subject is further administered at least one additional compound useful for preventing or treating the breathing disorder or disease. In another embodiment, the at least one additional compound is selected from the group consisting of doxapram, enantiomers of doxapram, acetazolamide, almitrine, theophylline, caffeine, methylprogesterone and related compounds, sedatives such as eszopiclone and zolpidem, sodium oxybate, benzodiazepine receptor agonists (e.g. zolpidem, zaleplon, eszopiclone, estazolam, flurazepam, quazepam, temazepam, triazolam), orexin antagonists (e.g. suvorexant), tricyclic antidepressants (e.f. doxepin), serotonergic modulators, adenosine and adenosine receptor and nucleoside transporter modulators, cannabinoids (such as but not limited to dronabinol), orexins, melatonin agonists (such as ramelteon) and compounds known as ampakines.

In yet another embodiment, the formulation is administered to the subject in conjunction with the use of a mechanical ventilation device or positive airway pressure device. In one embodiment, the formulation is administered to the subject by an inhalational, topical, oral, nasal, buccal, rectal, pleural, peritoneal, vaginal, intramuscular, subcutaneous, transdermal, epidural, intrathecal or intravenous route. In another embodiment, the subject is a bird or a mammal including but not limited to mouse, rat, ferret, guinea pig, non-human primate (such as monkey), dog, cat, horse, cow, pig and other farm animals. In one embodiment, the subject is a human.

In one embodiment, the compound of formula (I) is selected from the group consisting of: O,N-Dimethyl-N-[4(-n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; N-(4-Fluorobenzyl)-O-methyl-N-[4-(n-propylamino)-6-(prop-2-ynylamino)-[1,3,5]triazin-2-yl]-hydroxylamine; N-(4-Fluorobenzyl)-N′-n-propyl-N″-prop-2-ynyl-[1,3,5]triazine-2,4,6-triamine; N-[4-(4-Fluorobenzylamino)-6-(prop-2-ynylamino)-[1,3,5]triazin-2-yl]-O,N-dimethyl-hydroxylamine; N-(4-Fluoro-benzyl)-N-[4-(4-fluorobenzylamino)-6-(prop-2-ynylamino)-[1,3,5]triazin-2-yl]-O-methyl-hydroxylamine; N,N′-Bis-(4-fluorobenzyl)-N″-prop-2-ynyl-[1,3,5]triazine-2,4,6-triamine; N-(4,6-Bis-prop-2-ynylamino-[1,3,5]triazin-2-yl)-O,N-dimethyl-hydroxylamine; N-Methyl-N′,N″-di-prop-2-ynyl-[1,3,5]triazine-2,4,6-triamine; N,N′-Bis-(4-fluoro-benzyl)-N″-n-propyl-[1,3,5]triazine-2,4,6-triamine; O-(4-Fluorophenyl)-N-(4-n-propylamino-6-prop-2-ynylamino-[1,3,5]triazin-2-yl)-hydroxylamine; N-[4-(1,1-Dimethyl-prop-2-ynylamino)-6-n-propylamino-[1,3,5]triazin-2-yl]-O,N-dimethyl-hydroxylamine; O,N-Dimethyl-N-(4-n-propylamino-6-but-2-ynylamino-[1,3,5]triazin-2-yl)-hydroxylamine; O,N-Dimethyl-N-(6-n-propylamino-2-prop-2-ynylamino-pyrimidin-4-yl)-hydroxylamine; O,N-Dimethyl-N-(2-n-propylamino-6-prop-2-ynylamino-pyrimidin-4-yl)-hydroxylamine; O,N-Dimethyl-N-(4-methylamino-6-prop-2-ynylamino-[1,3,5]triazin-2-yl)-hydroxylamine; O,N-Dimethyl-N-(4-ethylamino-6-prop-2-ynylamino-[1,3,5]triazin-2-yl)-hydroxylamine; O,N-Dimethyl-N-(4-isopropylamino-6-prop-2-ynylamino-[1,3,5]triazin-2-yl)-hydroxylamine; O,N-Dimethyl-N-(4-cyclopropylamino-6-prop-2-ynylamino-[1,3,5]triazin-2-yl)-hydroxylamine; O,N-Dimethyl-N-(4-n-butylamino-6-prop-2-ynylamino-[1,3,5]triazin-2-yl)-hydroxylamine; O,N-Dimethyl-N-(4-cyclobutylamino-6-prop-2-ynylamino-[1,3,5]triazin-2-yl)-hydroxylamine; O,N-Dimethyl-N-(4-cyclopropylmethylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-cyclohexylamino-6-prop-2-ynylamino-[1,3,5]triazin-2-yl)-hydroxylamine; O,N-Dimethyl-N-(4-benzylamino-6-prop-2-ynylamino-[1,3,5]triazin-2-yl)-hydroxylamine; O,N-Dimethyl-N-[4-(1-methyl-prop-2-ynylamino)-6-n-propylamino-[1,3,5]triazin-2-yl]-hydroxylamine; O,N-Dimethyl-N-(4-but-3-ynylamino-6-n-propylamino-[1,3,5]triazin-2-yl)-hydroxylamine; N-But-3-ynyl-N′-methyl-N″-propyl-[1,3,5]triazine-2,4,6-triamine; O-tert-Butyl-N-(4-n-propylamino-6-prop-2-ynylamino-[1,3,5]triazin-2-yl)-hydroxylamine; O-Ethyl-N-methyl-N-(4-n-propylamino-6-prop-2-ynylamino-[1,3,5]triazin-2-yl)-hydroxylamine; O-Ethyl-N-(4-n-propylamino-6-prop-2-ynylamino-[1,3,5]triazin-2-yl)-hydroxylamine; O-Methyl-N-(4-n-propylamino-6-prop-2-ynylamino-[1,3,5]triazin-2-yl)-hydroxylamine; N-Methyl-N-(4-n-propylamino-6-prop-2-ynylamino-[1,3,5]triazin-2-yl)-hydroxylamine; N-(4-n-Propylamino-6-prop-2-ynylamino-[1,3,5]triazin-2-yl)-hydroxylamine; O-(2-Methoxy-ethyl)-N-methyl-N-(4-n-propylamino-6-prop-2-ynylamino-[1,3,5]triazin-2-yl)-hydroxylamine; N-Methyl-O-(4,4,5,5,5-pentafluoro-pentyl)-N-(4-n-propylamino-6-prop-2-ynylamino-[1,3,5]triazin-2-yl)-hydroxylamine; N-(4-Fluorophenyl)-N′-propyl-N″-prop-2-ynyl-[1,3,5]triazine-2,4,6-triamine; N-(3-Chloro-2-methyl-benzyl)-N′-n-propyl-N″-prop-2-ynyl-[1,3,5]triazine-2,4,6-triamine; N-(3,4-Dichlorobenzyl)-N′-n-propyl-N″-prop-2-ynyl-[1,3,5]triazine-2,4,6-triamine; O,N-Dimethyl-N-(2-prop-2-ynylamino-7H-pyrrolo[2,3-d]pyrimidin-4-yl)-hydroxylamine; N-(4,6-Bis-n-propylamino-[1,3,5]triazin-2-yl)-O-methyl-N-prop-2-ynyl-hydroxylamine; O-Methyl-N-(4-n-propylamino-6-prop-2-ynylamino-[1,3,5]triazin-2-yl)-N-prop-2-ynyl-hydroxylamine; N-(4,6-Bis-n-propylamino-[1,3,5]triazin-2-yl)-N-methyl-O-prop-2-ynyl-hydroxylamine; N-(4,6-Bis-n-propylamino-[1,3,5]triazin-2-yl)-O-prop-2-ynyl-hydroxylamine; N-Methyl-N-(4-n-propylamino-6-prop-2-ynylamino-[1,3,5]triazin-2-yl)-O-prop-2-ynyl-hydroxylamine; N-(4-n-Propylamino-6-prop-2-ynylamino-[1,3,5]triazin-2-yl)-O-prop-2-ynyl-hydroxylamine; N-(4-Allylamino-6-prop-2-ynylamino-[1,3,5]triazin-2-yl)-O,N-dimethyl-hydroxylamine; 1-[4-(N-methoxy-N-methyl-amino)-6-prop-2-ynylamino-[1,3,5]triazin-2-ylamino]-propan-2-ol; 3-[4-(N-methoxy-N-methyl-amino)-6-prop-2-ynylamino-[1,3,5]triazin-2-ylamino]-propan-1-ol; N-(4-Amino-6-prop-2-ynylamino-[1,3,5]triazin-2-yl)-O,N-dimethyl-hydroxylamine; 3-[4-(N-Methoxy-N-methylamino)-6-prop-2-ynylamino-[1,3,5]triazin-2-ylamino]-propionaldehyde; 3-[4-(N-Methoxy-N-methylamino)-6-prop-2-ynylamino-[1,3,5]triazin-2-ylamino]-propionic acid ethyl ester; N-Propyl-N′-prop-2-ynyl-[1,3,5]triazine-2,4,6-triamine; N-[4-(N′-Methoxy-N′-methyl-amino)-6-prop-2-ynylamino-[1,3,5]triazin-2-yl]-N-propyl acetamide; N-[4-(N′-Methoxy-N′-methyl-amino)-6-prop-2-ynylamino-[1,3,5]triazin-2-yl]-N-propyl adamantylamide; N-Ethyl-N′-methyl-N″-prop-2-ynyl-[1,3,5]triazine-2,4,6-triamine; N-Cyclopropyl-N′-methyl-N″-prop-2-ynyl-[1,3,5]triazine-2,4,6-triamine; N-Butyl-N′-methyl-N″-prop-2-ynyl-[1,3,5]triazine-2,4,6-triamine; N-Cyclopropylmethyl-N′-methyl-N″-prop-2-ynyl-[1,3,5]triazine-2,4,6-triamine; N-Methyl-N′-prop-2-ynyl-N″-(3,3,3-trifluoro-propyl)-[1,3,5]triazine-2,4,6-triamine; N-Methyl-N′-(2,2,3,3,3-pentafluoro-propyl)-N″-prop-2-ynyl-[1,3,5]triazine-2,4,6-triamine; N-(1-Ethyl-propyl)-N′-methyl-N″-prop-2-ynyl-[1,3,5]triazine-2,4,6-triamine; N,N-Dimethyl-N′-propyl-N″-prop-2-ynyl-[1,3,5]triazine-2,4,6-triamine; N,N-Ethyl-methyl-N′-propyl-N″-prop-2-ynyl-[1,3,5]triazine-2,4,6-triamine; N-Ethyl-N′-propyl-N″-prop-2-ynyl-[1,3,5]triazine-2,4,6-triamine; N-Propyl-N′-propyl-N″-prop-2-ynyl-[1,3,5]triazine-2,4,6-triamine; N-Cyclopropyl-N′-propyl-N″-prop-2-ynyl-[1,3,5]triazine-2,4,6-triamine; N-Isopropyl-N′-propyl-N″-prop-2-ynyl-[1,3,5]triazine-2,4,6-triamine; N-Butyl-N′-propyl-N″-prop-2-ynyl-[1,3,5]triazine-2,4,6-triamine; N-Cyclopropylmethyl-N′-propyl-N″-prop-2-ynyl-[1,3,5]triazine-2,4,6-triamine; a salt thereof, and any combinations thereof.

In a preferred embodiment, the compound of formula (I) is selected from the group consisting of O,N-Dimethyl-N-[4-(n-propylamino)-6-(prop-2-ynylamino-[1,3,5]triazin-2-yl]-hydroxylamine; N-Methyl-N′-n-propyl-N″-prop-2-ynyl-[1,3,5]triazine-2,4,6-triamine; a salt thereof; and any combinations thereof.

### Pharmaceutical Compositions and Formulations

The invention also encompasses the use of pharmaceutical compositions of at least one compound of the invention or a salt thereof to practice the methods of the invention. Such a pharmaceutical composition may consist of at least one compound of the invention or a salt thereof, in a form suitable for administration to a subject, or the pharmaceutical composition may comprise at least one compound of the invention or a salt thereof, and one or more pharmaceutically acceptable carriers, one or more additional ingredients, or some combination of these. The at least one compound of the invention may be present in the pharmaceutical composition in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In one embodiment, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g. cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g. Recombumin®), solubilized gelatins (e.g. Gelofusine®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a breathing disorder event. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a breathing control disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of breathing disorders in a patient.

In one embodiment, the compositions of the invention are administered to the patient in dosages that range from one to five times per day or more. In another embodiment, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of breathing disorder in a patient.

The term “container” includes any receptacle for holding the pharmaceutical composition or for managing stability or water uptake. For example, in one embodiment, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a breathing disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those skilled in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Application Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In one embodiment, the composition is designed to promote controlled release of the drug, such that the location, extent and rate of exposure of the compound when administered are modulated. Factors that affect the target zone for exposure of an orally administered drug may be the drug's pH and enzymatic stability, reactivity with other drugs (e.g., certain antibiotics), solubility as a salt or free base, ionization behavior, and pharmacodynamic and pharmacokinetic behaviors in specific environments.

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include targeted delivery within the gastrointestinal tract upon oral administration, extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example water, pH, temperature, enzymes, bacteria, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations. The active drug substance can also be coated on an implantable medical device to be eluted or be released using a remotely activated system.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation (drug embedded in polymeric matrices).

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 24 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

A drug may be better absorbed in the duodenum or other intestinal locations. A particularly useful mode of controlled release is one which minimizes release of drug in the stomach, while delivering drug in its most concentrated form to the duodenum or other intestinal locations. In one embodiment, the compounds of the present invention are formulated to promote delivery to the duodenum and, optionally, other intestinal locations. Controlled release that delivers drug to the duodenum or other intestinal regions may be achieved using compositions that include enteric coatings. Enteric coatings are insoluble in highly acidic environments, often comprising a polyacidic coating that remains non-ionized and intact at gastric pH. However, under mildly acidic (>pH 5.5) or neutral or mildly alkaline conditions (pH 6.5-7.6) of the duodenum or other intestinal regions, the coating ionizes, swells and breaks down, exposing the coated entity to the environment. Coating options exist to allow ionization at or near a specific pH (e.g. Eudragit L-110, ionization threshold pH 6.0; Eudragit S-100, ioization threshold pH 7.0). It is understood that similar type or grade of film coating or polymeric products from other companies may be used.

In one embodiment, compounds of the present invention are formulated with an enteric coating, which has been modified by adding plasticizers to the polymer before coating. The plasticizers may be added to adjust resistance to chipping or cracking of the coating, while also lowering the glass transition temperature of the coating to enable smoothness and even spreadability of the coating during its application. Suitable plasticizers include polyethylene glycol 8000 (PEG 8000), triethyl citrate (TEC), and triacetin, which may be incorporated into the polymeric enteric coating agent.

Compounds of the present invention may be enterically formulated under a variety of dosage forms, including (but not limited to) capsules, granules of the active drug itself, beads, micro spheres, and tablets. In one embodiment, the composition comprises a drug encapsulated in a capsule enterically coated to release the drug in the duodenum or other intestinal environment. In another embodiment, pharmaceutically acceptable capsules include hard capsules. In yet another embodiment, pharmaceutically acceptable capsules include soft gelatin capsules.

In one embodiment, a compound of the invention is encapsulated in pure granular or powdered form, with no carriers, excipients or other pharmaceutically acceptable additives. In another embodiment, a compound of the invention is encapsulated together with one or more pharmaceutically acceptable carriers, excipients, antioxidants, antifungals, (e.g., benzoic and ascorbic acids and their salts, and phenolic compounds such as methyl, ethyl, propyl and butyl p-hydroxybenzoate (parabens)), antimicrobial preservatives, colorants, and flavorants. The excipients may aid in capsule-filling behavior, stability, and in the distribution of the drug when the capsule disintegrates in the body. In another embodiment, granules and/or powders of a compound of the present invention are enterically coated before being placed in a capsule. The enterically coated granules and/or powders placed in the capsule may feature one or several types of enteric coating to enable delivery of the drug to different regions of the intestine. The capsule may lack enteric coating or may be coated with an enteric coating that is the same as or distinct from the coating applied to any of the enterically coated materials inside the capsule.

In one embodiment, a compound of the invention is encapsulated in a liquid in the form of a solution or suspension in water or various pharmaceutically acceptable oils or other dispersion medium, optionally with such excipients as cosolvents (e.g., PEG 300, PEG 400, propylene glycol, glycerol, tween 80, ethanol), solubility enhancers (e.g., sorbitol, dextrose), wetting agents (e.g., thickening agents), buffers (e.g., disodium hydrogen phosphate), antioxidants, antifungals, preservatives, colorants and flavorants. In one embodiment, a compound of the present invention is formulated for liquid filled capsules in the form of the pure drug as granules and/or powders in the liquid. In another embodiment, the capsule containing the compound in liquid is enterically coated. In yet another embodiment, granules and/or powders of a compound of the invention are enterically coated before being placed in a liquid and the combination placed in a capsule. The enterically coated granules and/or powder may feature one or several types of enteric coating to enable delivery of the drug to distinct regions of the intestine. The capsule may lack enteric coating or may be coated with an enteric coating that is the same as or distinct from the coating applied to any of the enterically coated materials inside the capsule.

In one embodiment, a compound of the present invention is encapsulated in a capsule comprised of material that affords post-gastric drug delivery without the need for the separate application of an enteric coating (e.g., Entericare enteric softgels). The compound may be encapsulated in such capsules as granules or powders with or without excipients, and as solutions or suspensions as described above.

In one embodiment, the solid particles of a compound of the present invention, as a variety of particle sizes and particle size distributions, are admixed with excipients such as microcrystalline cellulose or lactose and formed as a bead that comprises the drug-containing core onto which the enteric coating is applied. In another embodiment, a compound of the present invention is formed as a suspension or solution including, optionally, buffers (e.g., aq. 1 N HCl with tris(hydroxymethyl)aminomethane “TRIS”), and binders (e.g., Opadry Clear Coat Powder) and coated onto a base particle, for example sugar beads (e.g., Sugar Spheres, NF particles) to form a bead. In yet another embodiment, the beads are enterically coated. In yet another embodiment, a compound of the invention is formulated as enterically coated beads, as described above, and the beads further formulated by encapsulation. In yet another embodiment, a combination of beads with different types of enteric coating is encapsulated, such that once released from the capsule, the compound of the invention is made available in a controlled manner at different regions ranging from the duodenum to other parts of the intestine. The capsule may lack enteric coating or may be coated with an enteric coating that is the same as or distinct from the coating applied to any of the enterically coated materials inside the capsule.

In one embodiment, a compound of the present invention is formulated as tablets or caplets which alone or in combination with other formulation components deliver drug to the duodenum or other intestinal region. In another embodiment, a compound of the invention is formulated as tablets or caplets that are enterically coated and that constitute the dosage form administered. In yet another embodiment, tablets or caplets of suitable size and shape are placed inside a capsule. In yet another embodiment, the capsule is enterically coated and contains non-enterically coated tablets or caplets, which are released from the capsule in the duodenum or other intestinal region. In yet another embodiment, the capsule is designed to disintegrate in the stomach and release entericallly coated tablets or caplets for subsequent delivery to duodenum or other intestinal regions. In yet another embodiment, the capsule and tablets or caplets contained within are both enterically coated to provide further control over the release of the tablets or caplets from the capsule, and the subsequent release of the drug from the tablet or caplet. In yet another embodiment, tablets or caplets featuring a variety of enteric coating are combined and placed in a capsule which itself may optionally be enterically coated as well. Materials useful for enteric coatings for tablets and caplets include but are not limited to those described above for application to capsules.

Enteric coatings may permit premature drug release in acidic media. In one embodiment, a compound of the present invention is formulated such that a subcoating is applied before the enteric coating is applied. The subcoating may comprise application to the enteric substrate of a soluble subcoating agent, examples of which are hydroxypropylmethylcellulose, povidone, hydroxypropyl cellulose, polyethylene glycol 3350, 4500, 8000, methyl cellulose, pseudo ethylcellulose and amylopectin. It is understood that similar type of synthetic and semisynthetic polymeric products from other companies may be used. A thin subcoating layer on the enteric substrate impedes water penetration through the enteric coating on the capsule shell or into the core where the active ingredient is located, preventing premature drug release. The subcoating may also promote the release of the drug in a basic environment by moderating the acidic microenvironment at the interface between the core and the enteric coating. In one embodiment, a compound of the present invention is formulated with a subcoating containing organic acids intended to promote more rapid polymer dissolution of a capsule as the coating degrades in environments with pH 5-6, promoting a rapid release of the drug in basic media.

### Mechanical Devices

In one aspect of the invention, a method of treating a patient without normal ventilation and normal breathing control comprises administering the composition useful within the invention as described herein, and additionally treating the patient using a device to support breathing. Such devices include, but are not limited to, ventilation devices, CPAP and BiPAP devices.

Mechanical ventilation is a method to mechanically assist or replace spontaneous breathing. Mechanical ventilation is typically used after an invasive intubation, a procedure wherein an endotracheal or tracheostomy tube is inserted into the airway. It is normally used in acute settings, such as in the ICU, for a short period of time during a serious illness. It may also be used at home or in a nursing or rehabilitation institution, if patients have chronic illnesses that require long-term ventilation assistance. The main form of mechanical ventilation is positive pressure ventilation, which works by increasing the pressure in the patient's airway and thus forcing air into the lungs. Less common today are negative pressure ventilators (for example, the “iron lung”) that create a negative pressure environment around the patient's chest, thus sucking air into the lungs. Mechanical ventilation is often a life-saving intervention, but carries many potential complications including pneumothorax, airway injury, alveolar damage, and ventilator-associated pneumonia. For this reason the pressure and volume of gas used is strictly controlled, and discontinued as soon as possible. Types of mechanical ventilation are: conventional positive pressure ventilation, high frequency ventilation, non-invasive ventilation (non-invasive positive pressure ventilation or NIPPV), proportional assist ventilation (PAV), adaptive servo ventilation (ASV) and neurally adjusted ventilatory assist (NAVA).

Non-invasive ventilation refers to all modalities that assist ventilation without the use of an endotracheal tube. Non-invasive ventilation is primarily aimed at minimizing patient discomfort and the complications associated with invasive ventilation, and is often used in cardiac disease, exacerbations of chronic pulmonary disease, sleep apnea, and neuromuscular diseases. Non-invasive ventilation refers only to the patient interface and not the mode of ventilation used; modes may include spontaneous or control modes and may be either pressure or volume cycled modes. Some commonly used modes of NIPPV include:

(a) Continuous positive airway pressure (CPAP): This kind of machine has been used mainly by patients for the treatment of sleep apnea at home, but now is in widespread use across intensive care units as a form of ventilatory support. The CPAP machine stops upper airway obstruction by delivering a stream of compressed air via a hose to a nasal pillow, nose mask or full-face mask, splinting the airway open (keeping it open under air pressure) so that unobstructed breathing becomes possible, reducing and/or preventing apneas and hypopneas. When the machine is turned on, but prior to the mask being placed on the head, a flow of air comes through the mask. After the mask is placed on the head, it is sealed to the face and the air stops flowing. At this point, it is only the air pressure that accomplishes the desired result. This has the additional benefit of reducing or eliminating the extremely loud snoring that sometimes accompanies sleep apnea.

(b) Bi-level positive airway pressure (BIPAP): Pressures alternate between inspiratory positive airway pressure (IPAP) and a lower expiratory positive airway pressure (EPAP), triggered by patient effort. On many such devices, backup rates may be set, which deliver IPAP pressures even if patients fail to initiate a breath.

(c) Intermittent positive pressure ventilation (IPPV), via mouthpiece or mask.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all remaining starting materials were obtained from commercial suppliers and used without purification. Final products are typically isolated as salts unless noted otherwise.

### Example 1

### O,N-Dimethyl-N-[4(n-propylamino)-6-(prop-2-ynylamino)-[1,3,5]triazin-2-yl]-hydroxylamine (4), and corresponding salts: hydrochloride salt (5a) and hydrogen sulfate salt (5b) (Scheme 10)

### Example 1A, Stage 1

### 2,4-Dichloro-N-(6-n-propylamino)-[1,3,5]triazine (2); In-Process and Purity Method 3M-8 (In-Process and Purity Analysis, Method 3M-D)

A 2-liter jacketed glass reactor with a bottom drain valve, agitator (three-blade impeller), thermometer and dropping funnel (with a pressure equalizing arm) was charged with powdered cyanuric chloride (1) (120 g, 0.651 mol, 1 equiv.) and THF (540 mL). The temperature in the jacket was set to −25° C.

Separately, n-propylamine (53.4 mL, 0.651 mol, 1 equiv.) and DIPEA (113.3 mL, 0.651 mol, 1 equiv.) were dissolved in THF (960 mL). This mixture was added dropwise to the stirred solution of (1) over 4 h at −25° C. After this time, the reaction mixture was allowed to warm to room temperature and stirred for 16 h. The volatiles were removed under vacuum and the resultant oily residue was partitioned between EtOAc (1000 mL) and water (300 mL). The organic layer was washed with water (2×300 mL) and then with a brine solution (500 mL), and dried over anhydrous solid sodium sulfate. After filtering, the solvent was removed under reduced pressure. An oily residue was obtained, which solidified after drying under vacuum at 0.1 mbar for 5 h, to yield 2,4-dichloro-N-(6-n-propylamino)-[1,3,5]triazine (2) (125 g, 93%). ¹H NMR (400 MHz, CDCl₃, ppm): δ 6.62-6.15 (1H, br s), 3.45 (2H, dt, J=6.4 and 1 Hz), 1.70-1.58 (2H, m), 0.99-0.93 (3H, m). ESI-MS (m/z): 207, 209 [M+H]⁺.

### Example 1B, Stage 2

### 6-Chloro-N²-(prop-2-ynylamino)-N⁴-n-propylamino-1,3,5-triazine (3)

A mixture of 4,6-dichloro-[1,3,5]triazin-2-yl)-n-propyl-amine (2) (3.00 g, 14.49 mmol), propargylamine hydrochloride (1.46 g, 15.94 mmol) and N,N-diisopropylethylamine (5.3 mL, 31.88 mmol) in 1,4-dioxane (25 mL) was stirred at 55° C. for 2 h. The mixture was cooled to room temperature. The resultant precipitate was filtered, washed with water and dried to yield 6-chloro-N²-(prop-2-ynylamino)-N⁴-n-propylamino-1,3,5-triazine-2,4-diamine (3) (2.98 g, 91%). 400 MHz ¹H NMR (DMSO-d₆, ppm): δ 8.16-7.83 (2H, m), 4.01-3.93 (2H, m), 3.22-3.08 (2H, m), 3.08-3.03 (1H, m), 1.57-1.43 (2H, m), 0.90-0.81 (3H, m). ESI-MS (m/z): 226, 228 [M+H]⁺.

### Example 1C, Stage 3, Method 1

### O,N-Dimethyl-N-[4-(n-propylamino)-6-(prop-2-ynylamino)-[1,3,5]triazin-2-yl]-hydroxylamine (4); In-Process and Purity Method 3M-D

A mixture of 6-chloro-N²-(prop-2-ynylamino)-N⁴-n-propylamino-1,3,5-triazine (3) (2.68 g, 11.88 mmol), O,N-dimethylhydroxylamine hydrochloride (2.67 g, 27.32 mmol) and NaOH (1.10 g, 27.32 mmol) in 1,4-dioxane (30 mL) was heated at 90° C. for 4 h. The volatiles was removed under reduced pressure. A saturated NaHCO₃ solution (100 mL) was added to the residue and the mixture was extracted with EtOAc (3×50 mL). The combined organic extracts were washed with water (100 mL), then with a brine solution (100 mL), and lastly dried over anhydrous Na₂SO₄. The volatiles were removed under reduced pressure and the resultant residue was purified by flash column chromatography using gradient elution from CH₂Cl₂/EtOH (99:1) to CH₂Cl₂/EtOH (97:3) to yield O,N-dimethyl-N-[4-(n-propylamino)-6-(prop-2-ynylamino)-[1,3,5]triazin-2-yl]-hydroxylamine (4) (2.51 g, 85%). 400 MHz ¹H NMR (CDCl₃, ppm): δ 6.66-5.67 (1H, m), 5.64-4.98 (1H, m), 4.26-4.12 (2H, m), 3.82 (3H, s), 3.42-3.23 (5H, m), 2.25-2.19 (1H, m), 1.51 (2H, sextet, J=7.4 Hz), 0.96 (3H, t, J=7.4 Hz). ESI-MS (m/z): 251 [M+H]⁺.

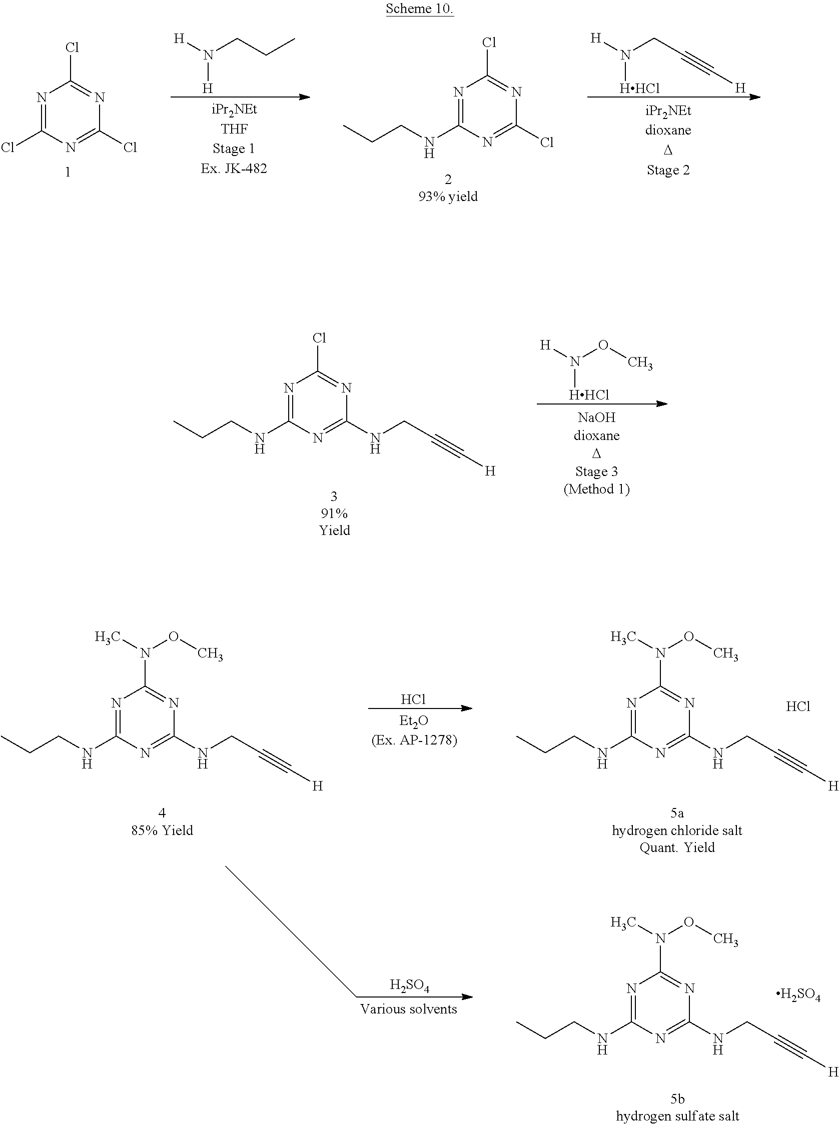

### Example 2

### Telescoped Route to O,N-Dimethyl-N-[4(n-propylamino)-6-(prop-2-ynylamino)-[1,3,5]triazin-2-yl]-hydroxylamine (4)(Scheme 11)

### Example 2A

### Stage 1 and 2 combined (“telescopic” method with propargylamine free base); 2,4-dichloro-N-(6-n-propylamino)-[1,3,5]triazine (2) and 6-chloro-N²-(prop-2-ynylamino)-N⁴-n-propylamino-[1,3,5]triazine (3) (In-Process and Purity Method 3M-B and 3M-C)

A 2-liter jacketed glass reactor with a bottom drain valve, agitator (three-blade impeller), thermometer and dropping funnel (with a pressure equalizing arm) was charged with powdered cyanuric chloride (1) (100 g, 0.542 mol, 1 equiv.). The temperature in the jacket was set to −2° C. Isopropanol (IPA) (440 mL) pre-cooled to 0° C. was added. The resultant mixture was stirred for 2 min, after which time all of the cyanuric chloride was observed to be in a slurry.

Separately, n-propylamine (40 mL, 0.488 mol, 0.9 equiv.) and DIPEA (94.5 mL, 0.542 mol, 1 equiv.) were dissolved in IPA (800 mL). This mixture was added dropwise to the slurry of cyanuric chloride in IPA over 4 h at 0-2° C., with stirring (400 rpm). After the addition was completed, the temperature in the jacket was set to ambient temperature (20° C.) and the reaction mixture stirred at this temperature for 30 min (bis-n-propyl amine adduct (20) undetected, Method 3M-B). This solution was telescoped into the next reaction (Stage 2) in the same vessel as described below.

To the reaction mixture from Stage 1, DIPEA (94.5 mL, 0.542 mol, 1 equiv.) was added in one portion and then stirred for 1 h at ambient temperature. N-Propargylamine (38.2 mL, 0.597 mol, 1.1 equiv.) was added in one portion. An exothermic reaction immediately took place, with temperature increase to 30-35° C. Once the exotherm dissipated, the temperature in the jacket was set to 65° C. and the reaction mixture was stirred for 16 h at this temperature, and then cooled to ambient temperature. The resulting propargylamino adduct was collected by filtration on a sintered glass funnel, washed with IPA (3×300 mL) and then with light petroleum ether (3×400 mL). The filtered and washed product was air dried 75° C. for 16 h to afford 6-chloro-N²-(prop-2-ynylamino)-N⁴-n-propylamino-1,3,5-triazine (3) (103.5 g, 94%) as colorless solid. The level of 2-chloro-(4,6-di-n-propylamino([1,3,5]triazine by-product was <0.1%. ¹H NMR (400 MHz, CDCl₃, ppm): δ 8.19-7.64 (2H, m), 4.07-3.92 (2H, m), 3.27-3.03 (3H, m), 1.8-1.41 (2H, m), 0.92-0.80 (3H, m). LC-MS: retention time 2.47 min; ESI-MS (m/z): 226, 228 [M+H]⁺; HPLC purity: 99% (Method 3M-C).

### Example 2b

### Stages 1 & 2 combined; “telescoped” method 2 with purified propargyl amine hemisulfate: 2,4-dichloro-N-(6-n-propylamino)-[1,3,5]triazine (2) and 6-chloro-N²-(prop-2-ynylamino)-N⁴-n-propylamino-1,3,5-triazine (3) (In Process Control and Purity Methods 3M-B and 3M-C)

Stage 1 was conducted in 2 separate batches, each starting from 130 g of cyanuric chloride (1). A 2-L jacketed glass reactor with a bottom drain valve, agitator (three-blade impeller), thermometer and dropping funnel (with a pressure equalizing arm) was charged with powdered (1) (130 g, 0.705 mol, 1 equiv.). The temperature in the jacket was set to −2° C. Isopropanol (570 mL), was added and the mixture was stirred for 2 min, during which time all cyanuric chloride was observed to exist as a slurry. Separately, n-propylamine (52.1 mL, 0.634 mol, 0.9 equiv.) and N,N-diisopropylethylamine (123 mL, 0.705 mol, 1 equiv.) were dissolved in isopropanol (1,040 mL). This mixture was added dropwise to the slurry of (1) in isopropanol over 4 h at 0-2° C., with stirring (400 rpm). After completion of the addition, reaction mixture was removed from the reactor and stored at −10° C. for 5 h, until the second batch of Stage 1 was finished.

The second batch was prepared identically in the same equipment setup and starting from the same amount of cyanuric chloride (130 g, 0.705 mol, 1 equiv.). Both batches of Stage 1 product were combined in a 5 L hastelloy reactor, equipped with a heating/cooling mantle, a thermocouple, an agitator (3-blade impeller) and a bottom drain valve. The temperature in the mantle was set to ambient (25° C.) and reaction mixture was stirred at this temperature for 30 min. To the mixture, neat N,N-diisopropylethylamine (491 mL, 2.820 mol, 2 equiv. vs. cyanuric chloride for combined batches) was added in one portion and then stirred 1 h at ambient temperature. Propargylamine hemisulfate (161.5 g, 0.776 mol, 0.55 eq, Example 2G, 0.0065% 2-chloroallyl amine, Method 3M-A) was added in one portion. An exothermic reaction immediately took place with temperature increase to 30-32° C. Once the exotherm subsided, the temperature in the mantle was set to 65° C. and the reaction mixture was stirred for 16 h. After cooling to ambient temperature (25° C.), the product (3) was collected by filtration on a sintered glass funnel and washed with isopropanol (2×600 mL). The wet filter cake was suspended in water (3 L) and stirred for 0.5 h at room temperature. The product was collected by filtration, washed with water (3×600 mL), then with isopropanol (600 mL) and lastly with light petroleum ether (BP 40-60° C.) (600 mL) and air dried at 75° C. for 16 h to afford 6-chloro-N²-(prop-2-ynylamino)-N⁴-n-propylamino-1,3,5-triazine (3): 268 g (93.6%, uncorr.), as a colorless solid; HPLC purity: 99%, Method 3M-C.

TABLE 1
Yields of 6-chloro-N²-(prop-2-ynylamino)-N⁴-n-propylamino-1,3,5-
triazine (3) using “telescoping” procedure (Examples 2A and 2B).

| Scale (g) | Yield (%) | Comment |
|---|---|---|
| 50 g | >95% | Method 1 |
| | | 0.9 mol equiv. n-Pr amine |
| 100 g | 99% | Method 1 |
| | | 0.9 mol equiv. n-Pr amine |
| 100 g | 94 | Method 1 |
| | | 0.9 mol equiv. n-Pr amine |
| 100 g | 94 | Method 1 |
| Example 2 | | 0.9 mol equiv. n-Pr amine |
| 268 g | 93.5 | telescoped, Method 2 |
| Example 2B | | 0.9 mol equiv. n-Pr amine |

### Example 2C

### Stage 3, Method 2; Isolation of solid O,N-dimethyl-N-[4-(n-propylamino)-6-(prop-2-ynylamino)-[1,3,5]triazin-2-yl]-hydroxylamine (4) from N,N-dimethyl acetamide and water (In Process Control and Purity Method 3M-D)

A 2-L jacketed glass reactor equipped with a bottom drain valve, agitator (3-blade impeller) and thermometer was charged with 6-chloro-N²-(prop-2-ynylamino)-N⁴-n-propylamino-1,3,5-triazine (3) (103.5 g, 0.459 mol, 1 equiv.) and K₂CO₃ (126.8 g, 0.917 mol, 2 equiv.), and then N,N-dimethylacetamide (620 mL) was added. O,N-dimethyhydroxylamine hydrochloride (67.1 g, 0.688 mol, 1.5 equiv.) was added in portions in order to reduce foaming. After the addition was complete, the reaction mixture was stirred for 2 h at 60° C. (in jacket). At this time, heating was discontinued and water (1,240 mL) was added dropwise over 2.5 h with stirring (850 rpm). After the addition of water was completed, a biphasic mixture was obtained, which was stirred for additional 1 h at ambient temperature. After this time, 100 mg of seed crystals of product (4) were introduced. Crystallization immediately began and the reaction mixture was stirred 16 h at ambient temperature to complete the process. The product (4) was collected by filtration, washed with water (3×300 mL) and dried under vacuum at 50° C. for 16 h, to yield O,N-dimethyl-N-[4-(n-propylamino)-6-(prop-2-ynylamino)-[1,3,5]triazin-2-yl]-hydroxylamine (4) as colorless solid (105 g, 91%). ¹H NMR (400 MHz, CDCl₃, ppm): δ 5.38-4.91 (2H, m), 4.26-4.09 (2H, m), 3.83-3.68 (3H, m), 3.39-3.19 (5H, m), 2.19 (1H, t, J=2.50 Hz), 1.63-1.50 (2H, m), 0.93 (3H, t, J=7.46 Hz). MP 79-81° C. ESI-MS (m/z): 251 [M+H]+; HPLC Purity: 99% (Method 3M-D); XRPD illustrated in FIG. 18.

TABLE 2
Elemental analysis of O,N-Dimethyl-N-[4-(n-propylamino)-6-(prop-2-
ynylamino)-[1,3,5]triazin-2-yl]-hydroxylamine (4) (Example 2C).

| | C | H | N |
|---|---|---|---|
| Calculated | 52.78 | 7.25 | 33.58 |
| Test 1 | 52.51 | 7.17 | 33.71 |
| Test 2 | 52.64 | 7.07 | 33.78 |

TABLE 3
¹H NMR analysis of O,N-dimethyl-N-(4-n-propylamino-6-prop-2-ynyl-
amino-[1,3,5]triazin-2-yl)-hydroxylamine (4). 400 MHz; CDCl₃; 10
mg/mL; 32 scans (FIG. 16).

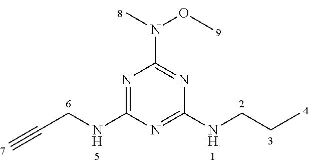 [SMILES: C#CCNc1nc(NCCC)nc(N(C)OC)n1]

| Delta | Peak | | Coupling | |
|---|---|---|---|---|
| (ppm) | description | Integration | (Hz) | Assignment |
| 5.40-4.90 | m | 2H | — | 1 + 5 |
| 4.27-4.08 | m | 2H | — | 6 |
| 3.83-3.67 | m | 3H | — | 9 |
| 3.39-3.18 | m | 5H | — | 2 + 8 |
| 2.19 | m | 1H | — | 7 |
| 1.62-1.50 | m | 2H | — | 3 |
| 0.93 | t | 3H | 7.47 | 4 |

TABLE 4
¹³C NMR analysis of O,N-dimethyl-N-(4-n-propylamino-6-prop-2-
ynylamino-[1,3,5]triazin-2-yl)-hydroxylamine (4). 100 MHz; CDCl₃; 20
mg/mL; 256 scans (FIG. 17).

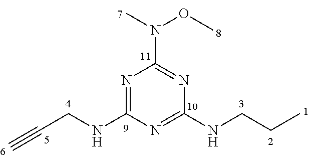 [SMILES: C#CCNc1nc(NCCC)nc(N(C)OC)n1]

| Delta | |
|---|---|
| (ppm) | Assignment |
| 168.49 | 11 |
| 166.40 | 9 + 10 |
| 165.94 | |
| 80.91 | 5 |
| 70.80 | 6 |
| 61.31 | 8 |
| 42.64 | 3 |
| 36.02 | 7 |
| 30.51 | 4 |
| 23.05 | 2 |
| 11.56 | 1 |

TABLE 5
O,N-Dimethyl-N-(4-n-propylamino-6-prop-2-ynylamino-[1,3,5]-triazin-
2-yl)-hydroxylamine (4) diffraction signals (Example 2C).

| Degrees 2θ | d space (Å) | Intensity (%) |
|---|---|---|
| 8.62 ± 0.20 | 10.253 ± 0.243 | 19 |
| 11.45 ± 0.20 | 7.729 ± 0.137 | 100 |
| 12.62 ± 0.20 | 7.015 ± 0.113 | 20 |
| 17.33 ± 0.20 | 5.117 ± 0.059 | 17 |
| 18.38 ± 0.20 | 4.826 ± 0.053 | 26 |
| 19.19 ± 0.20 | 4.626 ± 0.048 | 16 |
| 21.17 ± 0.20 | 4.196 ± 0.040 | 18 |
| 21.66 ± 0.20 | 4.103 ± 0.038 | 23 |
| 22.65 ± 0.20 | 3.927 ± 0.035 | 19 |

### Example 2D

### Recrystallization of O,N-dimethyl-N-[4-(n-propylamino)-6-(prop-2-ynylamino)-[1,3,5]triazin-2-yl]-hydroxylamine (4) from toluene and petroleum ether-40 (BP 40-60° C.)

O,N-Dimethyl-N-[4-(n-propylamino)-6-(prop-2-ynylamino)-[1,3,5]triazin-2-yl]-hydroxylamine (4) (1 g) was dissolved in toluene (2 mL) with gentle heating, and petroleum ether-40 (BP 40-60° C., PE 40) (10 mL) was added, causing immediate product precipitation of an oil that solidified upon standing. The solidified oil was dissolved in a mixture of PE 40 (10 mL) and toluene (2 mL), heated at reflux, and then the solution was allowed to cool to room temperature. O,N-Dimethyl-N-[4-(n-propylamino)-6-(prop-2-ynylamino)-[1,3,5]triazin-2-yl]-hydroxylamine (4) crystallized upon cooling to room temperature as a fine crystalline powder. Yield: 80%; XRPD as illustrated in FIG. 19.

TABLE 6
O,N-Dimethyl-N-(4-n-propylamino-6-prop-2-ynylamino-[1,3,5]-triazin-
2-yl)-hydroxylamine (4) diffraction signals (Example 2D).

| Pos. [°2Th.] | d-spacing [Å] | Rel. Int. [%] |
|---|---|---|
| 8.6174 | 10.25281 | 21.11 |
| 11.4346 | 7.73235 | 100.00 |
| 12.6026 | 7.01821 | 15.04 |
| 15.2379 | 5.80988 | 3.33 |
| 17.3449 | 5.10858 | 14.09 |
| 18.4032 | 4.81711 | 25.00 |
| 19.1585 | 4.62889 | 7.15 |
| 21.1774 | 4.19192 | 12.20 |
| 21.6560 | 4.10035 | 16.66 |
| 22.6169 | 3.92827 | 10.22 |
| 27.1264 | 3.28461 | 3.46 |
| 28.9878 | 3.07779 | 1.75 |

### Example 2E

### Recrystallization of O,N-Dimethyl-N-[4-(n-propylamino)-6-(prop-2-ynylamino)-[1,3,5]triazin-2-yl]-hydroxylamine (4) from toluene and heptanes

O,N-Dimethyl-N-[4-(n-propylamino)-6-(prop-2-ynylamino)-[1,3,5]triazin-2-yl]-hydroxylamine (4) (1 g) was dissolved in toluene (2 mL) with gentle heating, and then heptane (15 mL) was added at room temperature. Upon addition, an oil immediately precipitated, which was redissolved by heating at reflux, then cooled to room temperature and lastly seed crystals were added. The resulting oil was triturated by stirring with a glass rod to give rise to crystals. The resulting suspension was placed in an ice bath (0° C.) for 1 h. The solid product was collected by filtration, washed with a 2:15 v/v mixture of toluene/heptane (3×5 mL), and dried under vacuum at 50° C. to afford O,N-dimethyl-N-[4-(n-propylamino)-6-(prop-2-ynylamino)-[1,3,5]triazin-2-yl]-hydroxylamine (4) in 85% yield; XRPD as illustrated in FIG. 19.

### Example 2F

### Stage 3 Method 3; O,N-Dimethyl-N-[4-(n-propylamino)-6-(prop-2-ynylamino)-[1,3,5]triazin-2-yl]-hydroxylamine (4); Isolation of solid product from toluene and heptane (In Process Control Purity and Method 3M-D)

A 5-L hastelloy reactor with a glass lid, heating/cooling mantle, reflux condenser, bottom drain valve, agitator (3-blade impeller) and thermometer was charged with 6-chloro-N²-(prop-2-ynylamino)-N⁴-n-propylamino-1,3,5-triazine (3) (268 g, 1.188 mol, 1 equiv., Example 2B) and K₂CO₃ (328.3 g, 2.375 mol, 2 equiv.), and dimethylacetamide (1.6 L) was added. Stirring was started and O,N-dimethyhydroxylamine hydrochloride (196.9 g, 2.019 mol, 1.7 equiv.) was added portionwise over 2-3 minutes to reduce foaming. At this time, an additional amount of dimethylacetamide (0.2 L) was added in order to reach the minimum stirrable volume. The reaction mixture was stirred for 2 h at 60° C. (in the heating mantle). An aliquot of the reaction mixture was assayed by LC-MS, which revealed 99.7% conversion.

The reaction mixture was cooled to 30° C., and additional O,N-dimethyhydroxylamine hydrochloride (23.2 g, 0.238 mol, 0.2 equiv.) was added. The reaction mixture was stirred for 1 h at 60° C.; LC-MS assay showed 99.7% conversion. The reaction mixture was cooled to 30° C. (in solution) and water (3.6 L) was added at once, which caused foaming. After the foaming ceased, toluene (2 L) was added and mixture was stirred for 2 h at ambient temperature. The biphasic mixture was transferred to a barrel (HDPE) and left overnight at room temperature. The mixture was transferred to a 20-liter glass reactor, equipped with an agitator (2-blade anchor) and a bottom drain valve. The mixture was stirred (90 rpm) for 10 min at room temperature, and then layers were separated. The aqueous layer was extracted with toluene (2×0.6 L). The combined organic layers were washed with water (4×1.8 L). The toluene solution (˜3.5 L) was transferred to a 5 L reactor with a heating/cooling mantle, bottom drain valve, agitator (3-blade impeller) and thermometer. The mixture was heated to 112° C. and solvent was distilled with a Dean-Stark apparatus until no further water was collected. The toluene condensate was turbid, and additional toluene (˜1 L) was distilled off until the condensate became clear. The resulting solution was cooled and left overnight at room temperature. The next day the toluene solution (˜2.8 L) was placed in a 4 L round-bottom flask, equipped with a distillation head and a heating mantle. A portion of the toluene (2.2 L) was distilled off at atmospheric pressure. The hot residual toluene solution (˜0.6 L) was diluted with preheated (90° C.) n-heptane (2.5 L). The clear combined solution was left overnight to cool to ambient temperature, with stirring, during which time crystallization occurred. The crystallized product was collected by filtration.

The flask was rinsed with n-heptane (2×0.2 L) and the rinse was run through the product on the filter. The product was washed on the filter with n-heptane/toluene=10:1 v/v mixture (3×0.22 L) and n-heptane (0.2 L), then dried under vacuum (30 mbar) for 2 h at 55° C. to afford O,N-dimethyl-N-[4-(n-propylamino)-6-(prop-2-ynylamino)-[1,3,5]triazin-2-yl]-hydroxylamine (4) (239.2 g, 80%), as a colorless solid. Impurity IMP-A, O,N-dimethyl-N-[4-(n-propylamino)-6-(2-chloroallylamino)-[1,3,5]triazin-2-yl]-hydroxylamine: 0.015 wt % (Method 3M-F)); XRPD as illustrated in FIG. 19.

Following the same procedure as reported elsewhere herein (Example 2F), Stage 3, Method 3 was repeated (lineage includes propargyl amine sulfate with 0.0030% 2-chloroally amine) using 267 g of 6-chloro-N²-(prop-2-ynylamino)-N⁴-n-propylamino-1,3,5-triazine (3), affording 266 g of O,N-dimethyl-N-[4-(n-propylamino)-6-(prop-2-ynylamino)-[1,3,5]triazin-2-yl]-hydroxylamine (4) (90% yield). Impurity IMP-A, (O,N-dimethyl-N-[4-(n-propylamino)-6-(2-chloroallylamino)-[1,3,5]triazin-2-yl]-hydroxylamine): 0.009 wt % (Method 3M-F).

TABLE 7
Elemental analysis of O,N-dimethyl-N-[4-(n-propylamino)-6-(prop-2-
ynylamino)-[1,3,5]triazin-2-yl]-hydroxylamine (4) (Example 2F).

| | C | H | N |
|---|---|---|---|
| Calculated | 52.78 | 7.25 | 33.58 |
| Test 1 | 52.76 | 7.39 | 33.53 |
| Test 2 | 52.81 | 7.40 | 33.58 |

TABLE 8
Yields of O,N-dimethyl-N-[4-(n-propylamino)-6-(prop-2-ynylamino)-
[1,3,5]triazin-2-yl]-hydroxylamine (4).

| Scale Product | | | |
|---|---|---|---|
| Weight (g) | Yield (%) | mp (° C.) | Comment |
| 2.5 g | 85 | — | Stage 3, Method 1 |
| Exampe 1C | | | |
| 105 g | 91 | 76-78 | Stage 3, Method 2 |
| Example 2C | | | |
| 268 g | 80 | 76-78 | Stage 3, Method 3 |
| Example 2F | | | |
| 266 g | 90 | 76-78 | Stage 3, Method 3 |
| Example 2F | | | |

### Example 2G

### Purification of Propargyl Amine as the Hemisulfate Salt (PHS)

To a round-bottom flask (2 L), equipped with an overhead stirrer and a dropping funnel, were charged propargylamine (129 g, 2.342 mol, 2 equiv.) and 96% ethanol (1,500 mL). The mixture was placed in an ice-water bath and cooled for 20 min with stirring. After this time, H₂SO₄ (115 g, 1.171 mol, 1 equiv.) was added dropwise over 20 min. Addition of the sulfuric acid effected the formation of a precipitate and considerable heat release. The reaction mixture was stirred 2 h with the ice-water bath and then 20 h at room temperature. The resultant product was collected by filtration, washed with ethanol (2×200 mL) and dried in vacuo at room temperature to yield propargyl amine hemisulfate (PHS): (228.5 g, 93%) as colorless shiny crystals. Analysis by GC-FID showed 0.0065 wt % 2-chloroallylamine (Method 3M-A).

### Example 2H

### Optional Recrystallization of Propargyl Amine Hemisulfate (PHS)

Propargylamine hemisulfate (PHS) (28 g) was heated in 96% ethanol (730 mL, 26 mL/g) at reflux for 30 min until complete dissolution occurred, and then ethanol was partially distilled off at atmospheric pressure. Crystallization began after distilling approximately 400 mL of ethanol from the mixture. After approximately 500 mL of ethanol weres distilled off, the residual suspension was cooled and left overnight at room temperature. The resultant solid product was collected by filtration, washed with ethanol (2×30 mL) and dried under vacuum (20 mbar) over P₂O₅ at room temperature for 16 h to afford propargylamine hemisulfate (PHS) as a moisture-stable colorless crystalline solid (25.8 g, 92%).

TABLE 9
Removal of 2-chloroallylamine from propargylamine
via hemisulfate salt formation.

| | Wt % 2-chloroallyl amine in | Wt % 2-chloroallyl amine |
|---|---|---|
| | purchased propargylamine | after purification |
| Exp. No. | free base | (wt % vs. free base) |
| i | 0.126 | 0.0037 |
| Example 2G | 0.712 | 0.0065 |
| ii | 0.126 | 0.0030 |
| iii | 0.712 | Unrecryst. <0.0100 |
| iv | | Recryst. <0.0030 |

*Analytical Method 3M-A

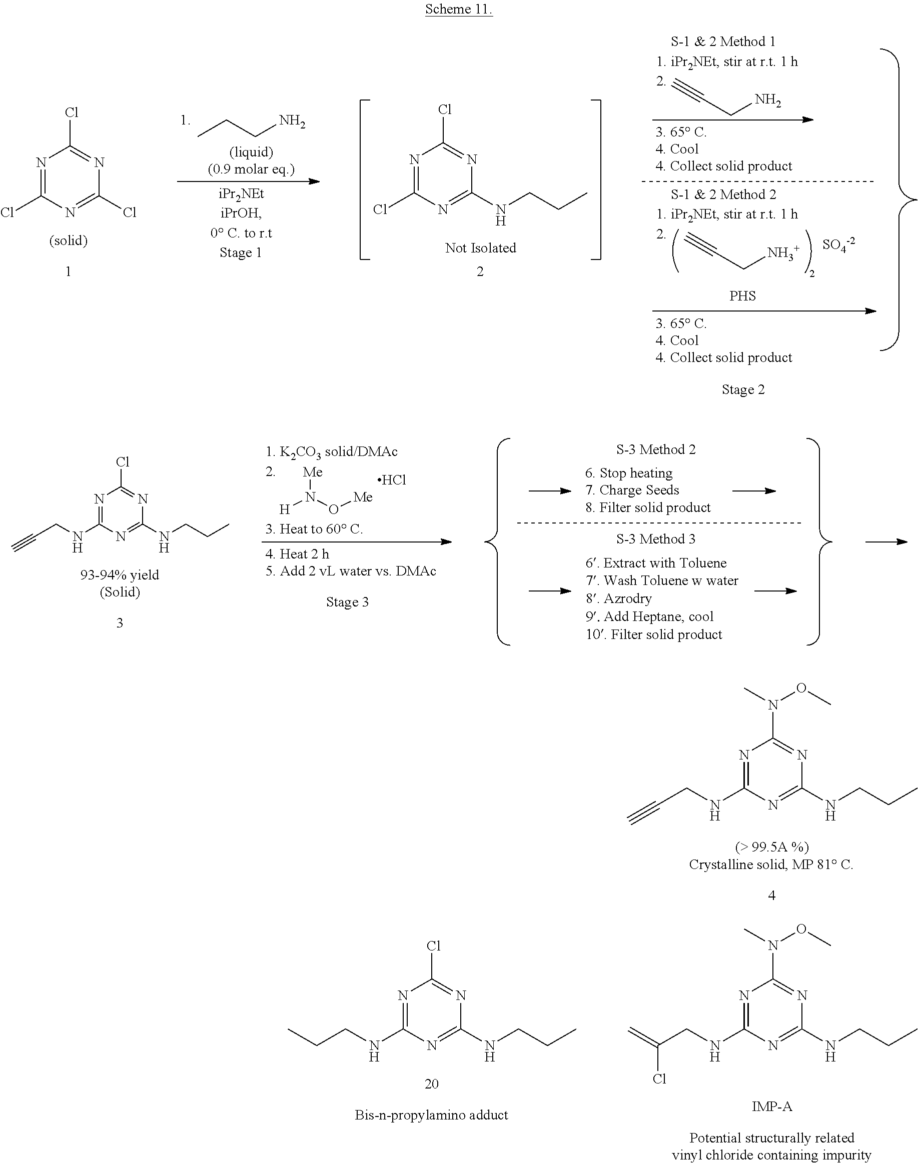

### Example 2I

### Alternative route to O,N-dimethyl-N-[4(n-propylamino)-6-(prop-2-ynylamino-[1,3,5]triazin-2-yl]-hydroxylamine with reduced impurity IMP-A (O,N-dimethyl-N-[4-(n-propylamino)-6-(2-chloroallylamino)-[1,3,5]triazin-2-yl]-hydroxylamine) (Scheme 12)

### Stage A-2: N-(4-Chloro-6-n-propylamino-[1,3,5]triazin-2-yl)-O,N-dimethyl-hydroxylamine (9)

2,4-Dichloro-N-(6-n-propylamino)-[1,3,5]triazine (2) (8.00 g, 38.6 mmol, 1 equiv., Example 1A, Stage 1) and O,N-dimethyhydroxylamine hydrochloride (3.84 g, 39.4 mmol, 1.02 equiv.) were placed in a 250 mL round-bottom flask, equipped with stirrer and septum. Acetonitrile (90 mL) and N,N-diisopropylethylamine (13.0 mL, 78.8 mmol, 2.04 equiv.) were added. The mixture was stirred for 2 h at 45-50° C. and then cooled and volatiles were removed under vacuum. The solid crystalline residue was partitioned between ethyl acetate (80 mL) and an aqueous saturated NaHCO₃ solution (180 mL). The organic layer was additionally washed with an aqueous saturated NaHCO₃ solution (2×80 mL) and then with water (100 mL), and lastly dried over solid anhydrous Na₂SO₄. After filtration the solvent was concentrated under vacuum to afford N-(4-chloro-6-n-propylamino-[1,3,5]triazin-2-yl)-O,N-dimethyl-hydroxylamine (9) (8.87 g, 99%). ¹H NMR (400 MHz, CDCl₃): δ 5.55-5.26 (m, 1H), 3.84-3.73 (m, 3H), 3.46-3.28 (m, 5H), 1.70-1.50 (m, 2H), 1.02-0.91 (m, 3H). HPLC purity: >99%.

### Stage A-3: [4-(N-MethoxyN-methyl-amino)-6-n-propylamino-[1,3,5]triazin-2-yl]-trimethyl-ammonium chloride

N-(4-Chloro-6-n-propylamino-[1,3,5]triazin-2-yl)-O,N-dimethyl-hydroxylamine (9) (5 g, 21.5 mmol, 1 equiv.) was placed in a 100 mL round-bottom flask with stirrer and septum. Dry diethyl ether (50 mL) and dry 1,4-dioxane (10 mL) were added to produce a clear solution. A solution of (CH₃)₃N (33% w/w ethanol, 3.87 g, 21.5 mmol, 1 equiv.) was added via a syringe. The reaction mixture was stirred for 24 at room temperature (21° C.), during which time a precipitate was gradually formed. The solid product was collected by filtration, washed with diethyl ether (3×7 mL) and dried underv vacuum (10 mbar) over P₂O₅ at room temperature to afford [4-(N-methoxy-N-methyl-amino)-6-n-propylamino-[1,3,5]triazin-2-yl]-trimethyl-ammonium chloride (5.80 g, 92%). ¹H NMR (400 MHz, CDCl₃): δ 6.63-5.74 (m, 1H), 3.88-3.73 (m, 12H), 3.48-3.33 (m, 5H), 1.74-1.56 (m, 2H), 1.02-0.91 (m, 3H). HPLC purity: >99%.

### Stage A-4: [4-(N-Methoxy-N-methyl-amino)-6-n-propylamino-[1,3,5]triazin-2-yl]-trimethyl-ammonium tetrafluoroborate

[4-(N-Methoxy-N-methyl-amino)-6-n-propylamino-[1,3,5]triazin-2-yl]-trimethyl-ammonium chloride (5 g, 17.2 mmol, 1 equiv.) was placed in a 100 mL round-bottom flask with stirrer and septum. Water (10 mL) was added to form a clear solution. Separately, a solution of NaBF₄ (1.98 g, 18.1 mmol, 1.05 equiv.) in water (5 mL) was prepared. The solution of NaBF₄ was added to the solution of ammonium chloride at once and the mixture was stirred 10 min. at room temperature. The reaction mixture was cooled with an ice-water bath and filtered to collect the solid. This product was washed on the filter with ice-cold water (4×4 mL) and dried under vacuum at 40° C. to afford [4-(N-methoxy-N-methyl-amino)-6-n-propylamino-[1,3,5]triazin-2-yl]-trimethyl-ammonium tetrafluoroborate (5.17 g, 87%). ¹H NMR (400 MHz, CDCl₃): δ 6.33-5.66 (m, 1H), 3.83-3.77 (m, 3H), 3.57 (s, 3H), 3.54-3.50 (m, 6H), 3.45-3.36 (m, 5H), 1.72-1.56 (m, 2H), 0.98 (t, J=7.2 Hz, 3H). HPLC purity>99%.

TABLE 10
Elemental analysis of [4-(N-methoxy-N-methyl-amino)-6-n-propylamino-
[1,3,5]triazin-2-yl]-trimethyl-ammonium tetrafluoroborate.

| | C | H | N |
|---|---|---|---|
| Calculated | 38.62 | 6.78 | 24.56 |
| Test 1 | 38.56 | 6.59 | 24.39 |
| Test 2 | 38.68 | 6.88 | 24.38 |

### Stage A-5, Method 1: O,N-Dimethyl-N-(4-n-propylamino-6-prop-2-ynylamino-[1,3,5]triazin-2-yl)-hydroxylamine (4) using purified propargyl amine free base

[4-(N-Methoxy-N-methyl-amino)-6-n-propylamino-[1,3,5]triazin-2-yl]-trimethyl-ammonium tetrafluoroborate (800 mg, 2.34 mmol, 1 equiv.) was placed in a 25 mL round-bottom flask with stirrer and septum. Propargylamine (386 mg, 7.01 mmol, 3 equiv.; <100 ppm 2-chloroallylamine (Method 3M-A)) and dimethyl sulfoxide (10 mL) were added. The reaction mixture was stirred 15 h at 45° C.; LC-MS showed a complete conversion.

The mixture was cooled to room temperature and partitioned between brine (40 mL) and toluene (20 mL). The layers were separated and the aqueous brine portion was extracted with toluene (2×20 mL). The combined organic extracts were dried over solid anhydrous Na₂SO₄, filtered and solvent was stripped under vacuum to give an oily residue (635 mg) which by LC-MS contained two major components, compound (4) and IMP-B (O,N-dimethyl-N-[4-(n-propylamino)-6-(dimethylamino)-[1,3,5]triazin-2-yl]-hydroxylamine) in a 9:1 ratio (Method 5E, UV @235 nm). This material was purified by column chromatography on silica using ethyl acetate in petroleum ether 40 (BP 40-60° C.) from 14% to 75% ratio (v/v) as eluent to afford O,N-dimethyl-N-(4-n-propylamino-6-prop-2-ynylamino-[1,3,5]triazin-2-yl)-hydroxylamine (4) (517 mg (88%). ¹H NMR (400 MHz, CDCl₃): δ 5.19-4.84 (m, 2H), 4.27-4.10 (m, 2H), 3.87-3.69 (m, 3H), 3.41-3.17 (m, 5H), 2.22-2.16 (m, 1H), 1.64-1.51 (m, 2H), 0.95 (t, J=7.2 Hz, 3H). HPLC purity: >99%. IMP-B, (O,N-dimethyl-N-[4-(n-propylamino)-6-(dimethylamino)-[1,3,5]triazin-2-yl]-hydroxylamine): undetected (Method 3M-E, UV at 235 nm). IMP-A: <5 ppm by LC-MS/MS (Method 3M-F).

### Stage A-5, Method 2: O,N-Dimethyl-N-(4-n-propylamino-6-prop-2-ynylamino-[1,3,5]triazin-2-yl)-hydroxylamine (4) using purified propargyl amine sulfate

[4-(N-Methoxy-N-methyl-amino)-6-n-propylamino-[1,3,5]triazin-2-yl]-trimethyl-ammonium tetrafluoroborate (800 mg, 2.34 mmol, 1 equiv.) and propargylamine hemisulfate (PHS) (730 mg, 3.51 mmol, 1.5 equiv., 2-chloroallyl amine impurity of 0.0065%) were charged to a 25 mL round-bottom flask equipped with a stirrer magnet and a septum. Dimethyl sulfoxide (10 mL) and N,N-diisopropylethylamine (906 mg, 1.16 mL, 7.01 mmol, 3 equiv.) were added. The mixture was stirred at 45° C. for 18 h, and was then diluted with brine (40 mL). The aqueous layer was separated and washed with toluene (2×20 mL). The combined organic extracts were washed with a brine solution (15 mL), dried over solid anhydrous Na₂SO₄, filtered and evaporated to dryness to afford crude O,N-dimethyl-N-(4-n-propylamino-6-prop-2-ynylamino-[1,3,5]triazin-2-yl)-hydroxylamine (4) (609 mg, 104%), as a resinous solid. UV-HPLC assay (Method 3M, UV @235 nm) shows ratio 4/IMP-B (O,N-dimethyl-N-[4-(n-propylamino)-6-(dimethylamino)-[1,3,5]triazin-2-yl]-hydroxylamine)=90.60:9.40 (Method 5E, UV @235 nm).

TABLE 11
Elemental analysis of O,N-Dimethyl-N-[4-n-propylamino-6-prop-
2-ynylamino-[1,3,5]-triazin-2-yl]-hydroxylamine (4).

| | C | H | N |
|---|---|---|---|
| Calculated | 52.78 | 7.25 | 33.58 |
| Test 1 | 52.81 | 7.28 | 33.73 |
| Test 2 | 52.78 | 7.30 | 33.76 |

An additional sequence of experiments following the same procedure and using purified propargyl amine hemisulfate (PHS) (136.8 mg, <0.0055 wt % 2-chloroallyl amine, Example 2G), and [4-(N-methoxy-N-methyl-amino)-6-n-propylamino-[1,3,5]-triazin-2-yl]-trimethyl-ammonium tetrafluoroborate (150 mg) afforded crude O,N-dimethyl-N-[4-n-propylamino-6-prop-2-ynylamino-[1,3,5]triazin-2-yl)-hydroxylamine (4) (70 mg, 96% yield) as free base with 0.0005 wt % impurity IMP-A (O,N-dimethyl-N-[4-(n-propylamino)-6-(2-chloroallylamino)-[1,3,5]triazin-2-yl]-hydroxylamine) and a 4/IMP-B (O,N-dimethyl-N-[4-(n-propylamino)-6-(dimethylamino)-[1,3,5]triazin-2-yl]-hydroxylamine) in a ratio of 91:9 (Method 5E, UV @235 nm). Recrystallization from toluene and light petroleum ether (BP 40-60° C.) afforded O,N-dimethyl-N-(4-n-propylamino-6-prop-2-ynylamino-[1,3,5]triazin-2-yl)-hydroxylamine (4) (50 mg, 69% yield), with 0.0003 wt % of impurity IMP-A (Method 3M-F) and a 4/IMP-B ratio of 99.70:0.30 (Method 3M-E, UV @235 nm).

### Stage A-5, Method 3: O,N-Dimethyl-N-(4-n-propylamino-6-prop-2-ynylamino-[1,3,5]triazin-2-yl)-hydroxylamine (4) Using Neat Purified Propargyl Amine as Solvent

In a 5 mL pressure tube with a magnet stir bar, [4-(N-methoxy-N-methyl-amino)-6-n-propylamino-[1,3,5]triazin-2-yl]-trimethyl-ammonium tetrafluoroborate (11) (50 mg, 0.146 mmol, 1 equiv.) was weighed. Purified propargylamine (0.9 mL, 774 mg, 14 mmol, 96 equiv.) was added. The tube was sealed and stirred at 45° C. for 2 h. The reaction mixture was cooled to room temperature. Analysis of an aliquot the reaction mixture by LC-MS showed no residual tetrafluoroborate starting material and conversion to O,N-dimethyl-N-(4-n-propylamino-6-prop-2-ynylamino-[1,3,5]triazin-2-yl)-hydroxylamine (4) with 4:IMP-A (O,N-dimethyl-N-[4-(n-propylamino)-6-(2-chloroallylamino)-[1,3,5]triazin-2-yl]-hydroxylamine) in a 96.65:3.35 ratio (Method 3M-E, UV @235 nm).

### Stage A-6: Purification of crude O,N-dimethyl-N-(4-n-propylamino-6-prop-2-ynylamino-[1,3,5]triazin-2-yl)-hydroxylamine (4) from Stage A-5 by recrystallization

Crude O,N-dimethyl-N-(4-n-propylamino-6-prop-2-ynylamino-[1,3,5]triazin-2-yl)-hydroxylamine (4) (609 mg) (PE-570) was dissolved in toluene (1.2 mL) at reflux. Light petroleum ether (bp 40-60° C., 6 mL) was added to the hot solution and the mixture was left to cool to ambient temperature with stirring. The precipitated product was collected by filtration and washed on filter with light petroleum ether to afford O,N-dimethyl-N-(4-n-propylamino-6-prop-2-ynylamino-[1,3,5]triazin-2-yl)-hydroxylamine (4) (419 mg, 71%) as light yellow crystals with a Compound 4/IMP-B (O,N-dimethyl-N-[4-(n-propylamino)-6-(dimethylamino)-[1,3,5]triazin-2-yl}-hydroxylamine) ratio of 99.82:0.18 (Method 3M-E, UV @235 nm); Impurity IMP-A (O,N-dimethyl-N-[4-(n-propylamino)-6-(2-chloroallylamino)-[1,3,5]triazin-2-yl]-hydroxylamine) by LC-MS/MS (Method 3M-F): 0.002 wt %.

### Example 2J

### Stage A-7 Method 1; Purification of O,N-dimethyl-N-(4-n-propylamino-6-prop-2-ynylamino-[1,3,5]triazin-2-yl)-hydroxylamine (4) as L(+) hydrogen tartrate salt (5c)

Following the procedure in Example 3F, 15 mg of O,N-dimethyl-N-[4-n-propylamino-6-prop-2-ynylamino-[1,3,5]triazin-2-yl-hydroxylamine (4) was treated with 9.1 mg L-(+)-tartaric acid in isopropanol to afford O,N-dimethyl-N-[4-n-propylamino-6-prop-2-ynylamino-[1,3,5]triazin-2-yl]-hydroxylamine monohydrogen-L-(+)-tartrate (5c) (15 mg, 62%) with <0.0003 wt % of O,N-dimethyl-N-[4-(n-propylamino)-6-(2-chloroallylamino)-[1,3,5]triazin-2-yl]-hydroxylamine (IMP-A) (Method 3M-F) and O,N-dimethyl-N-[4-(n-propylamino)-6-(dimethylamino)-[1,3,5]triazin-2-yl]-hydroxylamine (IMP-B) undetected (Method 5E, UV @235 nm).

TABLE 12
Elemental analysis of O,N-dimethyl-N-(4-n-propylamino-
6-prop-2-ynylamino-[1,3,5]triazin-2-yl)-hydroxylamine
as hydrogen-L(+)-tartrate salt (5c).

| | C | H | N |
|---|---|---|---|
| Calculated | 45.00 | 6.04 | 20.99 |
| Test 1 | 45.00 | 6.02 | 20.90 |
| Test 2 | 45.00 | 6.03 | 20.93 |

### Example 2K

### Stage A-7, Method 2; Purification of O,N-Dimethyl-N-(4-n-propylamino-6-prop-2-ynylamino-[1,3,5]triazin-2-yl)-hydroxylamine as L(+) hydrogen maleinate salt (5d)

Following the procedure in Example 3G, 15.1 mg of O,N-dimethyl-N-(4-n-propylamino-6-prop-2-ynylamino-[1,3,5]triazin-2-yl)-hydroxylamine (4) was treated with 7 mg maleic acid in methyl ethyl ketone to afford O,N-dimethyl-N-(4-n-propylamino-6-prop-2-ynylamino-[1,3,5]triazin-2-yl)-hydroxylamine monohydrogen hydrogen maleinate (17 mg, 77% yield) with <0.0003 wt % of O,N-dimethyl-N-[4-(n-propylamino)-6-(2-chloroallylamino)-[1,3,5]triazin-2-yl]-hydroxylamine (IMP-A) (Method 3M-F) and O,N-dimethyl-N-[4-(n-propylamino)-6-(dimethylamino)-[1,3,5]triazin-2-yl]-hydroxylamine (IMP-B): Undectected (Method 5E, UV @235 nm).

TABLE 13
Elemental analysis of O,N-dimethyl-N-(4-n-propylamino-
6-prop-2-ynylamino-[1,3,5]triazin-2-yl)-hydroxylamine
as hydrogen-maleinate salt (5d) (JK-630).

| | C | H | N |
|---|---|---|---|
| Calculated | 49.17 | 6.05 | 22.94 |
| Test 1 | 49.29 | 5.93 | 23.13 |
| Test 2 | 49.24 | 5.88 | 23.03 |

TABLE 14
Control of Impurity IMP-A (O,N-dimethyl-N-[4-(n-propylamino)-6-(2-
chloroallylamino)-[1,3,5]triazin-2-yl]-hydroxylamine).

| | Propargyl | | | |
|---|---|---|---|---|
| | Amine Sulfate | Resulting | Resulting | |
| | (PHS) + | Free | Tartrate | Resulting |
| | Wt. % 2- | Base (4) | (5c) | Maleinate |
| Synthesis | chloroallyl | Wt. % | Wt. % | (5d) |
| Method | amine* | Imp. A* | Imp A** | Imp A** |
| Ex 2G | 0.0065% | 0.015% | 0.0070% | 0.0070 |
| As per Ex. 2B | | | | |
| As per E. 2F | | | | |
| Ex. 3F Method 1 | | | | |
| As per Ex 3G, | | | | |
| Method 1 | | | | |
| As per Ex. 2G | 0.0030% | 0.0090% | 0.0030% | Not made |
| As per Ex. 2B | | | | |
| As per Ex. 2F | | | | |
| As per Ex. | | | | |
| 3F, Method 1 ( ) | | | | |
| As per Ex 2G | (not measured) | 0.0020% | Not made | Not made |
| As per Ex. 2I, | | | | |
| Stage A-5, | | | | |
| Method 2 | | | | |
| Ex 2I, Stage A-6 | | | | |
| As per Ex. 2G | <0.0055% | 0.0003% | <0.0003% | <0.0003% |
| As per Ex. 2I | | | | |
| Stage A-5, | | | | |
| Method 2 | | | | |
| As per Ex. 2I, | | | | |
| Stage A-6 | | | | |
| Ex. 2JEx. 2K | | | | |

*Analytical Method 3M-A
**Analytical Method 3M-F

### Example 3

### O,N-Dimethyl-N-[4(n-propylamino)-6-(prop-2-ynylamino)-[1,3,5]triazin-2-yl]-hydroxylamine salts

### Example 3A

### O,N-Dimethyl-N-[4-(n-propylamino)-6-(prop-2-ynylamino)-[1,3,5]triazin-2-yl]-hydroxylamine hydrochloride (5a)

A 2M HCl/diethyl ether solution (0.47 mL, 0.94 mmol) was added to the solution of O,N-dimethyl-N-[4-(n-propylamino)-6-(prop-2-ynylamino)-[[1,3,5]triazin-2-yl]-hydroxylamine (4) (220 mg, 0.88 mmol) in diethyl ether (15 mL) at 0° C. The mixture was stirred for 0.5 h at 0° C., and then the volatiles were removed under reduced pressure to yield the hydrochloride salt (5a) in quantitative yield. 400 MHz 1H NMR (dimethyl sulfoxide-d6, ppm): δ 13.0-12.0 (1H, m), 8.92-8.39 (2H, m), 4.24-4.06 (2H, m), 3.80-3.75 (3H, m), 3.55-3.16 (6H, m, overlapped with water), 1.61-1.46 (2H, m), 0.95-0.84 (3H, m). ESI-MS (m/z): 251 [M+H]+.

### Example 3B

### Method 1; O,N-Dimethyl-N-[4-(n-propylamino)-6-(prop-2-ynylamino)-[1,3,5]triazin-2-yl]-hydroxylamine hydrogen sulfate (5b)

To a solution of O,N-dimethyl-N-(4-n-propylamino-6-prop-2-ynylamino-[1,3,5]triazin-2-yl)-hydroxylamine (4) (1.92 g, 7.68 mmol) in diethyl ether (40 mL) at 0° C., was added 95% H₂SO₄ (0.41 mL, 7.68 mmol) in a dropwise manner. The mixture was stirred for 0.5 h at 0° C., then the volatiles were removed under reduced pressure. The residue was crystallized from a mixture of ethanol and diethyl ether to yield O,N-dimethyl-N-[(4-(n-propylamino)-6-(prop-2-ynylamino)-[1,3,5]triazin-2-yl]-hydroxylamine hydrogen sulfate (5b) (2.46 g, 92%). 400 MHz ¹H NMR (DMSO-d₆, ppm): δ 12.4-11.0 (1H, br s), 9.03-8.41 (1H, m), 8.22-7.43 (1H, m), 4.23-4.06 (2H, m), 3.84-3.72 (3H, m), 3.44-3.12 (6H, m), 1.66-1.45 (2H, m), 0.98-0.79 (3H, m). ESI-MS (m/z): 251 [M+H]+. Melting point: 144-147° C.

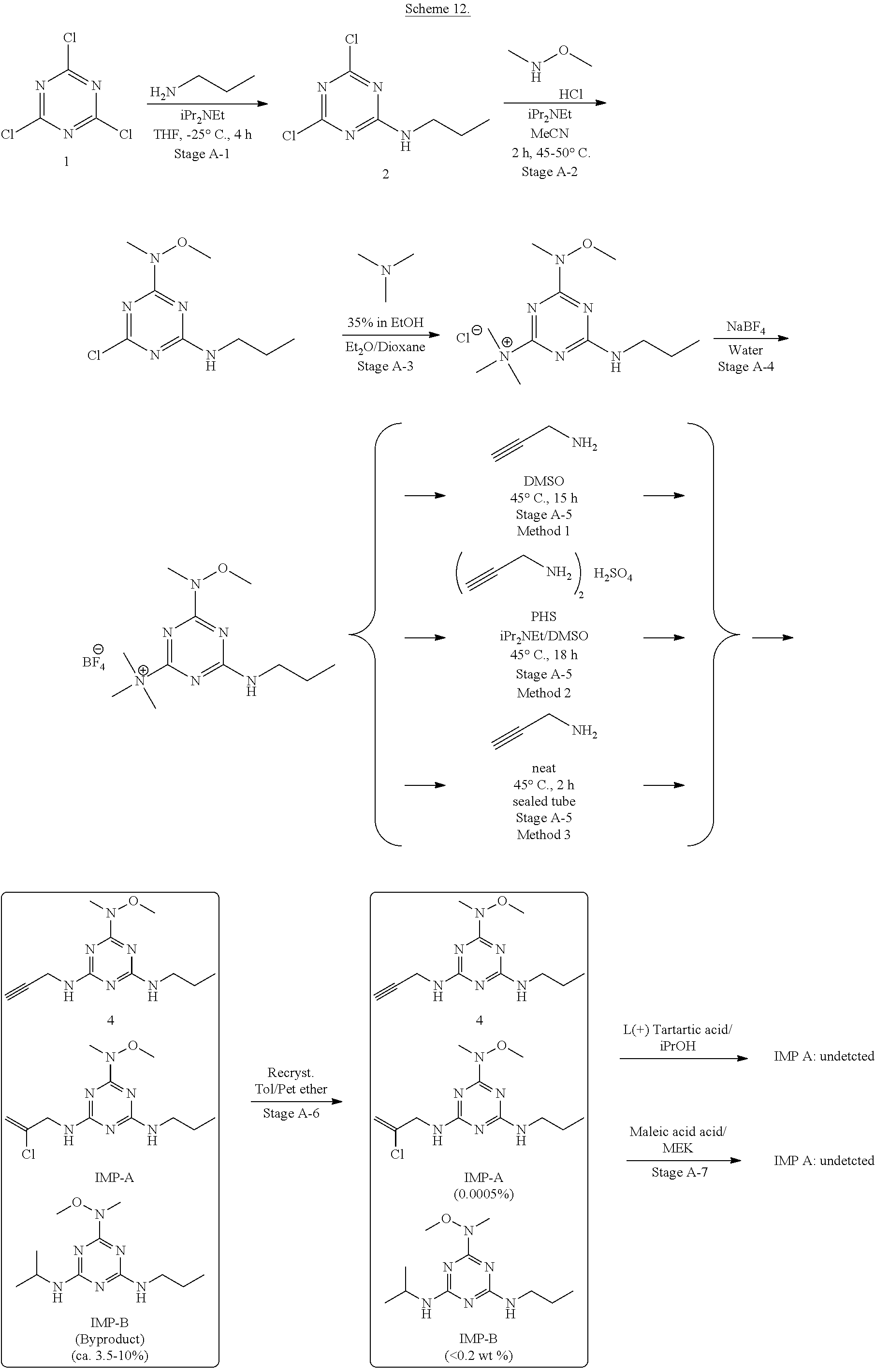

### Example 3C

### Method 2; O,N-Dimethyl-N-[4-(n-propylamino)-6-(prop-2-ynylamino)-[1,3,5]triazin-2-yl]-hydroxylamine hydrogen sulfate (5b)

O,N-Dimethyl-N-[4-(n-propylamino)-6-(prop-2-ynylamino)-[1,3,5]triazin-2-yl]-hydroxylamine free base (4) (25 g, 0.102 mol, 1 equiv.) was dissolved in methyl ethyl ketone (180 mL) at 50° C. and insoluble material was filtered off. The hot filtrate was placed in a 500 mL round bottom flask and 95% H₂SO₄ (5.7 mL, 0.107 mol, 1.05 equiv.) was added dropwise with stirring at 50° C. The salt started crystallizing upon addition of the final drops of sulfuric acid. The mixture was allowed to cool, and was stirred for 16 h at ambient temperature. The solid was collected by filtration, washed with methyl ethyl ketone (2×30 mL) and air dried at 75° C. for 3 days yielding O,N-dimethyl-N-[4-(n-propylamino)-6-(prop-2-ynylamino)-[1,3,5]-triazin-2-yl]-hydroxylamine hydrogen sulfate (5b) (32.5 g 91%) as colorless solid. ESI-MS (m/z): 251 [M+H]+. XRPD as illustrated in FIG. 22.

TABLE 15
Elemental analysis of O,N-dimethyl-N-[4-(n-propylamino)-6-
(prop-2-ynylamino)-[1,3,5]triazin-2-yl]-hydroxylamine
hydrogen sulfate (5b) obtained from methyl ethyl ketone (Example 3C).

| | C | H | N |
|---|---|---|---|
| Calculated | 37.82 | 5.78 | 24.06 |
| Test 1 | 37.84 | 5.85 | 24.08 |
| Test 2 | 37.80 | 5.84 | 24.02 |

TABLE 16
¹H NMR analysis for O,N-dimethyl-N-(4-n-propylamino-6-prop-2-ynyl-
amino-[1,3,5]triazin-2-yl)-hydroxylamine hydrogen sulfate (5b). 400
MHz; D₂O; 10 mg/mL; Number of scans: 32 (FIG. 20).

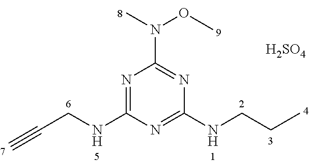

| Delta | Peak | | J | |
|---|---|---|---|---|
| (ppm) | description | Integration | (Hz) | Assignment |
| 4.79 | s | — | — | Water + 1 + 5 + H₂SO₄ |
| 4.31-4.11 | m | 2H | — | 2 |
| 3.80 | s | 3H | — | 9 |
| 3.49-3.25 | m | 5H | — | 6 + 8 |
| 2.65 | s | 1H | — | 7 |
| 1.68-1.48 | m | 2H | — | 3 |
| 0.90 | t | 3H | 7.41 | 4 |

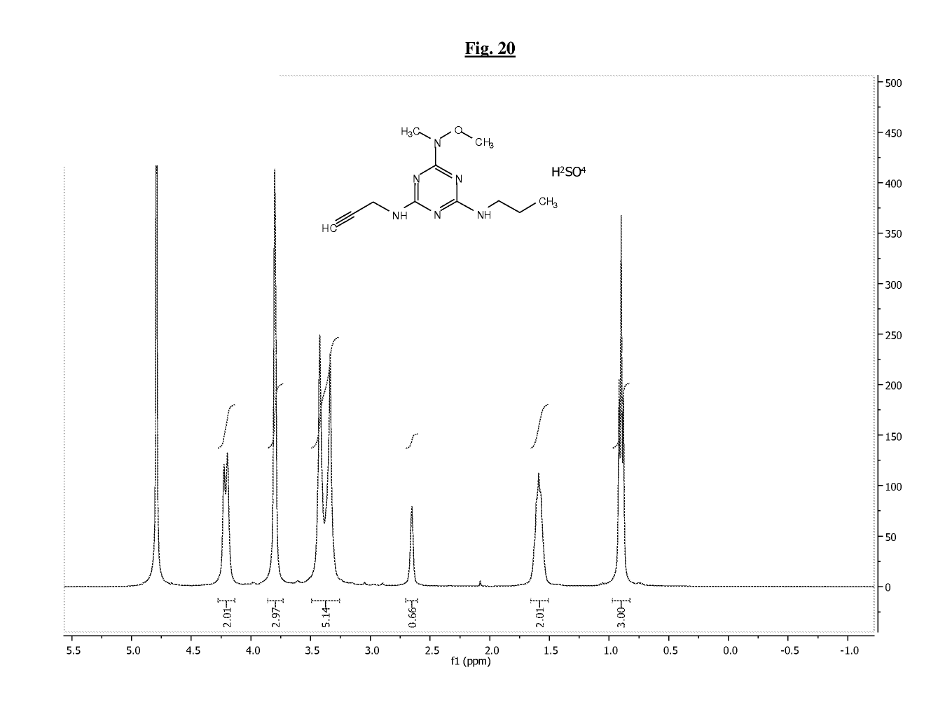

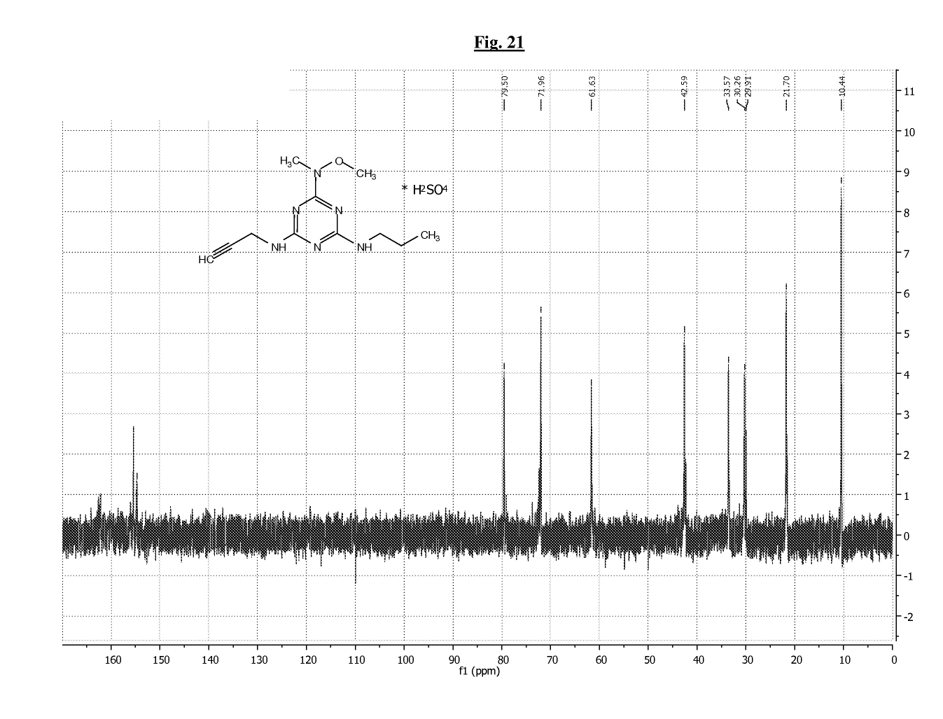

TABLE 17
¹³C NMR analysis of O,N-dimethyl-N-(4-n-propylamino-6-prop-2-ynyl
amino-[1,3,5]triazin-2-yl)-hydroxylamine hydrogensulfate (5b) (decoupled).
100 MHz; D₂O; 20 mg/mL; Number of scans: 600 (FIG. 21).

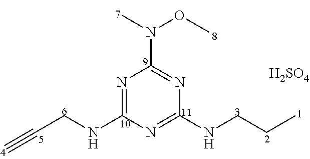

Delta
(ppm)
155.38
154.71
79.50
71.96
61.63
42.59
33.57
30.26
29.91
21.70
10.44

TABLE 18
O,N-Dimethyl-N-(4-n-propylamino-6-prop-2-ynylamino-[1,3,5]triazin-
2-yl)-hydroxylamine hydrogen sulfate (5b) diffraction signals.

| Pos. [°2Th.] | d-spacing [Å] | Rel. Int. [%] |
|---|---|---|
| 4.5472 | 19.41690 | 76.12 |
| 9.1582 | 9.64862 | 100.00 |
| 13.7830 | 6.41972 | 11.31 |
| 16.2515 | 5.44973 | 1.47 |
| 18.5468 | 4.78013 | 10.35 |
| 19.9222 | 4.45312 | 13.05 |
| 21.1920 | 4.18907 | 11.97 |
| 21.9983 | 4.03733 | 12.98 |
| 23.4378 | 3.79252 | 17.48 |
| 24.1682 | 3.67953 | 9.02 |
| 28.0717 | 3.17611 | 5.68 |

TABLE 19
Yields of O,N-dimethyl-N-[4-(n-propylamino)-6-(prop-2-ynylamino)-
[1,3,5]triazin-2-yl]-hydroxylamine hydrogen sulfate (5b).

| Scale (g) | Yield (%) | mp (° C.) | Comment |
|---|---|---|---|
| 44, free base | 99 | 132-134 | From free base |
| 25, free base | 91 | 136-138 | From free base in MEK |
| Example 3C | | | |
| 105, free base | 90 | 132-134 | From free base in MEK |

### Example 3D

### Recrystallizations of O,N-dimethyl-N-[4-(n-propylamino)-6-(prop-2-ynylamino)-[1,3,5]triazin-2-yl]-hydroxylamine hydrogen sulfate (5b) from various solvents

### Example 3D, Method 1

### Recrystallization of O,N-dimethyl-N-[4-(n-propylamino)-6-(prop-2-ynylamino)-[1,3,5]triazin-2-yl]-hydroxylamine hydrogen sulfate (5b) from isopropanol admixed with diethyl ether

O,N-Dimethyl-N-[4-(n-propylamino)-6-(prop-2-ynylamino)-[[1,3,5]triazin-2-yl]-hydroxylamine hydrogen sulfate (5b) (5 g) was dissolved in isopropanol (20 mL) at reflux and then cooled ambient temperature. Diethyl ether (3 mL) was added in order to initiate crystallization and wool-like voluminous crystals formed. The crystallization process was allowed to proceed for 16 h at ambient temperature. The resultant product was collected by filtration, washed with isopropanol (2×20 mL), then with light petroleum ether (2×25 mL) and air dried at 65° C. for 16 h yielding O,N-dimethyl-N-[4-(n-propylamino)-6-(prop-2-ynylamino)-[1,3,5]triazin-2-yl]-hydroxylamine hydrogen sulfate (5b) as colorless crystals (2.5 g, 50%).

### Example 3D, Method 2

### Recrystallization of O,N-dimethyl-N-[4-(n-propylamino)-6-(prop-2-ynylamino)-[1,3,5]triazin-2-yl]-hydroxylamine hydrogen sulfate (5b) from acetonitrile admixed with diethyl ether

A 5 g sample of (5b) was recrystallized from acetonitrile admixed with diethyl ether following the procedure used for Method 2, affording O,N-dimethyl-N-[4-(n-propylamino)-6-(prop-2-ynylamino)-[1,3,5]triazin-2-yl]-hydroxylamine hydrogen sulfate (5b) (2.3 g, 46%) as colorless crystals.

TABLE 20
Elemental analysis of O,N-dimethyl-N-[4-(n-propylamino)-
6-(prop-2-ynylamino)-[1,3,5]triazin-2-yl]-hydroxylamine
hydrogen sulfate (5b) as obtained from isopropanol admixed
with diethyl ether (Example 3D, Method 1).

| | C | H | N |
|---|---|---|---|
| Calculated | 37.92 | 5.79 | 24.12 |
| Test 1 | 38.04 | 5.80 | 24.28 |
| Test 2 | 37.98 | 5.70 | 24.25 |

TABLE 21
Elemental analysis of O,N-dimethyl-N-[4-(n-propylamino)-
6-(prop-2-ynylamino)-[1,3,5]triazin-2-yl]-hydroxylamine
hydrogen sulfate (5b) as obtained from acetonitrile admixed
with diethyl ether (Example 3D, Method 2).

| | C | H | N |
|---|---|---|---|
| Calculated | 37.92 | 5.79 | 24.12 |
| Test 1 | 38.04 | 5.58 | 24.08 |
| Test 2 | 38.01 | 5.61 | 24.04 |

### Example 3D, Method 3

### Recrystallization of O,N-dimethyl-N-[4-(n-propylamino)-6-(prop-2-ynylamino)-[1,3,5]triazin-2-yl]-hydroxylamine hydrogen sulfate (5b) from acetone

O,N-Dimethyl-N-[4-(n-propylamino)-6-(prop-2-ynylamino)-[[1,3,5]triazin-2-yl]-hydroxylamine hydrogen sulfate (5b) (5 g) was dissolved in acetone (60 mL) at reflux and then cooled to ambient temperature. The mixture was allowed to undergo recrystallization for 16 h at ambient temperature with partial evaporation of the solvent to the final volume of 40 mL. After this time, wool-like voluminous crystals were formed. The product was collected by filtration, washed with acetone (2×20 mL), then with light petroleum ether (BP 40-60° C.) (2×40 mL) and lastly, air dried at 70° C. for 16 h yielding O,N-dimethyl-N-[4-(n-propylamino)-6-(prop-2-ynylamino)-[1,3,5]triazin-2-yl]-hydroxylamine hydrogen sulfate (5b) as colorless crystals (2.1 g, 42%).

TABLE 22
Elemental analysis of O,N-dimethyl-N-[4-(n-propylamino)-6-
(prop-2-ynylamino)-[1,3,5]triazin-2-yl]-hydroxylamine
hydrogen sulfate (5b) obtained from acetone (Example 3D, Method 3).

| | C | H | N |
|---|---|---|---|
| Calculated | 37.92 | 5.79 | 24.12 |
| Test 1 | 38.00 | 5.71 | 24.19 |
| Test 2 | 37.97 | 5.74 | 24.17 |

### Example 3E

### Formation of O,N-dimethyl-N-[4-(n-propylamino)-6-(prop-2-ynyl amino)-[1,3,5]triazin-2-yl]-hydroxylamine sulfuric acid addition salts

### Example 3E-1

### 2:1 Mole/Mole Free Base:Acid (Fraction 1 of 3)

O,N-Dimethyl-N-[4-(n-propylamino)-6-(prop-2-ynylamino)-[1,3,5]triazin-2-yl]-hydroxylamine (4) (43.5 g) was dissolved in diethyl ether (800 mL), and cooled to 0° C. (ice bath). To this solution concentrated 95% H₂SO₄ (1 equiv., 9.3 mL) was added dropwise with stirring. The reaction mixture was stirred for 1 h, after which time the resultant solid product was collected by filtration and washed with diethyl ether to afford a product that was determined by elemental analysis to be a sulfate adduct with a 2:1 molar ratio of free base to H₂SO₄ (18.4 g, mp 102-104° C.). XRPD as illustrated in FIG. 23.

TABLE 23
Elemental analysis of O,N-dimethyl-N-[4-(n-propylamino)-6-(prop-2-
ynylamino)-[1,3,5]triazin-2-yl]-hydroxylamine sulfuric acid addition
salt in a 2:1 molar ratio (C₁₁H₁₈N₆O * 0.5 H₂SO₄) (Example 3E-1).

| | C | H | N |
|---|---|---|---|
| Calculated | 44.14 | 6.40 | 28.08 |
| Test 1 | 44.00 | 6.30 | 28.05 |
| Test 2 | 44.09 | 6.29 | 28.07 |

### Example 3E-2

### 1:2 Mole/Mole Free Base:Acid (Fraction 2 of 3)

The oily residue remaining in the flask from Example 3E-1 above was suspended in diethyl ether (200 mL), admixed with ethanol (30 mL), and sonicated for 1 h at ambient temperature. The resulting solids were collected by filtration and washed with Et₂O and lastly air dried at 60° C. to afford a product that was determined by elemental analysis to be a bis-sulfate with a 1:2 molar ratio of free base to H₂SO₄ (21.4 g, mp 165-167° C.). XRPD as illustrated in FIG. 24.

TABLE 24
Elemental analysis of O,N-dimethyl-N-[4-(n-propylamino)-6-(prop-2-
ynylamino)-[1,3,5]triazin-2-yl]-hydroxylamine sulfuric acid addition
salt in a 1:2 molar ratio (C₁₁H₁₈N₆O * 2 H₂SO₄) (Example 3E-2).

| | C | H | N |
|---|---|---|---|
| Calculated | 29.59 | 4.97 | 18.82 |
| Test 1 | 29.64 | 4.78 | 18.61 |
| Test 2 | 29.81 | 4.81 | 18.72 |

### Example 3E-3

### 4:3 Mole/Mole Free Base:Acid (Fraction 3 of 3)

The combined filtrates from example 3E-2 were evaporated to dryness and dried under vacuum (0.2 mbar) to afford a product that was determined by elemental analysis to be a sulfuric acid addition salt with molar 4:3 ratio of free base to H₂SO₄ (21.5 g, mp 53-57° C.). XRPD data (FIG. 11) suggest this substance is largely amorphous. XRPD as illustrated in FIG. 25.

TABLE 25
Elemental analysis of O,N-dimethyl-N-[4-(n-propylamino)-6-(prop-2-
ynylamino)-[1,3,5]triazin-2-yl]-hydroxylamine sulfuric acid addition
salt in a 4:3 molar ratio (4 C₁₁H₁₈N₆O *3 H₂SO₄) (Example 3E-3).

| | C | H | N |
|---|---|---|---|
| Calculated | 41.04 | 6.09 | 26.11 |
| Test 1 | 41.02 | 6.08 | 25.65 |
| Test 2 | 41.37 | 6.16 | 25.74 |

### Example 3E-4

### Conversion of 2:1, 1:2 and 4:3 Free Base:Acid Salts to 1:1 Free Base:Acid Salts

Fractions 1, 2 and 3 from 3E-1, 3E-2, and 3E-3 were combined, dissolved in ethanol (350 mL) and sonicated for 1 h to ensure complete dissolution. The solvent was removed under vacuum and the resultant semisolid residue was dried under vacuum (0.2 mbar) at ambient temperature for 3 h. The solidified residue was then air dried at 60° C. for 16 h to afford a product that was determined by elemental analysis to be an addition salt with a 1:1 molar ratio of free base:H₂SO₄. Yield: 60.5 g (99%), mp 130-132° C. XRPD as illustrated in FIG. 22.

TABLE 26
Elemental analysis for O,N-dimethyl-N-[4-(n-propylamino)-6-(prop-2-
ynylamino)-[1,3,5]triazin-2-yl]-hydroxylamine sulfuric acid addition
salt (5b), 1:1 molar ratio (C₁₁H₁₈N₆O * H₂SO₄) (Example 3E-4).

| | C | H | N |
|---|---|---|---|
| Calculated | 38.25 | 5.82 | 24.33 |
| Found 1 | 38.22 | 5.65 | 24.23 |
| Found 2 | 38.32 | 5.64 | 24.30 |

### Example 3F

### Preparation of O,N-dimethyl-N-[4-(n-propylamino)-6-(prop-2-ynylamino)-[1,3,5]triazin-2-yl]-hydroxylamine as hydrogen-L(+)-tartrate salt (5c)

### Example 3F, Method 1

### Preparation of O,N-dimethyl-N-[4-(n-propylamino)-6-(prop-2-ynylamino)-[1,3,5]triazin-2-yl]-hydroxylamine as hydrogen-L(+)-tartrate Salt (5c) from isopropanol

O,N-Dimethyl-N-[4-(n-propylamino)-6-(prop-2-ynylamino)-[1,3,5]triazin-2-yl]-hydroxylamine (4) (200 g, 0.796 mol, 1 equiv.) was dissolved in isopropanol (550 mL) with stirring and gentle heating (40-50° C. in solution). Insolubles were filtered off. L(+)-Tartaric acid (118.6 g, 0.796 mol, 1 equiv.) was suspended in isopropanol (850 mL) and heated to reflux for 15 min, at which point complete dissolution was achieved. The solution of O,N-dimethyl-N-[4-(n-propylamino)-6-(prop-2-ynylamino)-[1,3,5]triazin-2-yl]-hydroxylamine (4) in isopropanol (room temperature) was added to the hot solution of L(+)-tartaric acid in isopropanol (60° C.). Additional isopropanol (600 mL) was used to rinse flasks and filter, and the rinse was added to the bulk solution. The resulting mixture was brought to reflux and left to cool to ambient temperature (23° C.) without stirring for 16 h. To the clear yellow solution a small portion of seed crystals was added and the mixture was briefly stirred. Crystallization started immediately. After 6 h at room temperature the solid product was collected by filtration, washed with isopropanol (2×900 mL), and with light petroleum ether (bp 40-60° C.) (1,000 mL). The salt was air dried at 50° C. for 66 h to yield O,N-dimethyl-N-[4-(n-propylamino)-6-(prop-2-ynylamino-[1,3,5]triazin-2-yl]-hydroxylamine L(+) hydrogen tartrate salt (271 g, 85%). mp 127-128° C. Impurity IMP-A: 0.007 wt % (Method 3M-F) XRPD as illustrated in FIG. 27.

Following the same procedure as illustrated in Example 3F, Method 1; Example 3E, Method 1 was repeated using 230 g O,N-dimethyl-N-[4-(n-propylamino)-6-(prop-2-ynylamino)-[1,3,5]triazin-2-yl]-hydroxylamine (4) affording 295 g of O,N-dimethyl-N-[4-(n-propylamino)-6-(prop-2-ynylamino)-[1,3,5]triazin-2-yl]-hydroxylamine L(+) hydrogen tartrate (5c) (80% yield). Impurity IMP-A: 0.003 wt % (Method 3M-F).

TABLE 27
Elemental analysis for O,N-dimethyl-N-[4-(n-propylamino)-6-(prop-
2-ynylamino)-[1,3,5]triazin-2-yl]-hydroxylamine L(+) hydrogen
tartrate salt (5c) obtained from isopropanol (Example 3F, Method 1).

| | C | H | N |
|---|---|---|---|
| Calculated | 45.00 | 6.04 | 20.99 |
| Found 1 | 45.20 | 6.04 | 20.93 |
| Found 2 | 45.29 | 6.04 | 20.95 |

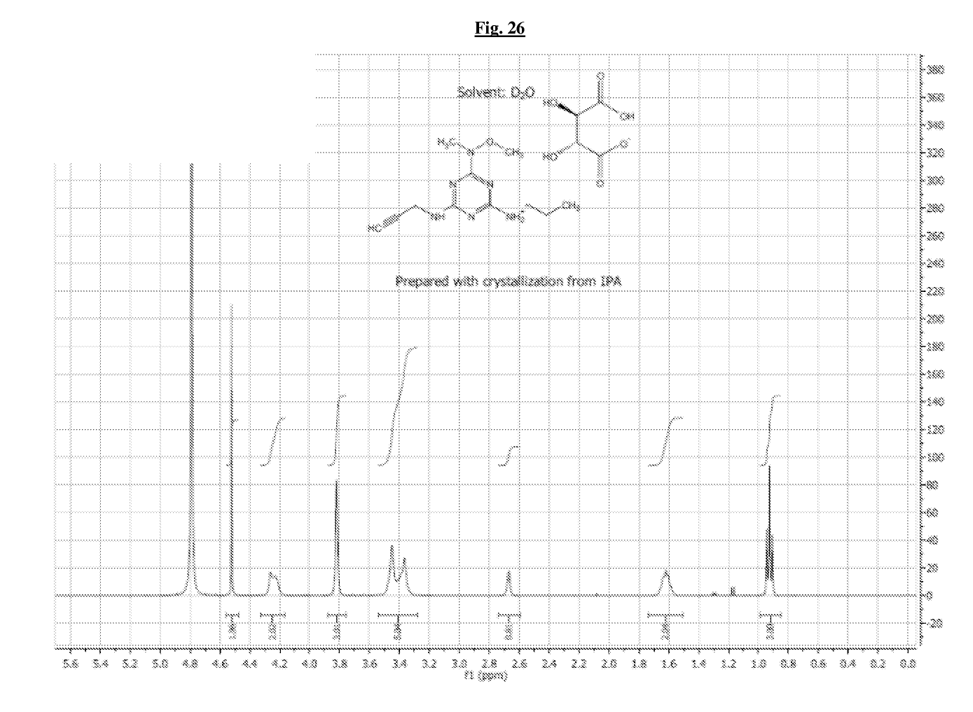

TABLE 28
¹H NMR analysis O,N-dimethyl-N-(4-n-propylamino-6-prop-2-
ynylamino-[1,3,5]triazin-2-yl)-hydroxylamine L(+) hydrogen tartrate.
400 MHz; DMSO-d₆; 10 mg/mL; Number of scans: 32 (FIG. 26).

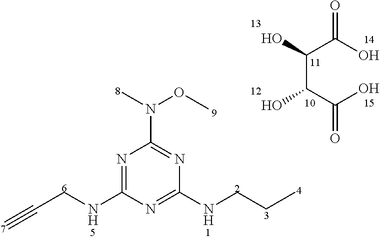 [SMILES: C#CCNc1nc(NCCC)nc(N(C)OC)n1 ; O=C(O)[C@H](O)[C@@H](O)C(=O)O]

| Delta | Peak | | J | |
|---|---|---|---|---|
| (ppm) | description | Integration | (Hz) | Assignment |
| 7.30-6.70 | m | 2H | — | 1 + 5 |
| 4.30 | s | 2H | — | 6 |
| 4.01-3.95 | m | 3H | — | 9 |
| 3.70-3.59 | m | 6H | — | 10 + 11 + 12 + 13 + 14 + 15 |
| 3.24-3.08 | m | 5H | — | 2 + 8 |
| 3.00-2.94 | m | 1H | — | 7 |
| 1.56-1.39 | m | 2H | — | 3 |
| 0.84 | t | 3H | 7.40 | 4 |

TABLE 29
O,N-Dimethyl-N-(4-n-propylamino-6-prop-2-ynylamino-[1,3,5]triazin-
2-yl)-hydroxylamine hydrogen-L(+)-tartrate (5c) diffraction dignals.

| Degrees 2θ | d space (Å) | Intensity (%) |
|---|---|---|
| 6.02 ± 0.20 | 14.687 ± 0.504 | 19 |
| 9.51 ± 0.20 | 9.300 ± 0.199 | 100 |
| 13.45 ± 0.20 | 6.581 ± 0.099 | 14 |
| 15.39 ± 0.20 | 5.757 ± 0.075 | 16 |
| 18.18 ± 0.20 | 4.879 ± 0.054 | 16 |
| 19.69 ± 0.20 | 4.509 ± 0.046 | 16 |
| 21.06 ± 0.20 | 4.219 ± 0.040 | 25 |
| 23.15 ± 0.20 | 3.843 ± 0.033 | 38 |

### Example 3F, Method 2

### Preparation of O,N-dimethyl-N-[4-(n-propylamino)-6-(prop-2-ynylamino)-[1,3,5]triazin-2-yl]-hydroxylamine as L(+) hydrogen tartrate salt (5c) obtained from ethyl acetate

O,N-Dimethyl-N-[4-(n-propylamino)-6-(prop-2-ynylamino)-[1,3,5]triazin-2-yl]-hydroxylamine (4) (1 g, 4 mmol, 1 equiv.) was dissolved in ethyl acetate (10 mL) with stirring. L(+)-Tartaric acid (0.6 g, 4 mmol, 1 equiv.) was added and mixture was stirred at room temperature for 18 h. The acid dissolved immediately. Product was collected by filtration, and washed with ethyl acetate (3×5 mL), and dried under vacuum at 50° C. for 12 h to yield O,N-dimethyl-N-[4-(n-propylamino)-6-(prop-2-ynylamino)-[1,3,5]-triazin-2-yl]-hydroxylamin hydrogen-L(+)-tartrate salt (5c) (1.46 g, 91%). mp 127-130° C. XRPD as illustrated in FIG. 27.

TABLE 30
Elemental analysis for O,N-dimethyl-N-(4-n-propylamino-6-prop-
2-ynylamino-[1,3,5]triazin-2-yl)-hydroxylamine hydrogen-
L(+)-tartrate (5c) from ethyl acetate (Example 3F, Method 2).

| | C | H | N |
|---|---|---|---|
| Calculated | 45.00 | 6.04 | 20.99 |
| Found 1 | 44.90 | 5.97 | 20.69 |
| Found 2 | 44.79 | 9.95 | 20.74 |

### Example 3F, Method 3

### Preparation of O,N-dimethyl-N-[4-(n-propylamino)-6-(prop-2-ynylamino)-[1,3,5]triazin-2-yl]-hydroxylamine as hydrogen-L(+)-tartrate salt (5c) obtained from acetonitrile

O,N-Dimethyl-N-[4-(n-propylamino)-6-(prop-2-ynylamino)-[1,3,5]triazin-2-yl]-hydroxylamine (4) (2 g, 8 mmol, 1 equiv.) was dissolved in acetonitrile (20 mL) with stirring. L(+)-tartaric acid (1.2 g, 8 mmol, 1 equiv.) was added, and the mixture was stirred at room temperature for 6 h. The acid dissolved immediately, and precipitate appeared after 1 h of stirring. This product was collected by filtration, washed with acetonitrile (3×5 mL), initially first dried under vacuum on P₂O₅ at room temperature for 16 h, then further dried under vacuum on P₂O₅ at 50° C. for 6 h to yield O,N-dimethyl-N-[4-(n-propylamino)-6-(prop-2-ynylamino)-[1,3,5]-triazin-2-yl]-hydroxylamine L(+) hydrogen tartrate salt (5c) (2.95 g, 92%). mp 127-130° C. XRPD as illustrated in FIG. 27.

TABLE 31
Elemental Analysis for O,N-Dimethyl-N-(4-n-propylamino-6-prop-
2-ynylamino-[1,3,5]triazin-2-yl)-hydroxylamine hydrogen-
L(+)-tartrate (5c)from acetonitrile (Example 3F, Method 3).

| | C | H | N |
|---|---|---|---|
| Calculated | 45.00 | 6.04 | 20.99 |
| Found 1 | 45.02 | 6.00 | 21.03 |
| Found 2 | 45.03 | 5.98 | 21.00 |

### Example 3G

### Preparation of O,N-dimethyl-N-[4-(n-propylamino)-6-(prop-2-ynylamino)-[1,3,5]triazin-2-yl]-hydroxylamine as hydrogen-maleinate Salt (5d)

### Example 3G, Method 1

### Preparation of O,N-dimethyl-N-[4-(n-propylamino)-6-(prop-2-ynylamino)-[1,3,5]triazin-2-yl]-hydroxylamine as hydrogen maleinate salt (5d) obtained from methyl ethyl ketone

O,N-Dimethyl-N-[4-(n-propylamino)-6-(prop-2-ynylamino)-[1,3,5]-triazin-2-yl]-hydroxylamine (4) (5 g, 20.0 mmol, 1 equiv.) and maleic acid (2.32 g, 20.0 mmol, 1 equiv.) were dissolved in methyl ethyl ketone (20 mL) with stirring at room temperature. A precipitate was formed immediately. The mixture was heated to 70° C., at which point it became homogeneous and then the solution was left to cool to ambient temperature without stirring. Stirring was then restarted in the clear solution at room temperature, and precipitation instantly occurred. After stirring for 1 h, the product was collected by filtration, washed with methyl ethyl ketone (2×8 mL) and air dried in air 60 C for 16 h to yield O,N-dimethyl-N-[4-(n-propylamino)-6-(prop-2-ynylamino)-[1,3,5]-triazin-2-yl]-hydroxylamine hydrogen maleinate (5d) (6.0 g, 82%). mp 123-125° C. XRPD as illustrated in FIG. 29.

TABLE 32
Elemental analysis for O,N-dimethyl-N-(4-n-propylamino-6-prop-
2-ynylamino-[1,3,5]triazin-2-yl)-hydroxylamine hydrogen
maleinate (5d) from methyl ethyl ketone (Example 3G, Method 1).

| | C | H | N |
|---|---|---|---|
| Calculated | 49.17 | 6.05 | 22.94 |
| Found 1 | 49.20 | 6.07 | 22.92 |
| Found 2 | 49.31 | 6.11 | 22.95 |

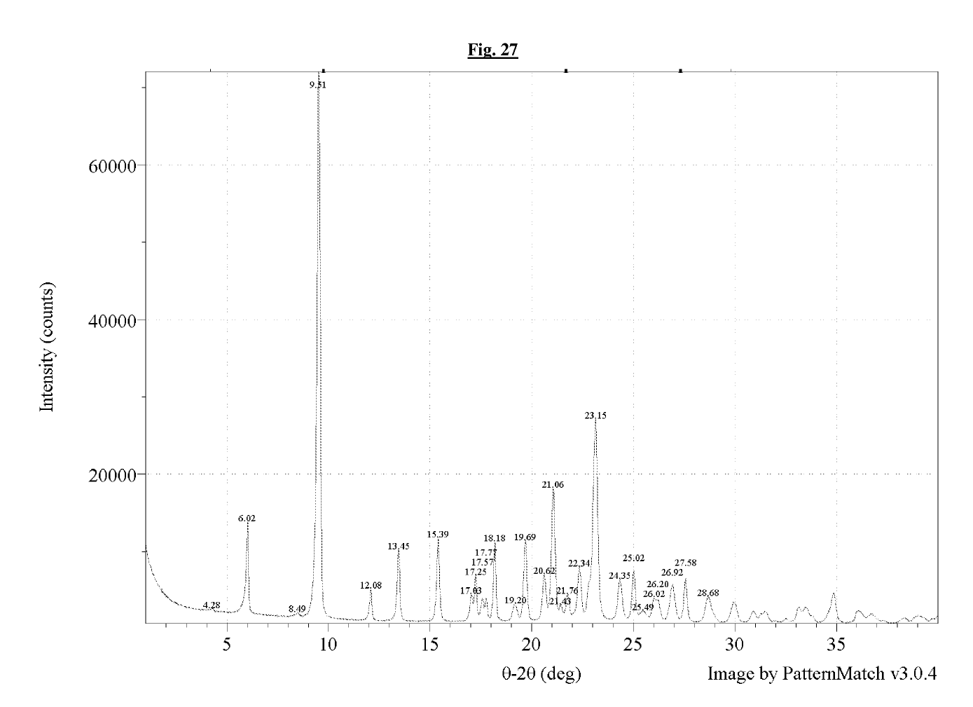

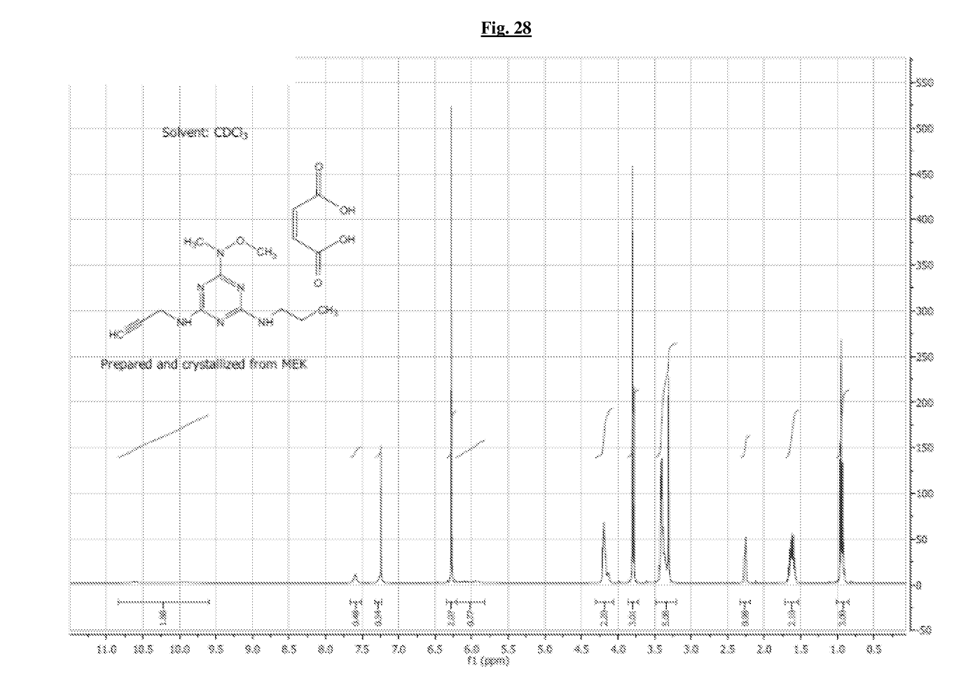

TABLE 33
¹H NMR analysis of O,N-dimethyl-N-(4-n-propylamino-6-prop-2-
ynylamino-[1,3,5]triazin-2-yl)-hydroxylamine hydrogen-maleinate (5d).
400 MHz; CDCl₃; 10 mg/mL; 32 scans (FIG. 28).

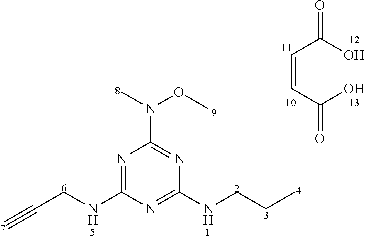 [SMILES: C#CCNc1nc(NCCC)nc(N(C)OC)n1 ; O=C(O)/C=C\C(=O)O]

| | Peak | | J | |
|---|---|---|---|---|
| Delta (ppm) | description | Integration | (Hz) | Assignment |
| 11.00-9.62 | m | 2H | — | 12 + 13 |
| 7.74-7.06 | m | 1H | — | CDCl₃ + 5 |
| 6.31 | S | 2H | — | 10 + 11 |
| 6.11-5.57 | m | 1H | — | 1 |
| 4.30-4.10 | m | 2H | — | 6 |
| 3.87-3.76 | m | 3H | — | 9 |
| 3.50-3.27 | m | 5H | — | 2 + 8 |
| 2.33-2.22 | m | 1H | — | 7 |
| 1.75-1.55 | m | 2H | — | 3 |
| 1.02-0.90 | m | 3H | — | 4 |

TABLE 34
O,N-Dimethyl-N-(4-n-propylamino-6-prop-2-ynylamino-[1,3,5]triazin-
2-yl)-hydroxylamine hydrogen maleinate (5d) diffraction signals.

| Degrees 2θ | d space (Å) | Intensity (%) |
|---|---|---|
| 8.36 ± 0.20 | 10.580 ± 0.259 | 34 |
| 9.43 ± 0.20 | 9.382 ± 0.203 | 100 |
| 12.03 ± 0.20 | 7.355 ± 0.124 | 25 |
| 20.46 ± 0.20 | 4.342 ± 0.042 | 15 |
| 24.28 ± 0.20 | 3.665 ± 0.030 | 54 |
| 25.49 ± 0.20 | 3.495 ± 0.027 | 17 |
| 26.76 ± 0.20 | 3.332 ± 0.025 | 29 |
| 27.88 ± 0.20 | 3.201 ± 0.023 | 16 |

### Example 3G, Method 2

### Crystallization of O,N-dimethyl-N-[4-(n-propylamino)-6-(prop-2-ynylamino)-[1,3,5]triazin-2-yl]-hydroxylamine as hydrogen maleinate salt (5d) obtained from ethyl acetate

O,N-Dimethyl-N-(4-n-propylamino-6-prop-2-ynylamino-[1,3,5]-triazin-2-yl)-hydroxylamine (4) (683 mg, 2.73 mmol, 1 equiv.) and maleic acid (317 mg, 2.73 mmol, 1 equiv.) were mixed with ethyl acetate (10 mL). The mixture was brought to reflux when all starting materials have completely dissolved. The mixture was allowed to cool to room temperature with stirring. After stirring at ambient temperature for 18 h, the product was collected by filtration and washed on filter with ethyl acetate (2×3 mL). The resultant solid was dried under vacuum at 50° C. for 16 h to yield O,N-dimethyl-N-[4-(n-propylamino)-6-(prop-2-ynylamino)-[1,3,5]-triazin-2-yl]-hydroxylamine hydrogen-maleinate (5d) (873 mg, 87%). mp 124-126° C. XRPD as illustrated in FIG. 29.

TABLE 35
Elemental analysis for O,N-Dimethyl-N-(4-n-propylamino-6-prop-
2-ynylamino-[1,3,5]triazin-2-yl)-hydroxylamine hydrogen maleinate
(5d) obtained from ethyl acetate (Example 3G, Method 2).

| | C | H | N |
|---|---|---|---|
| Calculated | 49.17 | 6.05 | 22.94 |
| Found 1 | 49.11 | 6.04 | 22.83 |
| Found 2 | 49.20 | 6.01 | 22.88 |

### Example 3H

### Preparation of O,N-dimethyl-N-[4-(n-propylamino)-6-(prop-2-ynylamino)-[1,3,5]triazin-2-yl]-hydroxylamine as DL-mandelate salt (5e)

### Example 3H, Method 1

### Preparation of O,N-dimethyl-N-[4-(n-propylamino)-6-(prop-2-ynylamino)-[1,3,5]triazin-2-yl]-hydroxylamine as DL-mandelate salt (5e) obtained from acetonitrile

O,N-Dimethyl-N-[4-(n-propylamino)-6-(prop-2-ynylamino)-[1,3,5]-triazin-2-yl]-hydroxylamine (4) (2 g, 8.0 mmol, 1 equiv.) was dissolved in acetonitrile (20 mL) at room temperature, and then DL-mandelic acid (1.22 g, 8.0 mmol, 1 equiv.) was added. The mixture was stirred for 18 h at room temperature. The product was collected by filtration, washed with acetonitrile (3×5 mL), and dried under vacuum at 50° C. for 16 h to yield O,N-dimethyl-N-[4-(n-propylamino)-6-(prop-2-ynylamino)-[1,3,5]triazin-2-yl]-hydroxylamine DL-mandelate (5e) (2.05 g, 64%). mp 98-101° C. XRPD as illustrated in FIG. 31.

TABLE 36
Elemental analysis for O,N-Dimethyl-N-(4-n-propylamino-6-prop-
2-ynylamino-[1,3,5]triazin-2-yl)-hydroxylamine DL-mandelate
(5e) obtained from acetonitrile (Example 3H, Method 1)

| | C | H | N |
|---|---|---|---|
| Calculated | 56.70 | 6.51 | 20.88 |
| Found 1 | 56.63 | 6.45 | 20.93 |
| Found 2 | 56.59 | 6.49 | 22.98 |

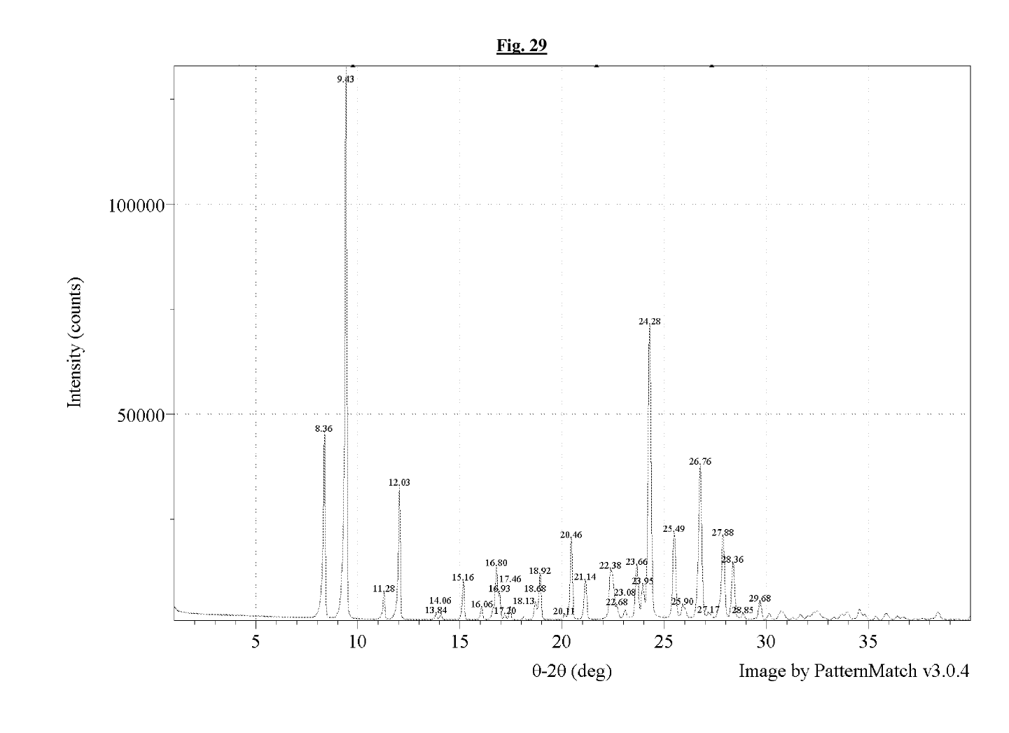

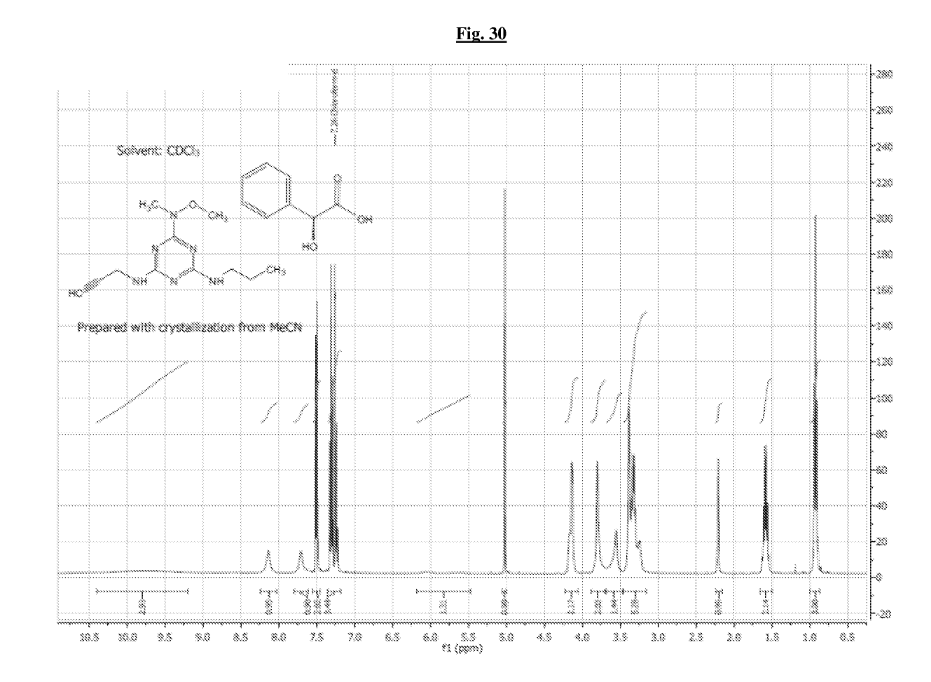

TABLE 37
¹H NMR analysis of O,N-dimethyl-N-(4-n-propylamino-6-prop-2-ynyl
amino-[1,3,5]triazin-2-yl)-hydroxylamine DL-mandelate. 400 MHz; CDCl₃;
10 mg/mL; Number of scans: 32 (FIG. 30).

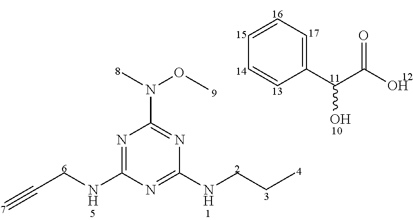

| Delta | Peak | | J | |
|---|---|---|---|---|
| (ppm) | description | Integration | (Hz) | Assignment |
| 10.7-8.8 | br s | 1H | — | Water + 12 |
| 8.27-7.59 | m | 2H | — | 1 + 5 |
| 7.57-7.17 | m | 5H | — | 13 + 14 + 15 + 16 + 17 |
| 6.26-5.42 | m | 1H | — | 10 |
| 5.02 | s | 1H | — | 11 |
| 4.23-4.05 | m | 2H | — | 6 |
| 3.90-3.47 | m | 3H | — | 9 |
| 3.46-3.14 | m | 5H | — | 2 + 8 |
| 2.24-2.17 | m | 1H | — | 7 |
| 1.66-1.50 | m | 2H | — | 3 |
| 0.92 | t | 3H | 7.50 | 4 |

TABLE 38
O,N-Dimethyl-N-(4-n-propylamino-6-prop-2-ynylamino-[1,3,5]triazin-
2-yl)-hydroxylamine DL-mandelate (5e) diffraction signals.

| Pos. [°2Th.] | d-spacing [Å] | Rel. Int. [%] |
|---|---|---|
| 8.7374 | 10.11232 | 100.00 |
| 10.6080 | 8.33299 | 20.59 |
| 12.1786 | 7.26159 | 18.31 |
| 13.3412 | 6.63132 | 13.66 |
| 16.7232 | 5.29706 | 13.25 |
| 17.8257 | 4.97184 | 28.26 |
| 19.6306 | 4.51861 | 22.86 |
| 21.7142 | 4.08950 | 90.64 |
| 22.0533 | 4.02738 | 42.90 |
| 24.0057 | 3.70407 | 8.21 |
| 27.0255 | 3.29664 | 20.35 |

### Example 3H, Method 2

### Formation of O,N-dimethyl-N-[4-(n-propylamino)-6-(prop-2-ynylamino)-[1,3,5]-triazin-2-yl]-hydroxylamine as DL-mandelate salt (15) obtained from methyl-tert-butyl ether

O,N-dimethyl-N-[4-(n-propylamino)-6-(prop-2-ynylamino)-[1,3,5]-triazin-2-yl]-hydroxylamine (4) (300 mg, 1.20 mmol, 1 equiv.) and DL-mandelic acid (182 mg, 1.20 mmol, 1 equiv.) were suspended in methyl-tert-butyl ether (5 mL). The stirred mixture was brought to reflux to form clear solution, then it was allowed to cool to ambient temperature and stirred for 15 min. The product was collected by filtration, washed with methyl-tert-butyl ether (3×1 mL), and dried under vacuum at 40° C. for 48 h, yielding O,N-dimethyl-N-[4-(n-propylamino)-6-(prop-2-ynylamino)-[1,3,5]-triazin-2-yl]-hydroxylamine DL-mandelate (5e) (320 mg, 66%). mp 95-97° C. XRPD as illustrated in FIG. 31.

TABLE 39
Elemental analysis for O,N-dimethyl-N-(4-n-propylamino-6-prop-2-
ynylamino-[1,3,5]-triazin-2-yl)-hydroxylamine DL-mandelate
(5e) obtained from methyl-tert-butyl ether (Example 3H, Method 2).

| | C | H | N |
|---|---|---|---|
| Calculated | 56.70 | 6.51 | 20.88 |
| Found 1 | 56.40 | 6.46 | 20.67 |
| Found 2 | 56.57 | 6.47 | 22.75 |

### Example 3H, Method 3

### Preparation of O,N-dimethyl-N-[4-(n-propylamino)-6-(prop-2-ynylamino)-[1,3,5]-triazin-2-yl]-hydroxylamine as DL-mandelate salt (5e) obtained from toluene admixed with petroleum ether-40

O,N-Dimethyl-N-[4-(n-propylamino)-6-(prop-2-ynylamino)-[1,3,5]-triazin-2-yl]-hydroxylamine (4) (4 g, 16.0 mmol, 1 equiv.) was dissolved in toluene (40 mL) at room temperature, and then DL-mandelic acid (2.43 g, 16.0 mmol, 1 equiv.) was added. To this solution light petroleum ether (BP 40-60° C.) was added, and the mixture was stirred for 16 h at room temperature. The product was collected by filtration, washed with light petroleum ether (3×15 mL) and air dried at 60° C. for 16 h to yield O,N-dimethyl-N-[4-(n-propylamino)-6-(prop-2-ynylamino)-[1,3,5]-triazin-2-yl]-hydroxylamine DL-mandelate (5e): (6.15 g, 95%). mp 95-97° C. XRPD as illustrated in FIG. 31.

TABLE 40
Elemental analysis for O,N-Dimethyl-N-(4-n-propylamino-
6-prop-2-ynylamino-[1,3,5]-triazin-2-yl)-hydroxylamine
DL-mandelate (5e) obtained from toluene admixed with light
petroleum ether (BP 40-60° C.) (Example 3H, Method 3).

| | C | H | N |
|---|---|---|---|
| Calculated* | 57.05 | 6.52 | 20.68 |
| Found 1 | 57.30 | 6.44 | 20.83 |
| Found 2 | 57.28 | 6.48 | 20.82 |

*Figure are corrected for 6 moles % toluene

### Example 3I

### Preparation of O,N-dimethyl-N-[4-(n-propylamino)-6-(prop-2-ynylamino)-[1,3,5]-triazin-2-yl]-hydroxylamine as hydrogen malonate salt (5f)

### Example 3I, Method 1

### Preparation of O,N-dimethyl-N-[4-(n-propylamino)-6-(prop-2-ynylamino)-[1,3,5]-triazin-2-yl]-hydroxylamine as hydrogen-malonate salt (5f) obtained from diethyl ether admixed with ethanol

O,N-dimethyl-N-[4-(n-propylamino)-6-(prop-2-ynylamino)-[1,3,5]triazin-2-yl]-hydroxylamine (4) (2 g, 8.0 mmol, 1 equiv.) was dissolved in a mixture of diethyl ether (35 mL) and ethanol (0.6 mL) at room temperature and then malonic acid (830 mg, 8.0 mmol, 1 equiv.) was added. A precipitate formed immediately, forming a thick gel. The mixture was stirred for 18 h at ambient temperature. The product was collected by filtration, washed with diethyl ether (10 mL) and dried under vacuum over P₂O₅ at 50° C. for 16 h to yield O,N-dimethyl-N-[4-(n-propylamino)-6-(prop-2-ynylamino)-[1,3,5]-triazin-2-yl]-hydroxylamine as hydrogen malonate (5f) (2.46 g, 87%). mp 111-113° C. XRPD as illustrated in FIG. 33.

TABLE 41
Elemental analysis for O,N-dimethyl-N-(4-n-propylamino-
6-prop-2-ynylamino-[1,3,5]triazin-2-yl)-hydroxylamine
hydrogen malonate (5f) obtained from diethyl ether
admixed with ethanol (Example 3I, Method 1).

| | C | H | N |
|---|---|---|---|
| Calculated | 47.45 | 6.26 | 23.72 |
| Found 1 | 47.67 | 6.25 | 23.74 |
| Found 2 | 47.52 | 6.22 | 23.68 |

TABLE 42
¹H NMR analysis of O,N-dimethyl-N-(4-n-propylamino-6-prop-2-
ynylamino-[1,3,5]triazin-2-yl)-hydroxylamine hydrogen malonate (5f).
400 MHz; D₂O; 10 mg/mL; Number of scans: 40 (FIG. 32).

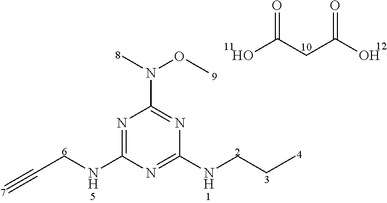

| | Peak | | J | |
|---|---|---|---|---|
| Delta (ppm) | description | Integration | (Hz) | Assignment |
| 4.79 | m | 7H | — | D₂O + 1 + 5 + 10 + 11 + 12 |
| 4.29-4.13 | m | 2H | — | 6 |
| 3.80 | s | 3H | — | 9 |
| 3.51-3.26 | m | 5H | — | 2 + 8 |
| 2.69-2.63 | m | 1H | — | 7 |
| 1.67-1.51 | m | 2H | — | 3 |
| 0.91 | t | 3H | 7.50 | 4 |

TABLE 43
O,N-Dimethyl-N-(4-n-propylamino-6-prop-2-ynylamino-[1,3,5]triazin-
2-yl)-hydroxylamine hydrogen malonate (5f) diffraction signals.

| Pos. [°2Th.] | d-spacing [Å] | Rel. Int. [%] |
|---|---|---|
| 4.2898 | 20.58129 | 34.89 |
| 8.7047 | 10.15019 | 100.00 |
| 9.9210 | 8.90843 | 24.22 |
| 11.6926 | 7.56233 | 13.94 |
| 18.5606 | 4.77663 | 18.76 |
| 20.0382 | 4.42761 | 23.31 |
| 22.2299 | 3.99578 | 39.69 |
| 24.5093 | 3.62909 | 68.36 |
| 26.3741 | 3.37656 | 12.14 |
| 29.5295 | 3.02255 | 21.72 |
| 32.5846 | 2.74579 | 6.25 |

### Example 3I, Method 2

### Preparation of O,N-dimethyl-N-[4-(n-propylamino)-6-(prop-2-ynylamino)-[1,3,5]triazin-2-yl]-hydroxylamine hydrogen malonate salt (5f) obtained from ethyl acetate

O,N-Dimethyl-N-[4-(n-propylamino)-6-(prop-2-ynylamino)-[1,3,5]triazin-2-yl]-hydroxylamine (4)(2 g, 8.0 mmol, 1 equiv.) and malonic acid (0.83 g, 8.0 mmol, 1 equiv.) were mixed with ethyl acetate. The suspension was stirred and heated to 70° C., becoming homogeneous. After this time, the mixture was allowed to cool to ambient temperature for 16 h, with stirring. After 2 h, a gel-like precipitate appeared and the product was collected by filtration, washed with ethyl acetate (3×3 mL) and dried under vacuum at 45° C. for 4 h to yield O,N-dimethyl-N-[4-(n-propylamino)-6-(prop-2-ynylamino)-[1,3,5]-triazin-2-yl]-hydroxylamine hydrogen malonate (5f) (2.41 g, 85%). mp 113-115° C. XRPD, FIG. 33.

TABLE 44
Elemental analysis for O,N-dimethyl-N-(4-n-propylamino-
6-prop-2-ynylamino-[1,3,5]-triazin-2-yl)-hydroxylamine
hydrogen malonate (5f) obtained from ethyl acetate.

| | C | H | N |
|---|---|---|---|
| Calculated | 47.45 | 6.26 | 23.72 |
| Found 1 | 47.62 | 6.30 | 23.78 |
| Found 2 | 47.78 | 6.26 | 23.85 |

### Example 3I, Method 3

### Crystallization of O,N-dimethyl-N-[4-(n-propylamino)-6-(prop-2-ynylamino)-[1,3,5]-triazin-2-yl]-hydroxylamine hydrogen malonate salt (5f) obtained from acetonitrile

O,N-Dimethyl-N-[4-(n-propylamino)-6-(prop-2-ynylamino)-[1,3,5]triazin-2-yl]-hydroxylamine (4) (5 g, 20.0 mmol, 1 equiv.) was dissolved in acetonitrile (50 mL) at room temperature, and then malonic acid (2.08 g, 20.0 mmol, 1 equiv.) was added. The mixture was stirred for 20 h at room temperature. The product was collected by filtration, washed with acetonitrile (15 mL), and dried under vacuum over P₂O₅ at rt for 16 h to yield O,N-dimethyl-N-[4-(n-propylamino)-6-(prop-2-ynylamino-[1,3,5]-triazin-2-yl]-hydroxylamine hydrogen-malonate (5f) (4.93 g, 70%). mp 114-117° C. XRPD matches FIG. 33.

TABLE 45
Elemental analysis for O,N-Dimethyl-N-(4-n-propylamino-6-prop-
2-ynylamino-[1,3,5]triazin-2-yl)-hydroxylamine hydrogen malonate
(5f) obtained from acetonitrile (Example 3I, Method 3).

| | C | H | N |
|---|---|---|---|
| Calculated | 47.45 | 6.26 | 23.72 |
| Found 1 | 47.39 | 6.24 | 23.69 |
| Found 2 | 47.48 | 6.21 | 23.70 |

### Example 3J

### Preparation of O,N-dimethyl-N-[4-(n-propylamino)-6-(prop-2-ynylamino)-[1,3,5]-triazin-2-yl]-hydroxylamine as hydrogen fumarate salt (5g)

### Example 3J, Method 1

### Preparation of O,N-dimethyl-N-[4-(n-propylamino)-6-(prop-2-ynylamino)-[1,3,5]-triazin-2-yl]-hydroxylamine as hydrogen fumarate salt (5g) from ethyl acetate admixed with ethanol

O,N-Dimethyl-N-[4-(n-propylamino)-6-(prop-2-ynylamino)-[1,3,5]triazin-2-yl]-hydroxylamine (4) (1 g, 4.0 mmol, 1 equiv.) and fumaric acid (0.464 g, 4.0 mmol, 1 equiv.) were suspended in ethyl acetate (25 mL) and ethanol (5 mL). The stirred mixture was brought to reflux and stirred 1 min until a clear solution was obtained. The solution was then concentrated to one half of its initial volume. To this mixture, ethyl acetate (15 mL) was added and the solution was again concentrated to one half of its volume. The remainder was allowed to cool to ambient temperature and then stirred at room temperature for 16 h. The resultant product was collected by filtration, washed with ethyl acetate (2×3 mL) and dried under vacuum over P₂O₅ at room temperature for 16 h to yield O,N-dimethyl-N-[4-(n-propylamino)-6-(prop-2-ynylamino)-[1,3,5]-triazin-2-yl]-hydroxylamine hydrogen fumarate (5g) 1.05 g (71%). mp 153-155° C. XRPD as illustrated in FIG. 35.

TABLE 46
Elemental analysis for O,N-dimethyl-N-(4-n-propylamino-
6-prop-2-ynylamino-[1,3,5]-triazin-2-yl)-hydroxylamine
hydrogen fumarate (5g) obtained from ethyl acetate
admixed with ethanol (Example 3J, Method 1).

| | C | H | N |
|---|---|---|---|
| Calculated | 49.17 | 6.05 | 22.94 |
| Found 1 | 49.14 | 6.07 | 23.00 |
| Found 2 | 49.27 | 6.04 | 23.04 |

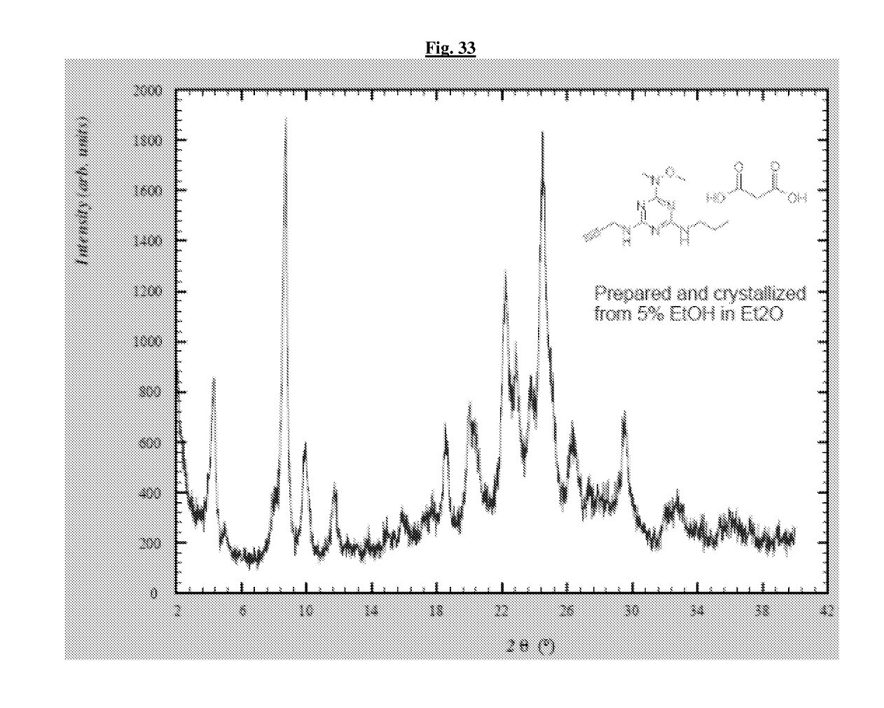

TABLE 47
¹H NMR analysis for O,N-dimethyl-N-(4-n-propylamino-6-prop-2-
ynylamino-[1,3,5]triazin-2-yl)-hydroxylaminehydrogen fumarate (5g).
400 MHz; dimethyl sulfoxide-d₆; mg/mL; Number of scans: 64 (FIG. 34).

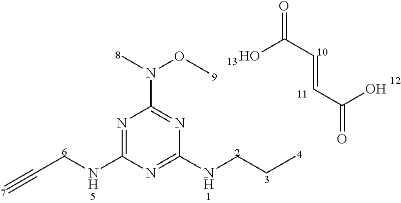 [SMILES: C#CCNc1nc(NCCC)nc(N(C)OC)n1 ; O=C(O)/C=C/C(=O)O]

| | Peak | | J | |
|---|---|---|---|---|
| Delta (ppm) | description | Integration | (Hz) | Assignment |
| 13.64-12.55 | br s | 4H | — | D₂O + 13 |
| 7.40-6.74 | m | 3H | — | 1 + 5 + 12 |
| 6.63 | s | 2H | — | 10 + 11 |
| 4.03-3.93 | m | 2H | — | 6 |
| 3.73-3.59 | m | 3H | — | 9 |
| 3.22-3.07 | m | 5H | — | 2 + 8 |
| 3.01-2.95 | m | 1H | — | 7 |
| 1.58-1.37 | m | 2H | — | 3 |
| 0.85 | t | 3H | 7.50 | 4 |

TABLE 48
O,N-Dimethyl-N-(4-n-propylamino-6-prop-2-ynylamino-[1,3,5]-triazin-
2-yl)-hydroxylamine hydrogen fumarate (5g) diffraction signals.

| Pos. [°2Th.] | d-spacing [Å] | Rel. Int. [%] |
|---|---|---|
| 9.1947 | 9.61040 | 100.00 |
| 10.4154 | 8.48662 | 7.24 |
| 12.2336 | 7.22910 | 8.97 |
| 12.9127 | 6.85038 | 8.06 |
| 19.0736 | 4.64929 | 16.54 |
| 19.6603 | 4.51185 | 5.26 |
| 20.5684 | 4.31466 | 8.90 |
| 22.3942 | 3.96684 | 13.16 |
| 22.8975 | 3.88078 | 26.66 |
| 24.1505 | 3.68218 | 36.04 |
| 27.6034 | 3.22892 | 14.53 |
| 30.7488 | 2.90542 | 4.15 |

### Example 3K

### Recrystallization of O,N-dimethyl-N-[4-(n-propylamino)-6-(prop-2-ynylamino)-[1,3,5]-triazin-2-yl]-hydroxylamine hydrogen fumarate (5g)

### Example 3K, Methods 2, 3, and 4

### Recrystallization of O,N-dimethyl-N-[4-(n-propylamino)-6-(prop-2-ynylamino)-[1,3,5]-triazin-2-yl]-hydroxylamine hydrogen fumarate (5g) from ethyl acetate, water, and isopropyl acetate

### Method 2

O,N-Dimethyl-N-[4-(n-propylamino)-6-(prop-2-ynylamino-[1,3,5]-triazin-2-yl]-hydroxylamine hydrogen fumarate (5g) (2 g) was dissolved in ethyl acetate (20 mL) at reflux and the solution was left to cool to ambient temperature and stirred for 16 h. The product was collected by filtration, washed with ethyl acetate (2×5 mL) and dried under vacuum at 40° C. for 16 h to yield O,N-dimethyl-N-[4-(n-propylamino)-6-(prop-2-ynylamino)-[1,3,5]-triazin-2-yl]-hydroxylamine hydrogen fumarate (17) (1.73 g, 86%). mp 153-155° C. XRPD: main signals as illustrated in FIG. 35.

Following a similar procedure, the hydrogen fumarate salt (5g) was also recrystallized at 2 gram scale from water (Method 3, product mp 153-155° C., 79% yield) and at 2 g scale from isopropyl acetate (Method 4, product mp 153-155° C., 90% yield) XRPD: Main signals for products from Method 3 and Method 4 were as illustrated in FIG. 35.

TABLE 49
Elemental analysis for O,N-dimethyl-N-(4-n-propylamino-6-prop-
2-ynylamino-[1,3,5]-triazin-2-yl)-hydroxylamine hydrogen
fumarate (5g) obtained from ethyl acetate (Example 3K, Method 2).

| | C | H | N |
|---|---|---|---|
| Calculated | 49.17 | 6.05 | 22.94 |
| Found 1 | 49.04 | 6.01 | 22.91 |
| Found 2 | 49.13 | 6.01 | 22.98 |

TABLE 50
Elemental Analysis for O,N-Dimethyl-N-(4-n-propylamino-6-prop-
2-ynylamino-[1,3,5]-triazin-2-yl)-hydroxylamine hydrogen
fumarate (5g) obtained from water (Example 3K, Method 3).

| | C | H | N |
|---|---|---|---|
| Calculated | 49.17 | 6.05 | 22.94 |
| Found 1 | 49.05 | 5.99 | 22.99 |
| Found 2 | 49.10 | 5.99 | 23.08 |

TABLE 51
Elemental analysis for O,N-dimethyl-N-(4-n-propylamino-6-prop-2-
ynylamino-[1,3,5]-triazin-2-yl)-hydroxylamine hydrogen fumarate
(5g) obtained from isopropyl acetate (Example 3K, Method 4).

| | C | H | N |
|---|---|---|---|
| Calculated | 49.17 | 6.05 | 22.94 |
| Found 1 | 49.01 | 6.06 | 22.85 |
| Found 2 | 49.06 | 6.05 | 22.91 |

### Example 3L

### Preparation of O,N-dimethyl-N-[4-(n-propylamino)-6-(prop-2-ynyl amino)-[1,3,5]-triazin-2-yl]-hydroxylamine as saccharinate salt (5h)

### Example 3L, Method 1

### Preparation of O,N-dimethyl-N-[4-(n-propylamino)-6-(prop-2-ynylamino)-[1,3,5]-triazin-2-yl]-hydroxylamine as saccharinate salt (5h) as obtained from toluene

O,N-Dimethyl-N-[4-(n-propylamino)-6-(prop-2-ynylamino)-[1,3,5]triazin-2-yl]-hydroxylamine (4) (1 g, 4.0 mmole) was treated with 1 molar equivalent of saccharine in toluene (10 mL) at room temperature. The mixture was stirred for 18 h at room temperature. The solid product was collected by filtration, washed with light petroleum ether (BP 40-60° C.) (3×5 mL), and air dried at 60° C. for 16 h to yield O,N-dimethyl-N-[4-(n-propylamino)-6-(prop-2-ynylamino)-[1,3,5]-triazin-2-yl]-hydroxylamine saccharinate (5h) (1.48 g, 85%). mp 117-120° C. XRPD as illustrated in FIG. 37.

TABLE 52
Elemental analysis for O,N-dimethyl-N-(4-n-propylamino-6-
prop-2-ynylamino-[1,3,5]-triazin-2-yl)-hydroxylamine
saccharinate (5h) obtained from toluene (Example 3L, Method 1).

| | C | H | N |
|---|---|---|---|
| Calculated | 49.87 | 5.35 | 22.62 |
| Found 1 | 49.70 | 5.38 | 22.59 |
| Found 2 | 49.79 | 5.39 | 22.65 |

### Example 3L, Method 2

### Preparation of O,N-dimethyl-N-[4-(n-propylamino)-6-(prop-2-ynylamino)-[1,3,5]-triazin-2-yl]-hydroxylamine as saccharinate salt (5h) obtained from isopropanol

O,N-Dimethyl-N-[4-(n-propylamino)-6-(prop-2-ynylamino)-[[1,3,5]triazin-2-yl]-hydroxylamine (4) (1 g, 4.0 mmol, 1 equiv.) was dissolved in isopropanol (10 mL) at room temperature and saccharin (0.73 g, 4.0 mmol, 1 equiv.) was added. The mixture was stirred for 18 h at room temperature. The solid product was collected by filtration, washed light petroleum ether (BP 40-60° C.) (3×5 mL) and air dried at 60° C. for 16 h to yield O,N-dimethyl-N-[4-(n-propylamino)-6-(prop-2-ynylamino-[1,3,5]triazin-2-yl]-hydroxylamine saccharinate (5h) (1.58 g, 91%). mp 117-120° C. XRPD is as illustrated in FIG. 37.

TABLE 53
Elemental analysis for O,N-dimethyl-N-(4-n-propylamino-6-prop-
2-ynylamino-[1,3,5]-triazin-2-yl)-hydroxylamine saccharinate
(5h) obtained from isopropanol (Example 3L, Method 2).

| | C | H | N |
|---|---|---|---|
| Calculated | 49.87 | 5.35 | 22.62 |
| Found 1 | 49.93 | 5.34 | 22.71 |
| Found 2 | 49.94 | 5.35 | 22.77 |

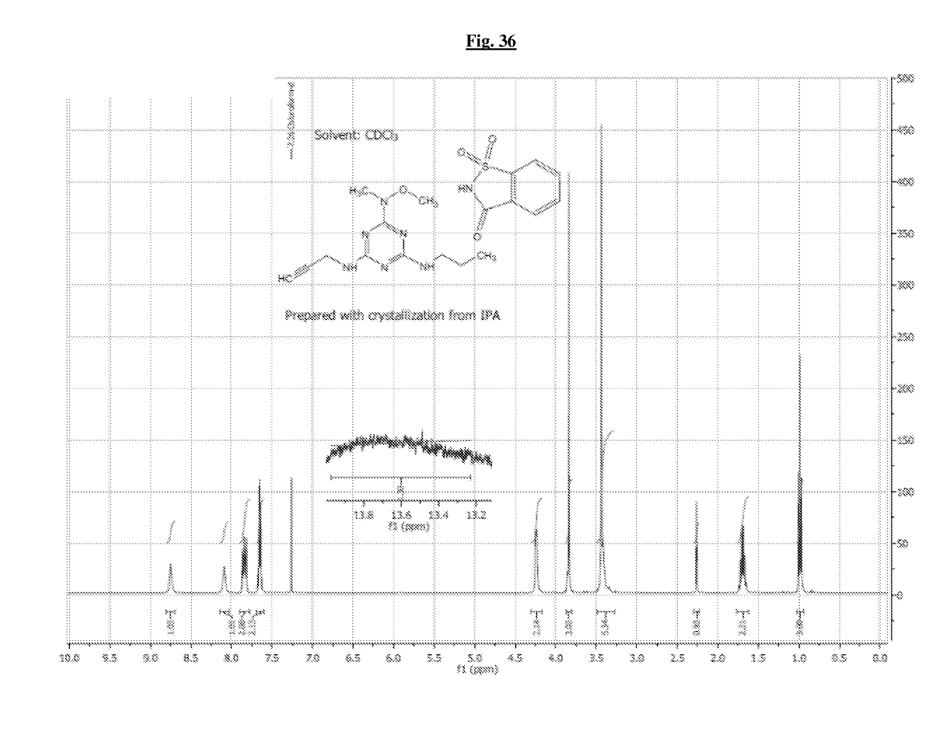

TABLE 54
¹H NMR analysis for O,N-dimethyl-N-(4-n-propylamino-6-prop-2-
ynylamino-[1,3,5]triazin-2-yl)-hydroxylamine saccharinate (5h).
400 MHz; CDCl₃; Concentration: 10 mg/mL; Number of scans: 32 (FIG. 36).

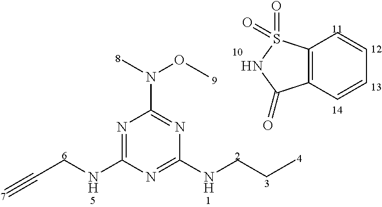

| | Peak | | J | |
|---|---|---|---|---|
| Delta (ppm) | description | Integration | (Hz) | Assignment |
| 14.00-13.20 | br s | 1H | — | 10 |
| 8.80-8.69 | m | 1H | — | 5 |
| 8.15-8.01 | m | 1H | — | 1 |
| 7.91-7.56 | m | 4H | — | 11 + 12 + 13 + 14 |
| 4.31-4.14 | m | 2H | — | 6 |
| 3.90-3.75 | m | 3H | — | 9 |
| 3.50-3.29 | m | 5H | — | 2 + 8 |
| 2.30-2.21 | m | 1H | — | 7 |
| 1.76-1.58 | m | 2H | — | 3 |
| 0.98 | t | 3H | 7.50 | 4 |

TABLE 55
O,N-Dimethyl-N-(4-n-propylamino-6-prop-2-ynylamino-[1,3,5]triazin-
2-yl)-hydroxylamine saccharinate (5h) XRPD diffraction.

| Pos. [°2Th.] | d-spacing [Å] | Rel. Int. [%] |
|---|---|---|
| 10.0036 | 8.83502 | 82.02 |
| 11.8515 | 7.46126 | 74.93 |
| 13.0717 | 6.76739 | 42.70 |
| 15.8614 | 5.58286 | 59.69 |
| 17.7054 | 5.00537 | 48.82 |
| 19.0513 | 4.65467 | 100.00 |
| 22.5878 | 3.93326 | 50.46 |
| 23.3160 | 3.81205 | 62.69 |
| 24.3965 | 3.64562 | 16.07 |
| 27.0242 | 3.29679 | 45.98 |

### Example 3L, Method 3

### Recrystallization of O,N-dimethyl-N-[4-(n-propylamino)-6-(prop-2-ynylamino)-[1,3,5]triazin-2-yl]-hydroxylamine saccharinate salt (5h) from water

O,N-Dimethyl-N-(4-n-propylamino-6-prop-2-ynylamino-[1,3,5]-triazin-2-yl)-hydroxylamine saccharinate (18) (0.2 g) was dissolved in water (5 mL) at reflux, and the solution was left to cool to ambient temperature and stirred for 16 h. The resultant product precipitated as an oil from a warm solution (40-50° C.), which then solidified. The crystalline product was collected by filtration, and washed with water (2×3 mL), and dried under vacuum over P₂O₅ at 60° C. for 48 h. Yield of O,N-dimethyl-N-[4-(n-propylamino)-6-(prop-2-ynylamino)-[1,3,5]-triazin-2-yl]-hydroxylamine saccharinate (18): 0.12 g (61%). mp 117-119° C. XRPD as illustrated in FIG. 37.

TABLE 56
Elemental analysis for O,N-dimethyl-N-(4-n-propylamino-6-
prop-2-ynylamino-[1,3,5]triazin-2-yl)-hydroxylamine saccharinate
(18) obtained from water (Example 3L, Method 3).

| | C | H | N | S |
|---|---|---|---|---|
| Calculated | 49.87 | 5.35 | 22.62 | 7.40 |
| Found 1 | 49.77 | 5.46 | 22.65 | 7.29 |
| Found 2 | 49.79 | 5.41 | 22.67 | 7.44 |

### Example 3M

### Analytical Methods

### Method 3M-A

### Headspace GC-FID Method for quantifying 2-chloroallylamine (CAA) in propargyl amine (PA)

### Sample Preparation Procedure:

1. In a 20 mL glass vial with a crimp cap, NaCl (3.8±0.1 g) was weighed.
2. PA sulfate (50 to 100 mg) was weighed with a precision±0.1 mg and placed in the vial.
3. Distilled or deionized water (9.0±0.1 mL) was added.
4. Tools for the sealing of the vial were prepared. A solution of 8M aq. NaOH or KOH (1.0±0.1 mL) was added to the vial and the vial was immediately sealed with a crimp cap.
5. Sealed vial was agitated for 30 seconds, then placed in a thermostated autosampler of a headspace injector.

### Chromatographic Conditions of the Analysis:

Temperature of the thermostat of the headspace injector: 90° C.; Temperature of the syringe: 105° C.; Stirring of the sample: continuous; Time of conditioning: 20 min; Volume of injected gas phase: 1 mL; Column: RTX VRX, 75 m, 0.46 mm ID, stationary phase-2.55 μm; Temperature gradient: 85° C. for 4 min, ramp at 15° C./min to 220° C. for 9 min, 220° C. for 7 min; Carrier gas: helium, 97.5 kPa, constant linear velocity: 35 cm/s; Injector: splitless (0.5 min), 200° C.; Detector: FID, 250° C., sampling rate-5 Hz;
Limit of Detection: 1 ug CAA; Criterion: 50-60% recovery error for CAA in presence of propargyl amine
2-Chloroallylamine Retention Time: 8.399 (neat CAA)-8.549 (increased with increasing presence of PA)

Calibration Procedure for Quantitation of CAA in 1-20 ug Range in PA:

### Stock Calibration Solution:

1. In a 10 mL glass vial 2-chloroallyl amine hydrochloride (14.40±0.02 mg) was weighed (10.36 mg of CAA free base equivalent).
2. Deionized water (2 mL) was added (complete dissolution): Solution Concentration: 5.18 mg/mL (μg/μL) as free base
3. Calibration samples with 1 g, 5 μg and 20 μg of CAA were prepared by appropriate dilutions of the stock solution in a 10 mL volumetric flask
   The Stock Calibration Solution and further diluted solutions were stored at +4° C. In these conditions the concentration of the diluted calibration solutions is stable for 1 to 2 weeks.
4. For each concentration two parallel analyses were done.
   For a 100 ug recovery test: 109 μg was determined.

### Method 3M-B

### GC-MS Method for Monitoring Stage 1 (Examples 1A, 2A and 2B) and for Monitoring Consumption of Excess Cyanuric Chloride by Reaction with Isopropanol (Examples 2A and 2B)

| Injector | Injection Volume | 1 | uL |
|---|---|---|---|
| Inlet | Inlet Mode | Split | |
| | Inlet Temperature | 200° C. | |
| | Split Ratio | 20:1 | |
| | Initial Pressure | 2.98 | psi |
| | Flow Rate (Constant Flow) | 13.5 | mL/min |
| | Carrier Gas | Helium | |
| Oven | Temperature Program | 50° C. for 3 minutes, | |
| | | 25° C./min to 250° C., | |
| | | Hold at 250° C. for 4 minutes | |
| | Maximum Temperature | 325° C. | |
| Column | Dimensions | HP-5MS; 30 m × 0.250 mm, | |
| | | 0.25 um film thickness | |
| Detector | Detector Type | Single Quadrupole MS | |
| | Auxiliary Temperature | 250° C. | |
| | MS Source Temperature | 230° C. | |
| | MS Quad Temperature | 150° C. | |
| | Solvent Delay | 2.0 | min |
| | Scan Type | Scan Mode | |
| | Scan Speed | Normal | |
| | Scan Range | 10 to 500 amu | |
| | Scan Rate | 5.6 | scans/sec |
| | Threshold | 50 | counts |
| Retention | Cyanuric Chloride | 7.4 | minutes |

| | | |
|---|---|---|
| Times and Molecular Weights | 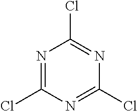 | MW 184.41 |
| | 1 | |
| | Stage 1 Product | 14.7 minutes |
| | 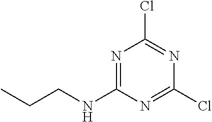 | MW 207.06 |
| | 2 | |
| | Bisamination byproduct | 17.4 minutes |
| | 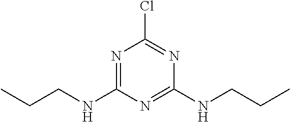 | MW 229.71 |

### Method 3M-C

### LC-MS method for monitoring Stage 2 reaction completion for Examples 1B, 2A and 2B and for a % Purity of 2,4-dichloro-N-(6-n-propylamino)-[1,3,5]triazine (2) and 6-chloro-N²-(prop-2-ynylamino)-N⁴-n-propylamino-1,3,5-triazine (3)

| Sample | Concentration | 1 mg/1 mL | |
|---|---|---|---|
| | Diluent | Acetonitrile | |
| Injector | Injection Volume | variable, 0.1-5 μL | |
| Inlet | Column Temperature | 30° C. | |
| | Flow Rate | 0.5 | mL/min |
| Gradient | Mobile phase A | 0.01% trifluoroacetic acid in water | |
| | Mobile phase B | Acetonitrile | |
| | Gradient | from 10% to 95% B for 4.3 min | |
| | | hold 95% B for 1.7 min | |
| | | Return to initial conditions | |
| | | Hold for 2 min | |
| Column | Type | Waters ACQUITY UPLC ® BEH | |
| | | C18 1.7 μm; | |
| | | ser. no. 02083218625720 | |
| | Dimensions | 2.1 × 50 mm | |
| UV/VIS | Detector Type | Waters PDA eλ Detector | |
| Detector | Wavelength | 220-320 | nm |
| MS | Detector Type | Waters SQ Detector 2 | |
| Detector | Source Temperature | 150° C. | |
| | Desolvation | 350° C. | |
| | Temperature | | |
| | Scan Range 1 | 50 to 150 amu ES+ | |
| | Scan Time 1 | 0.2 | s |
| | Scan Range 2 | 150 to 1200 amu ES+ | |
| | Scan Time 2 | 0.5 | s |

| | | |
|---|---|---|
| Retention | S-1 product | 2.72 min. |
| times | 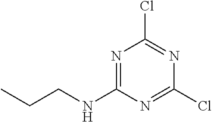 | MW 207.06 |
| | 2 | |
| | S-2 product | 2.43 min. |
| | 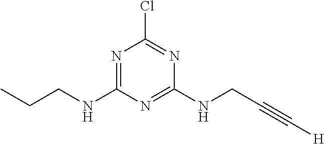 | MW 225.68 |
| | 3 | |

### Method 3M-D

### LC-MS Method for Monitoring Stage 3 (Examples 1C, 2C and 2F) and for A % Purity of O,N-dimethyl-N-[4-(n-propylamino)-6-(prop-2-ynylamino)-[1,3,5]-triazin-2-yl]-hydroxylamine (4)

| Sample | Concentration | 1 mg/1 mL | |
|---|---|---|---|
| | Diluent | Acetonitrile | |
| Injector | Injection Volume | 1 μL, partial loop with needle | |
| | | overfill | |
| Inlet | Column Temperature | 30° C. | |
| | Sample temperature | 10° C. | |
| | Flow Rate | 0.25 | mL/min |
| Gradient | Mobile phase A | 0.1% (v/v) formic acid in water | |
| | Mobile phase B | Acetonitrile | |
| | Run time | 6 | min |
| | Gradient | hold 20% of B for 1 min | |
| | | from 20% to 98% B for 1.5 min | |
| | | hold 98% B for 2 min | |
| | | return to initial conditions in | |
| | | 0.2 min hold for 1.3 min | |
| Column | Type | Waters ACQUITY UPLC® BEH | |
| | | C18 1.7 μm | |
| | Dimensions | 2.1 × 50 mm | |
| UV/VIS | Detector Type | Waters PDA eλ, Detector | |
| Detector | Wavelength | 220-320 nm | |
| MS | Detector Type | Waters SQ Detector 2 | |
| Detector | Ionization mode | ESI+ | |
| | Capillary voltage | 2.8 | kV |
| | Cone voltage | 30 | V |
| | Extractor voltage | 3.0 | V |
| | Source Temperature | 120° C. | |
| | Desolvation | 400° C. | |
| | Temperature | | |
| | Collision energy | 20 | eV |

| | | |
|---|---|---|
| | MS/MS transitions | 287 >> 221 Da |
| | | 289 >> 221 Da |
| Retention | Stage 3 Product | 1.94 minutes |
| Times and Molecular Masses | 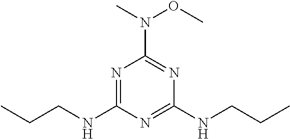 [SMILES: CCCNc1nc(NCCC)nc(N(C)OC)n1] | MW 250.31 |
| | 4 | |
| | Stage 2 Intermediate | 2.51 minutes (coelutes with other |
| | 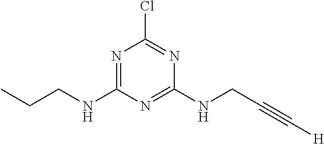 | trace byproducts) MW 225.68 |
| | 3 | |

### Method 5E

### LC/MS Method for a % Purity as Used for Examples 3D-3G

| Sample | Concentration | 1 mg/1 mL | |
|---|---|---|---|
| | Diluent | Acetonitrile | |
| Injector | Injection Volume | variable, 0.1-5 μL | |
| Inlet | Column Temperature | 30° C. | |
| | Flow Rate | 0.5 | mL/min |
| Gradient | Mobile phase A | 0.01% (v/v) trifluoroacetic acid in | |
| | | water | |
| | Mobile phase B | Acetonitrile | |
| | Gradient | from 10% to 95% B for 4.3 min | |
| | | hold 95% B for 1.7 min | |
| | | Return to initial conditions | |
| | | Hold for 2 min | |
| Column | Type | Waters ACQUITY UPLC ® BEH | |
| | | C18 1.7 μm; | |
| | | ser. no. 02083218625720 | |
| | Dimensions | 2.1 × 50 mm | |
| UV/VIS | Detector Type | Waters PDA eλ, Detector | |
| Detector | Wavelength | 220-320 | nm |
| MS | Detector Type | Waters SQ Detector 2 | |
| Detector | Source Temperature | 150° C. | |
| | Ionization mode | ESI+ | |
| | Capillary voltage | 2.8 | kV |
| | Cone voltage | 30 | V |
| | Extractor voltage | 3.0 | V |
| | Desolvation | 350° C. | |
| | Temperature | | |
| | Scan Range 1 | 50 to 150 amu ES+ | |
| | Scan Time 1 | 0.2 | s |
| | Scan Range 2 | 150 to 1200 amu ES+ | |
| | Scan Time 2 | 0.5 | s |

| | | |
|---|---|---|
| Retention | API Target | Retention Time: 1.82 minutes |
| times and Molecular Masses | 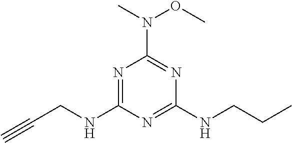 [SMILES: C#CCNc1nc(NCCC)nc(N(C)OC)n1] | MW: 250.31 |
| | 4 | |
| | Dimethyl amino | Retention Time: 2.09 minutes |
| | byproduct from | MW 254.19 |
| | Example 3 | |
| | 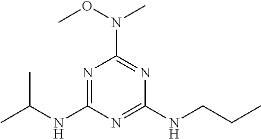 [SMILES: CCCNc1nc(NC(C)C)nc(N(C)OC)n1] | |
| | 12 | |

### Method 3M-F

### UPLC-MS/MS Method for quantitation of O,N-dimethyl-N-[4-(n-propylamino)-6-(2-chloroallylamino)-[1,3,5]triazin-2-yl]-hydroxylamine (IMP-A) (5-250 ppm) and other trace impurities in O,N-Dimethyl-N-[4-(n-propylamino)-6-(prop-2-ynylamino)-[1,3,5]-triazin-2-yl]-hydroxylamine (4)

### UPLC-MS/MS Conditions:

| Column | Acquity UPLC BEH C18, 2.1 × 50 mm, | |
|---|---|---|
| | 1.7 | μm |
| Column temperature | 30° C. | |
| Sample temperature | 10° C. | |
| Flow rate | 0.25 | mL/min |
| Run time | 6 | minutes |

| | |
|---|---|
| Injection volume, type* | 1 μL, partial loop with needle overfill |
| Mobile phase A | 0.1% (v/v) formic acid in water |
| Mobile phase B | Acetonitrile |
| Retention times and molecular masses | 2.48 minutes |
| 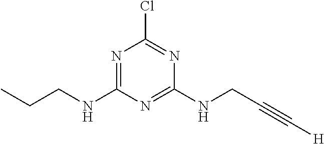 | MW 225.68 MS/MS: 226.2 >> 148.1 amu |
| 3 | |
| Structurally Related Vinyl Chloride | 2.27 minutes |
| Containing Byproduct | MW 286.16 |
| 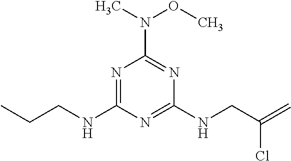 [SMILES: C=C(Cl)CNc1nc(NCCC)nc(N(C)OC)n1] | MS/MS: 287.2 >> 221.0 + 289.2 >> 221.0 amu |
| IMP-A | |
| Bis-n-Propylamino byproduct | 2.27 minutes |
| 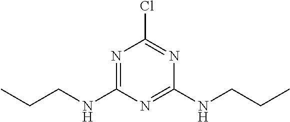 | MW 254.19 MS/MS: 255.3 >> 182.1 amu |
| 20 | |

*injection type may vary on different instruments

Gradient table:

| Time (minutes) | % A | % B |
|---|---|---|
| 0 | 80 | 20 |
| 1 | 80 | 20 |
| 2.5 | 2 | 98 |
| 4.5 | 2 | 98 |
| 4.7 | 80 | 20 |
| 6 | 80 | 20 |

MS/MS conditions table*:

| Ionization mode | ESI+ | |
|---|---|---|
| Capillary voltage | 2.8 | kV |
| Cone voltage | 30 | V |
| Extractor voltage | 3.0 | V |
| Source temperature | 120° | C. |
| Desolvation temperature | 400° | C. |
| Desolvation gas flow | 600 | L/H |
| Cone gas flow | 30 | L/H |
| Collision energy | 20 | eV |
| MS/MS transitions | 287 >> 221 | Da |
| | 289 >> 221 | Da |

*MS/MS parameters may vary on different instruments

### Example 3N

### Stage 5; Repetitive Recrystallization of O,N-dimethyl-N-[4-(n-propylamino)-6-(prop-2-ynylamino)-[1,3,5]triazin-2-yl]-hydroxylamine hydrogen sulfate (5b)

A. O,N-Dimethyl-N-[4-(n-propylamino)-6-(prop-2-ynylamino)-[1,3,5]triazin-2-yl]-hydroxylamine hydrogen sulfate (5b) was prepared in MEK by addition of H₂SO₄ (1.05 equiv.) to a hot solution of free base (melting point A in Table 57).

B. O,N-Dimethyl-N-[4-(n-propylamino)-6-(prop-2-ynylamino)-[1,3,5]triazin-2-yl]-hydroxylamine hydrogen sulfate (5b) from step A (10 g) was dissolved in IPA (60 mL) and heated to reflux. The solution was cooled to ambient temperature and Et₂O (5 mL) were added to initiate crystallization. The mixture was allowed to stand for 16 h at ambient temperature. The resultant product was collected by filtration, washed with IPA (3×7 mL) and air dried at 75° C. (5.5 g, 55%) (melting point B in Table 57).

C. O,N-Dimethyl-N-[4-(n-propylamino)-6-(prop-2-ynylamino)-[[1,3,5]triazin-2-yl]-hydroxylamine hydrogen sulfate (5b) from step B (4.8 g) was dissolved in MEK (120 mL) at reflux and activated charcoal (0.5 g) was added. The mixture was stirred for 16 h at 60° C. and then cooled to ambient temperature. The charcoal was filtered off and the filtrate was concentrated to 35 mL volume, at which time it was cooled to ambient temperature and allowed to stand for 16 h; crystallization occurred. The resultant product was collected by filtration, washed with MEK (3×15 mL) and dried under vacuum (0.2 mbar) (2.2 g, 46%) (melting point C in Table 57).

TABLE 57
Melting points of O,N-dimethyl-N-[4-(n-propylamino)-6-
(prop-2-ynylamino)-[1,3,5]triazin-2-yl]-hydroxylamine
hydrogen sulfate (5b) upon successive recrystallizations.

| Step | Solvent | mp (° C.) |
|---|---|---|
| A | MEK | 130-132 |
| B | IPA | 131-134 |
| C | MEK/charcoal | 129-131 |

### Example 3O

### Stage 5; Recrystallizations of O,N-dimethyl-N-[4-(n-propylamino)-6-(prop-2-ynylamino)-[1,3,5]triazin-2-yl]-hydroxylamine hydrogen sulfate (5b) from various solvents

O,N-Dimethyl-N-[4-(n-propylamino)-6-(prop-2-ynylamino)-[1,3,5]triazin-2-yl]-hydroxylamine hydrogen sulfate (5b) [prepared in MEK] (5 g) was dissolved in IPA (20 mL) at reflux and then cooled ambient temperature. No crystallization occurred. Et₂O (3 mL) was added in order to initiate crystallization and wool-like voluminous crystals formed. The crystallization process was allowed to proceed for 16 h at ambient temperature. The resultant product was collected by filtration, washed with IPA (2×20 mL), then with light petroleum ether (2×25 mL) and air dried at 65° C. for 16 h giving O,N-dimethyl-N-[4-(n-propylamino)-6-(prop-2-ynylamino-[1,3,5]triazin-2-yl]-hydroxylamine hydrogen sulfate (5b) as colorless crystals (2.5 g, 50%).

TABLE 58
Elemental analysis of O,N-dimethyl-N-[4-(n-propylamino)-
6-(prop-2-ynylamino)-[1,3,5]triazin-2-yl]-hydroxylamine
hydrogen sulfate (5b) recrystallized from IPA

| | C | H | N |
|---|---|---|---|
| Calculated | 37.92 | 5.79 | 24.12 |
| Test 1 | 38.04 | 5.80 | 24.28 |
| Test 2 | 37.98 | 5.70 | 24.25 |

O,N-Dimethyl-N-[4-(n-propylamino)-6-(prop-2-ynylamino)-[1,3,5]triazin-2-yl]-hydroxylamine hydrogen sulfate (5b) [prepared in MEK] (5 g) was dissolved in acetonitrile (20 mL) at reflux and then cooled to ambient temperature. No crystallization occurred. Et₂O (3 mL) was added in order to initiate crystallization. The crystallization process was allowed to proceed for 16 h at ambient temperature. The product was collected by filtration, washed with acetonitrile (2×20 mL), then with light petroleum ether (2×40 mL), and lastly air dried 70° C. for 16 h yielding O,N-dimethyl-N-[4-(n-propylamino)-6-(prop-2-ynylamino-[1,3,5]triazin-2-yl]-hydroxylamine hydrogen sulfate (5b) as colorless crystals (2.3 g, 46%).

TABLE 59
Elemental analysis of O,N-dimethyl-N-[4-(n-propylamino)-
6-(prop-2-ynylamino)-[1,3,5]triazin-2-yl]-hydroxylamine
hydrogen sulfate (5b) recrystallized from acetonitrile.

| | C | H | N |
|---|---|---|---|
| Calculated | 37.92 | 5.79 | 24.12 |
| Test 1 | 38.04 | 5.58 | 24.08 |
| Test 2 | 38.01 | 5.61 | 24.04 |

O,N-Dimethyl-N-[4-(n-propylamino)-6-(prop-2-ynylamino)-[1,3,5]triazin-2-yl]-hydroxylamine hydrogen sulfate (5b) [prepared in MEK] (5 g) was dissolved in acetone (60 mL) at reflux and then cooled to ambient temperature. The mixture was allowed to undergo recrystallized for 16 h at ambient temperature with partial evaporation of the solvent to the final volume of 40 mL. After this time, wool-like voluminous crystals were formed. The product was collected by filtration, washed with acetone (2×20 mL), then with light petroleum ether (2×40 mL), and lastly air dried 70° C. for 16 h yielding O,N-dimethyl-N-[4-(n-propylamino)-6-(prop-2-ynylamino-[1,3,5]triazin-2-yl]-hydroxylamine hydrogen sulfate (5b) as colorless crystals (2.1 g, 42%).

TABLE 60
Elemental analysis of O,N-dimethyl-N-[4-(n-propylamino)-
6-(prop-2-ynylamino)-[1,3,5]triazin-2-yl]-hydroxylamine
hydrogen sulfate (5b) recrystallized from acetone.

| | C | H | N |
|---|---|---|---|
| Calculated | 37.92 | 5.79 | 24.12 |
| Test 1 | 38.00 | 5.71 | 24.19 |
| Test 2 | 37.97 | 5.74 | 24.17 |

O,N-Dimethyl-N-[4-(n-propylamino)-6-(prop-2-ynylamino)-[1,3,5]triazin-2-yl]-hydroxylamine hydrogen sulfate (5b) [prepared in MEK] (5 g) was dissolved in MEK (55 mL) at reflux and then cooled to ambient temperature. The mixture was allowed to undergo recrystallized for 1 h at ambient temperature. After this time, wool-like voluminous crystals were formed. The product was collected by filtration, washed with MEK (2×25 mL), then with light petroleum ether (2×25 mL) and lastly air dried at 65° C. for 16 h yielding O,N-dimethyl-N-[4-(n-propylamino)-6-(prop-2-ynylamino)-[1,3,5]triazin-2-yl]-hydroxylamine hydrogen sulfate (5b) as colorless crystals (3.1 g, 62%).

TABLE 61
Elemental analysis of O,N-dimethyl-N-[4-(n-propylamino)-
6-(prop-2-ynylamino)-[1,3,5]triazin-2-yl]-hydroxylamine
hydrogen sulfate (5b) recrystallized from MEK.

| | C | H | N |
|---|---|---|---|
| Calculated | 37.92 | 5.79 | 24.12 |
| Test 1 | 37.96 | 5.70 | 24.30 |
| Test 2 | 38.01 | 5.74 | 24.32 |

O,N-Dimethyl-N-[4-(n-propylamino)-6-(prop-2-ynylamino)-[1,3,5]triazin-2-yl]-hydroxylamine hydrogen sulfate (5b) is freely soluble in water and MeOH.

TABLE 62
Recrystallizations of O,N-dimethyl-N-[4-(n-propylamino)-6-(prop-2-
ynylamino)-[1,3,5]triazin-2-yl]-hydroxylamine hydrogen sulfate (5b)

| | | Dilution | Yield | |
|---|---|---|---|---|
| # | Solvent | (mL/g) | (%) | Comment |
| 1 | IPA | 4 | 50 | Addition of Et₂O (10% v/v) was |
| | | | | needed to effect crystallization |
| 2 | MeCN | 4 | 46 | Addition of Et₂O (10% v/v) was |
| | | | | needed to effect crystallization |
| 3 | Acetone | 12 | 42 | |
| 4 | MEK | 12 | 62 | Cloud point: 57° C. At 52° C. |
| | | | | crystallization was significant. |

### Example 3P

### Crystallization of O,N-dimethyl-N-[4-(n-propylamino)-6-(prop-2-ynylamino-[1,3,5]triazin-2-yl]-hydroxylamine as Hydrogen Sulfate Salt (5b) with Various Additives

In each experiment, O,N-dimethyl-N-[4-(n-propylamino)-6-(prop-2-ynylamino-[1,3,5]triazin-2-yl]-hydroxylamine free base (4) (1 g) was dissolved in MEK (10 mL), heated to 70° C. and insoluble materials were filtered off. To the hot filtrate, a single portion of H₂SO₄ (260 μL, 1.1 equiv.) was added at once. At this time, the indicated amount of an additive was added and the mixture was allowed to cool to ambient temperature to allow for crystallization to occur. In the case of mixtures A, B and C no crystallization occur. They were placed in an ice bath, where mixtures B and C crystallized. Addition of water (mixture A) prevented crystallization completely. In all cases crystals had the same wool-like appearance.

TABLE 63
Crystallizations of O,N-dimethyl-N-[4-(n-propylamino)-
6-(prop-2-ynylamino)-[1,3,5]triazin-2-yl]-hydroxylamine
hydrogen sulfate (5b) from MEK via additives.

| | | Amount | Crystallized at temperature (° C.) | |
|---|---|---|---|---|
| # | Additive | (vol %) | r.t. | 0 (ice bath) |
| A | Water | 5 | no | no |
| B | EtOH | 5 | no | yes |
| C | IPA | 5 | no | yes |
| D | MTBE | 10 | yes | — |
| E | THF | 5 | yes | — |
| F | PE40 | 10 | yes | — |

### Example 3Q

### Formation of O,N-dimethyl-N-[4-(n-propylamino)-6-(prop-2-ynyl amino)-[1,3,5]triazin-2-yl]-hydroxylamine salts upon treatment with sulfuric acid

O,N-Dimethyl-N-[4-(n-propylamino)-6-(prop-2-ynylamino)-[1,3,5]triazin-2-yl]-hydroxylamine free base (43.5 g) was dissolved in Et₂O (800 mL), cooled to 0° C. (ice bath). To this solution concentrated 95% H₂SO₄ (1 eq, 9.3 mL) was added dropwise with stirring. The reaction mixture stirred for 1 h, after which time the resultant solid crystalline product was collected by filtration and washed with Et₂O to afford a product that was determined by elemental analysis to be a half-sulfate adduct with a triazine/H₂SO₄=2:1 molar ratio (18.4 g, mp 102-104° C.).

TABLE 64
Elemental analysis of O,N-dimethyl-N-[4-(n-propylamino)-6-(prop-
2-ynylamino)-[1,3,5]triazin-2-yl]-hydroxylamine/sulfuric acid addition salt
in a 2:1 molar ratio (C₁₁H₁₈N₆O * 0.5 H₂SO₄).

| | C | H | N |
|---|---|---|---|
| Calculated | 44.14 | 6.40 | 28.08 |
| Test 1 | 44.00 | 6.30 | 28.05 |
| Test 2 | 44.09 | 6.29 | 28.07 |

The oily residue remaining in the flask from procedure A above was suspended in Et₂O (200 mL) with EtOH (30 mL), and sonicated for 1 h at ambient temperature. The resulting solids were collected by filtration and washed with Et₂O and lastly air dried at 60° C. to afford a product that was determined by elemental analysis to be a bis-sulfate with a triazine/H₂SO₄=1:2 molar ratio (21.4 g, mp 165-167° C.).

TABLE 65
Elemental analysis of O,N-dimethyl-N-[4-(n-propylamino)-6-(prop-
2-ynylamino)-[1,3,5]triazin-2-yl]-hydroxylamine/sulfuric acid addition salt
in a 1:2 molar ratio (C₁₁H₁₈N₆O * 2 H₂SO₄).

| | C | H | N |
|---|---|---|---|
| Calculated | 29.59 | 4.97 | 18.82 |
| Test 1 | 29.64 | 4.78 | 18.61 |
| Test 2 | 29.81 | 4.81 | 18.72 |

The combined filtrates from procedure B above were evaporated to dryness and dried under vacuum (0.2 mbar) to afford a product that was determined by elemental analysis to be an addition salt with triazine/H₂SO₄=4:3 molar ratio (21.5 g, mp 53-57° C.). The XRPD data indicated that this substance is completely amorphous.

TABLE 66
Elemental analysis of O,N-dimethyl-N-[4-(n-propylamino)-6-(prop-
2-ynylamino)-[1,3,5]triazin-2-yl]-hydroxylamine/sulfuric acid addition salt
in a 4:3 molar ratio (4 C₁₁H₁₈N₆O *3 H₂SO₄).

| | C | H | N |
|---|---|---|---|
| Calculated | 29.59 | 4.97 | 18.82 |
| Test 1 | 29.64 | 4.78 | 18.61 |
| Test 2 | 29.81 | 4.81 | 18.72 |

Fractions 1-3 were combined, dissolved in EtOH (350 mL) and sonicated for 1 h to ensure complete dissolution. The solvent was removed under vacuum and the resultant semisolid residue was dried under vacuum (0.2 mbar) at ambient temperature for 3 h. The completely solidified residue was then air dried at 60° C. for 16 h to afford a product that was determined by elemental analysis to be an addition salt with triazine/H₂SO₄=4:3 molar ratio. Yield: 60.5 g (99%), mp 130-132° C., elemental analysis showed it to be a monohydrogen sulfate with triazine/H₂SO₄.

TABLE 67
Elemental analysis for triazine/H₂SO₄ =
4:3 molar ratio (4 C₁₁H₁₈N₆O * 3 H₂SO₄).

| | C | H | N |
|---|---|---|---|
| Calculated | 38.25 | 5.82 | 24.33 |
| Found 1 | 38.22 | 5.65 | 24.23 |
| Found 2 | 38.32 | 5.64 | 24.30 |

TABLE 68
O,N-Dimethyl-N-[4-(n-propylamino)-6-(prop-2-ynylamino)-
[1,3,5]triazin-2-yl]-hydroxylamine/sulfuric acid
addition salts molar ratios (x C₁₁H₁₈N₆O * y H₂SO₄).

| | | mp | Yield | Triazine/H₂SO₄ |
|---|---|---|---|---|
| Fraction | Conditions | (° C.) | (%) | molar ratio |
| A | Precipitated as a slightly | 102-104 | 30 | 2:1 |
| | yellow solid | | | |
| B | Precipitated as a clean | 165-167 | 35 | 1:2 |
| | slightly yellow oil, stick | | | |
| | to the bottom of the flask; | | | |
| | solidified upon trituration | | | |
| | with Et₂O | | | |
| C | Filtrate from fraction 1 | 53-57 | 36 | 4:3 |
| | was evaporated to dryness | | | |
| | to yield yellowish solid | | | |

### Example 3R

### Reactivity of Cyanuric Chloride

The reactivity of cyanuric chloride (1) in various solvents was examined, to determine whether this reactive starting material would form undesired by-products by solvolysis. Two products were obtained upon the reaction of cyanuric chloride with isopropanol (IPA) (Scheme 13), namely 6-isopropoxy-2,4-dichloro-1,3,5-triazine (or [4,6-dichloro-1,3,5-triazin-2-yl]-isopropyl ether] a, and N-[4,6-dichloro-1,3,5-triazin-2-yl]-N-ethyl-N-isopropylamine] b.

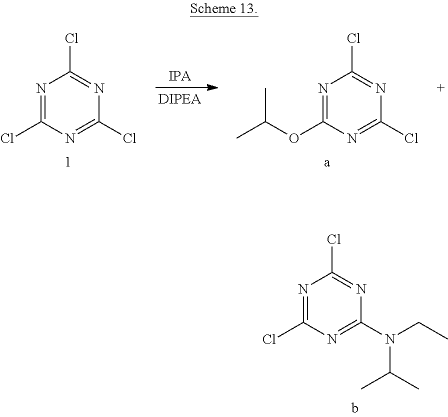

Cyanuric chloride (0.5 g) was suspended in IPA (5 mL) and stirred at ambient temperature. GC-MS Analysis was conducted after 1 h and 4 h of stirring (Table 21). The reaction mixture became clear after 7 h.

Cyanuric chloride (5 g) was suspended in cold IPA (25 mL), cooled in an ice bath and stirred at 0° C. GC-MS analysis was conducted after 1 and 4 h of stirring (Table 21). After 4 h, the reaction mixture was filtered and washed with mixture IPA/light petroleum ether (2×20 mL). The filtrate was evaporated to dryness to yield 0.54 g of a colorless solid.

Cyanuric chloride (0.5 g) was suspended in IPA (5 mL) and DIPEA (0.47 mL, 1 equiv.) was added. The reaction mixture was stirred at ambient temperature. GC-MS analysis was conducted after 1 and 4 h of stirring (Table 21). The reaction mixture became clear after 2 h.

TABLE 69
Reaction of cyanuric chloride in IPA with
and without the amine base DIPEA.

| | | | Result (GC-MS assay) | |
|---|---|---|---|---|
| Synthesis | Base | Temp (° C.) | 1 h | 4 h |
| 1 | no | r.t. | 1:a = 10:1 | 1:a = 1:2 |
| 2 | no | 0 to −2 | 1:a = 50:1 | 1:a = 10:1 |
| 3 | DIPEA | r.t. | 1:a:b = 12:3:1 | 1:a:b = 0:95:5 |

### Example 3S

### Solubility of Cyanuric Chloride

The solubility of cyanuric chloride was examined as a way to limit its reactivity and the formation of undesired by-products. Cyanuric chloride (10 g) was mixed with 50 mL of indicated solvent (Table 22) and sonicated for 1 h with occasional swirling. If solid material remained as a suspension, it was filtered off and weighed.

TABLE 70
Solubility of cyanuric chloride in various solvents.

Temperature (° C.)

| Solvent | r.t. | 0 |
|---|---|---|
| Dioxane | soluble | soluble |
| THF | soluble | soluble |
| Toluene | soluble | soluble |
| IPA | 0.07 mg/mL | insoluble |
| MeCN | 0.15 mg/mL | insoluble |
| Acetone | soluble | partial crystallization |
| DMSO | decomposition | decomposition |
| DMF | decomposition | decomposition |
| DMAc | soluble | soluble |

### Example 3T

### Analytics

TABLE 71
GC-MS conditions.

| Injector | Injection Volume | 1 | μL |
|---|---|---|---|
| Inlet | Inlet Mode | Split | |
| | Inlet Temperature | 200° | C. |
| | Split Ratio | 20:1 | |
| | Initial Pressure | 2.98 | psi |
| | Flow Rate (Constant Flow) | 13.5 | mL/min |
| | Carrier Gas | Helium | |
| Oven | Temperature Program | 50° C. for 3 minutes, | |
| | | 25° C./min to 250° C., | |
| | | Hold at 250° C. for 4 minutes | |
| | Maximum Temperature | 325° | C. |
| Column | Dimensions | HP-5MS; 30 m × 0.250 mm, | |
| | | 0.25 μm film thickness | |
| Detector | Detector Type | Single Quadrupole MS | |
| | Auxiliary Temperature | 250° | C. |
| | MS Source Temperature | 230° | C. |
| | MS Quad Temperature | 150° | C. |
| | Solvent Delay | 2.0 | min |
| | Scan Type | Scan Mode | |
| | Scan Speed | Normal | |
| | Scan Range | 10 to 500 | amu |
| | Scan Rate | 5.6 | scans/sec |
| | Threshold | 50 | counts |

TABLE 72
GC-MS retention times and ionization of major components from Stage 1
to Stage 4 reaction mixtures.

| | Retention | |
|---|---|---|
| | time | |
| Component | (min) | m/z |
| 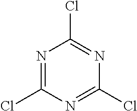 | 9.40 | 183, 185, 187 |
| 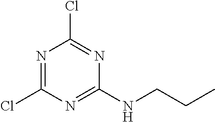 | 14.78 | 206, 208 177, 179 |
| 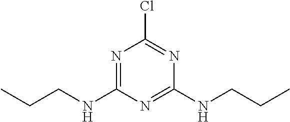 | 17.10 | 229, 231 200, 202 |
| 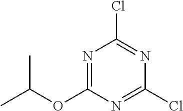 | 12.24 | 207, 209 166, 168 |

### Example 4

### N-Methyl-N′-n-propyl-N″-prop-2-ynyl-[1,3,5]triazine-2,4,6-triamine (6) and corresponding hydrochloride salt (7a) (Scheme 14)

### N-Methyl-N′-n-propyl-N″-prop-2-ynyl-[1,3,5]triazine-2,4,6-triamine (6)

A solution of 6-chloro-N²-(prop-2-ynyl)-N⁴-n-propyl-1,3,5-triazine-2,4-diamine (3) (350 mg, 1.55 mmol), 2M MeNH₂/THF (7.8 mL, 15.60 mmol) and NaOH (74 mg, 1.86 mmol) in 1,4-dioxane (10 mL) was heated at 70° C. for 5 h. The volatiles were removed under reduced pressure. Water (20 mL) was added to the residue, and the mixture was extracted with EtOAc (3×20 mL). The combined organic extracts were washed with water (30 mL), and then with a brine solution (30 mL), and dried over anhydrous Na₂SO₄. The volatiles were removed under reduced pressure and the resultant residue was purified by flash column chromatography using gradient elution from CH₂Cl₂/EtOH (99:1) to CH₂Cl₂/EtOH (95:5) to yield N-methyl-N′-n-propyl-N″-prop-2-ynyl-[1,3,5]triazine-2,4,6-triamine (6) (310 mg, 91%). 400 MHz ¹H NMR (CDCl₃, ppm): δ 5.04-4.62 (3H, m), 4.16-4.05 (2H, m), 3.31-3.16 (2H, m), 2.84 (3H, d, J=4.2 Hz), 2.13 (1H, t, J=2.5 Hz), 1.50 (2H, sextet, J=7.4 Hz), 0.87 (3H, t, J=7.4 Hz). ESI-MS (m/z): 221 [M+H]⁺.

### N-Methyl-N′-n-propyl-N″-prop-2-ynyl-[1,3,5]triazine-2,4,6-triamine hydrochloride (7a)

A 2M HCl/diethyl ether (0.68 mL, 1.36 mmol) solution was added to the solution of N-methyl-N′-n-propyl-N″-prop-2-ynyl-[1,3,5]triazine-2,4,6-triamine (6) (300 mg, 1.36 mmol) in diethyl ether (15 mL) at 0° C. The mixture was stirred for 0.5 h at 0° C. and then the volatiles were removed under reduced pressure to yield N-methyl-N′-n-propyl-N″-prop-2-ynyl-[1,3,5]triazine-2,4,6-triamine hydrochloride (7a) in quantitative yield. 400 MHz ¹H NMR (DMSO-d₆, ppm): δ 12.6-11.4 (1H, m), 8.85-8.05 (3H, m), 4.19-4.00 (2H, m), 3.60-3.08 (3H, m, overlapped with water), 2.91-2.77 (3H, m), 1.60-1.45 (2H, m), 0.93-0.81 (3H, m). ESI-MS (m/z): 221 [M+H]⁺.

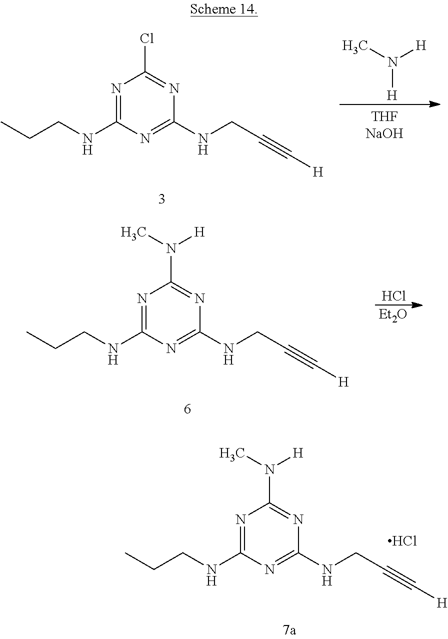

### Example 4a

### N-Methyl-N′-n-propyl-N″-prop-2-ynyl-[1,3,5]triazine-2,4,6-triamine (6) and corresponding hydrochloride salt (7a) (Scheme 14)

### N-Methyl-N′-n-propyl-N″-prop-2-ynyl-[1,3,5]triazine-2,4,6-triamine (6)

A mixture of 6-chloro-N²-(prop-2-ynyl)-N⁴-propyl-1,3,5-triazine-2,4-diamine (3) (5.00 g, 22.16 mmol), and MeNH₂/water solution (40%) (30 mL) in 1,4-dioxane (30 mL) was heated at 60° C. for 4 h in a closed vial. Saturated NaHCO₃ solution (100 mL) was added, and the resulting suspension was extracted with EtOAc (3×100 mL). The combined organic extracts were washed with water (150 mL), then with a brine solution (150 mL) and lastly dried over anhydrous Na₂SO₄. After filtration, the solvent was removed under reduced pressure and the residue was filtered through a silica gel column using CH₂Cl₂/EtOH (97:3) as an eluent. The volatiles were removed to yield N-methyl-N′-propyl-N″-prop-2-ynyl-[1,3,5]triazine-2,4,6-triamine (6) (4.65 g, 95%). 400 MHz ¹H NMR (CDCl₃, ppm): δ 5.25 (1H, br s), 4.95 (2H, br s), 4.25-4.11 (2H, m), 3.39-3.23 (2H, m), 2.91 (3H, d, J=4.0 Hz), 2.19 (1H, t, J=2.6 Hz), 1.62-1.50 (2H, m), 0.94 (3H, t, J=7.4 Hz). ESI-MS (m/z): 221 [M+H]⁺.

### N-Methyl-N′-n-propyl-N″-prop-2-ynyl-[1,3,5]triazine-2,4,6-triamine hydrochloride (7a)

To a solution of N-methyl-N′-propyl-N″-prop-2-ynyl-[1,3,5]triazine-2,4,6-triamine (6) (3.00 g, 13.62 mmol) in diethyl ether (30 mL) and ethanol (3 mL) at 0° C., was added 95% H₂SO₄ (0.76 mL, 13.62 mmol) in a dropwise manner. The mixture was stirred for 0.5 h at ambient temperature, and the volatiles were removed under reduced pressure to yield N-methyl-N′-propyl-N″-prop-2-ynyl-[1,3,5]triazine-2,4,6-triamine hydrogen sulfate (7a) in quantitative yield. 400 MHz ¹H NMR (D₂O, ppm): δ 4.18-3.91 (2H, m), 3.34-3.08 (2H, m), 2.89-2.70 (3H, m), 2.54-2.46 (1H, m), 1.52-1.37 (2H, m), 0.76 (3H, t, J=7.3 Hz). ESI-MS (m/z): 221 [M+H]⁺.

### Example 5

### N-(4-Fluorobenzyl)-O-methyl-N-[4-(n-propylamino)-6-(prop-2-ynylamino)-[1,3,5]triazin-2-yl]-hydroxylamine (8) and corresponding hydrochloride salt (9a) (Scheme 15)

### N-(4-Fluorobenzyl)-O-methyl-N-[4-(n-propylamino)-6-(prop-2-ynylamino)-[1,3,5]triazin-2-yl]-hydroxylamine (8)

A mixture of 6-chloro-N²-(prop-2-ynyl)-N⁴-n-propyl-1,3,5-triazine-2,4-diamine (3) (250 mg, 1.11 mmol) and N-(4-fluorobenzyl)-O-methylhydroxylamine (347 mg, 2.22 mmol) in 1,4-dioxane (3 mL) was heated at 90° C. for 20 h, after which the volatiles was removed under reduced pressure. A saturated NaHCO₃ solution (20 mL) was added to the residue and the mixture was extracted with EtOAc (3×20 mL). The combined organic extracts were washed with water (40 mL), then with a brine solution (40 mL) and lastly dried over anhydrous Na₂SO₄. The volatiles were removed under reduced pressure and the resultant residue was purified by flash column chromatography using gradient elution from CH₂Cl₂/EtOH (99:1) to CH₂Cl₂/EtOH (95:5) to yield N-(4-fluoro-benzyl)-O-methyl-N-[4-(n-propylamino)-6-(prop-2-ynylamino)-[1,3,5]triazin-2-yl]-hydroxylamine (8) (325 mg, 85%). 400 MHz ¹H NMR (DMSO-d₆, ppm): δ 7.44-7.28 (2H, m), 7.04-6.93 (2H, m), 5.1-4.9 (2H, br s), 4.86 (2H, s), 4.18 (2H, s), 3.68 (3H, s), 3.41-3.26 (2H, m), 2.20 (1H, t, J=2.4 Hz), 1.67-1.50 (2H, m), 0.94 (3H, t, J=7.4 Hz). ESI-MS (m/z): 345 [M+H]⁺.

### N-(4-Fluorobenzyl)-O-methyl-N-[4-(n-propylamino)-6-(prop-2-ynylamino)-[[1,3,5]triazin-2-yl]-hydroxylamine hydrochloride (9a)

N-(4-Fluoro-benzyl)-O-methyl-N-[4-(n-propylamino)-6-(prop-2-ynylamino-[1,3,5]triazin-2-yl]-hydroxylamine (8) and 2M HCl/diethyl ether were reacted using the procedure described for Compound 7 above to afford N-(4-fluoro-benzyl)-O-methyl-N-(4-n-propylamino-6-prop-2-ynylamino-[1,3,5]triazin-2-yl)-hydroxylamine hydrochloride (9a). 400 MHz ¹H NMR (CDCl₃, ppm) δ 13.76-13.30 (1H, m), 9.83-9.14 (1H, m), 7.39-7.27 (2H, m), 7.07-6.97 (2H, m), 5.99-5.65 (1H, m), 4.98-4.78 (2H, m), 4.31-4.08 (2H, m), 3.96-3.72 (3H, m), 3.48-3.24 (2H, m), 2.33-2.18 (1H, m), 1.73-1.53 (2H, m), 1.04-0.88 (3H, m). ESI-MS (m/z): 345 [M+H]⁺.

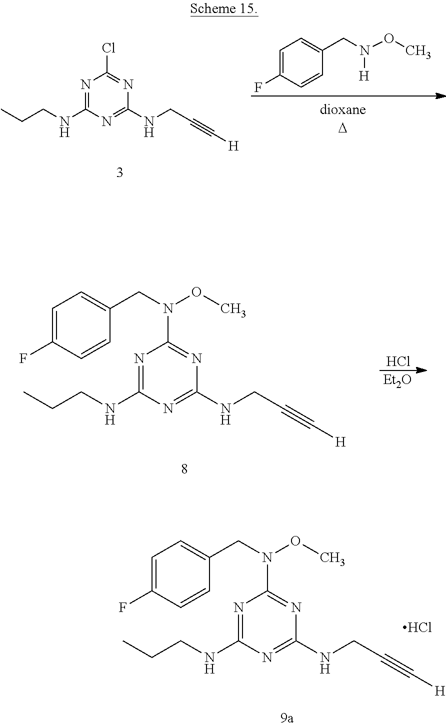

### Example 6

### N-(4-Fluoro-benzyl)-N′-n-propyl-N″-prop-2-ynyl-[1,3,5]triazine-2,4,6-triamine (11) and corresponding hydrochloride salt (12a) (Scheme 16)

### 6-Chloro-N²-(4-fluorobenzyl)-N⁴-(prop-2-ynyl)-1,3,5-triazine-2,4-diamine (10)

(4-Fluorophenyl)methanamine (0.75 mL, 6.52 mmol) and N,N-diisopropylethylamine (1.14 mL, 6.52 mmol) was added to a cooled solution (0° C.) of cyanuric chloride (1) (1.2 g, 6.52 mmol) in acetonitrile (60 mL). The reaction mixture was stirred at 0° C. for 2 h. Propargylamine hydrochloride (597 mg, 6.52 mmol) and N,N-diisopropylethylamine (2.28 mL, 13.04 mmol) were added, and the reaction mixture was heated at 50° C. for 4 h. The mixture was cooled to room temperature. The resultant precipitate was filtered, washed with water and acetonitrile and then dried to afford 6-chloro-N²-(4-fluorobenzyl)-N⁴-(prop-2-ynyl)-1,3,5-triazine-2,4-diamine (10) (1.77 g, 93%). 400 MHz ¹H NMR (DMSO-d₆, ppm): δ 8.52-8.32 (1H, m) 8.26-8.12 (1H, m) 7.43-7.27 (2H, m) 7.17-7.10 (2H, m) 4.47-4.36 (2H, m) 4.06-3.96 (2H, m) 3.15-3.09 (1H, m). ESI-MS (m/z): 292, 294 [M+H]⁺. N-(4-Fluoro-benzyl)-N′-n-propyl-N″-prop-2-ynyl-[1,3,5]triazine-2,4,6-triamine (11):

A mixture of 6-chloro-N²-(4-fluorobenzyl)-N⁴-(prop-2-ynyl)-1,3,5-triazine-2,4-diamine (10) (250 mg, 0.86 mmol) and n-propylamine (0.5 mL) in 1,4-dioxane (20 mL) was heated at 90° C. for 16 h, after which time the volatiles was removed under reduced pressure. A saturated NaHCO₃ solution (20 mL) was added to the residue and the mixture was extracted with EtOAc (3×20 mL). The combined organic extracts were washed with water (40 mL), then with a brine solution (40 mL) and lastly dried over anhydrous Na₂SO₄. The volatiles were removed and the residue was purified by flash column chromatography using gradient elution from PE/EtOAc (3:1) to PE/EtOAc (1:1) to yield N-(4-fluoro-benzyl)-N′-n-propyl-N″-prop-2-ynyl-[1,3,5]triazine-2,4,6-triamine (11) (242 mg, 90%). 400 MHz ¹H NMR (CDCl₃, ppm): δ 7.29-7.24 (2H, m), 7.00-6.93 (2H, m), 5.27-5.00 (1H, m), 5.00-4.71 (2H, m), 4.51 (2H, d, J=5.2 Hz), 4.13 (2H, s), 3.45-3.21 (2H, m), 2.17 (1H, t, J=2.5 Hz), 1.59-1.48 (2H, m), 0.91 (3H, t, J=7.4 Hz). ESI-MS (m/z): 315 [M+H]⁺.

### N-(4-Fluoro-benzyl)-N′-n-propyl-N″-prop-2-ynyl-[1,3,5]triazine-2,4,6-triamine hydrochloride (12a)

N-(4-Fluoro-benzyl)-N′-n-propyl-N″-prop-2-ynyl-[1,3,5]triazine-2,4,6-triamine (11) and 2M HCl/diethyl ether were reacted using procedure described for Compound 7, to yield N-(4-fluoro-benzyl)-N′-n-propyl-N″-prop-2-ynyl-[1,3,5]triazine-2,4,6-triamine hydrochloride (12). 400 MHz ¹H NMR (CDCl₃, ppm): δ 13.61 (1H, br s), 8.01 (0.5H, br s), 7.68 (0.5H, br s), 7.52-7.36 (1H, m), 7.34-7.15 (2H, m), 7.06-6.93 (2H, m), 5.93-5.55 (1H, m), 4.61-4.46 (2H, m), 4.23-4.08 (2H, m), 3.43-3.24 (2H, m), 2.29-2.19 (1H, m), 1.68-1.52 (2H, m), 0.99-0.88 (3H, m). ESI-MS (m/z): 315 [M+H]⁺.

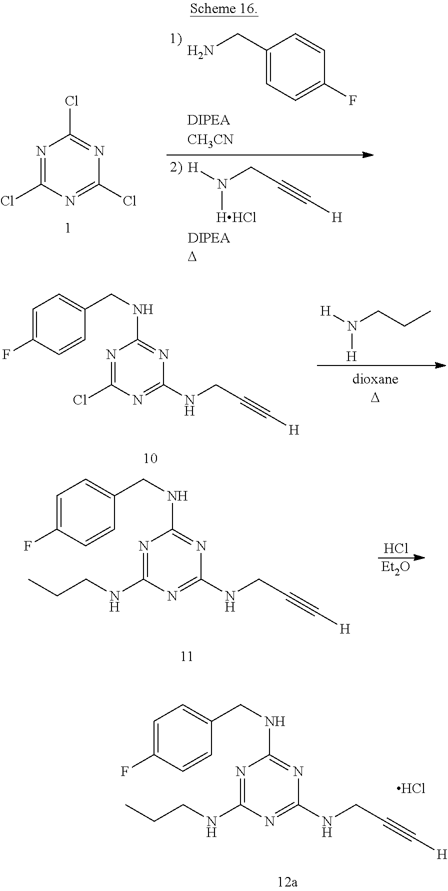

### Example 7

### N-[4-(4-Fluoro-benzylamino)-6-(prop-2-ynylamino)-[1,3,5]triazin-2-yl]-O,N-dimethyl-hydroxylamine (13) and corresponding hydrochloride salt (14a) (Scheme 17)

### N-[4-(4-Fluorobenzylamino)-6-(prop-2-ynylamino)-[1,3,5]triazin-2-yl]-O,N-dimethyl-hydroxylamine (17)

6-Chloro-N²-(4-fluorobenzyl)-N⁴-(prop-2-ynyl)-1,3,5-triazine-2,4-diamine (10) and O,N-dimethylhydroxylamine hydrochloride were reacted using procedure described for Compound 4 to yield the desired product in 98% yield. 400 MHz ¹H NMR (CDCl₃, ppm): δ 7.33-7.27 (2H, m), 7.03-6.94 (2H, m), 5.45-5.18 (1H, m), 5.18-4.95 (1H, m), 4.55 (2H, d, J=5.7 Hz), 4.26-4.11 (2H, m), 3.75 (3H, s), 3.28 (3H, s), 2.19 (1H, t, J=2.4 Hz). ESI-MS (m/z): 317 [M+H]⁺.

### N-[4-(4-Fluoro-benzylamino)-6-(prop-2-ynylamino)-[1,3,5]triazin-2-yl]-O,N-dimethyl-hydroxylamine hydrochloride (14a)

N-[4-(4-fluoro-benzylamino)-6-prop-2-ynylamino-[1,3,5]triazin-2-yl]-O,N-dimethyl-hydroxylamine (13) was reacted with 2M HCl/diethyl ether using procedure described for Compound 7 to yield the desired product. 400 MHz ¹H NMR (CDCl₃, ppm): δ 13.8-13.5 (1H, br s), 10.16-9.45 (1H, m), 7.41-7.27 (2H, m), 7.10-6.92 (2H, m), 6.19-5.62 (1H, m), 4.67-4.48 (2H, m), 4.26-4.10 (2H, m), 4.00-3.87 (3H, m), 3.47-3.28 (3H, m), 2.33-2.18 (1H, m). ESI-MS (m/z): 317 [M+H]⁺. Melting point: 103-105° C.

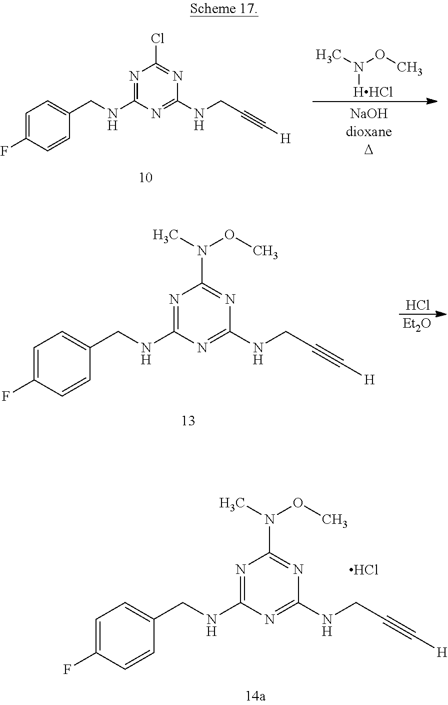

### Example 8

### N-(4-Fluoro-benzyl)-N-[4-(4-fluorobenzylamino)-6-(prop-2-ynylamino)-[1,3,5]triazin-2-yl]-O-methyl-hydroxylamine (15) and corresponding hydrochloride salt (16a) (Scheme 18)

### N-(4-Fluoro-benzyl)-N-[4-(4-fluorobenzylamino)-6-(prop-2-ynylamino)-[1,3,5]triazin-2-yl]-O-methyl-hydroxylamine (15)

6-Chloro-N²-(4-fluorobenzyl)-N⁴-(prop-2-ynyl)-1,3,5-triazine-2,4-diamine (10) and N-(4-fluorobenzyl)-O-methylhydroxylamine were reacted using procedure described for Compound 8 to yield N-(4-fluoro-benzyl)-N-[4-(4-fluoro-benzylamino)-6-prop-2-ynylamino-[1,3,5]triazin-2-yl]-O-methyl-hydroxylamine (15) in 84% yield. 400 MHz ¹H NMR (CDCl₃, ppm): δ 7.39-7.19 (4H, m, overlapped with CDCl₃), 7.06-6.88 (4H, m), 5.43-5.21 (1H, m), 5.09 (1H, br s), 4.85 (2H, s), 4.61-4.48 (2H, m), 4.23-4.13 (2H, m), 3.66 (3H, s), 2.20 (1H, t, J=2.3 Hz). ESI-MS (m/z): 411 [M+H]⁺.

### (N-(4-Fluoro-benzyl)-N-[4-(4-fluorobenzylamino)-6-(prop-2-ynylamino)-[1,3,5]triazin-2-yl]-O-methyl-hydroxylamine hydrochloride (16a)

N-(4-Fluoro-benzyl)-N-[4-(4-fluorobenzylamino)-6-prop-2-ynylamino-[1,3,5]triazin-2-yl]-O-methyl-hydroxylamine (15) and 2M HCl/diethyl ether were reacted using procedure described for compound 7. 400 MHz ¹H NMR (CDCl₃, ppm): δ 13.74 (1H, br s), 10.5-9.3 (1H, m), 7.43-7.27 (3H, m), 7.25-7.13 (1H, m), 7.11-6.92 (4H, m), 6.4-5.4 (1H, m), 4.98-4.81 (2H, m), 4.66-4.49 (2H, m), 4.26-4.12 (2H, m), 3.95-3.61 (3H, m), 2.33-2.21 (1H, m). ESI-MS (m/z): 411 [M+H]⁺.

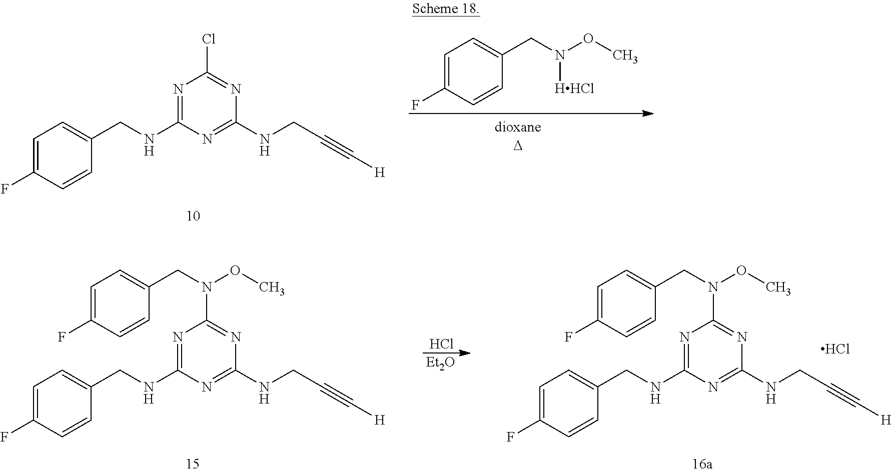

### Example 9

### N,N′-Bis-(4-fluoro-benzyl)-N″-prop-2-ynyl-[1,3,5]triazine-2,4,6-triamine (18) and corresponding hydrochloride salt (19) (Scheme 19)

### 6-Chloro-N²,N⁴-bis(4-fluorobenzyl)-1,3,5-triazine-2,4-diamine (17)

A mixture of cyanuric chloride (1) (600 mg, 3.26 mmol), (4-fluorophenyl)methanamine (0.75 mL, 6.52 mmol) and N,N-diisopropylethylamine (1.14 mL, 6.52 mmol) in acetonitrile (40 mL) was heated at 60° C. for 16 h. The mixture was cooled to room temperature and the precipitate was filtered, washed with water and MeCN and dried to yield 6-chloro-N²,N⁴-bis(4-fluorobenzyl)-1,3,5-triazine-2,4-diamine (17) (1.12 g, 95%). 400 MHz ¹H NMR (DMSO-d₆, ppm) δ 8.4 (1H, t, J=6.5 Hz), 8.34-8.27 (0.8H, m), 8.18-8.13 (0.2H, m), 7.35-7.27 (1.6H, m), 7.24-7.18 (2.4H, m), 7.17-7.10 (1.6H, m), 7.08-7.01 (2.4H, m), 4.44-4.36 (4H, m).

### N,N′-Bis-(4-fluorobenzyl)-N″-prop-2-ynyl-[1,3,5]triazine-2,4,6-triamine (18)

A mixture of 6-chloro-N²,N⁴-bis(4-fluorobenzyl)-1,3,5-triazine-2,4-diamine (17) (362 mg, 1.00 mmol), propargylamine hydrochloride (220 mg, 2.40 mmol) and NaOH (128 mg, 3.20 mmol) in 1,4-dioxane (25 mL) was heated at 105° C. for 24 h. After cooling, water (50 mL) was added and the resulting suspension was extracted with EtOAc (3×30 mL). The combined organic extracts were washed with water (50 mL), then with a brine solution (50 mL) and lastly dried over anhydrous Na₂SO₄. The volatiles were removed and the residue was purified by flash column chromatography using gradient elution from CH₂Cl₂/EtOH (99:1) to CH₂Cl₂/EtOH (97:3) to yield N,N′-bis-(4-fluorobenzyl)-N″-prop-2-ynyl-[1,3,5]triazine-2,4,6-triamine (18) (235 mg, 62%). 400 MHz ¹H NMR (CDCl₃, ppm): δ 7.35-7.17 (4H, m, overlapped with CDCl₃), 7.04-6.91 (4H, m), 5.35-5.11 (2H, m), 5.05 (1H, br s), 4.52 (4H, d, J=4.4 Hz), 4.20-4.09 (2H, m), 2.19 (1H, t, J=2.4 Hz). ESI-MS (m/z): 381 [M+H]⁺.

### N,N′-Bis-(4-fluorobenzyl)-N″-prop-2-ynyl-[1,3,5]triazine-2,4,6-triamine hydrochloride (19a)

N,N′-Bis-(4-fluoro-benzyl)-N″-prop-2-ynyl-[1,3,5]triazine-2,4,6-triamine (18) and 2M HCl/diethyl ether were reacted using procedure described for Compound 7. The product was crystallized from diethyl ether/ethanol to afford N,N′-bis-(4-fluoro-benzyl)-N″-prop-2-ynyl-[1,3,5]triazine-2,4,6-triamine hydrochloride (19) in 81% yield. 400 MHz ¹H NMR (CDCl₃, ppm): δ 8.28-8.10 (1H, m), 8.06-7.87 (1H, m), 7.65-7.55 (0.6H, m), 7.34-7.14 (4H, m), 7.06-6.91 (4H, m), 6.68-6.62 (0.4H, m), 6.20-6.08 (1H, m), 4.63-4.51 (4H, m), 4.23-4.12 (2H, m), 2.28 (0.4H, t, J=2.3 Hz), 2.23 (0.6H, t, J=2.3 Hz). ESI-MS (m/z): 381 [M+H]⁺. Melting point: 137-139° C.

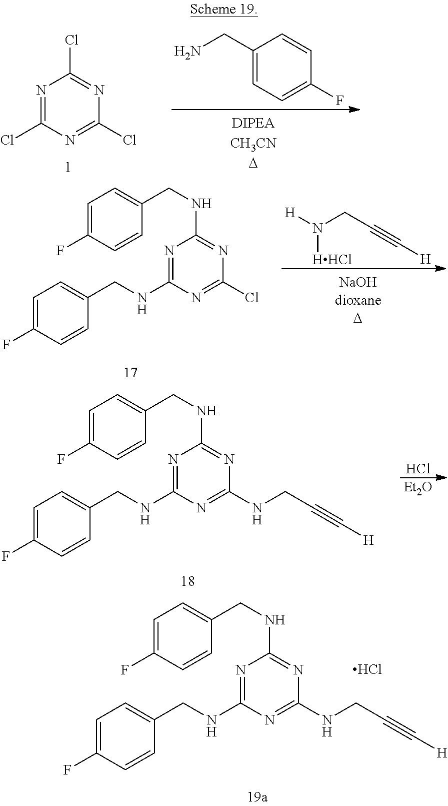

### Comparative Example 10

### N-(4-Fluorobenzyl)-N′,N″-n-dipropyl-[1,3,5]triazine-2,4,6-triamine (21) and corresponding hydrochloride salt (22a) (Scheme 20)

### 6-Chloro-N,N′-n-dipropyl-[1,3,5]triazine-2,4-diamine (20)

A 2M NaOH solution (163 mL, 325.36 mmol) was added in a dropwise fashion to a suspension of cyanuric chloride (1) (30.0 g, 162.68 mmol) and n-propylamine (26.8 mL, 325.36 mmol) in acetone (600 mL) and water (30 mL) at 0° C. The reaction mixture was heated at 50° C. for 3 h and then cooled. Water (200 mL) was added to the mixture. The resultant precipitate was filtered, washed with water (200 mL) and dried over P₂O₅ at 40° C. for 20 h to yield 6-chloro-N,N′-n-dipropyl-[1,3,5]triazine-2,4-diamine (20) (33.6 g, 90%). 400 MHz ¹H NMR (DMSO-d₆, ppm): δ 7.80 (0.85H, t, J=5.5 Hz), 7.76-7.66 (1H, m), 7.49 (0.15H, t, J=5.5 Hz), 3.22-3.11 (4H, m), 1.55-1.42 (4H, m), 0.88-0.82 (6H, m). ESI-MS (m/z): 230, 232 [M+H]⁺.

### N-(4-Fluorobenzyl)-N′,N″-n-dipropyl-[1,3,5]triazine-2,4,6-triamine (21)

A mixture of 6-chloro-N,N′-n-dipropyl-[1,3,5]triazine-2,4-diamine (20) (1.00 g, 4.35 mmol), (4-fluorophenyl)methanamine (1.0 mL, 8.70 mmol) and N,N-diisopropylethylamine (0.75 mL, 4.35 mmol) in 1,4-dioxane (20 mL) was heated at 110° C. for 20 h, after which time, the volatiles was removed under reduced pressure. A saturated NaHCO₃ solution (50 mL) was added to the residue and the mixture was extracted with CH₂Cl₂ (3×30 mL). The combined organic extracts were washed with water (100 mL) and dried over anhydrous Na₂SO₄. The solvent was removed and the residue was purified by flash column chromatography using gradient elution from CH₂Cl₂/EtOH (99:1) to CH₂Cl₂/EtOH (97:3) to yield N-(4-fluorobenzyl)-N′,N″-di-n-propyl-[1,3,5]triazine-2,4,6-triamine (21) (1.27 g, 92%). 400 MHz ¹H NMR (DMSO-d₆, ppm): δ 7.35-7.25 (2H, m), 7.14-7.05 (2H, m), 6.69-6.24 (2H, m), 4.36 (2H, d, J=4.8 Hz), 3.18-3.06 (4H, m), 1.54-1.32 (4H, m), 0.89-0.73 (6H, m). ESI-MS (m/z): 319 [M+H]⁺.

### N-(4-Fluorobenzyl)-N′,N″-di-n-propyl-[1,3,5]triazine-2,4,6-triamine hydrochloride (22a)

N-(4-Fluorobenzyl)-N′,N″-di-n-propyl-[1,3,5]triazine-2,4,6-triamine (21) and 2M HCl/diethyl ether were reacted using the procedure described for compound 7 to yield N-(4-fluoro-benzyl)-N′,N″-di-n-propyl-[1,3,5]triazine-2,4,6-triamine hydrochloride (22a). 400 MHz ¹H NMR (DMSO-d₆, ppm): 12.71-11.32 (1H, m), 9.01-8.54 (1H, m), 8.54-8.13 (2H, m), 7.44-7.30 (2H, m), 7.21-7.10 (2H, m), 4.55-4.40 (2H, m), 3.29-3.14 (4H, m), 1.59-1.35 (4H, m), 0.92-0.78 (6H, m). ESI-MS (m/z): 319 [M+H]⁺.

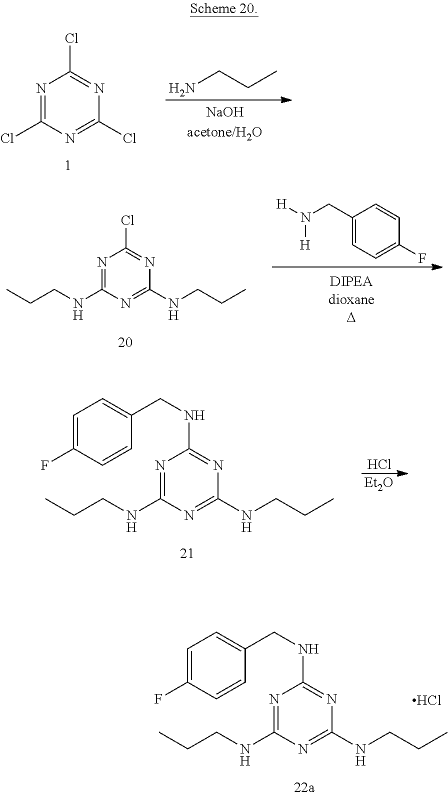

### Comparative Example 11

### N-(4,6-Bis-n-propylamino-[1,3,5]triazin-2-yl)-N-(4-fluorobenzyl)-O-methyl-hydroxylamine (23) and corresponding hydrochloride salt (24a) (Scheme 21)

### N-(4,6-Bis-n-propylamino-[1,3,5]triazin-2-yl)-N-(4-fluoro-benzyl)-O-methyl-hydroxylamine (23)

6-Chloro-N,N′-di-n-propyl-[1,3,5]triazine-2,4-diamine (20) and N-(4-fluorobenzyl)-O-methylhydroxylamine were reacted using procedure described for Compound 8 to produce the desired compound in 85% yield. 400 MHz ¹H NMR (CDCl₃, ppm): δ 7.37-7.31 (2H, m), 7.00-6.95 (2H, m), 4.93 (2H, br s), 4.85 (2H, s), 3.67 (3H, s), 3.37-3.27 (4H, m), 1.60-1.52 (4H, m), 0.94 (6H, t, J=7.4 Hz). ESI-MS (m/z): 349 [M+H]⁺.

### N-(4,6-Bis-n-propylamino-[1,3,5]triazin-2-yl)-N-(4-fluorobenzyl)-O-methyl-hydroxylamine hydrochloride (24a)

N-(4,6-Bis-n-propylamino-[1,3,5]triazin-2-yl)-N-(4-fluoro-benzyl)-O-methyl-hydroxylamine (23) and 2M HCl/diethyl ether were reacted using procedure described for Compound 7 to produce the desired compound. 400 MHz ¹H NMR (CDCl₃, ppm): δ 13.80-13.10 (0.6H, m), 9.8-8.4 (1H, m), 7.34-7.28 (2H, m), 7.17-7.07 (0.4H, m), 7.05-6.99 (2H, m), 6.5-5.2 (1H, m), 4.93 (0.4H, s), 4.90 (0.8H, s), 4.83 (0.8H, s), 3.86 (1.2H, s), 3.84 (1.2H, s), 3.73 (0.6H, s), 3.43-3.34 (4H, m), 1.70-1.56 (4H, m), 1.00-0.93 (6H, m). MS (m/z): 349 [M+H]⁺.

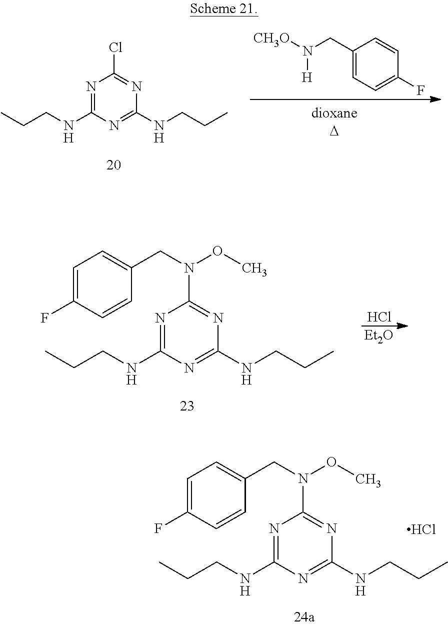

### Example 12

### N-(4,6-Bis-prop-2-ynylamino-[1,3,5]triazin-2-yl)-O,N-dimethyl-hydroxylamine (26) and corresponding hydrochloride salt (27a) (Scheme 22)

### 6-Chloro-N²,N⁴-di(prop-2-ynyl)-1,3,5-triazine-2,4-diamine (25)

A mixture of cyanuric chloride (1) (2.00 g, 10.85 mmol), propargylamine hydrochloride (1.99 g, 21.70 mmol) and K₂CO₃ (5.25 g, 37.98 mmol) in acetonitrile (50 mL) was heated at 95° C. for 22 h in a closed vial. The reaction mixture was cooled and water (50 mL) was added. The resultant precipitate was filtered, washed with water (50 mL), then with acetonitrile (50 mL) and lastly dried over P₂O₅ to yield 6-chloro-N²,N⁴-di(prop-2-ynyl)-1,3,5-triazine-2,4-diamine (25) (1.87 g, 78%). 400 MHz ¹H NMR (DMSO-d₆, ppm): δ 8.34-8.26 (1H, m), 8.22 (0.7H, t, J=5.8 Hz), 8.08 (0.3H, t, J=5.8 Hz), 4.10-3.97 (4H, m), 3.13-3.08 (2H, m). ESI-MS (m/z): 222, 224 [M+H]⁺.

### N-(4,6-Bis-prop-2-ynylamino-[1,3,5]triazin-2-yl)-O,N-dimethyl-hydroxylamine (26)

6-Chloro-N²,N⁴-di(prop-2-ynyl)-1,3,5-triazine-2,4-diamine (25) and O,N-dimethylhydroxylamine hydrochloride were reacted using the procedure described for Compound 4 to yield the desired compound in 78% yield. 400 MHz ¹H NMR (CDCl₃, ppm): δ 5.1-4.9 (2H, br s), 4.27-4.12 (4H, m), 3.77 (3H, s), 3.29 (3H, s), 2.22-2.18 (2H, m). ESI-MS (m/z): 247 [M+H]⁺.

### N-(4,6-Bis-prop-2-ynylamino-[1,3,5]triazin-2-yl)-O,N-dimethyl-hydroxylamine hydrochloride (27a)

N-(4,6-Bis-prop-2-ynylamino-[1,3,5]triazin-2-yl)-O,N-dimethyl-hydroxylamine (26) and 2M HCl/diethyl ether were reacted using the procedure described for Compound 7 to yield the desired product. 400 MHz ¹H NMR (DMSO-d₆, ppm): δ 8.9-8.5 (2H, br s), 8.46-8.20 (1H, m), 4.26-4.05 (4H, m), 3.81-3.75 (3H, m), 3.37-3.27 (3H, m), 3.27-3.12 (2H, m). ESI-MS (m/z): 247 [M+H]⁺. Melting point: 85-87° C.

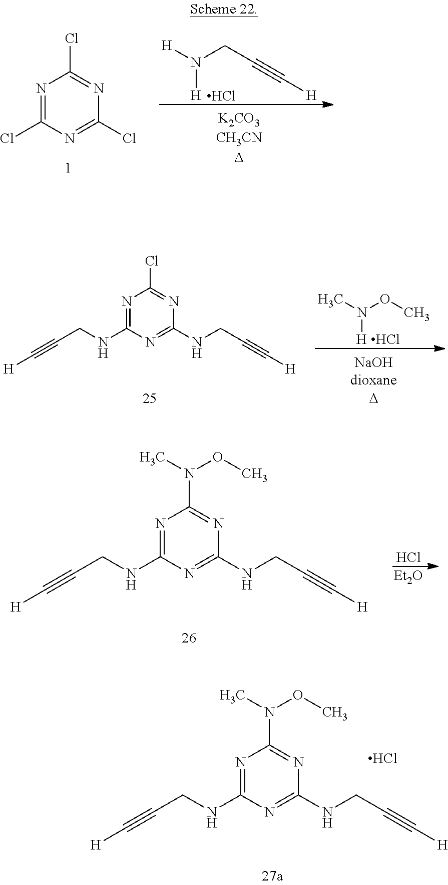

### Example 13

### N-Methyl-N′,N″-di-prop-2-ynyl-[1,3,5]triazine-2,4,6-triamine (28) and corresponding hydrochloride salt (29a) (Scheme 23)

### N-Methyl-N′,N″-di-prop-2-ynyl-[1,3,5]triazine-2,4,6-triamine (28)

6-Chloro-N²,N⁴-di(prop-2-ynyl)-1,3,5-triazine-2,4-diamine (25) and 2M MeNH₂/THF were reacted using the procedure described for compound 6 to yield the desired compound in 92% yield. 400 MHz ¹H NMR (CDCl₃, ppm): δ 4.99 (2H, br s), 4.83 (1H, br s), 4.28-4.09 (4H, m), 2.92 (3H, d, J=4.4 Hz), 2.21 (2H, t, J=2.5 Hz). ESI-MS (m/z): 217 [M+H]⁺.

### N-Methyl-N′,N″-di-prop-2-ynyl-[1,3,5]triazine-2,4,6-triamine hydrochloride (29a)

N-Methyl-N′,N″-di-prop-2-ynyl-[1,3,5]triazine-2,4,6-triamine (28) and 2M HCl/diethyl ether were reacted using the procedure described for compound 7 to yield the desired product. 400 MHz ¹H NMR (DMSO-d₆, ppm): δ 13.5-11.5 (1H, br s), 8.84-8.51 (2H, m), 8.46-8.23 (1H, m), 4.23-4.02 (4H, m), 3.29-3.13 (2H, m), 2.92-2.77 (3H, m). ESI-MS (m/z): 217 [M+H]⁺. Melting point: 90-93° C.

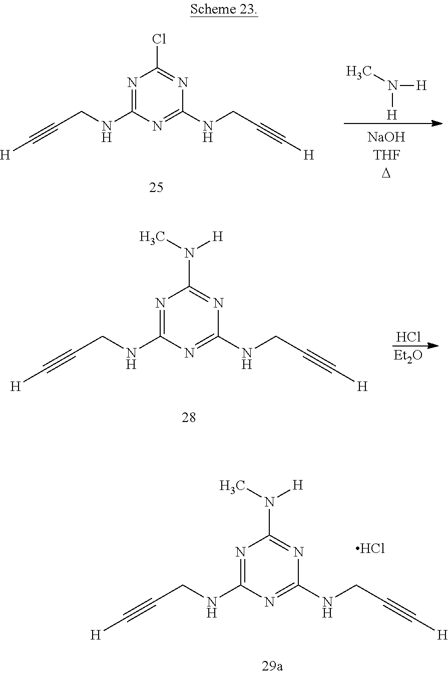

### Example 14

### N-(4-Fluorobenzyl)-N′,N″-di-prop-2-ynyl-[1,3,5]triazine-2,4,6-triamine (30) and corresponding hydrochloride salt (31a) (Scheme 24)

### N-(4-Fluorobenzyl)-N′,N″-di-prop-2-ynyl-[1,3,5]triazine-2,4,6-triamine (30)

6-Chloro-N²,N⁴-di(prop-2-ynyl)-1,3,5-triazine-2,4-diamine (25) and (4-fluorophenyl)methanamine were reacted using the procedure described for Compound 21 to yield the desired compound in 70% yield. 400 MHz ¹H NMR (DMSO, ppm): δ 7.5-6.7 (7H, m), 4.38 (2H, d, J=6.4 Hz), 4.05-3.97 (4H, m), 3.04-2.95 (2H, m). ESI-MS (m/z): 311 [M+H]⁺.

### N-(4-Fluoro-benzyl)-N′,N″-di-prop-2-ynyl-[1,3,5]triazine-2,4,6-triamine hydrochloride (31a)

N-(4-Fluoro-benzyl)-N′,N″-di-prop-2-ynyl-[1,3,5]triazine-2,4,6-triamine (30) and 2M HCl/diethyl ether were reacted using procedure described for Compound 7 to yield the desired product. 400 MHz ¹H NMR (DMSO-d₆, ppm): 9.01-8.38 (3H, m), 7.49-7.35 (2H, m), 7.20-7.12 (2H, m), 4.57-4.54 (2H, m), 4.22-4.05 (4H, m), 3.67-3.09 (2H, m, overlapped with water). ESI-MS (m/z): 311 [M+H]⁺.

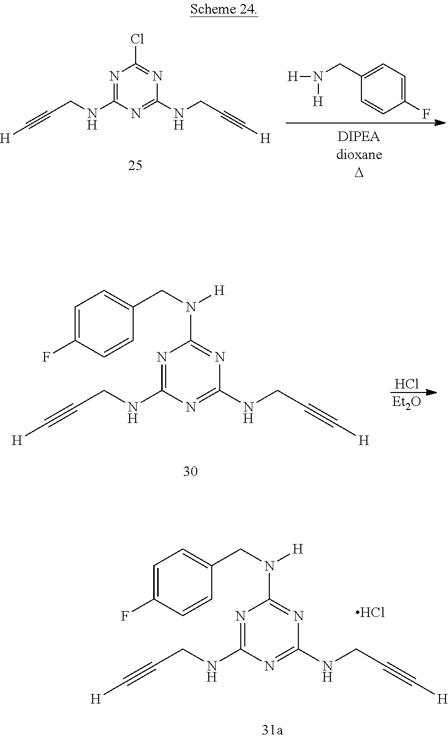

### Comparative Example 15

### N,N′-Bis-(4-fluorobenzyl)-N″-n-propyl-[1,3,5]triazine-2,4,6-triamine (32) and corresponding hydrochloride salt (33a) (Scheme 25)

### N,N′-Bis-(4-fluorobenzyl)-N″-n-propyl-[1,3,5]triazine-2,4,6-triamine (32)

A mixture of 2,4-dichloro-N-(6-n-propylamino)-[1,3,5]triazine (2) (200 mg, 0.97 mmol), (4-fluorophenyl)methanamine (0.28 mL, 2.43 mmol) and N,N-diisopropylethylamine (0.32 mL, 1.93 mmol) in 1,4-dioxane (10 mL) was stirred at 105° C. for 24 h. Water (20 mL) was the added and the resulting suspension was extracted with EtOAc (3×20 mL). The combined organic extracts were washed with water (50 mL), then with a brine solution (50 mL) and lastly dried over anhydrous Na₂SO₄. The volatiles were removed and the residue was purified by flash column chromatography using PE/EtOAc (3:1) as eluent to yield N,N′-bis-(4-fluoro-benzyl)-N″-n-propyl-[1,3,5]triazine-2,4,6-triamine (32) (341 mg, 92%). 400 MHz ¹H NMR (CDCl₃, ppm): δ 7.30-7.20 (4H, m), 7.00-6.94 (4H, m), 5.27-5.00 (2H, m), 4.90-4.66 (1H, m), 4.51 (4H, d, J=4.8 Hz), 3.34-3.23 (2H, m), 1.54 (2H, sextet, J=7.4 Hz), 0.92 (3H, t, J=7.4 Hz). ESI-MS (m/z): 385 [M+H]⁺.

### N,N′-Bis-(4-fluorobenzyl)-N″-n-propyl-[1,3,5]triazine-2,4,6-triamine hydrochloride (33a)

N,N′-bis-(4-fluoro-benzyl)-N″-n-propyl-[1,3,5]triazine-2,4,6-triamine (32) and 2M HCl/diethyl ether were reacted using the procedure described for compound 7 to yield the desired product. 400 MHz ¹H NMR (CDCl₃, ppm): δ 13.5 (1H, br s), 7.87 (1H, br s), 7.58 (1H, br s), 7.43-7.24 (2H, m), 7.23-7.17 (2H, m), 7.06-7.92 (4H, m), 6.18-5.70 (1H, m), 4.63-4.50 (4H, m), 3.51-3.29 (2H, m), 1.68-1.54 (2H, m), 1.02-0.91 (3H, m). ESI-MS (m/z): 385 [M+H]⁺.

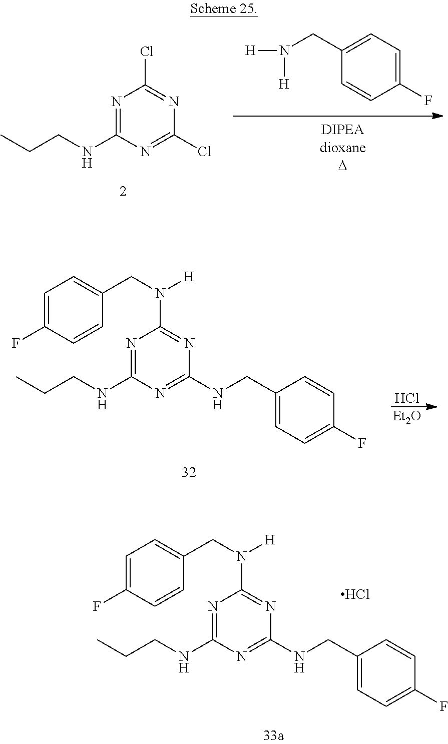

### Example 16

### O-(4-Fluorophenyl)-N-(4-n-propylamino-6-prop-2-ynylamino-[1,3,5]triazin-2-yl)-hydroxylamine (36), and corresponding hydrochloride salt (37a) (Scheme 26)

### 2-(4-Fluorophenoxy)-isoindole-1,3-dione

A mixture of 4-fluorophenylboronic acid (2.00 g, 14.29 mmol), N-hydroxyphthalimide (1.17 g, 7.15 mmol), Cu(OAc)₂ (1.30 g, 7.15 mmol), pyridine (635 μL, 7.87 mmol) and 4 Å molecular sieves (1.00 g) in CH₂Cl₂ were vigorously stirred at room temperature for 16 h. The mixture was filtered through a Celite pad and evaporated. The resultant residue was purified by flash column chromatography using gradient elution from PE/EtOAc (5:1) to PE/EtOAc (5:2) to yield 2-(4-fluorophenoxy)-isoindole-1,3-dione (1.57 g, 86%). 400 MHz ¹H NMR (CDCl₃, ppm): δ 7.94-7.89 (2H, m), 7.84-7.79 (2H, m), 7.25-7.19 (2H, m), 7.06-6.99 (2H, m).

### O-(4-Fluorophenyl)-hydroxylamine

Hydrazine hydrate (760 μL, 15.60 mmol) was added to a solution of 2-(4-fluorophenoxy)-isoindole-1,3-dione (1.34 g, 5.21 mmol) in CHCl₃ (25 mL) and MeOH (5 mL). The reaction mixture was stirred at room temperature for 20 h and then filtered. The filtrate was washed with saturated NaHCO₃ solution (2×30 mL) and then with water (30 mL). The organic phase was dried over anhydrous Na₂SO₄ and the solvent was removed to yield O-(4-fluorophenyl)-hydroxylamine (520 mg, 78%). 400 MHz ¹H NMR (CDCl₃, ppm): δ 7.11-7.05 (2H, m), 7.00-6.92 (2H, m), 5.87 (2H, s).

### N-(4,6-Dichloro-[1,3,5]triazin-2-yl)-O-(4-fluorophenyl)-hydroxylamine (34)

Cyanuric chloride (1) (406 mg, 2.20 mmol) was added portionwise to a cooled solution (−10° C.) of O-(4-fluorophenyl)-hydroxylamine (420 mg, 3.30 mmol) in CH₂Cl₂ (60 mL). The reaction mixture was stirred at −10° C. for 3 h. The mixture was then poured into water (20 mL). The layers were separated and the water phase was extracted with CH₂Cl₂ (2×20 mL). The combined organic extracts were washed with water (100 mL), and dried over anhydrous Na₂SO₄. The solvent was removed under reduced pressure, and the resultant residue was purified by flash column chromatography using gradient elution from PE/EtOAc (10:1) to PE/EtOAc (5:1) to yield N-(4,6-dichloro-[1,3,5]triazin-2-yl)-O-(4-fluorophenyl)-hydroxylamine (34) (520 mg, 57%). 400 MHz ¹H NMR (CDCl₃, ppm): δ 8.70 (1H, s), 7.13-7.01 (4H, m).

### N-(4-Chloro-6-n-propylamino-[1,3,5]triazin-2-yl)-O-(4-fluorophenyl)-hydroxylamine (35)

n-Propylamine (450 μL, 5.45 mmol) was added to a solution of N-(4,6-dichloro-[1,3,5]triazin-2-yl)-O-(4-fluorophenyl)-hydroxylamine (34) in CH₂Cl₂ (15 mL) at room temperature and the mixture was stirred for 3 h. The resultant precipitate was filtered, washed with water (10 mL) and dried to yield N-(4-chloro-6-n-propylamino-[1,3,5]triazin-2-yl)-O-(4-fluorophenyl)-hydroxylamine (35) (250 mg, 46%). 400 MHz ¹H NMR (DMSO-d₆, ppm): δ 11.56 (1H, br s), 8.22 (1H, t, J=5.6 Hz), 7.18-7.11 (2H, m), 7.10-7.04 (2H, m), 3.19-3.13 (1H, m), 3.11-3.03 (1H, m), 1.47 (1H, sextet, J=7.4 Hz), 1.41-1.29 (1H, m), 0.84 (1.5H, t, J=7.4 Hz), 0.73 (1.5H, t, J=7.1 Hz). ESI-MS (m/z): 298, 300 [M+H]⁺.

### O-(4-Fluorophenyl)-N-(4-n-propylamino-6-prop-2-ynylamino-[1,3,5]triazin-2-yl)-hydroxylamine (36)

A solution of N-(4-chloro-6-n-propylamino-[1,3,5]triazin-2-yl)-O-(4-fluorophenyl)-hydroxylamine (35) (230 mg, 0.77 mmol) and propargylamine (400 μL, 6.18 mmol) in 1,4-dioxane (5 mL) was heated at 90° C. for 24 h. The mixture was cooled to room temperature and water (15 mL) was added. The resulting suspension was extracted with CH₂Cl₂ (3×15 mL). The combined organic extracts were washed with water (30 mL) and dried over anhydrous Na₂SO₄. The solvent was removed and the residue was purified by flash column chromatography using gradient elution from CH₂Cl₂/MeOH (99:1) to CH₂Cl₂/MeOH (9:1) to yield O-(4-fluorophenyl)-N-(4-n-propylamino-6-prop-2-ynylamino-[1,3,5]triazin-2-yl)-hydroxylamine (36) (140 mg, 57%). ESI-MS (m/z): 317 [M+H]⁺.

### O-(4-Fluorophenyl)-N-(4-n-propylamino-6-prop-2-ynylamino-[1,3,5]triazin-2-yl)-hydroxylamine hydrochloride (37a)

O-(4-Fluorophenyl)-N-(4-n-propylamino-6-prop-2-ynylamino-[1,3,5]triazin-2-yl)-hydroxylamine (36) was reacted with 2M HCl/diethyl ether using the procedure described for Compound 7. 400 MHz ¹H NMR (CDCl₃, ppm): δ 11.5-10.4 (2H, m), 8.98-8.69 (1H, m), 8.26-8.92 (1H, m), 7.08-7.01 (2H, m), 6.87-6.81 (2H, m), 4.28-4.06 (2H, m), 3.45-3.22 (2H, m), 2.35-2.26 (1H, m), 1.73-1.52 (2H, m), 1.04-0.82 (3H, m). ESI-MS (m/z): 317 [M+H]⁺.

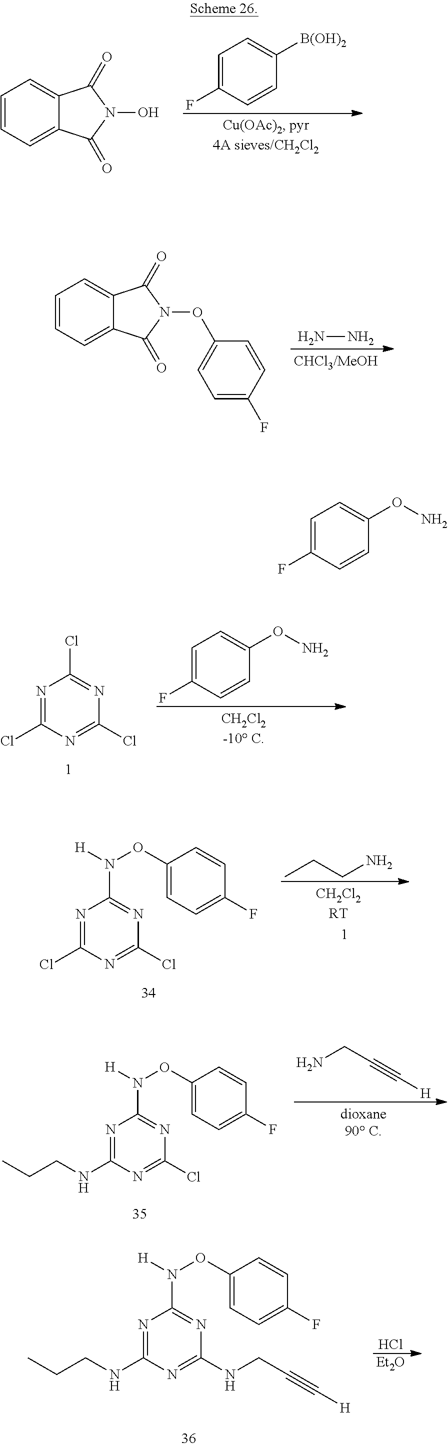

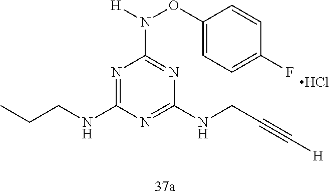 [SMILES: C#CCNc1nc(NCCC)nc(NOc2ccc(F)cc2)n1 ; Cl]

### Example 17

### N-[4-(1,1-Dimethyl-prop-2-ynylamino)-6-n-propylamino-[1,3,5]triazin-2-yl]-O,N-dimethyl-hydroxylamine hydrochloride (39), and corresponding hydrochloride salt (40a) (Scheme 27)

### 6-Chloro-N-(1,1-dimethyl-prop-2-ynyl)-N′-n-propyl-[1,3,5]triazine-2,4-diamine (38)

A mixture of 2,4-dichloro-N-(6-n-propylamino)-[1,3,5]triazine (2) (400 mg, 1.93 mmol), 1,1-dimethyl-prop-2-ynylamine (203 μL, 1.93 mmol) and N,N-diisopropylethylamine (385 μL, 2.32 mmol) in 1,4-dioxane (7 mL) was heated at 90° C. for 7 h. The mixture was cooled to room temperature and water (15 mL) was added. The resulting suspension was extracted with EtOAc (3×15 mL). The combined organic extracts were washed with water (30 mL), then with brine (30 mL) and lastly, dried over anhydrous Na₂SO₄. The volatiles were removed under vacuum to yield 6-chloro-N-(1,1-dimethyl-prop-2-ynyl)-N′-n-propyl-[1,3,5]triazine-2,4-diamine (38), which was used in the next step without purification. 400 MHz ¹H NMR (CDCl₃, ppm): δ 5.78 (0.7H, br s), 5.43 (1H, s), 5.32 (0.3H, br s), 3.47-3.33 (2H, m), 2.32-2.26 (1H, m), 1.73-1.67 (6H, m), 1.66-1.57 (2H, m), 0.95 (3H, t, J=7.5 Hz). ESI-MS (m/z): 254, 256 [M+H]⁺.

### N-[4-(1,1-Dimethyl-prop-2-ynylamino)-6-n-propylamino-[1,3,5]triazin-2-yl]-O,N-dimethyl-hydroxylamine (39)

6-Chloro-N-(1,1-dimethyl-prop-2-ynyl)-N′-n-propyl-[1,3,5]triazine-2,4-diamine (38) and O,N-dimethylhydroxylamine hydrochloride were reacted using procedure described for Compound 4 to yield N-[4-(1,1-dimethyl-prop-2-ynylamino-[6-n-propylamino-[1,3,5]triazin-2-yl]-O,N-dimethyl-hydroxylamine (39) in 72% yield. 400 MHz ¹H NMR (CDCl₃, ppm): δ 5.16-4.83 (2H, m), 3.77 (3H, s), 3.39-3.30 (2H, m), 3.27 (3H, s), 2.27-2.23 (1H, m), 1.70 (6H, s), 1.58 (2H, sextet, J=7.3 Hz), 0.94 (3H, t, J=7.3 Hz). ESI-MS (m/z): 279 [M+H]⁺.

### N-[4-(1,1-Dimethyl-prop-2-ynylamino)-6-n-propylamino-[1,3,5]triazin-2-yl]-O,N-dimethyl-hydroxylamine hydrochloride (40a)

N-[4-(1,1-dimethyl-prop-2-ynylamino)-6-n-propylamino-[1,3,5]triazin-2-yl]-O,N-dimethyl-hydroxylamine (39) and 2M HCl/diethyl ether were reacted using the procedure described for Compound 7 to yield N-[4-(1,1-dimethyl-prop-2-ynylamino)-6-n-propylamino-[1,3,5]triazin-2-yl]-O,N-dimethyl-hydroxylamine hydrochloride (40a). 400 MHz ¹H NMR (CDCl₃, ppm): δ 13.57-13.34 (0.5H, m), 13.32-13.07 (0.5H, m), 9.76-9.16 (1H, m), 5.81-5.53 (1H, m), 3.99-3.79 (3H, m), 3.48-3.23 (5H, m), 2.35-2.25 (1H, m), 1.78-1.67 (6H, m), 1.67-1.53 (2H, m), 1.07-0.87 (3H, m). ESI-MS (m/z): 279 [M+H]⁺.

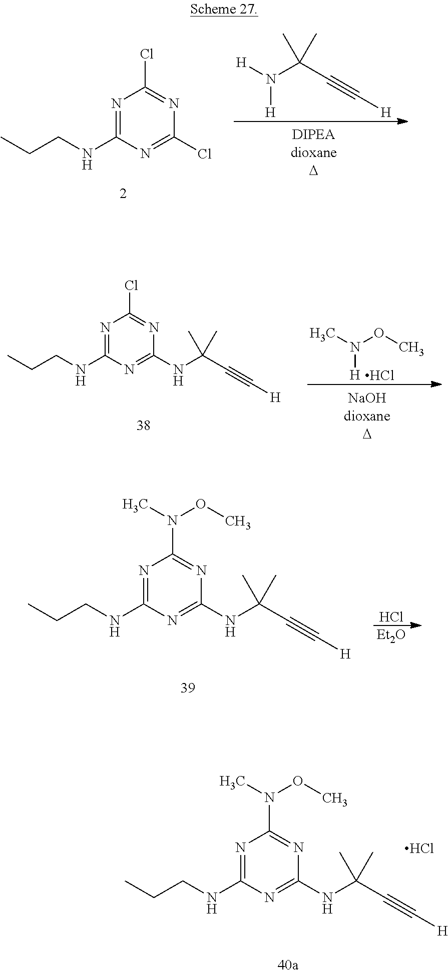

### Example 18

### O,N-Dimethyl-N-(4-n-propylamino-6-but-2-ynylamino-[1,3,5]triazin-2-yl)-hydroxylamine (42) and corresponding hydrochloride salt (43a) (Scheme 28)

### N-But-2-ynyl-6-chloro-N′-n-propyl-[1,3,5]triazine-2,4-diamine (41)

A mixture of 2,4-dichloro-N-(6-n-propylamino)-[1,3,5]triazine (2) (414 mg, 2.00 mmol), but-2-ynylamine hydrochloride (211 mg, 2.00 mmol) and N,N-diisopropylethylamine (520 μL, 3.00 mmol) in 1,4-dioxane (15 mL) was stirred at 55° C. for 6 h. The mixture was cooled to room temperature and water (10 mL) was added. The resultant precipitate was filtered, washed with water and dried to yield N-but-2-ynyl-6-chloro-N′-n-propyl-[1,3,5]triazine-2,4-diamine (41) (410 mg, 85%). δ 400 MHz ¹H NMR (DMSO-d₆, ppm): 8.08-7.76 (2H, m), 4.01-3.92 (2H, m), 3.24-3.11 (2H, m), 1.78-1.73 (3H, m), 1.57-1.43 (2H, m), 0.89-0.92 (3H, m). MS (m/z): 240, 242 [M+H]⁺.

### O,N-Dimethyl-N-(4-n-propylamino-6-but-2-ynylamino-[1,3,5]triazin-2-yl)-hydroxylamine (42)

N-but-2-ynyl-6-chloro-N′-n-propyl-[1,3,5]triazine-2,4-diamine (41) and O,N-dimethylhydroxylamine hydrochloride were reacted using the procedure described for Compound 4 to yield O,N-dimethyl-N-(4-n-propylamino-6-prop-1-ynylamino-[1,3,5]triazin-2-yl)-hydroxylamine in 96% yield. 400 MHz ¹H NMR (CDCl₃, ppm): δ 5.22-4.94 (2H, m), 4.20-4.05 (2H, m), 3.76 (3H, s), 3.39-3.18 (5H, m), 1.79 (3H, t, J=2.4 Hz), 1.57 (2H, sextet, J=7.4 Hz), 0.94 (3H, t, J=7.4 Hz). MS (m/z): 265 [M+H]⁺.

### O,N-Dimethyl-N-(4-n-propylamino-6-but-2-ynylamino-[1,3,5]triazin-2-yl)-hydroxylamine hydrochloride (43a)

O,N-Dimethyl-N-(4-n-propylamino-6-but-2-ynylamino-[1,3,5]triazin-2-yl)-hydroxylamine (42) and 2M HCl/diethyl ether were reacted using procedure described for Compound 7 to yield O,N-dimethyl-N-(4-n-propylamino-6-but-2-ynyl amino-[1,3,5]triazin-2-yl)-hydroxylamine hydrochloride (43a). 400 MHz ¹H NMR (CDCl₃, ppm): δ 9.01 (1H, br s), 5.95 (1H, br s), 4.23-4.04 (2H, m), 3.99-3.86 (3H, m), 3.52-3.26 (5H, m), 1.86-1.73 (3H, m), 1.73-1.54 (2H, m), 1.04-0.89 (3H, m). MS (m/z): 265 [M+H]⁺.

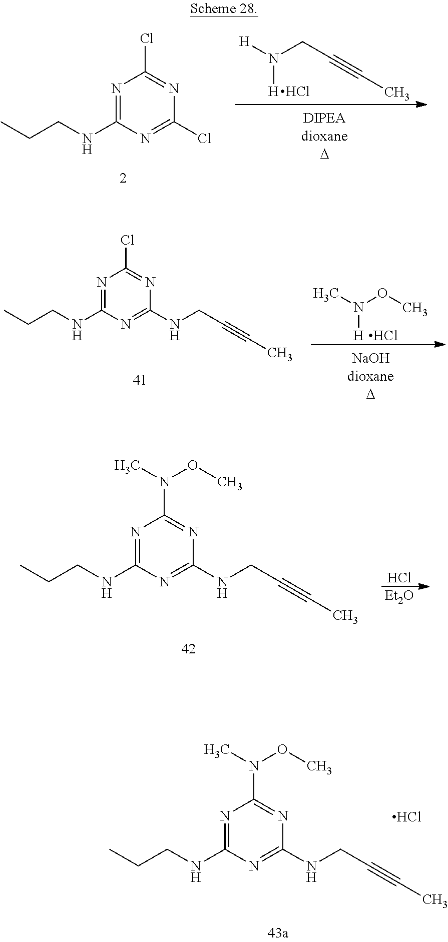

### Examples 19 & 20

### O,N-Dimethyl-N-(6-n-propylamino-2-prop-2-ynylamino-pyrimidin-4-yl)-hydroxylaminie (48) and corresponding hydrochloride salt (50a); and O,N-dimethyl-N-(2-n-propylamino-6-prop-2-ynylamino-pyrimidin-4-yl)-hydroxylamine (49) and corresponding hydrochloride salt (51a) (Scheme 29)

### (2,6-Dichloro-pyrimidin-4-yl)-n-propyl-amine (44) and (4,6-dichloro-pyrimidin-2-yl)-n-propylamine (45)

2,4,6-Trichloro-pyrimidine (5.00 g, 27.26 mmol) and n-propylamine (3.14 mL, 57.33 mmol) in EtOH (40 mL) was stirred at room temperature for 10 h. The volatiles were removed under reduced pressure. A saturated NaHCO₃ solution (50 mL) was added and the resulting suspension was extracted with CH₂Cl₂ (3×50 mL). The combined organic extracts were washed with water (50 mL) and dried over anhydrous Na₂SO₄. The volatiles were removed under reduced pressure and the mixture was purified by flash column chromatography using gradient elution from petroleum ether/EtOAc (20:1) to petroleum ether/EtOAc (5:1) to yield (2,6-dichloro-pyrimidin-4-yl)-n-propylamine (44) (2.50 g, 44%) and (4,6-dichloro-pyrimidin-2-yl)-n-propyl-amine (44) (1.90 g, 34%).

Compound 44: 400 MHz ¹H NMR (CDCl₃, ppm): δ 6.26 (1H, s), 5.66 (0.7H, br s), 5.11 (0.3H, br s), 3.53-3.04 (2H, m), 1.64 (2H, sextet, J=7.4 Hz), 0.99 (3H, t, J=7.4 Hz). ESI-MS (m/z): 206, 208, 210 [M+H]⁺.

Compound 45: 400 MHz H NMR (CDCl₃, ppm): δ 6.57 (1H, s), 5.49 (1H, br s), 3.41-3.45 (2H, m), 1.61 (2H, sextet, J=7.3 Hz), 0.96 (3H, t, J=7.3 Hz). ESI-MS (m/z): 206, 208, 210 [M+H]⁺.

### 6-Chloro-N⁴n-propyl-N²-prop-2-ynyl-pyrimidine-2,4-diamine (46)

The mixture of (2,6-dichloro-pyrimidin-4-yl)-n-propylamine (44) (1.50 g, 7.28 mmol), propargylamine hydrochloride (2.00 g, 21.84 mmol) and N,N-diisopropylethylamine (5.2 mL, 29.12 mmol) in 1,4-dioxane (20 mL) was heated at 100° C. for 48 h. After cooling, water (20 mL) was added and the resulting suspension was extracted with CH₂Cl₂ (3×30 mL). The combined organic extracts were washed with water (50 mL) and dried over anhydrous Na₂SO₄. The volatiles were removed under reduced pressure and the mixture was purified by flash column chromatography using gradient elution from petroleum ether/EtOAc (5:1) to petroleum ether/EtOAc (5:2) to give 6-chloro-N⁴-n-propyl-N²-prop-2-ynyl-pyrimidine-2,4-diamine (46) (500 mg, 30%). 400 MHz ¹H NMR (CDCl₃, ppm): δ 5.76 (1H, s), 5.09 (1H, s), 5.00-4.72 (1H, br s), 4.16 (2H, dd, J=5.7, 2.5 Hz), 3.34-3.11 (2H, m), 2.20 (1H, t, J=2.5 Hz), 1.62 (2H, sextet, J=7.4 Hz), 0.97 (3H, t, J=7.4 Hz). ESI-MS (m/z): 225, 227 [M+H]⁺.

### O,N-Dimethyl-N-(6-n-propylamino-2-prop-2-ynylamino-pyrimidin-4-yl)-hydroxylamine (48)

A mixture of 6-chloro-N⁴-n-propyl-N²-prop-2-ynyl-pyrimidine-2,4-diamine (46) (500 mg, 2.22 mmoL) and O,N-dimethylhydroxylamine hydrochloride (1.30 g, 13.33 mmol) in pyridine (10 mL) was heated at 130° C. for 30 min. After cooling, water (20 mL) was added and the resulting suspension was extracted with CH₂Cl₂ (3×30 mL). The combined organic extracts were washed with water (50 mL) and dried over anhydrous Na₂SO₄. The resultant residue was purified by flash column chromatography using gradient elution from CH₂Cl₂/EtOH (99:1) to CH₂Cl₂/EtOH (95:5) to yield O,N-dimethyl-N-(6-n-propylamino-2-prop-2-ynylamino-pyrimidin-4-yl)-hydroxylamine (48) (230 mg, 42%). 400 MHz ¹H NMR (CDCl₃, ppm): δ 5.45 (1H, s), 4.73 (1H, t, J=5.7 Hz), 4.63-4.54 (1H, m), 4.14 (2H, dd, J=5.7, 2.5 Hz), 3.69 (3H, s), 3.23-3.15 (5H, m), 2.16 (1H, t, J=2.5 Hz), 1.60 (2H, sextet, J=7.2 Hz), 0.97 (3H, t, J=7.2 Hz). 100 MHz ¹³C NMR (CDCl₃, ppm): 168.3, 164.9, 161.2, 110.2, 81.8, 70.4, 60.9, 43.5, 37.9, 31.3, 22.9, 11.7. ESI-MS (m/z): 250 [M+H]⁺.

### O,N-Dimethyl-N-(6-n-propylamino-2-prop-2-ynylamino-pyrimidin-4-yl)-hydroxylamine hydrochloride (50a)

A solution of 2M HCl/diethyl ether (0.68 mL, 1.36 mmol) and O,N-dimethyl-N-(6-n-propylamino-2-prop-2-ynylamino-pyrimidin-4-yl)-hydroxylamine (48) (210 mg, 0.84 mmol) in diethyl ether (10 mL) was stirred for 0.5 h at 0° C. The volatiles were removed under reduced pressure to yield O,N-dimethyl-N-(6-n-propylamino-2-prop-2-ynylamino-pyrimidin-4-yl)-hydroxylamine hydrochloride (50a) in quantitative yield. 400 MHz ¹H NMR (CDCl₃, ppm): δ 13.92 (1H, br s), 6.90-6.29 (2H, m), 5.30 (1H, s), 4.21-4.10 (2H, m), 3.78-3.72 (3H, m), 3.37 (3H, s), 3.18 (2H, q, J=6.5 Hz), 2.22 (1H, t, J=2.5 Hz), 1.68 (2H, sextet, J=7.3 Hz), 1.01 (3H, t, J=7.3 Hz). ESI-MS (m/z): 250 [M+H]⁺.

### 6-Chloro-N²-n-propyl-N⁴-prop-2-ynyl-pyrimidine-2,4-diamine (47)

A mixture of (4,6-dichloro-pyrimidin-2-yl)-n-propylamine (45) (1.80 g, 8.73 mmol), propargylamine hydrochloride (1.60 g, 17.46 mmol) and N,N-diisopropylethylamine (4.5 mL, 26.19 mmol) in 1,4-dioxane (15 mL) was heated at 100° C. for 24 h. After cooling, water (20 mL) was added and the resulting suspension was extracted with CH₂Cl₂ (3×30 mL). The combined organic extracts were washed with water (50 mL) and dried over anhydrous Na₂SO₄. The volatiles were removed under reduced pressure; and the mixture was purified by flash column chromatography using gradient elution from petroleum ether/EtOAc (5:1) to petroleum ether/EtOAc (1:1) to yield 6-chloro-N²-n-propyl-N⁴-prop-2-ynyl-pyrimidine-2,4-diamine (47) (720 mg, 36%). ESI-MS (m/z): 225, 227 [M+H]⁺.

### O,N-Dimethyl-N-(2-n-propylamino-6-prop-2-ynylamino-pyrimidin-4-yl)-hydroxylamine (49)

6-Chloro-N²-n-propyl-N⁴-prop-2-ynyl-pyrimidine-2,4-diamine (47) and O,N-dimethylhydroxylamine hydrochloride were reacted using the procedure described for compound 48 to yield O,N-dimethyl-N-(2-n-propylamino-6-prop-2-ynylamino-pyrimidin-4-yl)-hydroxylamine (49) in 31% yield. 400 MHz ¹H NMR (CDCl₃, ppm): 5.47 (1H, s), 4.69 (1H, t, J=5.6 Hz), 4.64 (1H, t, J=5.5 Hz), 4.09 (2H, dd, J=5.6, 2.5 Hz), 3.68 (3H, s), 3.31-3.25 (2H, m), 3.19 (3H, s), 2.21 (1H, t, J=2.6 Hz), 1.57 (2H, sextet, J=7.5 Hz), 0.93 (3H, t, J=7.5 Hz). 100 MHz ¹³C NMR (CDCl₃, ppm): 168.2, 163.9, 161.9, 80.6, 75.6, 71.1, 60.8, 43.2, 37.7, 31.0, 23.1, 11.5. ESI-MS (m/z): 250 [M+H]⁺.

### O,N-Dimethyl-N-(2-n-propylamino-6-prop-2-ynylamino-pyrimidin-4-yl)-hydroxylamine hydrochloride (51a)

O,N-Dimethyl-N-(2-n-propylamino-6-prop-2-ynylamino-pyrimidin-4-yl)-hydroxylamine (49) and 2M HCl/diethyl ether were reacted using procedure described for Compound 50a to yield O,N-dimethyl-N-(2-n-propylamino-6-prop-2-ynylamino-pyrimidin-4-yl)-hydroxylamine hydrochloride (51a). 400 MHz ¹H NMR (CDCl₃, ppm): δδ 13.56 (1H, br s), 6.97 (1H, br s), 6.64 (1H, br s), 5.46-5.27 (1H, m), 4.30-4.14 (0.3H, m), 4.07-3.97 (1.7H, m), 3.74 (3H, s), 3.41-3.27 (5H, m), 2.32-2.27 (1H, m), 1.72-1.53 (2H, m), 0.95 (3H, t, J=6.8 Hz). ESI-MS (m/z): 250 [M+H]⁺.

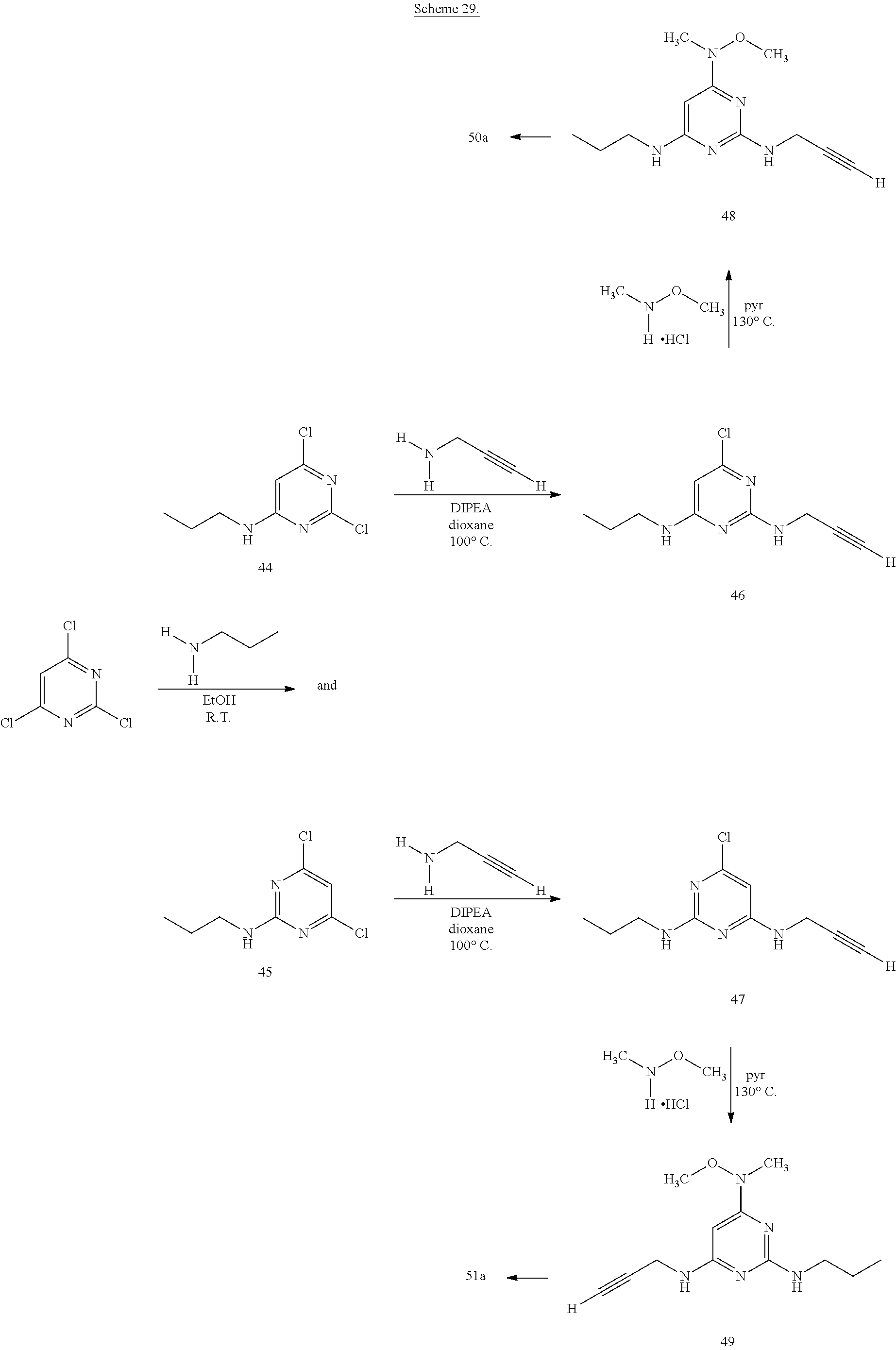

### Example 21

### O,N-Dimethyl-N-(4-methylamino-6-prop-2-ynylamino-[1,3,5]triazin-2-yl)-hydroxylamine (54) and corresponding hydrochloride salt (55a) (Scheme 30)

### N-(4,6-Dichloro-[1,3,5]triazin-2-yl)-N-prop-2-ynyl-amine (52)

To a solution of cyanuric chloride (1) (5.00 g, 27.11 mmol) in tetrahydrofuran (25 mL), a mixture of propargylic amine (1.74 mL, 27.11 mmol) and N,N-diisopropylethylamine (4.48 mL, 27.11 mmol) in tetrahydrofuran (50 mL) was added gradually during 2 h (syringe pump) at −20° C. The reaction mixture was stirred for 3 h (reaction temperature from −20° C. to 0° C.). After this time, the volatiles were removed by evaporation, and the residue was partitioned between EtOAc (100 mL) and water (30 mL). The EtOAc layer was washed with water (2×30 mL), then with a brine solution (100 mL), and lastly dried over solid anhydrous Na₂SO₄. After filtration the solvent was removed under reduced pressure to afford N-(4,6-dichloro-[1,3,5]triazin-2-yl)-N-prop-2-ynyl-amine (52) (5.33 g, 97%). 400 MHz ¹H NMR (CDCl₃, ppm): δ 6.06 (1H, br s), 4.31 (2H, dd, J=5.7, 2.5 Hz), 2.34 (1H, t, J=2.5 Hz). ESI-MS (m/z): 203, 205, 207 [M+H]⁺.

### 6-Chloro-N-methyl-N′-prop-2-ynyl-[1,3,5]triazine-2,4-diamine (53)

N-(4,6-Dichloro-[1,3,5]triazin-2-yl)-N-prop-2-ynyl-amine (52) and 2 M MeNH₂/THF were reacted using procedure described for Compound 6 to yield 6-chloro-N-methyl-N′-prop-2-ynyl-[1,3,5]triazine-2,4-diamine (53) in 95% yield. 400 MHz ¹H NMR (DMSO-d₆, ppm): δ 8.18-8.03 (1H, m), 7.93-7.71 (1H, m), 4.09-4.05 (0.8H, m), 4.00-3.97 (1.2H, m), 3.10 (1H, t, J=2.5 Hz), 2.79 (1.2H, d, J=4.8 Hz), 2.75-2.72 (1.8H, m). ESI-MS (m/z): 198, 200 [M+H]⁺.

### O,N-Dimethyl-N-(4-methylamino-6-prop-2-ynylamino-[1,3,5]triazin-2-yl)-hydroxylamine (54)

6-Chloro-N-methyl-N′-prop-2-ynyl-[1,3,5]triazine-2,4-diamine (53) and O,N-dimethylhydroxylamine hydrochloride were reacted using procedure described for Compound 4 to yield O,N-dimethyl-N-(4-methylamino-6-prop-2-ynylamino-[1,3,5]triazin-2-yl)-hydroxylamine (54) in 97% yield. 400 MHz ¹H NMR (CDCl₃, ppm): δ 5.2-4.8 (2H, m), 4.20 (2H, br s), 3.77 (3H, s), 3.29 (3H, s), 2.94 (3H, d, J=5.0 Hz), 2.20 (1H, t, J=2.5 Hz). ESI-MS (m/z): 223 [M+H]⁺.

### O,N-Dimethyl-N-(4-methylamino-6-prop-2-ynylamino-[1,3,5]triazin-2-yl)-hydroxylamine hydrochloride (55a)

O,N-Dimethyl-N-(4-methylamino-6-prop-2-ynylamino-[1,3,5]triazin-2-yl)-hydroxylamine (54) and 2 M HCl/diethyl ether were reacted using the procedure described for Compound 5a to yield O,N-dimethyl-N-(4-methylamino-6-prop-2-ynyl amino-[1,3,5]triazin-2-yl)-hydroxylamine hydrochloride (55a) in quantitative yield. 400 MHz ¹H NMR (D₂O, ppm): δ 4.33-4.20 (2H, m), 3.83 (3H, s), 3.50-3.35 (3H, m), 3.06-2.93 (3H, m), 2.69-2.65 (1H, m). ESI-MS (m/z): 223 [M+H]⁺.

Scheme 30.

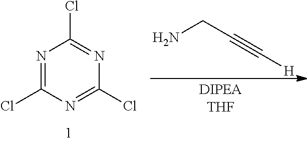

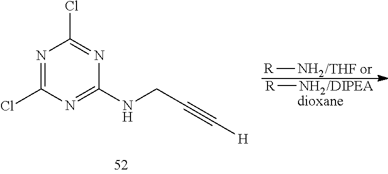

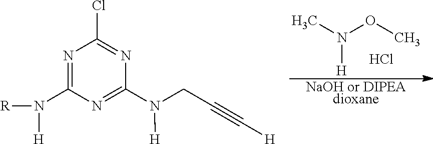

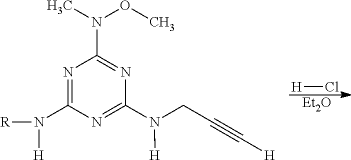

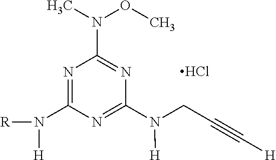

| cmpd | R |
|---|---|
| 53 | CH₃ |
| 54 | CH₃ |
| 55a | CH₃ |
| 56 | Et |
| 57 | Et |
| 58a | Et |
| 59 | iPr |
| 60 | iPr |
| 61a | iPr |
| 62 | cPr |
| 63 | cPr |
| 64a | cPr |
| 65 | nBu |
| 66 | nBu |
| 67a | nBu |
| 68 | cBu |
| 69 | cBu |
| 70a | cBu |
| 71 | cPrCH₂— |
| 72 | cPrCH₂— |
| 73a | cPrCH₂— |
| 74 | cHex |
| 75 | cHex |
| 76a | cHex |
| 77 | cHexCH₂— |
| 78 | cHexCH₂— |
| 79a | cHexCH₂— |
| 80 | Benzyl |
| 81 | Benzyl |
| 82a | Benzyl |

### Example 22

### O,N-Dimethyl-N-(4-ethylamino-6-prop-2-ynylamino-[1,3,5]triazin-2-yl)-hydroxylamine (57) and corresponding hydrochloride salt (58a) (Scheme 30)

### 6-Chloro-N-ethyl-N′-prop-2-ynyl-[1,3,5]triazine-2,4-diamine (56)

N-(4,6-Dichloro-[1,3,5]triazin-2-yl)-N-prop-2-ynyl-amine (52) and 2 M EtNH₂/THF were reacted using procedure described for Compound 6 to yield 6-chloro-N-ethyl-N′-prop-2-ynyl-[1,3,5]triazine-2,4-diamine (56) in 89% yield. 400 MHz ¹H NMR (DMSO-d₆, ppm): δ 8.17-8.02 (1H, m), 7.97-7.80 (1H, m), 4.03 (1H, dd, J=5.9, 2.4 Hz), 4.01-3.97 (1H, m), 3.30-3.18 (2H, m), 3.11-3.07 (1H, m), 1.13-1.03 (3H, m). ESI-MS (m/z): 212, 214 [M+H]⁺.

### N-(4-Ethylamino-6-prop-2-ynylamino-[1,3,5]triazin-2-yl)-O,N-dimethyl-hydroxylamine (57)

6-Chloro-N-ethyl-N′-prop-2-ynyl-[1,3,5]triazine-2,4-diamine (56) and O,N-dimethylhydroxylamine hydrochloride were reacted using the procedure described for Compound 4 to yield N-(4-ethylamino-6-prop-2-ynylamino-[1,3,5]triazin-2-yl)-O,N-dimethyl-hydroxylamine (57) in 99% yield. 400 MHz ¹H NMR (CDCl₃, ppm): δ 5.05 (1H, br s), 4.94 (1H, br s), 4.23-4.13 (2H, m), 3.77 (3H, s), 3.45-3.36 (2H, m), 3.28 (3H, s), 2.20 (1H, t, J=2.5 Hz), 1.18 (3H, t, J=7.2 Hz). ESI-MS (m/z): 237 [M+H]⁺.

### N-(4-Ethylamino-6-prop-2-ynylamino-[1,3,5]triazin-2-yl)-O,N-dimethyl-hydroxylamine hydrochloride (58)

N-(4-Ethylamino-6-prop-2-ynylamino-[1,3,5]triazin-2-yl)-O,N-dimethyl-hydroxylamine (57) and 2 M HCl/diethyl ether were reacted using the procedure described for Compound 5a to yield N-(4-ethylamino-6-prop-2-ynylamino-[1,3,5]triazin-2-yl)-O,N-dimethyl-hydroxylamine hydrochloride (58a) in quantitative yield. 400 MHz ¹H NMR (D₂O, ppm): δ 4.32-4.19 (2H, m), 3.83 (3H, s), 3.58-3.34 (5H, m), 2.70-2.65 (1H, m), 1.26-1.17 (3H, m). ESI-MS (m/z): 237 [M+H]⁺.

### Example 23

### O,N-Dimethyl-N-(4-isopropylamino-6-prop-2-ynylamino-[1,3,5]triazin-2-yl)-hydroxylamine (60) and corresponding hydrochloride salt (61a) (Scheme 30)

### 6-Chloro-N-isopropyl-N′-prop-2-ynyl-[1,3,5]triazine-2,4-diamine (59)

A mixture of N-(4,6-dichloro-[1,3,5]triazin-2-yl)-N-prop-2-ynyl-amine (52) (534 mg, 2.63 mmol), isopropylamine (215 μL, 2.63 mmol) and N,N-diisopropyl ethylamine (458 μL, 2.63 mmol) in 1,4-dioxane (15 mL) was stirred at 50° C. for 3 h. The volatiles were evaporated and water (30 mL) was added. The resulting suspension was extracted with EtOAc (3×30 mL). The combined organic extracts were washed with water (30 mL), then with a brine solution (30 mL) and lastly dried over solid anhydrous Na₂SO₄. After filtration, the solvent was removed to yield 6-chloro-N-isopropyl-N′-prop-2-ynyl-[1,3,5]triazine-2,4-diamine (59) (564 mg, 95%). 400 MHz ¹H NMR (CDCl₃, ppm): δ 5.86 (0.7H br s), 5.68 (0.3H, br s), 5.38-5.09 (1H, m), 4.28-4.00 (3H, m), 2.30-2.20 (1H, m), 1.27-1.17 (6H, m). ESI-MS (m/z): 226, 228 [M+H]⁺.

### N-(4-Isopropylamino-6-prop-2-ynylamino-[1,3,5]triazin-2-yl)-O,N-dimethyl-hydroxylamine (60)

6-Chloro-N-isopropyl-N′-prop-2-ynyl-[1,3,5]triazine-2,4-diamine (59) and O,N-dimethylhydroxylamine hydrochloride were reacted using the procedure described for Compound 4 to yield N-(4-isopropylamino-6-prop-2-ynylamino-[1,3,5]triazin-2-yl)-O,N-dimethyl-hydroxylamine (60) in 94% yield. 400 MHz ¹H NMR (CDCl₃, ppm): δ 5.08 (1H, br s), 5.0-4.7 (1H, m), 4.29-4.05 (3H, m), 3.77 (3H, s), 3.27 (3H, s), 2.19 (1H, t, J=2.5 Hz), 1.19 (6H, d, J=6.5 Hz). ESI-MS (m/z): 251 [M+H]⁺.

### N-(4-Isopropylamino-6-prop-2-ynylamino-[1,3,5]triazin-2-yl)-O,N-dimethyl-hydroxylamine hydrochloride (61a)

N-(4-Isopropylamino-6-prop-2-ynylamino-[1,3,5]triazin-2-yl)-O,N-dimethyl-hydroxylamine (60) and 2 M HCl/diethyl ether were reacted using the procedure described for Compound 5a to yield N-(4-isopropylamino-6-prop-2-ynylamino-[1,3,5]triazin-2-yl)-O,N-dimethyl-hydroxylamine hydrochloride (61a) in quantitative yield. 400 MHz ¹H NMR (CDCl₃, ppm): δ 9.49-8.73 (0.7H, m), 7.46-7.29 (0.3H, m), 6.87-6.62 (0.3H, m), 6.27-5.53 (0.7H, m), 4.29-3.99 (3H, m), 3.91-3.85 (2.6H, m), 3.76 (0.4H, s), 3.39-3.32 (1.7H, m), 3.29 (1.3H, m), 2.25-2.17 (1H, m), 1.28-1.17 (6H, m). ESI-MS (m/z): 251 [M+H]⁺.

### Example 24

### O,N-Dimethyl-N-(4-cyclopropylamino-6-prop-2-ynylamino-[1,3,5]triazin-2-yl)-hydroxylamine (63) and corresponding hydrochloride salt (64a) (Scheme 30)

### 6-Chloro-N-cyclopropyl-N′-prop-2-ynyl-[1,3,5]triazine-2,4-diamine (62)

A mixture of N-(4,6-dichloro-[1,3,5]triazin-2-yl)-N-prop-2-ynyl-amine (52) (450 mg, 2.22 mmol), cyclopropylamine (139 mg, 2.44 mmol) and N,N-diisopropyl ethylamine (425 μL, 2.44 mmol) in 1,4-dioxane (5 mL) was stirred at ambient temperature for 4 h. The volatiles were then evaporated, the residue was taken up in water (150 mL) and stirred for 1 hour. After this time the mixture was filtered and dried to yield 6-chloro-N-cyclopropyl-N′-prop-2-ynyl-[1,3,5]triazine-2,4-diamine (62) (405 mg, 82%). 400 MHz ¹H NMR (DMSO-d₆, ppm): δ 8.26-8.15 (0.5H, m), 8.11-7.98 (1H, m), 7.93-7.83 (0.5H, m), 4.09 (1H, dd, J=5.8, 2.4 Hz), 4.01-3.96 (1H, m), 3.10 (1H, t, J=2.4 Hz), 2.85-2.68 (1H, m), 0.71-0.62 (2H, m), 0.53-0.46 (2H, m). ESI-MS (m/z): 224, 226 [M+H]⁺.

### O,N-Dimethyl-N-(4-cyclopropylamino-6-prop-2-ynylamino-[1,3,5]triazin-2-yl)-hydroxylamine (63)

A mixture of 6-chloro-N-cyclopropyl-N′-prop-2-ynyl-[1,3,5]triazine-2,4-diamine (62) (380 mg, 1.70 mmol), O,N-dimethylhydroxylamine hydrochloride (380 mg, 3.91 mmol) and NaOH (156 mg, 3.91 mmol) in 1,4-dioxane (15 mL) was heated at 90° C. for 2 h. The mixture was cooled to ambient temperature. A saturated NaHCO₃ solution (30 mL) was added to the residue and the mixture was extracted with CH₂Cl₂ (3×30 mL). The combined organic extracts were washed with water (100 mL) and dried over solid anhydrous Na₂SO₄. After filtration, the solvent was removed under reduced pressure and the residue was purified by flash column chromatography using gradient elution from CH₂Cl₂/EtOH (99:1) to CH₂Cl₂/EtOH (95:5) to yield O,N-dimethyl-N-(4-cyclopropylamino-6-prop-2-ynylamino-[1,3,5]triazin-2-yl)-hydroxylamine (63) (340 mg, 81%). 400 MHz ¹H NMR (CDCl₃, ppm): δ 5.30-5.07 (2H, m), 4.31-4.10 (2H, m), 3.77 (3H, s), 3.29 (3H, s), 2.82-2.70 (1H, m), 2.19 (1H, t, J=2.5 Hz), 0.80-0.66 (2H, m), 0.57-0.48 (2H, m). ESI-MS (m/z): 249 [M+H]⁺.

### O,N-Dimethyl-N-(4-cyclopropylamino-6-prop-2-ynylamino-[1,3,5]triazin-2-yl)-hydroxylamine hydrochloride (64a)

O,N-Dimethyl-N-(4-cyclopropylamino-6-prop-2-ynylamino-[1,3,5]triazin-2-yl)-hydroxylamine (63) (333 mg, 1.34 mmol) and 2 M HCl/diethyl ether (0.67 mL, 1.34 mmol) were reacted in diethyl ether (20 mL) at 0° C. The mixture was stirred for 0.5 h at 0° C. and then the volatiles were removed under vacuum to yield O,N-dimethyl-N-(4-cyclopropylamino-6-prop-2-ynylamino-[1,3,5]triazin-2-yl)-hydroxylamine hydrochloride (64a) in quantitative yield. 400 MHz ¹H NMR (D₂O, ppm): δ 4.39-4.25 (2H, m), 3.81 (3H, s), 3.56-3.29 (3H, m), 3.02-2.58 (2H, m), 1.02-0.82 (2H, m), 0.80-0.58 (2H, m). ESI-MS (m/z): 249 [M+H]⁺.

### Example 25

### O,N-Dimethyl-N-(4-n-butylamino-6-prop-2-ynylamino-[1,3,5]triazin-2-yl)-hydroxylamine (66) and corresponding hydrochloride salt (67a) (Scheme 30)

### N-Butyl-6-chloro-N′-prop-2-ynyl-[1,3,5]triazine-2,4-diamine (65)

N-(4,6-Dichloro-[1,3,5]triazin-2-yl)-N-prop-2-ynyl-amine (52) and butylamine were reacted using the procedure described for Compound 59 to yield N-butyl-6-chloro-N′-prop-2-ynyl-[1,3,5]triazine-2,4-diamine (65) in quantitative yield. 400 MHz ¹H NMR (DMSO-d₆, ppm): δ 8.16-8.02 (1H, m), 7.98-7.82 (1H, m), 4.04-3.96 (2H, m), 3.29-3.15 (2H, m), 3.11-3.06 (1H, m), 1.54-1.40 (2H, m), 1.35-1.23 (2H, m), 0.92-0.84 (3H, m). ESI-MS (m/z): 240, 242 [M+H]⁺.

### N-(4-Butylamino-6-prop-2-ynylamino-[1,3,5]triazin-2-yl)-O,N-dimethyl-hydroxylamine hydrochloride (66)

N-Butyl-6-chloro-N′-prop-2-ynyl-[1,3,5]triazine-2,4-diamine (65) and O,N-dimethylhydroxylamine hydrochloride were reacted using the procedure described for Compound 4 to yield N-(4-butylamino-6-prop-2-ynylamino-[1,3,5]triazin-2-yl)-O,N-dimethyl-hydroxylamine hydrochloride (66) in 93% yield. 400 MHz ¹H NMR (CDCl₃, ppm): δ 5.11-4.82 (2H, br s), 4.27-4.13 (2H, m), 3.77 (3H, s), 3.43-3.33 (2H, m), 3.28 (3H, s), 2.19 (1H, t, J=2.6 Hz), 1.58-1.48 (2H, m), 1.43-1.33 (2H, m), 0.93 (3H, t, J=7.4 Hz). ESI-MS (m/z): 265 [M+H]⁺.

### N-(4-Butylamino-6-prop-2-ynylamino-[1,3,5]triazin-2-yl)-O,N-dimethyl-hydroxylamine hydrochloride (67a)

N-(4-Butylamino-6-prop-2-ynylamino-[1,3,5]triazin-2-yl)-O,N-dimethyl-hydroxylamine hydrochloride (66) and 2M HCl/diethyl ether were reacted using procedure described for Compound 5a to yield N-(4-butylamino-6-prop-2-ynylamino-[1,3,5]triazin-2-yl)-O,N-dimethyl-hydroxylamine hydrochloride (67a) in quantitative yield. 400 MHz ¹H NMR (D₂O, ppm): δ 4.13-3.99 (2H, m), 3.70-3.59 (3H, m), 3.37-3.12 (5H, m), 2.55-2.45 (1H, m), 1.48-1.33 (2H, m), 1.18 (2H, sextet, J=7.3 Hz), 0.74 (3H, t, J=7.3 Hz). ESI-MS (m/z): 265 [M+H]⁺.

### Example 26

### O,N-Dimethyl-N-(4-cyclobutylamino-6-prop-2-ynylamino-[1,3,5]triazin-2-yl)-hydroxylamine (69) and corresponding hydrochloride salt (70a) (Scheme 30)

### 6-Chloro-N-cyclobutyl-N′-prop-2-ynyl-[1,3,5]triazine-2,4-diamine (68)

N-(4,6-Dichloro-[1,3,5]triazin-2-yl)-N-prop-2-ynyl-amine (52) and cyclobutylamine were reacted using the procedure described for Compound 59 to yield 6-chloro-N-cyclobutyl-N′-prop-2-ynyl-[1,3,5]triazine-2,4-diamine (68) in quantitative yield. 400 MHz ¹H NMR (DMSO-d₆, ppm): δ 8.29-8.19 (1H, m), 8.09-8.04 (1H, m), 4.38-4.24 (1H, m), 4.06-3.94 (2H, m), 3.11-3.08 (1H, m), 2.27-2.10 (2H, m), 2.03-1.87 (2H, m), 1.71-1.54 (2H, m). ESI-MS (m/z): 238, 240 [M+H]⁺.

### N-(4-Cyclobutylamino-6-prop-2-ynylamino-[1,3,5]triazin-2-yl)-O,N-dimethyl-hydroxylamine (69)

6-Chloro-N-cyclobutyl-N′-prop-2-ynyl-[1,3,5]triazine-2,4-diamine (68) and O,N-dimethylhydroxylamine hydrochloride were reacted using the procedure described for Compound 4 to yield N-(4-cyclobutylamino-6-prop-2-ynylamino-[1,3,5]triazin-2-yl)-O,N-dimethyl-hydroxylamine (69) in 77% yield. 400 MHz ¹H NMR (DMSO-d₆, ppm): δ 7.41-6.97 (2H, m), 4.41-4.27 (1H, m), 4.05-3.92 (2H, m), 3.74-3.59 (3H, m), 3.23-3.08 (3H, m), 3.02-2.96 (1H, m), 2.25-2.09 (2H, m), 2.03-1.86 (2H, m), 1.67-1.48 (2H, m). ESI-MS (m/z): 263 [M+H]⁺.

### N-(4-Cyclobutylamino-6-prop-2-ynylamino-[1,3,5]triazin-2-yl)-O,N-dimethyl-hydroxylamine hydrochloride (70a)

N-(4-Cyclobutylamino-6-prop-2-ynylamino-[1,3,5]triazin-2-yl)-O,N-dimethyl-hydroxylamine (69) and 2 M HCl/diethyl ether were reacted using the procedure described for Compound 5a to yield N-(4-cyclobutylamino-6-prop-2-ynylamino-[1,3,5]triazin-2-yl)-O,N-dimethyl-hydroxylamine hydrochloride (70a) in quantitative yield. 400 MHz ¹H NMR (DMSO-d₆, ppm): δ 12.9-11.9 (1H, br s), 9.04-8.34 (2H, m), 4.45-4.26 (1H, m), 4.22-4.06 (2H, m), 3.76 (3H, s), 3.38-3.15 (4H, m), 2.35-2.13 (2H, m), 2.08-1.89 (2H, m), 1.77-1.58 (2H, m). ESI-MS (m/z): 263 [M+H]⁺; melting point: 105-107° C.

### Example 27

### O,N-Dimethyl-N-(4-cyclopropylmethylamino-6-prop-2-ynylamino-[1,3,5]triazin-2-yl)-hydroxylamine (72) and corresponding hydrochloride salt (73a) (Scheme 30)

### 6-Chloro-N-cyclopropylmethyl-N′-prop-2-ynyl-[1,3,5]triazine-2,4-diamine (71)

N-(4,6-Dichloro-[1,3,5]triazin-2-yl)-N-prop-2-ynyl-amine (52) and cyclopropylmethylamine were reacted using the procedure described for Compound 59 to yield 6-chloro-N-cyclopropylmethyl-N′-prop-2-ynyl-[1,3,5]triazine-2,4-diamine (71) in 83% yield. 400 MHz ¹H NMR (DMSO-d₆, ppm): δ 8.18-8.00 (1.5H, m), 7.98-7.79 (0.5H, m), 4.04-3.97 (2H, m), 3.14 (1H, t, J=6.4 Hz), 3.11-3.05 (2H, m), 1.10-0.93 (1H, m), 0.44-0.37 (2H, m), 0.25-0.16 (2H, m). ESI-MS (m/z): 238, 240 [M+H]⁺.

### O,N-Dimethyl-N-(4-cyclopropylmethylamino-6-prop-2-ynylamino-[1,3,5]triazin-2-yl)-hydroxylamine (72)

6-Chloro-N-cyclopropylmethyl-N′-prop-2-ynyl-[1,3,5]triazine-2,4-diamine (71) and O,N-dimethylhydroxylamine hydrochloride were reacted using the procedure described for Compound 4 to yield O,N-dimethyl-N-(4-cyclopropylmethyl amino-6-prop-2-ynylamino-[1,3,5]triazin-2-yl)-hydroxylamine (72) in 91% yield. 400 MHz ¹H NMR (CDCl₃, ppm): δ 5.24-4.95 (2H, m), 4.26-4.11 (2H, m), 3.77 (3H, s), 3.44-3.05 (5H, m), 2.19 (1H, t, J=2.5 Hz), 1.09-0.96 (1H, m), 0.57-0.43 (2H, m), 0.28-0.16 (2H, m). ESI-MS (m/z): 263 [M+H]⁺.

### O,N-Dimethyl-N-(4-cyclopropylmethylamino-6-prop-2-ynylamino-[1,3,5]triazin-2-yl)-hydroxylamine hydrochloride (73a)

O,N-Dimethyl-N-(4-cyclopropylmethylamino-6-prop-2-ynylamino-[1,3,5]triazin-2-yl)-hydroxylamine (72) and 2M HCl/diethyl ether were reacted using the procedure described for Compound 5a to yield O,N-dimethyl-N-(4-cyclopropylmethylamino-6-prop-2-ynylamino-[1,3,5]triazin-2-yl)-hydroxylamine hydrochloride (73a) in quantitative yield. 400 MHz ¹H NMR (D₂O, ppm): δ 4.34-4.15 (2H, m), 3.83 (3H, s), 3.55-3.14 (5H, m), 2.67 (1H, s), 1.24-1.01 (1H, m), 0.68-0.45 (2H, m), 0.40-0.16 (2H, m). ESI-MS (m/z): 263 [M+H]⁺.

### Example 28

### O,N-Dimethyl-N-(4-cyclohexylamino-6-prop-2-ynylamino-[1,3,5]triazin-2-yl)-hydroxylamine (75) and corresponding hydrochloride salt (76a) (Scheme 30)

### 6-Chloro-N-cyclohexyl-N′-prop-2-ynyl-[1,3,5]triazine-2,4-diamine (74)

N-(4,6-Dichloro-[1,3,5]triazin-2-yl)-N-prop-2-ynyl-amine (52) and cyclohexylamine were reacted using the procedure described for Compound 59 to yield 6-chloro-N-cyclohexyl-N′-prop-2-ynyl-[1,3,5]triazine-2,4-diamine (74) in quantitative yield. 400 MHz ¹H NMR (CDCl₃, ppm): δ 5.81-5.55 (1H, m), 5.38-5.12 (1H, m), 4.28-4.10 (2H, m), 3.95-3.79 (1H, m), 2.28-2.21 (1H, m), 2.04-1.90 (2H, m), 1.78-1.68 (2H, m), 1.67-1.57 (1H, m), 1.47-1.31 (2H, m), 1.30-1.12 (3H, m). ESI-MS (m/z): 266, 268 [M+H]⁺.

### O,N-Dimethyl-N-(4-cyclohexylamino-6-prop-2-ynylamino-[1,3,5]triazin-2-yl)-hydroxylamine (75)

6-Chloro-N-cyclohexyl-N′-prop-2-ynyl-[1,3,5]triazine-2,4-diamine (74) and O,N-dimethylhydroxylamine hydrochloride were reacted using procedure described for Compound 4 to yield O,N-dimethyl-hydroxylamine-N-(4-cyclohexylamino-6-prop-2-ynylamino-[1,3,5]triazin-2-yl)-hydroxylamine (75) in 80% yield. 400 MHz ¹H NMR (CDCl₃, ppm): δ 5.05-4.80 (2H, m), 4.22-4.16 (2H, m), 3.87-3.73 (4H, m), 3.28 (3H, s), 2.20 (1H, t, J=2.5 Hz), 2.03-1.95 (2H, m), 1.77-1.68 (2H, m), 1.65-1.57 (1H, m), 1.42-1.31 (2H, m), 1.25-1.12 (3H, m). ESI-MS (m/z): 291 [M+H]⁺.

### O,N-Dimethyl-N-(4-cyclohexylamino-6-prop-2-ynylamino-[1,3,5]triazin-2-yl)-hydroxylamine hydrochloride (76a)

O,N-dimethyl-N-(4-cyclohexylamino-6-prop-2-ynylamino-[1,3,5]triazin-2-yl)-hydroxylamine (75) and 2 M HCl/diethyl ether were reacted using the procedure described for Compound 5a to yield O,N-dimethyl-N-(4-cyclohexylamino-6-prop-2-ynylamino-[1,3,5]triazin-2-yl)-hydroxylamine hydrochloride (76a) in quantitative yield. 400 MHz ¹H NMR (D₂O, ppm): δ 4.29-4.19 (2H, m), 4.04-3.78 (4H, m), 3.46 (1.8H, s), 3.37 (1.2H, s), 2.69-2.66 (1H, m), 2.00-1.88 (2H, m), 1.78-1.68 (2H, m), 1.65-1.57 (1H, m), 1.46-1.18 (5H, m). ESI-MS (m/z): 291 [M+H]⁺.

### Example 29

### O,N-Dimethyl-N-(4-cyclohexylmethylamino-6-prop-2-ynylamino-[1,3,5]triazin-2-yl)-hydroxylamine (78) and corresponding hydrochloride salt (79a) (Scheme 30)

### 6-Chloro-N-cyclohexylmethyl-N′-prop-2-ynyl-[1,3,5]triazine-2,4-diamine (77)

N-(4,6-Dichloro-[1,3,5]triazin-2-yl)-N-prop-2-ynyl-amine (52) and cyclohexyl-methylamine using procedure described for Compound 59. The crude product was crystallized from EtOAc to yield pure 6-chloro-N-cyclohexylmethyl-N′-prop-2-ynyl-[1,3,5]triazine-2,4-diamine (77) in 73% yield. 400 MHz ¹H NMR (DMSO-d₆, ppm): δ 8.14-7.70 (2H, m), 4.05-3.94 (2H, m), 3.17-3.01 (3H, m), 1.73-1.42 (6H, m), 1.30-1.07 (3H, m), 0.97-0.80 (2H, m). ESI-MS (m/z): 280, 282 [M+H]⁺.

### O,N-Dimethyl-N-[4-(cyclohexylmethyl-amino)-6-prop-2-ynylamino-[1,3,5]triazin-2-yl]-hydroxylamine (78)

6-Chloro-N-cyclohexylmethyl-N′-prop-2-ynyl-[1,3,5]triazine-2,4-diamine (77) and O,N-dimethylhydroxylamine hydrochloride were reacted using the procedure described for Compound 4 to yield O,N-dimethyl-N-[4-(cyclohexylmethyl-amino)-6-prop-2-ynylamino-[1,3,5]triazin-2-yl]-hydroxylamine (78) in 94% yield. 400 MHz ¹H NMR (CDCl₃, ppm): 5.26-4.88 (2H, m), 4.18 (2H, s), 3.76 (3H, s), 3.28 (3H, s), 3.26-3.17 (2H, m), 2.19 (1H, t, J=2.5 Hz), 1.81-1.59 (5H, m), 1.58-1.44 (1H, m), 1.31-1.07 (3H, m), 1.02-0.83 (2H, m). ESI-MS (m/z): 305 [M+H]⁺.

### O,N-Dimethyl-N-[4-(cyclohexylmethyl-amino)-6-prop-2-ynylamino-[1,3,5]triazin-2-yl]-hydroxylamine hydrochloride (79a)

O,N-Dimethyl-N-[4-(cyclohexylmethyl-amino)-6-prop-2-ynylamino-[1,3,5]triazin-2-yl]-hydroxylamine (78) and 2 M HCl/diethyl ether were reacted using the procedure described for Compound 5a to yield O,N-dimethyl-N-[4-(cyclohexylmethyl-amino)-6-prop-2-ynylamino-[1,3,5]triazin-2-yl]-hydroxylamine hydrochloride (79a) in quantitative yield. 400 MHz ¹H NMR (CDCl₃, ppm): δ 9.51-8.85 (1H, m), 6.13-5.81 (1H, m), 4.32-4.12 (2H, m), 3.97-3.91 (2.6H, m), 3.82 (0.4H, s), 3.48-3.20 (5H, m), 2.32-2.20 (1H, m), 1.86-1.47 (6H, m), 1.35-1.08 (3H, m), 1.07-0.88 (2H, m). ESI-MS (m/z): 305 [M+H]⁺.

### Example 30

### O,N-Dimethyl-N-(4-benzylamino-6-prop-2-ynylamino-[1,3,5]triazin-2-yl)-hydroxylamine (81) and corresponding hydrochloride salt (82a) (Scheme 30)

### N-Benzyl-6-chloro-N′-prop-2-ynyl-[1,3,5]triazine-2,4-diamine (80)

N-(4,6-Dichloro-[1,3,5]triazin-2-yl)-N-prop-2-ynyl-amine (52) and benzylamine were reacted using the procedure described for Compound 59 to yield N-benzyl-6-chloro-N′-prop-2-ynyl-[1,3,5]triazine-2,4-diamine (80) in 83% yield. 400 MHz ¹H NMR (DMSO-d₆, ppm): δ 8.52-8.32 (1H, m), 8.26-8.11 (1H, m), 7.38-7.20 (5H, m), 4.49-4.42 (2H, m), 4.05-3.96 (2H, m), 3.12-3.09 (1H, m). ESI-MS (m/z): 274, 276 [M+H]⁺.

### O,N-Dimethyl-N-(4-benzylamino-6-prop-2-ynylamino-[1,3,5]triazin-2-yl)-hydroxylamine (81)

N-Benzyl-6-chloro-N′-prop-2-ynyl-[1,3,5]triazine-2,4-diamine (80) and O,N-dimethylhydroxylamine hydrochloride were reacted using the procedure described for Compound 4 to yield O,N-dimethyl-N-(4-benzylamino-6-prop-2-ynylamino-[1,3,5]triazin-2-yl)-hydroxylamine (81) in 98% yield. 400 MHz ¹H NMR (CDCl₃, ppm): δ 7.34-7.23 (5H, m, overlapped with CHCl₃), 5.44-5.18 (1H, m), 5.07 (1H, br s), 4.60 (2H, d, J=5.9 Hz), 4.22-4.16 (2H, m), 3.76 (3H, s), 3.29 (3H, s), 2.20 (1H, t, J=2.6 Hz). ESI-MS (m/z): 299 [M+H]⁺.

### O,N-Dimethyl-N-(4-benzylamino-6-prop-2-ynylamino-[1,3,5]triazin-2-yl)-hydroxylamine hydrochloride (82a)

O,N-Dimethyl-N-(4-benzylamino-6-prop-2-ynylamino-[1,3,5]triazin-2-yl)-hydroxylamine (81) and 2M HCl/diethyl ether were reacted using the procedure described for Compound 5a to yield O,N-dimethyl-N-(4-benzylamino-6-prop-2-ynylamino-[1,3,5]triazin-2-yl)-hydroxylamine hydrochloride (82a) in quantitative yield. 400 MHz ¹H NMR (D₂O, ppm): δ 7.47-7.33 (5H, m), 4.70-4.58 (2H, m), 4.23-4.20 (2H, m), 3.84-3.77 (3H, m), 3.46-3.37 (3H, m), 2.69-2.63 (1H, m). ESI-MS (m/z): 299 [M+H]⁺.

### Example 31

### O,N-Dimethyl-N-[4-(1-methyl-prop-2-ynylamino)-6-n-propylamino-[1,3,5]triazin-2-yl]-hydroxylamine (84) and corresponding hydrochloride salt (85a) (Scheme 31)

### 6-Chloro-N-(1-methyl-prop-2-ynyl)-N′-propyl-[1,3,5]triazine-2,4-diamine (83)

A mixture of 4,6-dichloro-[1,3,5]triazin-2-yl)-n-propyl-amine (2) (476 mg, 2.30 mmol), 1-methyl-prop-2-ynylamine hydrochloride (243 mg, 2.30 mmol) and N,N-diisopropylethylamine (849 μL, 4.60 mmol) in 1,4-dioxane (15 mL) was stirred at 55° C. for 6 h. The mixture was cooled to ambient temperature and water (20 mL) was added. The resulting suspension was extracted with EtOAc (3×30 mL). The combined organic extracts were washed with water (30 mL), then with a brine solution (30 mL) and lastly dried over solid anhydrous Na₂SO₄. After filtration, the solvent was removed under vacuum; and the residue was purified by flash column chromatography using CH₂Cl₂/MeOH (98:2) to yield 6-chloro-N-(1-methyl-prop-2-ynyl)-N′-n-propyl-[1,3,5]triazine-2,4-diamine (83) (530 mg, 96%). 400 MHz ¹H NMR (CDCl₃, ppm): δ 5.74-5.45 (1H, m), 5.41-5.25 (1H, m), 5.00-4.78 (1H, m), 3.43-3.25 (2H, m), 2.30-2.24 (1H, m), 1.67-1.53 (2H, m), 1.52-1.43 (3H, m), 1.00-0.89 (3H, m).

### O,N-Dimethyl-N-[4-(1-methyl-prop-2-ynylamino)-6-n-propylamino-[1,3,5]triazin-2-yl]-hydroxylamine (84)

A mixture of 6-chloro-N-(1-methyl-prop-2-ynyl)-N′-n-propyl-[1,3,5]triazine-2,4-diamine (83) (400 mg, 1.67 mmol), O,N-dimethylhydroxylamine hydrochloride (326 mg, 3.34 mmol) and NaOH (120 mg, 3.00 mmol) in 1,4-dioxane (15 mL) was heated at 90° C. for 3 h. The mixture was cooled to ambient temperature and a saturated NaHCO₃ solution (30 mL) was added to the residue. The mixture was extracted with CH₂Cl₂ (3×30 mL). The combined organic extracts were washed with water (100 mL) and dried over solid anhydrous Na₂SO₄. After filtration, the solvent was removed under reduced pressure and the residue was purified by flash column chromatography using gradient elution from CH₂Cl₂/EtOH (99:1) to CH₂Cl₂/EtOH (96:4) to yield O,N-dimethyl-N-[4-(1-methyl-prop-2-ynylamino)-6-n-propylamino-[1,3,5]triazin-2-yl]-hydroxylamine (84) (430 mg, 97%). 400 MHz ¹H NMR (CDCl₃, ppm): 5.17-4.80 (3H, m), 3.81-3.72 (3H, br s), 3.39-3.17 (5H, m), 2.26 (1H, d, J=2.1 Hz), 1.57 (2H, sextet, J=7.4 Hz), 1.46 (3H, d, J=6.8 Hz), 0.94 (3H, t, J=7.4 Hz). ESI-MS (m/z): 265 [M+H]⁺.

### O,N-Dimethyl-N-[4-(1-methyl-prop-2-ynylamino)-6-n-propylamino-[1,3,5]triazin-2-yl]-hydroxylamine hydrochloride (85a)

A 2M HCl/diethyl ether solution (0.75 mL, 1.50 mmol) was added to a solution of O,N-dimethyl-N-[4-(1-methyl-prop-2-ynylamino)-6-n-propylamino-[1,3,5]triazin-2-yl]-hydroxylamine (84) (400 mg, 1.51 mmol) in diethyl ether (15 mL) at 0° C. The mixture was stirred for 0.5 h at 0° C. and then volatiles were removed under reduced pressure to yield O,N-dimethyl-N-[4-(1-methyl-prop-2-ynylamino-[6-n-propylamino-[1,3,5]triazin-2-yl]-hydroxylamine hydrochloride (85a) in quantitative yield. 400 MHz ¹H NMR (CDCl₃, ppm): δ 9.60-8.94 (1H, m), 6.18-5.78 (1H, br s), 4.98-4.74 (1H, m), 3.98-3.88 (2.5H, m), 3.89 (0.5H, s), 3.48-3.27 (5H, m), 2.34-2.28 (1H, m), 1.74-1.56 (2H, m), 1.53 (3H, t, J=7.5 Hz), 1.03-0.93 (3H, m). ESI-MS (m/z): 265 [M+H]⁺.

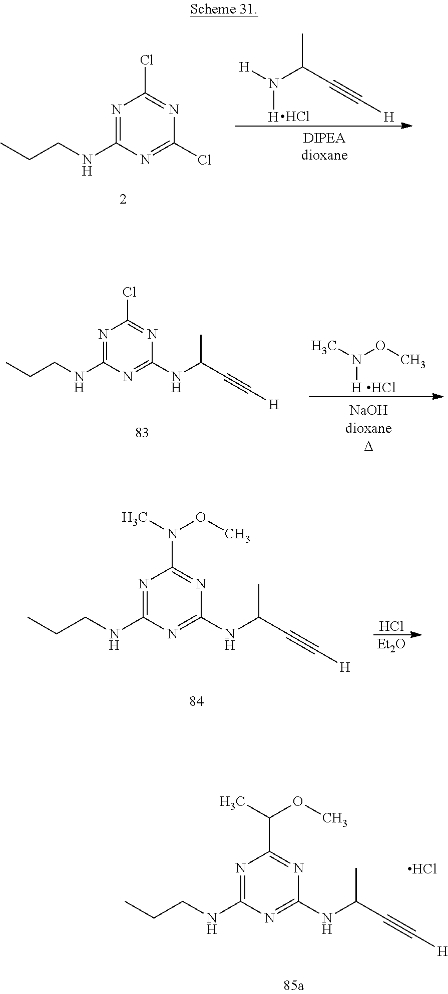

### Example 32

### O,N-Dimethyl-N-(4-but-3-ynylamino-6-n-propylamino-[1,3,5]triazin-2-yl)-hydroxylamine (87) and corresponding hydrochloride salt (88a) (Scheme 32)

### N-But-3-ynyl-6-chloro-N′-propyl-[1,3,5]triazine-2,4-diamine (86)

A mixture of 4,6-dichloro-[1,3,5]triazin-2-yl)-propyl-amine (2) (1.40 g, 6.73 mmol), but-3-ynylamine (465 mg, 6.73 mmol) and N,N-diisopropylethylamine (1.30 mL, 8.08 mmol) in 1,4-dioxane (10 mL) was stirred at 90° C. for 24 h. The mixture was cooled to ambient temperature and water (50 mL) was added. The resulting suspension was extracted with EtOAc (3×75 mL). The combined organic extracts were washed with water (70 mL), then with a brine solution (70 mL) and lastly dried over solid anhydrous Na₂SO₄. After filtration, the solvent was removed under vacuum and the resultant residue was purified by flash column chromatography using gradient elution from PE/EtOAc (3:1) to PE/EtOAc (1:99) to yield N-but-3-ynyl-6-chloro-N′-propyl-[1,3,5]triazine-2,4-diamine (86) (1.14 g, 70%). 400 MHz ¹H NMR (DMSO-d₆, ppm): δ 7.89 (1H, t, J=5.2 Hz), 7.86-7.75 (1H, m), 3.39-3.28 (2H, m), 3.23-3.11 (2H, m), 2.85-2.81 (1H, m), 2.44-2.31 (2H, m), 1.56-1.42 (2H, m), 0.89-0.82 (3H, m). ESI-MS (m/z): 240, 242 [M+H]⁺.

### O,N-Dimethyl-N-(4-but-3-ynylamino-6-n-propylamino-[1,3,5]triazin-2-yl)-hydroxylamine (87)

N-But-3-ynyl-6-chloro-N′-propyl-[1,3,5]triazine-2,4-diamine (86) and O,N-dimethylhydroxylamine hydrochloride were reacted using the procedure described for Compound 84 to yield O,N-dimethyl-N-(4-but-3-ynylamino-6-n-propylamino-[1,3,5]triazin-2-yl)-hydroxylamine (87) in 99% yield. 400 MHz ¹H NMR (CDCl₃, ppm): δ 5.6-4.9 (2H, br s), 3.80 (3H, s), 3.61-3.51 (2H, m), 3.39-3.42 (5H, m), 2.53-2.42 (2H, m), 2.04-1.97 (1H, m), 1.59 (2H, sextet, J=7.4 Hz), 0.96 (3H, t, J=7.4 Hz). ESI-MS (m/z): 265 [M+H]⁺.

### O,N-Dimethyl-N-(4-but-3-ynylamino-6-n-propylamino-[1,3,5]triazin-2-yl)-hydroxylamine hydrochloride (88a)

O,N-Dimethyl-N-(4-but-3-ynylamino-6-n-propylamino-[1,3,5]triazin-2-yl)-hydroxylamine (87) and 2 M HCl/diethyl ether were reacted using procedure described for Compound 85a to yield O,N-dimethyl-N-(4-but-3-ynylamino-6-n-propylamino-[1,3,5]triazin-2-yl)-hydroxylamine hydrochloride (88a) in quantitative yield. 400 MHz ¹H NMR (CDCl₃, ppm): δ 13.72-13.19 (1H, m), 9.73-9.03 (1H, m), 6.08-5.41 (1H, m), 3.98-3.79 (3H, m), 3.67-3.54 (2H, m), 3.46-3.29 (5H, m), 2.57-2.46 (2H, m), 2.09-2.02 (1H, m), 1.76-1.55 (2H, m), 1.04-0.94 (3H, m). ESI-MS (m/z): 265 [M+H]⁺.

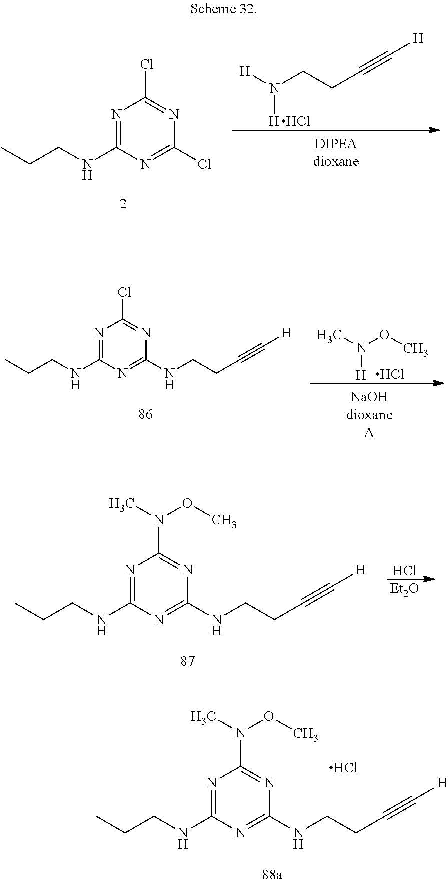

### Example 33

### N-But-3-ynyl-N′-methyl-N″-propyl-[1,3,5]triazine-2,4,6-triamine (89) and corresponding hydrochloride salt (90a) (Scheme 33)

### N-But-3-ynyl-N′-methyl-N″-propyl-[1,3,5]triazine-2,4,6-triamine (89)

A solution of N-but-3-ynyl-6-chloro-N′-propyl-[1,3,5]triazine-2,4-diamine (86) (320 mg, 1.33 mmol), 2M MeNH₂/THF (6.7 mL, 13.40 mmol) in 1,4-dioxane (5 mL) was heated at 90° C. for 3 h in a closed vial. The volatiles were removed under reduced pressure and a saturated NaHCO₃ solution (10 mL) was added to the residue. The mixture was extracted with EtOAc (3×15 mL). The combined organic extracts were washed with water (30 mL), then with a brine solution (30 mL) and lastly dried over solid anhydrous Na₂SO₄. After the solvent was removed under reduced pressure, the resulting residue was purified by flash column chromatography using CH₂Cl₂/EtOH (98:2) to yield N-but-3-ynyl-N′-methyl-N″-propyl-[1,3,5]triazine-2,4,6-triamine (89) (300 mg, 96%). 400 MHz ¹H NMR (CDCl₃, ppm): δ 8.53-8.16 (3H, m), 3.49-3.35 (2H, m), 3.34-3.18 (2H, m), 2.93-2.77 (4H, m), 2.48-2.37 (2H, m), 1.60-1.45 (2H, m), 0.88 (3H, t, J=7.4 Hz). ESI-MS (m/z): 265 [M+H]⁺.

### N-But-3-ynyl-N′-methyl-N″-propyl-[1,3,5]triazine-2,4,6-triamine hydrochloride (90a)

A 2M HCl/diethyl ether solution (0.64 mL, 1.28 mmol) was added to a solution of N-but-3-ynyl-N′-methyl-N″-propyl-[1,3,5]triazine-2,4,6-triamine (89) (300 mg, 1.28 mmol) in diethyl ether (5 mL) at 0° C. The mixture was stirred for 0.5 h at 0° C., after which time, the precipitate were filtered, washed with diethyl ether (5 mL) to yield N-but-3-ynyl-N′-methyl-N″-propyl-[1,3,5]triazine-2,4,6-triamine hydrochloride (90a) (330 mg, 95%). 400 MHz ¹H NMR (DMSO-d₆, ppm): δ 8.53-8.16 (3H, m), 3.49-3.35 (2H, m), 3.34-3.18 (2H, m), 2.93-2.77 (4H, m), 2.48-2.37 (2H, m), 1.60-1.45 (2H, m), 0.88 (3H, t, J=7.4 Hz). ESI-MS (m/z): 265 [M+H]⁺; melting point: 142-145° C.

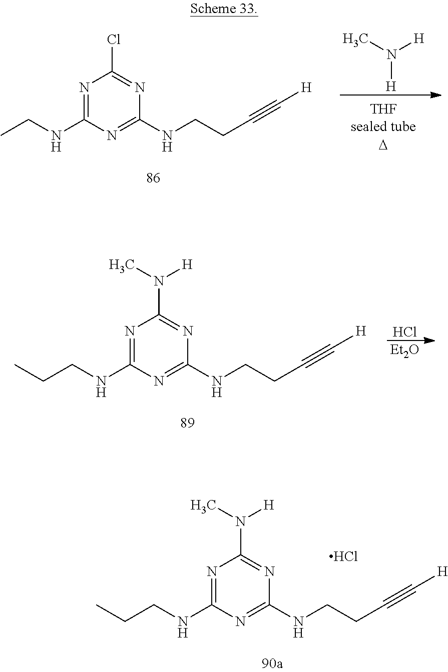

### Example 34

### O-tert-Butyl-N-(4-n-propylamino-6-prop-2-ynylamino-[1,3,5]triazin-2-yl)-hydroxylamine (91) and corresponding hydrochloride salt (92a) (Scheme 34)

### O-tert-Butyl-N-(4-n-propylamino-6-prop-2-ynylamino-[1,3,5]triazin-2-yl)-hydroxylamine (91)

A mixture of 6-chloro-N-n-propyl-N′-prop-2-ynyl-[1,3,5]triazine-2,4-diamine (3) (120 mg, 0.53 mmol), O-tert-butyl-hydroxylamine hydrochloride (140 mg, 1.11 mmol) and NaOH (44 mg, 1.11 mmol) in 1,4-dioxane (5 mL) was heated at 90° C. for 3 h. The mixture was cooled to ambient temperature. A saturated NaHCO₃ solution (15 mL) was added and the mixture was extracted with EtOAc (4×20 mL). The combined organic extracts were washed with water (30 mL), then with a brine solution (30 mL) and lastly dried over solid anhydrous Na₂SO₄. After filtration, the solvent was removed under reduced pressure and the resultant residue was purified by flash column chromatography using gradient elution from CH₂Cl₂/EtOH (99:1) to CH₂Cl₂/EtOH (97:3) to yield O-tert-butyl-N-(4-n-propylamino-6-prop-2-ynylamino-[1,3,5]triazin-2-yl)-hydroxylamine (91) (93 mg, 63%). 400 MHz ¹H NMR (CDCl₃, ppm): δ 7.37-7.25 (1H, m), 5.19 (1H, s), 5.13-4.93 (1H, br s), 4.26-4.10 (2H, m), 3.41-3.25 (2H, m), 2.22-2.19 (1H, m), 1.57 (2H, sextet, J=7.4 Hz), 1.29 (9H, s), 0.94 (3H, t, J=7.4 Hz). ESI-MS (m/z): 279 [M+H]⁺.

### O-tert-Butyl-N-(4-n-propylamino-6-prop-2-ynylamino-[1,3,5]triazin-2-yl)-hydroxylamine hydrochloride (92a)

A 2M HCl/diethyl ether solution (160 μL, 0.32 mmol) was added to a solution of O-tert-butyl-N-(4-n-propylamino-6-prop-2-ynylamino-[1,3,5]triazin-2-yl)-hydroxylamine (91) (88 mg, 0.32 mmol) in diethyl ether (3 mL) at 0° C. The mixture was stirred for 0.5 h at 0° C., after which time the volatiles were removed under reduced pressure to yield O-tert-butyl-N-(4-n-propylamino-6-prop-2-ynylamino-[1,3,5]triazin-2-yl)-hydroxylamine hydrochloride (92a) in quantitative yield. 400 MHz ¹H NMR (CDCl₃, ppm): δ 13.3-12.6 (1H, br s), 10.1-9.2 (1H, br s), 8.98-7.91 (1H, m), 6.0-5.4 (1H, br s), 4.29-4.09 (2H, m), 3.49-3.28 (2H, m), 2.33-2.21 (1H, m), 1.81-1.54 (2H, m), 1.45-1.30 (9H, m), 1.03-0.94 (3H, m). ESI-MS (m/z): 279 [M+H]⁺.

Scheme 34.

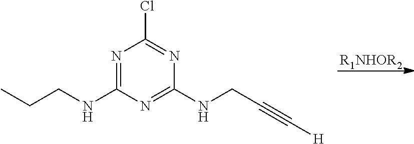

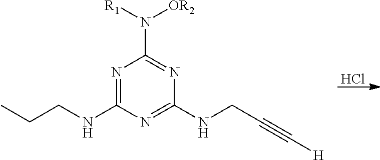

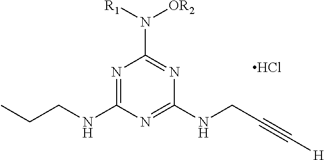

| cmpd | R₁ | R₂ |
|---|---|---|
| 91 | H | t-Bu |
| 92a | H | t-Bu |
| 93 | CH₃ | Et |
| 94a | CH₃ | Et |
| 95 | H | Et |
| 96a | H | Et |
| 97 | H | CH₃ |
| 98a | H | CH₃ |
| 99 | CH₃ | H |
| 100 | H | H |
| 101 | CH₃ | CH₂CH₂OCH₃ |
| 102a | CH₃ | CH₂CH₂OCH₃ |
| 103 | CH₃ | CH₂CH₂CH₂CF₂CF₃ |
| 104a | CH₃ | CH₂CH₂CH₂CF₂CF₃ |

### Example 35

### O-Ethyl-N-methyl-N-(4-n-propylamino-6-prop-2-ynylamino-[1,3,5]triazin-2-yl)-hydroxylamine (93) and corresponding hydrochloride salt (94a) (Scheme 34)

### O-Ethyl-N-methyl-N-(4-n-propylamino-6-prop-2-ynylamino-[1,3,5]triazin-2-yl)-hydroxylamine (93)

A mixture of 6-chloro-N-propyl-N′-prop-2-ynyl-[1,3,5]triazine-2,4-diamine (3) (398 mg, 1.76 mmol), O-ethyl-N-methyl-hydroxylamine hydrochloride (140 mg, 3.53 mmol) and NaOH (141 mg, 3.15 mmol) in 1,4-dioxane (15 mL) was heated at 90° C. for 16 h. The mixture was cooled to the room temperature. A saturated NaHCO₃ solution (15 mL) was added and the mixture was extracted with CH₂Cl₂ (3×250 mL). The combined organic extracts were washed with water (50 mL) and dried over solid anhydrous Na₂SO₄. After filtration, the solvent was removed under reduced pressure and the resultant residue was purified by flash column chromatography using gradient elution from CH₂Cl₂/EtOH (99:1) to CH₂Cl₂/EtOH (97:3) to yield O-ethyl-N-methyl-N-(4-n-propylamino-6-prop-2-ynylamino-[1,3,5]triazin-2-yl)-hydroxylamine (93) (440 mg, 94%). 400 MHz ¹H NMR (CDCl₃, ppm): δ 5.33-4.90 (2H, m), 4.26-4.10 (2H, br s), 4.08-3.91 (2H, m), 3.41-3.18 (5H, m), 2.19 (1H, t, J=2.4 Hz), 1.57 (2H, sextet, J=7.4 Hz), 1.28 (3H, t, J=7.1 Hz), 0.93 (3H, t, J=7.4 Hz). ESI-MS (m/z): 265 [M+H]⁺.

### O-Ethyl-N-methyl-N-(4-n-propylamino-6-prop-2-ynylamino-[1,3,5]triazin-2-yl)-hydroxylamine hydrochloride (94b)

O-Ethyl-N-methyl-N-(4-n-propylamino-6-prop-2-ynylamino-[1,3,5]triazin-2-yl)-hydroxylamine (93) and 2 M HCl/diethyl ether were reacted using the procedure described for Compound 92a to yield O-ethyl-N-methyl-N-(4-n-propylamino-6-prop-2-ynylamino-[1,3,5]triazin-2-yl)-hydroxylamine hydrochloride (94b) in quantitative yield. 400 MHz ¹H NMR (CDCl₃, ppm): δ 5.33-4.90 (2H, m), 4.26-4.10 (2H, br s), 4.08-3.91 (2H, m), 3.41-3.18 (5H, m), 2.19 (1H, t, J=2.4 Hz), 1.57 (2H, sextet, J=7.4 Hz), 1.28 (3H, t, J=7.1 Hz), 0.93 (3H, t, J=7.4 Hz). ESI-MS (m/z): 265 [M+H]⁺.

### Example 36

### O-Ethyl-N-(4-n-propylamino-6-prop-2-ynylamino-[1,3,5]triazin-2-yl)-hydroxylamine (95) and corresponding hydrochloride salt (96a) (Scheme 34)

### O-Ethyl-N-(4-n-propylamino-6-prop-2-ynylamino-[1,3,5]triazin-2-yl)-hydroxylamine hydrochloride (95)

6-Chloro-N-propyl-N′-prop-2-ynyl-[1,3,5]triazine-2,4-diamine (3) and O-ethyl-hydroxylamine hydrochloride were reacted using the procedure described for Compound 93 to yield O-ethyl-N-(4-n-propylamino-6-prop-2-ynylamino-[1,3,5]triazin-2-yl)-hydroxylamine hydrochloride (95) in 85% yield. 400 MHz ¹H NMR (CDCl₃, ppm): δ 8.72-7.96 (1H, m), 5.88-4.88 (2H, m), 4.27-4.12 (2H, m), 4.08-3.94 (2H, m), 3.42-3.23 (2H, m), 2.21 (1H, t, J=2.5 Hz), 1.57 (2H, sextet, J=7.5 Hz), 1.29 (3H, t, J=7.5 Hz), 0.94 (3H, t, J=7.5 Hz). ESI-MS (m/z): 251 [M+H]⁺.

### O-Ethyl-N-(4-n-propylamino-6-prop-2-ynylamino-[1,3,5]triazin-2-yl)-hydroxylamine hydrochloride (96a)

O-Ethyl-N-(4-n-propylamino-6-prop-2-ynylamino-[1,3,5]triazin-2-yl)-hydroxylamine (95) and 2 M HCl/diethyl ether were reacted using the procedure described for Compound 92a to yield O-ethyl-N-(4-n-propylamino-6-prop-2-ynylamino-[1,3,5]triazin-2-yl)-hydroxylamine hydrochloride (96a) in quantitative yield. 400 MHz ¹H NMR (CDCl₃, ppm): δ 4.31-3.96 (4H, m), 3.50-3.30 (2H, m), 2.31-2.22 (1H, m), 1.73-1.54 (2H, m), 1.41-1.28 (3H, m), 0.98 (3H, t, J=7.5 Hz). ESI-MS (m/z): 251 [M+H]⁺.

### Example 37

### O-Methyl-N-(4-n-propylamino-6-prop-2-ynylamino-[1,3,5]triazin-2-yl)-hydroxylamine (97) and corresponding hydrochloride salt (98a) (Scheme 34)

### O-Methyl-N-(4-n-propylamino-6-prop-2-ynylamino-[1,3,5]triazin-2-yl)-hydroxylamine (97)

6-Chloro-N-n-propyl-N′-prop-2-ynyl-[1,3,5]triazine-2,4-diamine (3) and O-methyl-hydroxylamine hydrochloride were reacted using the procedure described for Compound 93 to yield O-methyl-N-(4-n-propylamino-6-prop-2-ynylamino-[1,3,5]triazin-2-yl)-hydroxylamine (97) in 98% yield. 400 MHz ¹H NMR (CDCl₃, ppm): δ 8.02-7.60 (1H, m), 5.48-4.96 (2H, m), 4.25-4.14 (2H, m), 3.81 (3H, s), 3.40-3.27 (2H, m), 2.22 (1H, t, J=2.5 Hz), 1.58 (2H, sextet, J=7.3 Hz), 0.95 (3H, t, J=7.3 Hz). ESI-MS (m/z): 237 [M+H]⁺.

### O-Methyl-N-(4-n-propylamino-6-prop-2-ynylamino-[1,3,5]triazin-2-yl)-hydroxylamine hydrochloride (98a)

O-Methyl-N-(4-n-propylamino-6-prop-2-ynylamino-[1,3,5]triazin-2-yl)-hydroxylamine (97) and 2 M HCl/diethyl ether were reacted using the procedure described for Compound 92a to yield O-methyl-N-(4-n-propylamino-6-prop-2-ynylamino-[1,3,5]triazin-2-yl)-hydroxylamine hydrochloride (98a) in quantitative yield. 400 MHz ¹H NMR (D₂O, ppm): δ 4.27 (1H, s), 4.21 (1H, s), 3.85-3.79 (3H, m), 3.51-3.43 (1H, m), 3.42-3.33 (1H, m), 2.69-2.66 (1H, m), 1.68-1.58 (2H, m), 0.94 (3H, t, J=7.4 Hz). ESI-MS (m/z): 237 [M+H]⁺.

### Example 38

### N-Methyl-N-(4-n-propylamino-6-prop-2-ynylamino-[1,3,5]triazin-2-yl)-hydroxylamine (99) (Scheme 34)

6-Chloro-N-n-propyl-N′-prop-2-ynyl-[1,3,5]triazine-2,4-diamine (3) and N-methyl-hydroxylamine hydrochloride were reacted using the procedure described for Compound 93 to yield N-methyl-N-(4-n-propylamino-6-prop-2-ynylamino-[1,3,5]triazin-2-yl)-hydroxylamine (99) in 81% yield. 400 MHz ¹H NMR (CDCl₃, ppm): 10.5-7.7 (1H, br s), 5.18 (1H, s), 5.05 (1H, s), 4.26-4.10 (2H, m), 3.44-3.26 (5H, m), 2.21 (1H, t, J=2.5 Hz), 1.58 (2H, sextet, J=7.3 Hz), 0.95 (3H, t, J=7.3 Hz). ESI-MS (m/z): 237 [M+H]⁺.

### Example 39

### N-(4-n-Propylamino-6-prop-2-ynylamino-[1,3,5]triazin-2-yl)-hydroxylamine (100) (Scheme 34)

6-Chloro-N-n-propyl-N′-prop-2-ynyl-[1,3,5]triazine-2,4-diamine (3) and hydroxylamine hydrochloride were reacted using the procedure described for Compound 93 to yield N-(4-n-propylamino-6-prop-2-ynylamino-[1,3,5]triazin-2-yl)-hydroxylamine (100) in 73% yield. 400 MHz ¹H NMR (DMSO-d₆, ppm): δ 9.26-8.70 (1H, m), 8.31 (1H, s), 7.18-6.53 (2H, m), 4.04-3.93 (2H, m), 3.23-3.07 (2H, m), 3.01-2.96 (1H, m), 1.56-1.38 (2H, m), 0.84 (3H, t, J=7.4 Hz). ESI-MS (m/z): 223 [M+H]⁺.

### Example 40

### O-(2-Methoxy-ethyl)-N-methyl-N-(4-n-propylamino-6-prop-2-ynyl amino-[1,3,5]triazin-2-yl)-hydroxylamine (101) and Corresponding Hydrochloride Salt (102a) (Scheme 34)

### O-(2-Methoxy-ethyl)-N-methyl-N-(4-n-propylamino-6-prop-2-ynylamino-[1,3,5]triazin-2-yl)-hydroxylamine (101)

6-Chloro-N-n-propyl-N′-prop-2-ynyl-[1,3,5]triazine-2,4-diamine (3) and O-(2-methoxy-ethyl)-N-methyl-hydroxylamine hydrochloride were reacted using the procedure described for Compound 93 to yield O-(2-methoxy-ethyl)-N-methyl-N-(4-n-propylamino-6-prop-2-ynylamino-[1,3,5]triazin-2-yl)-hydroxylamine (101) in 67% yield. 400 MHz ¹H NMR (CDCl₃, ppm): δ 5.18-4.86 (2H, m), 4.23-4.11 (4H, m), 3.66-3.63 (2H, m), 3.41 (3H, s), 3.60-3.29 (5H, m), 2.19 (1H, t, J=2.5 Hz), 1.57 (2H, sextet, J=7.5 Hz), 0.94 (3H, t, J=7.5 Hz). ESI-MS (m/z): 295 [M+H]⁺.

### O-(2-Methoxy-ethyl)-N-methyl-N-(4-n-prop-2-ynylamino-[1,3,5]triazin-2-yl)-hydroxylamine hydrochloride (102a)

O-(2-Methoxy-ethyl)-N-methyl-N-(4-n-propylamino-6-prop-2-ynylamino-[1,3,5]triazin-2-yl)-hydroxylamine (101) and 2 M HCl/diethyl ether were reacted using the procedure described for Compound 92a to yield O-(2-methoxy-ethyl)-N-methyl-N-(4-n-propylamino-6-prop-2-ynylamino-[1,3,5]triazin-2-yl)-hydroxylamine hydrochloride (102a) in quantitative yield. 400 MHz ¹H NMR (CDCl₃, ppm): δ 13.84 (0.2H, br s), 13.27 (0.8H, br s), 9.8-9.2 (1H, m), 5.9-5.5 (1H, br s), 4.31-4.27 (2H, m), 4.23-4.14 (2H, m), 3.85-3.83 (2H, m), 3.45-3.32 (8H, m), 2.29-2.23 (1H, m), 1.71-1.58 (2H, m), 1.00-0.94 (3H, m). ESI-MS (m/z): 295 [M+H]⁺.

### Example 41

### N-Methyl-O-(4,4,5,5,5-pentafluoro-pentyl)-N-(4-n-propylamino-6-prop-2-ynylamino-[1,3,5]triazin-2-yl)-hydroxylamine (103) and corresponding hydrochloride salt (104a) (Scheme 34)

### N-Methyl-O-(4,4,5,5,5-pentafluoro-pentyl)-N-(4-n-propylamino-6-prop-2-ynylamino-[1,3,5]triazin-2-yl)-hydroxylamine (103)

6-Chloro-N-propyl-N′-prop-2-ynyl-[1,3,5]triazine-2,4-diamine (3) and N-methyl-O-(4,4,5,5,5-pentafluoro-pentyl)-hydroxylamine hydrochloride were reacted using the procedure described for Compound 93 to yield N-methyl-O-(4,4,5,5,5-pentafluoro-pentyl)-N-(4-n-propylamino-6-prop-2-ynylamino-[1,3,5]triazin-2-yl)-hydroxylamine (103) in 40% yield. 400 MHz ¹H NMR (DMSO-d₆, ppm): δ 7.31-6.82 (2H, m), 4.05-3.91 (4H, m), 3.24-3.10 (5H, m), 3.02-2.96 (1H, m), 2.46-2.31 (2H, m), 1.88-1.77 (2H, m), 1.54-1.42 (2H, m), 0.84 (3H, t, J=7.3 Hz). ESI-MS (m/z): 397 [M+H]⁺.

### N-Methyl-O-(4,4,5,5,5-pentafluoro-pentyl)-N-(4-n-propylamino-6-prop-2-ynylamino-[1,3,5]triazin-2-yl)-hydroxylamine hydrochloride (104a)

N-Methyl-O-(4,4,5,5,5-pentafluoro-pentyl)-N-(4-n-propylamino-6-prop-2-ynylamino-[1,3,5]triazin-2-yl)-hydroxylamine (103) and 2 M HCl/diethyl ether were reacted using the procedure described for Compound 92a to yield N-methyl-O-(4,4,5,5,5-pentafluoro-pentyl)-N-(4-n-propylamino-6-prop-2-ynylamino-[1,3,5]triazin-2-yl)-hydroxylamine hydrochloride (104a) in quantitative yield. 400 MHz ¹H NMR (DMSO-d₆, ppm): δ 9.1-8.5 (2H, m), 8.4-7.8 (1H, m), 4.21-4.04 (4H, m), 3.41-3.13 (6H, m), 2.46-2.26 (2H, m), 2.08-1.83 (2H, m), 1.62-1.45 (2H, m), 0.95-0.81 (3H, m). ESI-MS (m/z): 397 [M+H]⁺.

### Example 42

### N-(4-Fluorophenyl)-N′-propyl-N″-prop-2-ynyl-[1,3,5]triazine-2,4,6-triamine (106) and corresponding hydrochloride salt (107a) (Scheme 35)

### 6-Chloro-N-(4-fluorophenyl)-N′-n-propyl-[1,3,5]triazine-2,4-diamine (105)

A mixture of 4,6-dichloro-[1,3,5]triazin-2-yl)-n-propyl-amine (2) (700 mg, 3.38 mmol), 4-fluoroaniline (413 mg, 3.72 mmol) and N,N-diisopropylethylamine (614 μL, 3.72 mmol) in 1,4-dioxane (15 mL) was stirred at ambient temperature for 24 h. A saturated NaHCO₃ solution (30 mL) was added and the resulting suspension was extracted with EtOAc (3×30 mL). The combined organic extracts were washed with water (30 mL), then with a brine solution (30 mL) and lastly dried over solid anhydrous Na₂SO₄. After filtration, the solvent was removed under vacuum and the residue was purified by flash column chromatography using gradient elution from PE/EtOAc (9:1) to PE/EtOAc (1:1) to yield 6-chloro-N-(4-fluorophenyl)-N′-n-propyl-[1,3,5]triazine-2,4-diamine (105) (959 mg, 99%). 400 MHz ¹H NMR (CDCl₃, ppm): δ 7.55-7.43 (2H, m), 7.20-7.05 (1H, br s), 7.08-6.99 (2H, m), 5.65 (0.7H, br s), 5.36 (0.3H, br s), 3.45-3.35 (2H, m), 1.64 (2H, sextet, J=7.3 Hz), 0.97 (3H, t, J=7.3 Hz). ESI-MS (m/z): 282, 284 [M+H]⁺.

### N-(4-Fluorophenyl)-N′-propyl-N″-prop-2-ynyl-[1,3,5]triazine-2,4,6-triamine (106)

6-Chloro-N-(4-fluorophenyl)-N′-n-propyl-[1,3,5]triazine-2,4-diamine (105) (300 mg, 1.06 mmol) and propargylamine (273 μL, 4.26 mmol) in 1,4-dioxane (10 mL) was heated at 90° C. for 9 h. The mixture was cooled to ambient temperature. A saturated NaHCO₃ solution (20 mL) was added and the resulting suspension was extracted with EtOAc (3×20 mL). The combined organic extracts were washed with water (30 mL), then with a brine solution (30 mL) and lastly dried over solid anhydrous Na₂SO₄. After filtration, the solvent was removed and the residue was purified by flash column chromatography using gradient elution from PE/EtOAc (9:1) to PE/EtOAc (1:1) to give N-(4-fluorophenyl)-N′-n-propyl-N″-prop-2-ynyl-[1,3,5]triazine-2,4,6-triamine (106) (303 mg, 94%). 400 MHz ¹H NMR (CDCl₃, ppm): δ 7.59-7.44 (2H, m), 7.02-6.95 (2H, m), 6.78 (1H, br s), 5.16-4.94 (2H, m), 4.25-4.14 (2H, m), 3.38-3.30 (2H, m), 2.23 (1H, t, J=2.5 Hz), 1.60 (2H, sextet, J=7.3 Hz), 0.96 (3H, t, J=7.3 Hz). ESI-MS (m/z): 301 [M+H]⁺.

### N-(4-Fluorophenyl)-N′-n-propyl-N″-prop-2-ynyl-[1,3,5]triazine-2,4,6-triamine hydrochloride (107a)

A 2M HCl/diethyl ether solution (500 μL, 1.00 mmol) was added to a solution of N-(4-fluorophenyl)-N′-n-propyl-N″-prop-2-ynyl-[1,3,5]triazine-2,4,6-triamine (106) (301 mg, 1.00 mmol) in diethyl ether (10 mL) at 0° C. The mixture was stirred for 0.5 h at 0° C. The resultant precipitate was filtered, washed with diethyl ether and dried to yield N-(4-fluorophenyl)-N′-n-propyl-N″-prop-2-ynyl-[1,3,5]triazine-2,4,6-triamine hydrochloride (107a) (314 mg, 93%). 400 MHz ¹H NMR (CDCl₃, ppm): δ 9.87 (0.5H, br s), 9.69 (0.5H, br s), 8.07 (0.2H, br s), 7.85 (0.3H, br s), 7.76-7.40 (3.5H, m), 7.10-6.99 (2H, m), 6.27-5.71 (1H, m), 4.26-4.15 (2H, m), 3.47-3.35 (2H, m), 2.32-2.27 (1H, m), 1.71-1.61 (2H, m), 1.02-0.94 (3H, m). ESI-MS (m/z): 301 [M+H]⁺; melting point: 133-136° C.

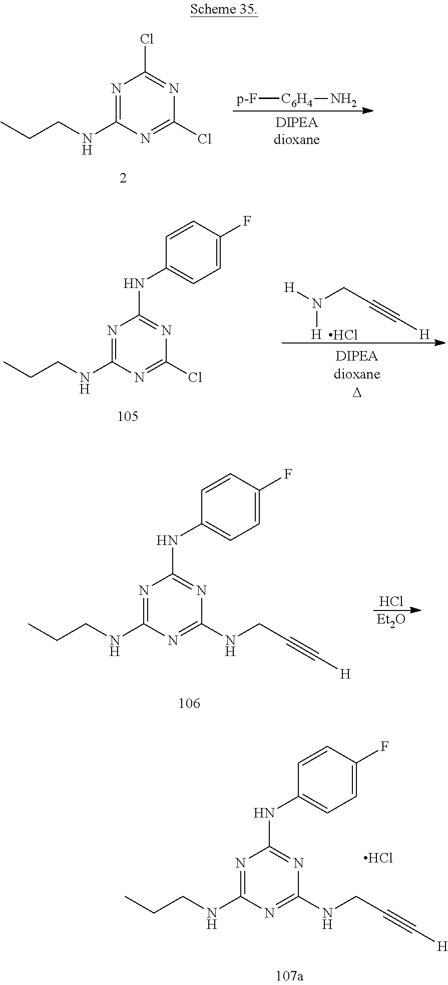

### Example 43

### N-(3-Chloro-2-methyl-benzyl)-N′-n-propyl-N″-prop-2-ynyl-[1,3,5]triazine-2,4,6-triamine (108) and corresponding hydrochloride salt (109a) (Scheme 36)

### N-(3-Chloro-2-methyl-benzyl)-N′-n-propyl-N″-prop-2-ynyl-[1,3,5]triazine-2,4,6-triamine (108)

A mixture of 6-chloro-N-n-propyl-N′-prop-2-ynyl-[1,3,5]triazine-2,4-diamine (3) (300 mg, 1.33 mmol) and 3-chloro-2-methyl-benzylamine (363 μL, 2.66 mmol) in 1,4-dioxane (5 mL) was heated at 100° C. for 24 h. The mixture was cooled to ambient temperature. A saturated NaHCO₃ solution (15 mL) was added and the resulting suspension was extracted with EtOAc (3×20 mL). The combined organic extracts were washed with water (20 mL), then with a brine solution (20 mL) and lastly dried over solid anhydrous Na₂SO₄. After filtration, the solvent was removed and the resulting residue was purified by flash column chromatography using gradient elution from PE/EtOAc (2:1) to PE/EtOAc (1:1) to yield N-(3-chloro-2-methyl-benzyl)-N′-n-propyl-N″-prop-2-ynyl-[1,3,5]triazine-2,4,6-triamine (108) (391 mg, 85%). 400 MHz ¹H NMR (CDCl₃, ppm): δ 7.30-7.26 (1H, m), 7.19 (1H, d, J=7.8 Hz), 7.08 (1H, dd, J=7.8, 7.8 Hz), 5.14-4.69 (3H, m), 4.65-4.47 (2H, m), 4.25-4.11 (2H, m), 3.40-3.19 (2H, m), 2.38 (3H, s), 2.19 (1H, t, J=2.5 Hz), 1.62-1.47 (2H, m), 0.93 (3H, t, J=7.3 Hz). ESI-MS (m/z): 345, 347 [M+H]⁺.

### N-(3-Chloro-2-methyl-benzyl)-N′-propyl-N″-prop-2-ynyl-[1,3,5]triazine-2,4,6-triamine hydrochloride (109a)

N-(3-Chloro-2-methyl-benzyl)-N′-n-propyl-N″-prop-2-ynyl-[1,3,5]triazine-2,4,6-triamine (108) and 2 M HCl/diethyl ether were reacted using the procedure described for Compound 92a to yield N-(3-chloro-2-methyl-benzyl)-N′-n-propyl-N″-prop-2-ynyl-[1,3,5]triazine-2,4,6-triamine hydrochloride (109a) in quantitative yield. 400 MHz ¹H NMR (CDCl₃, ppm): δ 13.74-13.31, (0.7H, m), 8.14-7.95 (0.3H, m), 7.83-7.48 (1H, m), 7.37-7.28 (1H, m), 7.24-7.05 (2H, m), 5.85-5.53 (1H, m), 4.71-4.49 (2H, m), 4.26-4.05 (2H, m), 3.46-3.22 (2H, m), 2.45-2.33 (3H, m), 2.32-2.18 (1H, m), 1.74-1.48 (2H, m), 1.05-0.85 (3H, m). ESI-MS (m/z): 345, 347 [M+H]⁺.

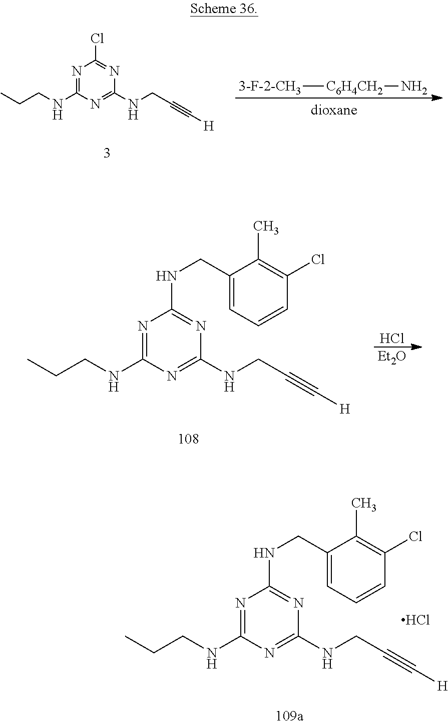

### Example 44

### N-(3,4-Dichlorobenzyl)-N′-n-propyl-N″-prop-2-ynyl-[1,3,5]triazine-2,4,6-triamine (110) and corresponding hydrochloride salt (111a) (Scheme 37)

### N-(3,4-Dichlorobenzyl)-N′-n-propyl-N″-prop-2-ynyl-[1,3,5]triazine-2,4,6-triamine (110)

6-Chloro-N-n-propyl-N′-prop-2-ynyl-[1,3,5]triazine-2,4-diamine (3) and 3,4-dichlorobenzylamine were reacted using the procedure described for Compound 108 to yield N-(3,4-dichlorobenzyl)-N′-n-propyl-N″-prop-2-ynyl-[1,3,5]triazine-2,4,6-triamine (110) in 87% yield. 400 MHz ¹H NMR (CDCl₃, ppm): δ 7.44-7.39 (1H, m), 7.37 (1H, d, J=8.3 Hz), 7.15 (1H, d, J=8.3 Hz), 5.13-5.01 (1H, m), 5.01-4.67 (2H, m), 4.52 (2H, d, J=6.2 Hz), 4.20-4.13 (2H, m), 3.36-3.21 (2H, m), 2.23-2.18 (1H, m), 1.58-1.46 (2H, m), 0.93 (3H, t, J=7.3 Hz). ESI-MS (m/z): 365, 367, 369 [M+H]⁺.

### N-(3,4-Dichlorobenzyl)-N′-n-propyl-N″-prop-2-ynyl-[1,3,5]triazine-2,4,6-triamine hydrochloride (111a)

N-(3,4-dichloro-benzyl)-N′-n-propyl-N″-prop-2-ynyl-[1,3,5]triazine-2,4,6-triamine (110) and 2 M HCl/diethyl ether were reacted using the procedure described for Compound 92a to yield N-(3,4-dichloro-benzyl)-N′-n-propyl-N″-prop-2-ynyl-[1,3,5]triazine-2,4,6-triamine hydrochloride (111a) in quantitative yield. 400 MHz ¹H NMR (CDCl₃, ppm): δ 3.70-13.06 (0.7H, m), 8.67-8.38 (0.3H, m), 8.29-8.06 (0.3H, m), 7.78-7.54 (0.7H, m), 7.48-7.34 (2H, m), 7.24-7.10 (1H, m), 6.10-5.89 (0.3H, m), 5.79-5.48 (0.7H, m), 4.65-4.45 (2H, m), 4.26-4.07 (2H, m), 3.47-3.24 (2H, m), 2.34-2.20 (1H, m), 1.75-1.49 (2H, m), 1.06-0.88 (3H, m). ESI-MS (m/z): 365, 367, 369 [M+H]⁺.

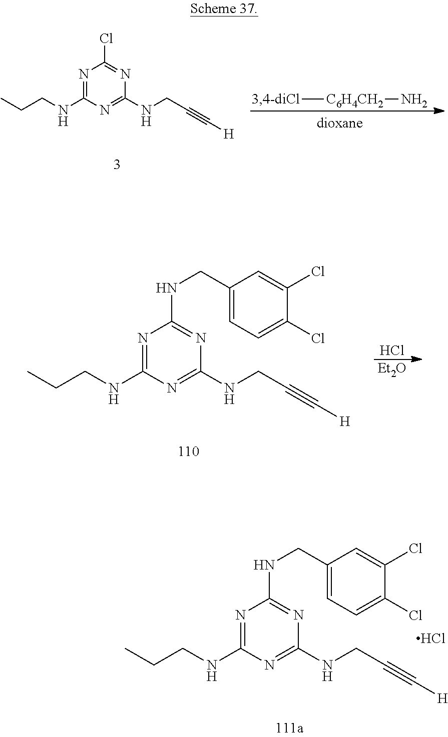

### Example 45

### O,N-Dimethyl-N-(2-prop-2-ynylamino-7H-pyrrolo[2,3-d]pyrimidin-4-yl)-hydroxylamine (113) and corresponding hydrochloride salt (114a) (Scheme 38)

### (4-Chloro-7H-pyrrolo[2,3-d]pyrimidin-2-yl)-prop-2-ynyl-amine (112)

To a solution of 4-chloro-7H-pyrrolo[2,3-d]pyrimidin-2-ylamine (500 mg, 2.97 mmol) and propargyl aldehyde (500 mg, 9.25 mmol) (freshly prepared from propargyl alcohol; Org. Synth. Coll. 1963, 4:813) in MeOH (10 mL) was added AcOH (˜35 mg) at 0° C. The mixture was stirred for 1 h and NaCNBH₃ (187 mg, 2.97 mmol) was added. The reaction mixture was stirred for 16 h at ambient temperature. An additional amount of propargyl aldehyde (500 mg, 9.25 mmol) and NaCNBH₃ (187 mg, 2.97 mmol) were added and the pH of the reaction mixture was adjusted to ˜3 by the addition of AcOH (˜35 mg). The resulting mixture was stirred for 20 h at ambient temperature. The volatiles were removed and the residue was partitioned between CHCl₃ (50 mL) and saturated NaHCO₃ solution (50 mL). The water phase was filtered and the resultant precipitate was washed with water (2×20 mL) to yield (4-chloro-7H-pyrrolo[2,3-d]pyrimidin-2-yl)-prop-2-ynyl-amine (112) as a brown solid (170 mg, 28%). 400 MHz ¹H NMR (DMSO-d₆, ppm): δ 11.77 (1H, br s), 7.43 (1H, t, J=5.7 Hz), 7.16 (1H, d, J=3.6 Hz), 6.31 (1H, d, J=3.6 Hz), 4.05 (2H, dd, J=5.7, 2.3 Hz), 3.01 (1H, t, J=2.3 Hz). ESI-MS (m/z): 207, 209 [M+H]⁺.

### O,N-Dimethyl-N-(2-prop-2-ynylamino-7H-pyrrolo[2,3-d]pyrimidin-4-yl)-hydroxylamine (113)

A mixture of (4-chloro-7H-pyrrolo[2,3-d]pyrimidin-2-yl)-prop-2-ynyl-amine (112) (160 mg, 0.77 mmol), potassium carbonate (53 mg, 3.83 mmol) and O,N-dimethylhydroxylamine hydrochloride (300 mg, 3.10 mmol) in n-BuOH (4 mL) was heated at 80° C. for 30 min. After cooling to ambient temperature, water (20 mL) was added and the mixture was extracted with EtOAc (3×20 mL). The combined organic extracts were washed with water (20 mL), then with a brine solution (20 mL) and lastly dried over solid anhydrous Na₂SO₄. After filtration, the solvent was removed under vacuum and the resultant residue was purified by flash column chromatography (PE/EtOAc-1:1) to yield O,N-dimethyl-N-(2-prop-2-ynylamino-7H-pyrrolo[2,3-d]pyrimidin-4-yl)-hydroxylamine (113) (90 mg, 50%). 400 MHz ¹H NMR (CDCl₃, ppm): δ 8.98 (1H, br s), 6.80 (1H, dd, J=3.6, 2.2 Hz), 6.50 (1H, dd, J=3.6, 2.2 Hz), 4.87 (1H, t, J=5.7 Hz), 4.22 (2H, dd, J=5.8, 2.4 Hz), 3.83 (3H, s), 3.41 (3H, s), 2.19 (1H, t, J=2.4 Hz). ESI-MS (m/z): 232 [M+H]⁺.

### O,N-Dimethyl-N-(2-prop-2-ynylamino-7H-pyrrolo[2,3-d]pyrimidin-4-yl)-hydroxylamine hydrochloride (114a)

A 2M HCl/diethyl ether solution (185 μL, 0.37 mmol) was added to a solution of O,N-dimethyl-N-(2-prop-2-ynylamino-7H-pyrrolo[2,3-d]pyrimidin-4-yl)-hydroxylamine (113) (85 mg, 0.37 mmol) in diethyl ether (15 mL) and EtOH (1 mL) at 0° C. The mixture was stirred for 0.5 h at 0° C. The resultant precipitate was filtered and washed with diethyl ether (5 mL) to yield O,N-dimethyl-N-(2-prop-2-ynylamino-7H-pyrrolo[2,3-d]pyrimidin-4-yl)-hydroxylamine hydrochloride (114a) (82 mg, 83%). 400 MHz ¹H NMR (D₂O, ppm): δ 6.98 (1H, d, J=3.6 Hz), 6.59 (1H, d, J=3.6 Hz), 4.24 (2H, d, J=2.4 Hz), 3.90 (3H, s), 3.61 (3H, s), 2.68 (1H, t, J=2.4 Hz). ESI-MS (m/z): 232 [M+H]⁺; melting point: 182-184° C.

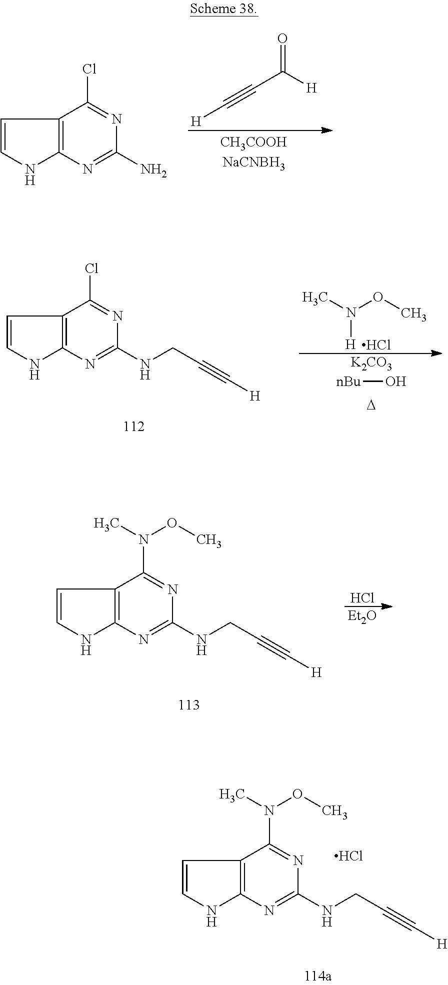

### Example 46

### N-(4,6-Bis-n-propylamino-[1,3,5]triazin-2-yl)-O-methyl-N-prop-2-ynyl-hydroxylamine (115) and corresponding hydrochloride salt (116a) (Schemes 39-40)

### O-Benzyl-N-methoxycarbamate (c)

To a pre-cooled, 0° C. solution of O-methyl-hydroxylamine hydrochloride (5.00 g, 59.87 mmol) in CH₂Cl₂ (250 mL) was added N, N-diisopropylethylamine (24.73 mL, 149.65 mmol) and benzyl chloroformate (8.54 mL, 59.87 mmol). The resulting solution was stirred at ambient temperature for 5 h. At this time the solution was washed twice with a saturated aqueous NaHCO₃ solution (70 mL) and dried over solid anhydrous Na₂SO₄. The solvent was removed under vacuum to yield O-benzyl-N-methoxycarbamate (c) in quantitative yield. 400 MHz ¹H NMR (CDCl₃, ppm): δ 7.43-7.30 (5H, m), 5.19 (2H, s), 3.75 (3H, s).

### O-Benzyl-N-methoxy-N-prop-2-ynyl-carbamate (d)

An ACE® pressure tube was charged with O-benzyl-N-methoxycarbamate (c) (10.84 g, 59.87 mmol), anhydrous K₂CO₃ (12.41 g, 89.79 mmol), propargyl bromide (80 wt. % in toluene; 13.35 mL, 89.79 mmol), and anhydrous acetone (30 mL). The reaction mixture was heated at 70° C. for 24 h. The reaction mixture was filtered, and the acetone was evaporated. The resulting slurry was dissolved in EtOAc (50 mL), washed with water (3×50 mL), then with a brine solution (40 mL) and lastly dried over solid anhydrous Na₂SO₄. The product was purified by flash column chromatography using gradient elution from petroleum ether/EtOAc (9:1) to petroleum ether/EtOAc (4:1) to yield O-benzyl-N-methoxy-N-prop-2-ynyl-carbamate (d) (8.87 g, 67%). 400 MHz ¹H NMR (DMSO-d₆, ppm): δ 7.41-7.31 (5H, m), 5.23 (2H, s), 4.27 (2H, d, J=2.4 Hz), 3.81 (3H, s), 2.26 (1H, t, J=2.4 Hz).

### O-Methyl-N-prop-2-ynyl-hydroxylamine hydrochloride (e)

O-Benzyl-N-methoxy-N-prop-2-ynyl-carbamate (d) (8.87 g, 40.46 mmol) and 33% HBr/AcOH (45 mL) was stirred at room temperature for 1 h. A saturated solution of NaHCO₃ (400 mL) was added and the suspension was extracted with CH₂Cl₂ (3×200 mL). The combined organic extracts were dried over solid anhydrous Na₂SO₄. A 2 M HCl/diethyl ether solution (22.25 mL, 44.50 mmol) was added, and the volatiles were removed under reduced pressure. The product was crystallized from acetonitrile/diethyl ether (1:10 (v/v)) to yield O-methyl-N-prop-2-ynyl-hydroxylamine hydrochloride (e) (3.06 g, 62%). 400 MHz ¹H NMR (DMSO-d₆, ppm): δ 8.5-5.5 (2H, br s), 3.98 (2H, d, J=2.4 Hz), 3.73 (3H, s), 3.47 (1H, t, J=2.4 Hz).

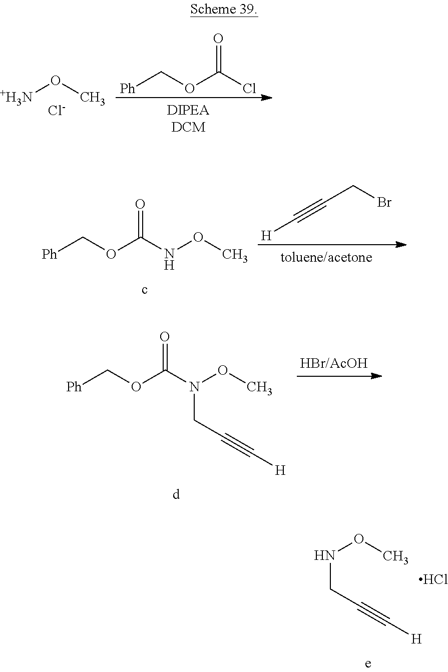

### N-(4,6-Bis-n-propylamino-[1,3,5]triazin-2-yl)-O-methyl-N-prop-2-ynyl-hydroxylamine (115)

A mixture of 6-chloro-N,N′-di-n-propyl-[1,3,5]triazine-2,4-diamine (20) (300 mg, 1.31 mmol), O-methyl-N-prop-2-ynyl-hydroxylamine hydrochloride (e) (365 mg, 3.00 mmol) and NaOH (120 mg, 3.00 mmol) in 1,4-dioxane (5 mL) was heated at 90° C. for 3 h. The mixture was cooled to ambient temperature. A saturated NaHCO₃ solution (15 mL) was added and the mixture was extracted with EtOAc (3×20 mL). The combined organic extracts were washed with water (30 mL), then with a brine solution (30 mL) and lastly dried over solid anhydrous Na₂SO₄. After filtration, the solvent was removed under reduced pressure and the resultant residue was purified by flash column chromatography using gradient elution from CH₂Cl₂/EtOH (99:1) to CH₂Cl₂/EtOH (97:3) to yield N-(4,6-bis-propylamino-[1,3,5]triazin-2-yl)-O-methyl-N-prop-2-ynyl-hydroxylamine (115) (362 mg, 99%). 400 MHz ¹H NMR (CDCl₃, ppm): δ 5.1-4.8 (2H, m), 4.55-4.40 (2H, m), 3.88 (3H, br s), 3.42-3.23 (4H, m), 2.18 (1H, t, J=2.4 Hz), 1.58 (4H, sextet, J=7.4 Hz), 0.95 (6H, t, J=7.4 Hz). ESI-MS (m/z): 279 [M+H]⁺.

### N-(4,6-Bis-n-propylamino-[1,3,5]triazin-2-yl)-O-methyl-N-prop-2-ynyl-hydroxylamine hydrochloride (116a)

A 2 M HCl/diethyl ether (650 μL, 1.30 mmol) was added to a solution of N-(4,6-bis-n-propylamino-[1,3,5]triazin-2-yl)-O-methyl-N-prop-2-ynyl-hydroxylamine (115) (362 mg, 1.30 mmol) in diethyl ether (10 mL) at 0° C. The mixture was stirred for 0.5 h at 0° C. The resultant precipitate were filtered and washed with diethyl ether (5 mL) to yield N-(4,6-bis-n-propylamino-[1,3,5]triazin-2-yl)-O-methyl-N-prop-2-ynyl-hydroxylamine hydrochloride (116a) (344 mg, 83%). 400 MHz ¹H NMR (D₂O, ppm): δ 4.71-4.57 (2H, m), 3.91-3.86 (3H, m), 3.47-3.30 (4H, m), 2.74-2.70 (1H, m), 1.61 (4H, sextet, J=7.3 Hz), 0.92 (6H, t, J=7.3 Hz). ESI-MS (m/z): 279 [M+H]⁺; melting point: 110-113° C.

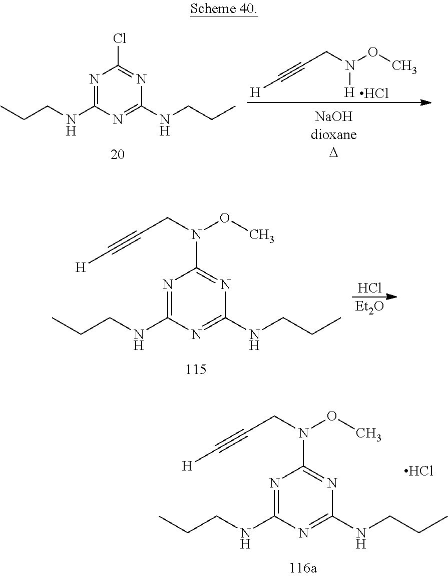

### Example 47

### O-Methyl-N-(4-n-propylamino-6-prop-2-ynylamino-[1,3,5]triazin-2-yl)-N-prop-2-ynyl-hydroxylamine (117) and corresponding hydrochloride salt (118a) (Scheme 41)

### O-Methyl-N-(4-n-propylamino-6-prop-2-ynylamino-[1,3,5]triazin-2-yl)-N-prop-2-ynyl-hydroxylamine (117)

6-Chloro-N-n-propyl-N′-prop-2-ynyl-[1,3,5]triazine-2,4-diamine (3) and O-methyl-N-prop-2-ynyl-hydroxylamine hydrochloride (e) were reacted according to the procedure described for Compound 115 to yield O-methyl-N-(4-n-propylamino-6-prop-2-ynylamino-[1,3,5]triazin-2-yl)-N-prop-2-ynyl-hydroxylamine (117) in 99% yield. 400 MHz ¹H NMR (CDCl₃, ppm): δ 5.25-4.95 (2H, m), 4.54-4.42 (2H, m), 4.25-4.13 (2H, m), 3.88 (3H, s), 3.38-3.29 (2H, m), 2.20 (1H, t, J=2.5 Hz), 2.19 (1H, t, J=2.3 Hz), 1.58 (2H, sextet, J=7.3 Hz), 0.95 (3H, t, J=7.3 Hz). ESI-MS (m/z): 275 [M+H]⁺.

### O-Methyl-N-(4-n-propylamino-6-prop-2-ynylamino-[1,3,5]triazin-2-yl)-N-prop-2-ynyl-hydroxylamine hydrochloride (118a)

A 2M HCl/diethyl ether solution (665 μL, 1.33 mmol) was added to a solution of O-methyl-N-(4-n-propylamino-6-prop-2-ynylamino-[1,3,5]triazin-2-yl)-N-prop-2-ynyl-hydroxylamine (117) (365 mg, 1.33 mmol) in diethyl ether (10 mL) at 0° C. The mixture was stirred for 0.5 h at 0° C. The volatiles were removed under reduced pressure to yield O-methyl-N-(4-n-propylamino-6-prop-2-ynylamino-[1,3,5]triazin-2-yl)-N-prop-2-ynyl-hydroxylamine hydrochloride (118a) in quantitative yield. 400 MHz ¹H NMR (D₂O, ppm): δ 4.75-4.58 (2H, m), 4.31-4.20 (2H, m), 3.95-3.85 (3H, m), 3.52-3.33 (2H, m), 2.75-2.72 (1H, m), 2.69-2.65 (1H, m), 1.68-1.57 (2H, m), 0.93 (3H, t, J=7.4 Hz). ESI-MS (m/z): 275 [M+H]⁺.

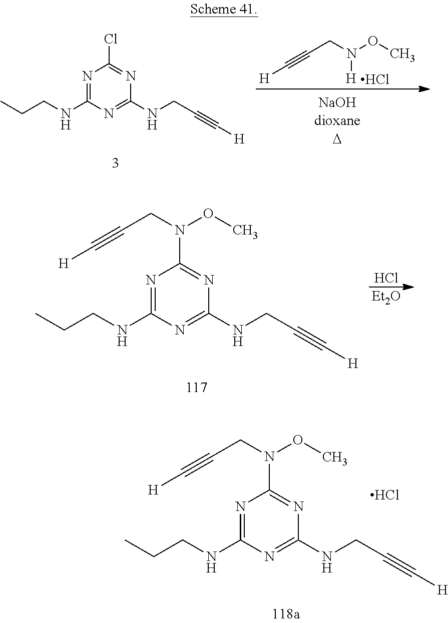

### Example 48

### N-(4,6-Bis-n-propylamino-[1,3,5]triazin-2-yl)-N-methyl-O-prop-2-ynyl-hydroxylamine (119) and corresponding hydrochloride salt (120a) (Schemes 42-43)

### 2-Prop-2-ynyloxy-isoindole-1,3-dione (f)

Diethyl azodicarboxylate (29.4 mL, 187.30 mmol) was added dropwise at 0° C. to a stirred suspension of prop-2-yn-1-ol (10.3 mL, 178.38 mmol), triphenylphosphine (19.30 g, 187.30 mmol), and N-hydroxyphthalimide (49.13 g, 178.38 mmol) in THF (500 mL). The mixture was stirred at ambient temperature for 20 h and evaporated to dryness. The product was purified by flash column chromatography using gradient elution from petroleum ether/EtOAc (9:1) to petroleum ether/EtOAc (5:1) to yield 2-prop-2-ynyloxy-isoindole-1,3-dione (f) (26.31 g, 73%). 400 MHz ¹H NMR (CDCl₃, ppm): δ 7.88-784 (2H, m), 7.79-7.74 (2H, m), 4.88 (2H, d, J=2.4 Hz), 2.59 (1H, t, J=2.4 Hz).

### O-Prop-2-ynyl-hydroxylamine hydrochloride (g)

A mixture of 2-prop-2-ynyloxy-isoindole-1,3-dione (f) (26.31 g, 130.78 mmol) and hydrazine monohydrate (12.7 mL, 261.56 mmol) in CH₂Cl₂ (400 mL) was stirred at room temperature for 20 h. The reaction mixture was filtered. The filtrate was washed with water (100 mL), then with a brine solution (70 mL) and lastly dried over solid anhydrous Na₂SO₄. A 4 M HCl/1,4-dioxane solution (34.0 mL, 136.00 mmol) was added, and the volatiles were removed under reduced pressure to yield O-prop-2-ynyl-hydroxylamine hydrochloride (g) (5.05 g, 36%). 400 MHz ¹H NMR (DMSO-d₆, ppm): δ 11.5-9.5 (2H, br s), 8.98 (1H, s), 4.76 (2H, d, J=2.4 Hz), 3.86 (1H, t, J=2.4 Hz).

### O-Benzyl-N-prop-2-ynyloxy-carbamate (h)

To a pre-cooled, 0° C. solution of O-prop-2-ynyl-hydroxylamine hydrochloride (g) (5.00 g, 46.49 mmol) in CH₂Cl₂ (200 mL) was added N,N-diisopropyl ethylamine (20.1 mL, 116.23 mmol) and benzyl chloroformate (7.0 mL, 46.49 mmol). The resulting solution was stirred at ambient temperature for 14 h. The reaction mixture was then washed with saturated aqueous NaHCO₃ solution (2×50 mL), then with water (50 mL) and lastly, dried over solid anhydrous Na₂SO₄. The volatiles were removed under vacuum to yield O-benzyl-N-prop-2-ynyloxy-carbamate (h) (8.06 g, 84%). 400 MHz ¹H NMR (CDCl₃, ppm): δ 7.39-7.30 (5H, m), 5.19 (2H, s), 5.17 (1H, s), (2H, d, J=2.4 Hz), 2.50 (1H, t, J=2.4 Hz).

### O-Benzyl-N-methyl-N-prop-2-ynyloxy-carbamate (i)

A pressure tube was charged with O-benzyl-N-prop-2-ynyloxy-carbamate (h) (8.06 g, 39.27 mmol), anhydrous K₂CO₃ (8.16 g, 59.06 mmol), methyl iodide (6.5 mL, 176.74), and anhydrous acetone (30 mL). The reaction mixture was heated at 70° C. for 24 h. The reaction mixture was filtered, and the acetone was evaporated. The resulting slurry was dissolved in EtOAc (70 mL), washed with water (2×30 mL), then with a brine solution (30 mL) and lastly, dried over solid anhydrous Na₂SO₄. The product was purified by flash column chromatography using gradient elution from petroleum ether/EtOAc (9:1) to petroleum ether/EtOAc (4:1) to yield 3.18 g (37%) of O-benzyl-N-methyl-N-prop-2-ynyloxy-carbamate (i). 400 MHz ¹H NMR (CDCl₃, ppm): δ 7.40-7.30 (5H, m), 5.19 (2H, s), 4.50 (2H, d, J=2.4 Hz), 3.26 (3H, s), 2.47 (1H, t, J=2.4 Hz).

### N-Methyl-O-prop-2-ynyl-hydroxylamine hydrochloride (j)

O-Benzyl-N-methyl-N-prop-2-ynyloxy-carbamate (i) (3.18 g, 14.50 mmol) and 33% HBr/AcOH (16 mL) were stirred at room temperature for 2 h. A saturated solution of NaHCO₃ (275 mL) was added and the mixture was extracted with CH₂Cl₂ (3×75 mL). The combined organic extracts were dried over solid anhydrous Na₂SO₄. A 4 M HCl/1,4-dioxane solution (3.75 mL, 15.00 mmol) was added, and the volatiles were removed under reduced pressure to yield N-methyl-O-prop-2-ynyl-hydroxylamine hydrochloride (j) (1.15 g, 65%). 400 MHz ¹H NMR (CDCl₃, ppm): δ 12.8-11.5 (2H, br s), 4.97 (2H, d, J=2.4 Hz), 3.05 (3H, s), 2.85 (1H, t, J=2.4 Hz).

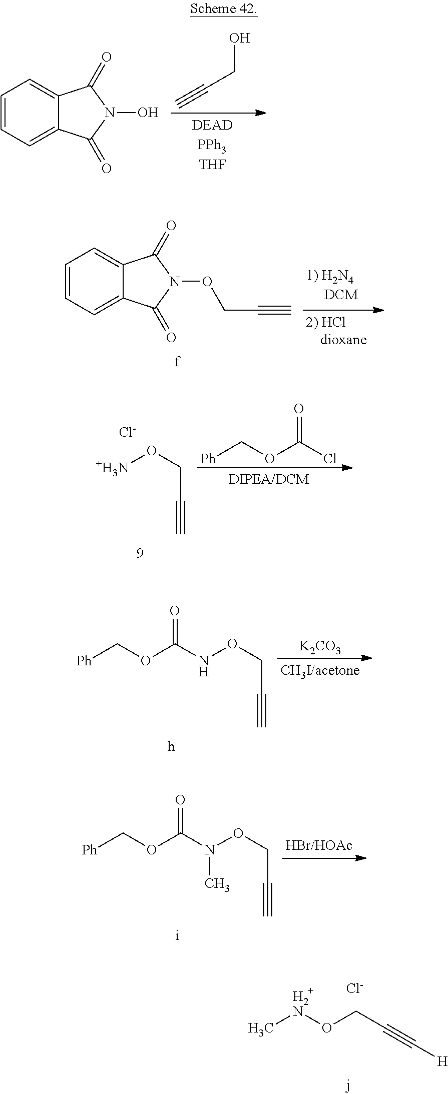

### N-(4,6-Bis-n-propylamino-[1,3,5]triazin-2-yl)-N-methyl-O-prop-2-ynyl-hydroxylamine (119)

A mixture of 6-chloro-N,N′-n-dipropyl-[1,3,5]triazine-2,4-diamine (20) (300 mg, 1.31 mmol), N-methyl-O-prop-2-ynyl-hydroxylamine hydrochloride (j) (365 mg, 3.00 mmol) and NaOH (120 mg, 3.00 mmol) in 1,4-dioxane (5 mL) was heated at 60° C. for 5 h. The mixture was cooled to the room temperature. A saturated NaHCO₃ solution (15 mL) was added and the mixture was extracted with EtOAc (3×20 mL). The combined organic extracts were washed with water (30 mL), then with a brine solution (30 mL) and lastly, dried over solid anhydrous Na₂SO₄. After filtration, the solvent was removed under reduced pressure. The resultant residue was purified by flash column chromatography using gradient elution from CH₂Cl₂/EtOH (99:1) to CH₂Cl₂/EtOH (97:3), then additionally purified by preparative HPLC using gradient elution from hexanes/EtOAc (99:1) to hexanes/EtOAc (1:99) to yield (4,6-bis-n-propylamino-[1,3,5]triazin-2-yl)-N-methyl-O-prop-2-ynyl-hydroxylamine (119) (140 mg, 39%). 400 MHz ¹H NMR (CDCl₃, ppm): δ 5.04-4.79 (2H, m), 4.65 (2H, s), 3.41-3.27 (7H, m), 2.48 (1H, t, J=2.4 Hz), 1.58 (4H, septet, J=7.3 Hz), 0.95 (6H, t, J=7.3 Hz). ESI-MS (m/z): 279 [M+H]⁺.

### N-(4,6-Bis-n-propylamino-[1,3,5]triazin-2-yl)-N-methyl-O-prop-2-ynyl-hydroxylamine hydrochloride (120a)

N-(4,6-bis-n-propylamino-[1,3,5]triazin-2-yl)-N-methyl-O-prop-2-ynyl-hydroxylamine (119) and 2 M HCl/diethyl ether were reacted using procedure described for Compound 116a to yield N-(4,6-bis-n-propylamino-[1,3,5]triazin-2-yl)-N-methyl-O-prop-2-ynyl-hydroxylamine hydrochloride (120a) in quantitative yield. 400 MHz ¹H NMR (D₂O, ppm): δ 4.76-4.72 (2H, m), 3.56-3.30 (7H, m), 3.06-3.01 (1H, m), 1.68-1.56 (4H, m), 0.93 (6H, t, J=7.4 Hz). ESI-MS (m/z): 279 [M+H]⁺; melting point: 105-107° C.

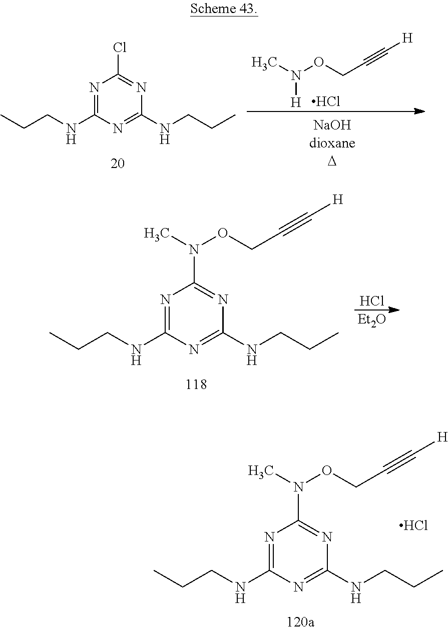

### Example 49

### N-(4,6-Bis-n-propylamino-[1,3,5]triazin-2-yl)-O-prop-2-ynyl-hydroxylamine (121) and corresponding hydrochloride salt (122a) (Scheme 44)

### N-(4,6-Bis-n-propylamino-[1,3,5]triazin-2-yl)-O-prop-2-ynyl-hydroxylamine (121)

A mixture of 6-chloro-N,N′-n-dipropyl-[1,3,5]triazine-2,4-diamine (20) (300 mg, 1.31 mmol), O-prop-2-ynyl-hydroxylamine hydrochloride (g) (323 mg, 3.00 mmol) and NaOH (120 mg, 3.00 mmol) in 1,4-dioxane (5 mL) was heated at 90° C. for 8 h. The mixture was cooled to ambient temperature. A saturated NaHCO₃ solution (15 mL) was added and the mixture was extracted with EtOAc (3×20 mL). The combined organic extracts were washed with water (30 mL), then with a brine solution (30 mL) and lastly, dried over solid anhydrous Na₂SO₄. The solvent was removed under reduced pressure and the resultant residue was purified by flash column chromatography using gradient elution from CH₂Cl₂/EtOH (99:1) to CH₂Cl₂/EtOH (9:1) to yield N-(4,6-bis-n-propylamino-[1,3,5]triazin-2-yl)-O-prop-2-ynyl-hydroxylamine hydrochloride (121) (118 mg, 34%). 400 MHz ¹H NMR (CDCl₃, ppm): δ 7.73 (1H, br s), 5.14-4.92 (2H, m), 4.60 (2H, s), 3.41-3.25 (4H, m), 2.50 (1H, t, J=2.4 Hz), 1.58 (4H, sextet, J=7.3 Hz), 0.95 (6H, t, J=7.3 Hz). ESI-MS (m/z): 265 [M+H]⁺.

### N-(4,6-Bis-n-propylamino-[1,3,5]triazin-2-yl)-O-prop-2-ynyl-hydroxylamine hydrochloride (122a)

N-(4,6-bis-n-propylamino-[1,3,5]triazin-2-yl)-O-prop-2-ynyl-hydroxylamine (121) and 2 M HCl/diethyl ether were reacted using the procedure described for Compound 116a to yield N-(4,6-bis-n-propylamino-[1,3,5]triazin-2-yl)-O-prop-2-ynyl-hydroxylamine hydrochloride (122a) in quantitative yield. 400 MHz ¹H NMR (D₂O, ppm): δ 4.67-4.60 (2H, m), 3.47-3.32 (4H, m), 3.03-2.98 (1H, m), 1.62 (4H, sextet, J=7.4 Hz), 0.93 (6H, t, J=7.4 Hz). ESI-MS (m/z): 265 [M+H]⁺.

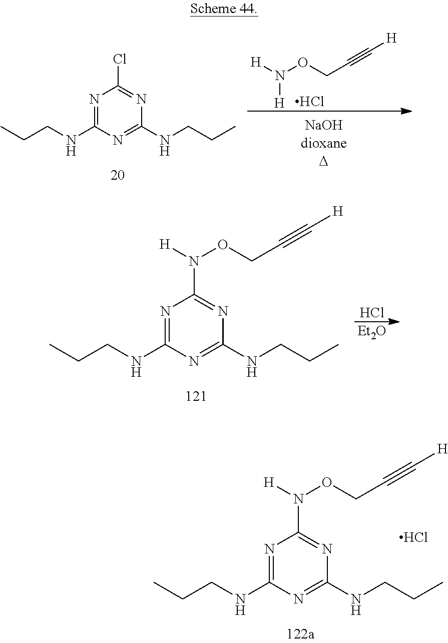

### Example 50

### N-Methyl-N-(4-n-propylamino-6-prop-2-ynylamino-[1,3,5]triazin-2-yl)-O-prop-2-ynyl-hydroxylamine (123) and corresponding hydrochloride salt (124a) (Scheme 45)

### N-Methyl-N-(4-n-propylamino-6-prop-2-ynylamino-[1,3,5]triazin-2-yl)-O-prop-2-ynyl-hydroxylamine (123)

6-Chloro-N-n-propyl-N′-prop-2-ynyl-[1,3,5]triazine-2,4-diamine (3) and N-methyl-O-prop-2-ynyl-hydroxylamine hydrochloride (j) were reacted using the procedure described for Compound 119 to yield N-methyl-N-(4-n-propylamino-6-prop-2-ynylamino-[1,3,5]triazin-2-yl)-O-prop-2-ynyl-hydroxylamine (123) in 23% yield. 400 MHz ¹H NMR (CDCl₃, ppm): δ 5.14-4.90 (2H, m), 4.71-4.56 (2H, m), 4.26-4.10 (2H, m), 3.44-3.26 (5H, m), 2.48 (1H, t, J=2.3 Hz), 2.20 (1H, t, J=2.5 Hz), 1.58 (2H, sextet, J=7.4 Hz), 0.95 (3H, t, J=7.4 Hz). ESI-MS (m/z): 275 [M+H]⁺.

### N-Methyl-N-(4-n-propylamino-6-prop-2-ynylamino-[1,3,5]triazin-2-yl)-O-prop-2-ynyl-hydroxylamine hydrochloride (122a)

N-methyl-N-(4-n-propylamino-6-prop-2-ynylamino-[1,3,5]triazin-2-yl)-O-prop-2-ynyl-hydroxylamine (123) and 2 M HCl/diethyl ether were reacted using the procedure described for Compound 116a to yield N-methyl-N-(4-n-propylamino-6-prop-2-ynylamino-[1,3,5]triazin-2-yl)-O-prop-2-ynyl-hydroxylamine hydrochloride (124a) in quantitative yield. 400 MHz ¹H NMR (D₂O, ppm): δ 4.77-4.72 (2H, m), 4.34-4.15 (2H, m), 3.61-3.30 (5H, m), 3.03 (1H, s), 2.68 (1H, s), 1.71-1.51 (2H, m), 0.94 (3H, t, J=7.4 Hz). ESI-MS (m/z): 275 [M+H]⁺; melting point: 84-86° C.

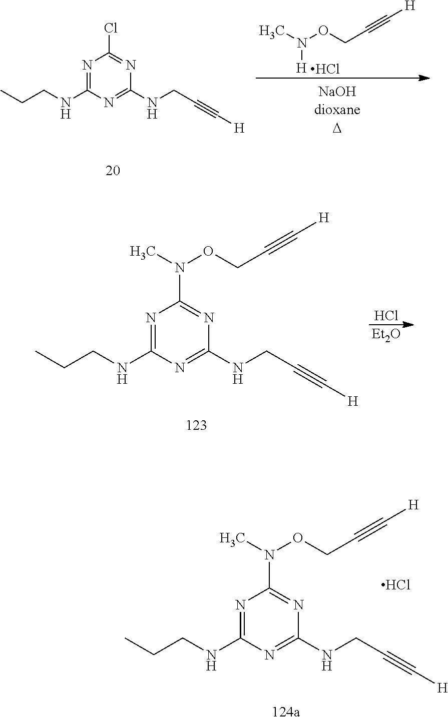

### Example 51

### N-(4-n-Propylamino-6-prop-2-ynylamino-[1,3,5]triazin-2-yl)-O-prop-2-ynyl-hydroxylamine (125) and corresponding hydrochloride salt (126a) (Scheme 46)

### N-(4-n-Propylamino-6-prop-2-ynylamino-[1,3,5]triazin-2-yl)-O-prop-2-ynyl-hydroxylamine (125)

6-Chloro-N-n-propyl-N′-prop-2-ynyl-[1,3,5]triazine-2,4-diamine (3) and O-prop-2-ynyl-hydroxylamine hydrochloride (j) were reacted using the procedure described for Compound 121 in 84% yield. 400 MHz ¹H NMR (CDCl₃, ppm): δ 8.20-7.78 (1H, m), 5.40-5.02 (2H, m), 4.66-4.56 (2H, m), 4.25-4.14 (2H, m), 3.41-3.28 (2H, m), 2.52 (1H, t, J=2.4 Hz), 2.22 (1H, t, J=2.5 Hz), 1.65-1.53 (2H, m), 0.95 (3H, t, J=7.4 Hz). ESI-MS (m/z): 261 [M+H]⁺.

### N-(4-n-Propylamino-6-prop-2-ynylamino-[1,3,5]triazin-2-yl)-O-prop-2-ynyl-hydroxylamine hydrochloride (126a)

N-(4-n-Propylamino-6-prop-2-ynylamino-[1,3,5]triazin-2-yl)-O-prop-2-ynyl-hydroxylamine (125) and 2 M HCl/diethyl ether were reacted using procedure described for Compound 116a to yield N-(4-n-propylamino-6-prop-2-ynylamino-[1,3,5]triazin-2-yl)-O-prop-2-ynyl-hydroxylamine hydrochloride (126a) in quantitative yield. 400 MHz ¹H NMR (D₂O, ppm): δ 4.70-4.60 (2H, m), 4.30-4.19 (2H, m), 3.51-3.33 (2H, m), 3.04-2.99 (1H, m), 2.70-2.67 (1H, m), 1.69-1.57 (2H, m), 0.94 (3H, t, J=7.4 Hz). ESI-MS (m/z): 261 [M+H]⁺.

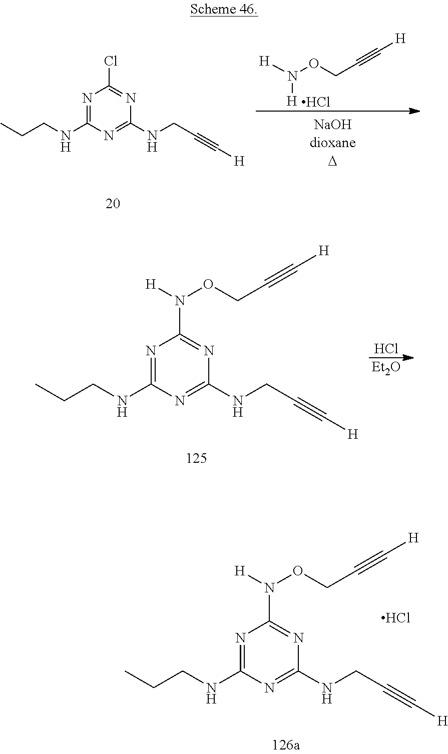

### Examples 52-54

### N-(4-Allylamino-6-prop-2-ynylamino-[1,3,5]triazin-2-yl)-O,N-dimethyl-hydroxylamine (128) and corresponding hydrochloride salt (129a) (Scheme 44)

### 1-[4-(N-Methoxy-N-methyl-amino)-6-prop-2-ynylamino-[1,3,5]triazin-2-ylamino]-propan-2-ol (130) (Scheme 44)

### 3-[4-(N-Methoxy-N-methyl-amino)-6-prop-2-ynylamino-[1,3,5]triazin-2-ylamino]-propan-1-ol (132) (Scheme 47)

### N-(4-Chloro-6-prop-2-ynylamino-[1,3,5]triazin-2-yl)-O,N-dimethyl-hydroxylamine (127)

Propargylamine (1.73 mL, 27.11 mmol) and N,N-diisopropylethylamine (4.72 mL, 27.11 mmol) was added gradually to the cooled solution (0° C.) of cyanuric chloride (1) (5.00 g, 27.11 mmol) in acetonitrile (120 mL). The reaction mixture was stirred at 0° C. for 2 h. To this mixture, O,N-dimethyl-hydroxylamine hydrochloride (2.64g, 27.11 mmol) and N,N-diisopropylethylamine (9.44 mL, 54.22 mmol) were added, and the reaction mixture was heated at 50° C. for 2 h. The mixture was cooled to room temperature. A saturated NaHCO₃ solution (150 mL) was added, and the resulting suspension was extracted with EtOAc (3×75 mL). The combined organic extracts were washed with water (100 mL), then with a brine solution (100 mL) and lastly dried over solid anhydrous Na₂SO₄. After filtration, the solvent was removed under vacuum and the residue was crystallized from EtOAc to yield N-(4-chloro-6-prop-2-ynylamino-[1,3,5]triazin-2-yl)-O,N-dimethyl-hydroxylamine (127) (4.20 g, 68%). 400 MHz ¹H NMR (DMSO-d₆, ppm): δ 8.50-8.40 (1H, m), 4.08-3.99 (2H, m), 3.74-3.67 (3H, m), 3.31-3.25 (3H, m), 3.13-3.10 (1H, m). ESI-MS (m/z): 228, 230 [M+H]⁺.

### N-(4-Allylamino-6-prop-2-ynylamino-[1,3,5]triazin-2-yl)-O,N-dimethyl-hydroxylamine (128)

A mixture of N-(4-chloro-6-prop-2-ynylamino-[1,3,5]triazin-2-yl)-O,N-dimethyl-hydroxylamine (127) (500 mg, 2.20 mmol) and allylamine (823 μL, 11.00 mmol) in 1,4-dioxane (5 mL) was heated at 60° C. for 2 h. The mixture was cooled to room temperature. A saturated NaHCO₃ solution (15 mL) was added and the resulting suspension was extracted with EtOAc (3×20 mL). The combined organic extracts were washed with water (30 mL), then with a brine solution (30 mL) and lastly dried over solid anhydrous Na₂SO₄. After filtration, the solvent was removed and the residue was purified by flash column chromatography using petroleum ether/EtOAc (1:1) to yield N-(4-allylamino-6-prop-2-ynylamino-[1,3,5]triazin-2-yl)-O,N-dimethyl-hydroxylamine (128) (490 mg, 90%). 400 MHz ¹H NMR (CDCl₃, ppm): δ 5.97-5.85 (1H, m), 5.26-5.18 (1H, m), 5.16-4.88 (3H, m), 4.26-4.11 (2H, m), 4.09-3.94 (2H, m), 3.77 (3H, s), 3.29 (3H, s), 2.20 (1H, t, J=2.5 Hz). ESI-MS (m/z): 249 [M+H]⁺.

### N-(4-Allylamino-6-prop-2-ynylamino-[1,3,5]triazin-2-yl)-O,N-dimethyl-hydroxylamine hydrochloride (129a)

N-(4-allylamino-6-prop-2-ynylamino-[1,3,5]triazin-2-yl)-O,N-dimethyl-hydroxylamine (128) and 2 M HCl/diethyl ether were reacted using the procedure described for Compound 116a in quantitative yield. 400 MHz ¹H NMR (D₂O, ppm): 6.06-5.84 (1H, m), 5.37-5.14 (2H, m), 4.35-4.18 (2H, m), 4.18-3.99 (2H, m), 3.83 (3H, s), 3.54-3.31 (3H, m), 2.67 (1H, s). ESI-MS (m/z): 249 [M+H]⁺.

### 1-[4-(N-Methoxy-N-methyl-amino)-6-prop-2-ynylamino-[1,3,5]triazin-2-ylamino]-propan-2-ol (130)

A mixture of N-(4-chloro-6-prop-2-ynylamino-[1,3,5]triazin-2-yl)-O,N-dimethyl-hydroxylamine (127) (500 mg, 2.20 mmol) and 1-amino-propan-2-ol (860 μL, 11.00 mmol) in 1,4-dioxane (5 mL) was heated at 60° C. for 2 h. The mixture was cooled to room temperature. A saturated NaHCO₃ solution (15 mL) was added and the resulting suspension was extracted with EtOAc (3×20 mL). The combined organic extracts were washed with water (30 mL), then with a brine solution (30 mL) and lastly dried over solid anhydrous Na₂SO₄. After filtration, the solvent was removed under vacuum and the residue was filtered through silica gel using CH₂Cl₂/EtOH (95:5) to yield 1-[4-(N-methoxy-N-methyl-amino)-6-prop-2-ynylamino-[1,3,5]triazin-2-ylamino]-propan-2-ol (130) (530 mg, 90%). 400 MHz ¹H NMR (CDCl₃, ppm): δ 5.72-4.97 (3H, m), 4.21-4.12 (2H, m), 4.03-3.90 (1H, m), 3.76 (3H, s), 3.53-3.40 (1H, m), 3.36-3.22 (4H, m), 2.21 (1H, t, J=2.4 Hz), 1.19 (3H, d, J=6.3 Hz). ESI-MS (m/z): 267 [M+H]⁺.

### 3-[4-(N-Methoxy-N-methyl-amino)-6-prop-2-ynylamino-[1,3,5]triazin-2-ylamino]-propan-1-ol (131)

A mixture of N-(4-chloro-6-prop-2-ynylamino-[1,3,5]triazin-2-yl)-O,N-dimethyl-hydroxylamine (127) (500 mg, 2.20 mmol) and 3-amino-propan-1-ol (860 μL, 11.00 mmol) in 1,4-dioxane (5 mL) was heated at 60° C. for 2 h. The mixture was cooled to room temperature. A saturated NaHCO₃ solution (15 mL) was added and resulting suspension was extracted with EtOAc (3×20 mL). The combined organic extracts were washed with water (30 mL), then with a brine solution (30 mL) and lastly dried over solid anhydrous Na₂SO₄. After filtration, the solvent was removed under vacuum and the residue was purified by flash column chromatography using gradient elution from CH₂Cl₂/EtOH (99:1) to CH₂Cl₂/EtOH (99:5) to yield 3-[4-(N-methoxy-N-methyl-amino)-6-prop-2-ynylamino-[1,3,5]triazin-2-ylamino]-propan-1-ol (131) (260 mg, 44%). 400 MHz ¹H NMR (CDCl₃, ppm): δ 5.38-4.79 (3H, m), 4.21-4.12 (2H, m), 3.77 (3H, s), 3.68-3.50 (4H, m), 3.29 (3H, s), 2.21 (1H, t, J=2.3 Hz), 1.77-1.60 (2H, m). ESI-MS (m/z): 267 [M+H]⁺.

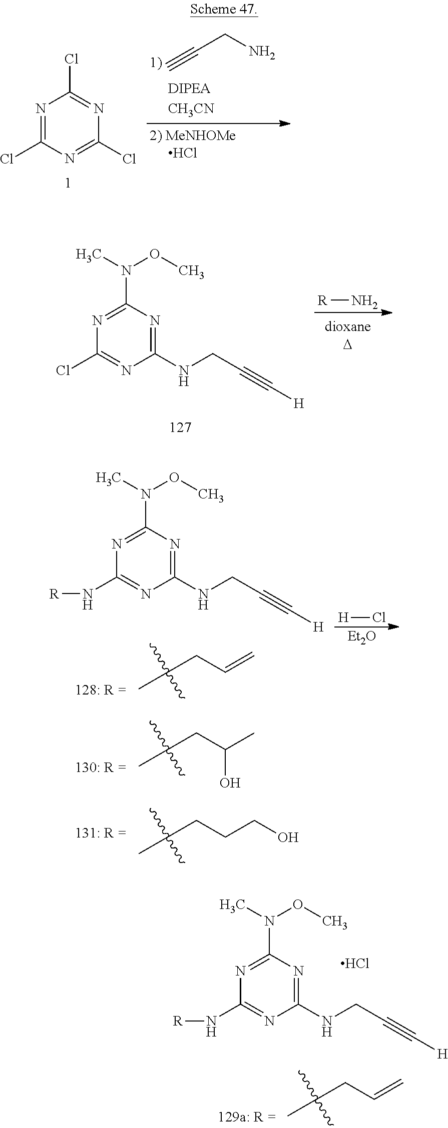

### Example 55

### N-(4-Amino-6-prop-2-ynylamino-[1,3,5]triazin-2-yl)-O,N-dimethyl-hydroxylamine (132) (Scheme 48)

### N-(4-Chloro-6-prop-2-ynylamino-[1,3,5]triazin-2-yl)-O,N-dimethyl-hydroxylamine (127)

To a solution of cyanuric chloride (1) (5.00 g, 27.11 mmol) in acetonitrile (50 mL) was gradually added a mixture of propargylamine (1.74 mL, 27.11 mmol) and N,N-diisopropylethylamine (4.69 mL, 27.11 mmol) in acetonitrile (50 mL) at −20° C. The mixture was stirred for 2 h, during which time the reaction warmed from −20° C. to 0° C. After this time, O,N-dimethylhydroxylamine hydrochloride (2.64 g, 27.11 mmol) was added to the reaction mixture, followed by N,N-diisopropylethylamine (9.38 mL, 54.22 mmol). The mixture was heated at 50° C. for 2 h, after which time the volatiles were removed by evaporation. A saturated NaHCO₃ solution (100 mL) was added to the residue, and the resulting suspension was extracted with EtOAc (2×75 mL). The combined organic extracts were washed with water (100 mL), then with a brine solution (100 mL), and lastly dried over solid anhydrous Na₂SO₄. After filtration, the solvent was removed under reduced pressure and the residue was crystallized from EtOAc to afford N-(4-chloro-6-prop-2-ynylamino-[1,3,5]triazin-2-yl)-O,N-dimethyl-hydroxylamine (2) (4.20 g, 68%). 400 MHz ¹H NMR (DMSO-d₆, ppm): δ 8.52-8.42 (1H, m), 4.07 (1.3H, dd, J=5.5, 2.4 Hz), 4.04 (0.7H, dd, J=5.5, 2.4 Hz), 3.75 (2H, s), 3.70 (1H, s), 3.32 (2H, s), 3.28 (1H, s), 3.15-3.12 (1H, m). ESI-MS (m/z): 228, 230 [M+H]⁺.

### N-(4-Amino-6-prop-2-ynylamino-[1,3,5]triazin-2-yl)-O, N-dimethyl-hydroxylamine (132)

A solution of N-(4-chloro-6-prop-2-ynylamino-[1,3,5]triazin-2-yl)-O,N-dimethyl-hydroxylamine (127) (300 mg, 1.32 mmol) and NH₄OH (25% solution, 1.5 mL) in 1,4-dioxane (5 mL) was heated at 60° C. for 2 h in a closed vial. The solvent was removed under reduced pressure, water (5 mL) was added and the precipitate was filtered and washed with water. The crude product was crystallized from methanol to yield N-(4-amino-6-prop-2-ynylamino-[1,3,5]triazin-2-yl)-O,N-dimethyl-hydroxylamine (132) (170 mg, 62%). 400 MHz ¹H NMR (DMSO-d₆, ppm): δ 7.36-6.96 (1H, m), 6.71-6.29 (2H, m), 3.98 (2H, dd, J=5.9, 2.2 Hz), 3.74-3.57 (3H, m), 3.22-3.09 (3H, m), 3.00 (1H, t, J=2.2 Hz). ESI-MS (m/z): 209 [M+H]⁺; melting point: 172-174° C.

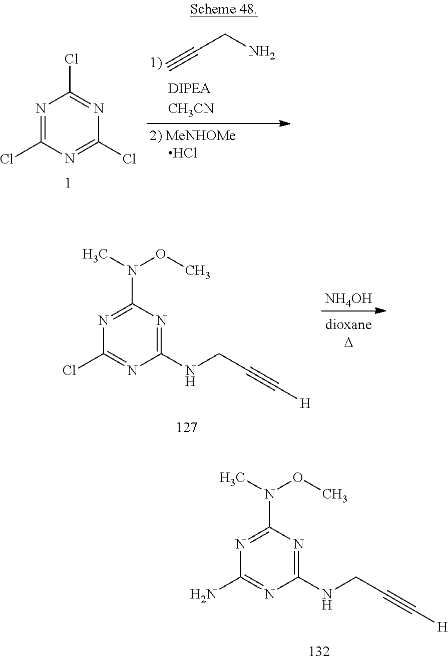

### Example 56

### 3-[4-(N-Methoxy-N-methylamino)-6-prop-2-ynylamino-[1,3,5]triazin-2-ylamino]-propionaldehyde (134) (Scheme 49)

### N-[4-(3,3-Diethoxy-propylamino)-6-prop-2-ynylamino-[1,3,5]triazin-2-yl)-O,N-dimethyl-hydroxylamine (133)

A mixture of of N-(4-chloro-6-prop-2-ynylamino-[1,3,5]triazin-2-yl)-O,N-dimethyl-hydroxylamine (127) (350 mg, 1.54 mmol), N,N-diisopropylethylamine (266 μL, 1.54 mmol) and 3,3-diethoxy-propylamine (498 μL, 3.08 mmol) in 1,4-dioxane (6 mL) was heated at 60° C. for 2 h. A saturated NaHCO₃ solution (50 mL) was added, and the resulting suspension was extracted with EtOAc (2×50 mL). The combined organic extracts were washed with water (75 mL), then with a brine solution (75 mL), and lastly dried over solid anhydrous Na₂SO₄. After filtration, the solvent was removed under reduced pressure to yield N-[4-(3,3-diethoxy-propylamino)-6-prop-2-ynylamino-[1,3,5]triazin-2-yl]-O,N-dimethyl-hydroxylamine (133) in quantitative yield. 400 MHz ¹H NMR (CDCl₃, ppm) 5.28 (1H, s), 5.02 (1H, br s), 4.59 (1H, t, J=5.6 Hz), 4.25-4.13 (2H, m), 3.76 (3H, s), 3.71-3.62 (2H, m), 3.55-3.42 (4H, m), 3.28 (3H, s), 2.19 (1H, t, J=2.5 Hz), 1.93-1.86 (2H, m), 1.22 (6H, t, J=7.0 Hz). ESI-MS (m/z): 3 39 [M+H]⁺.

### 3-[4-(N-Methoxy-N-methylamino)-6-prop-2-ynylamino-[1,3,5]triazin-2-ylamino]-propionaldehyde (134)

A solution of N-[4-(3,3-diethoxy-propylamino)-6-prop-2-ynylamino-[1,3,5]triazin-2-yl]-O,N-dimethyl-hydroxylamine (133) (450 mg, 1.33 mmol) in trifluoroacetic acid (50% water solution, 5 mL) and CHCl₃ (10 mL) was heated at 40° C. for 2 h. A saturated NaHCO₃ solution (50 mL) was then added, and the resulting suspension was extracted with CH₂Cl₂ (3×30 mL). The combined organic extracts were washed with water (75 mL) and dried over solid anhydrous Na₂SO₄. After filtration the solvent was removed under reduced pressure, and the residue was purified by flash column chromatography using gradient elution from CH₂Cl₂/EtOH (99:1) to CH₂Cl₂/EtOH (95:5) to yield 3-[4-(N-methoxy-N-methylamino)-6-prop-2-ynylamino-[1,3,5]triazin-2-ylamino]-propionaldehyde (134) (260 mg, 74%). 400 MHz ¹H NMR (CDCl₃, ppm): δ 9.82 (1H, s), 5.55-5.09 (2H, m), 4.26-4.07 (2H, m), 3.76 (3H, s), 3.73-3.61 (2H, m), 3.27 (3H, s), 2.83-2.71 (2H, m), 2.19 (1H, t, J=2.3 Hz). ESI-MS (m/z): 265 [M+H]⁺.

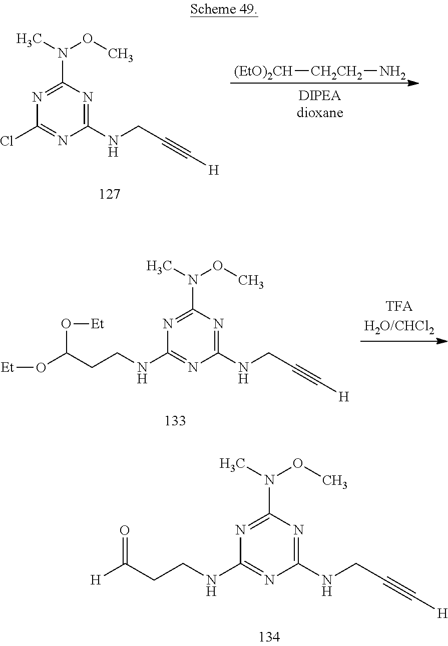

### Example 57

### 3-[4-(N-Methoxy-N-methylamino)-6-prop-2-ynylamino-[1,3,5]triazin-2-ylamino]-propionic acid ethyl ester hydrochloride (137) (Scheme 50)

### 3-[4-(N-methoxy-N-methyl-amino)-6-prop-2-ynylamino-[1,3,5]triazin-2-ylamino]-propionic acid ethyl ester (135)

A mixture of of N-(4-chloro-6-prop-2-ynylamino-[1,3,5]triazin-2-yl)-O,N-dimethyl-hydroxylamine (2) (500 mg, 2.20 mmol), β-alanine ethyl ester hydrochloride (676 mg, 4.40 mmol) and N,N-diisopropylethylamine (1.14 mL, 6.60 mmol) in 1,4-dioxane (10 mL) was heated at 90° C. for 24 h. A saturated NaHCO₃ solution (30 mL) was added and the resulting suspension was extracted with CH₂Cl₂ (3×30 mL). The combined organic extracts were washed with water (75 mL) and dried over solid anhydrous Na₂SO₄. After filtration, the solvent was removed under reduced pressure and the residue was purified by flash column chromatography using gradient elution from CH₂Cl₂/EtOH (99:1) to CH₂Cl₂/EtOH (95:5) to yield 3-[4-(N-methoxy-N-methyl-amino)-6-prop-2-ynylamino-[1,3,5]triazin-2-ylamino]-propionic acid ethyl ester (135) (580 mg, 86%). 400 MHz ¹H NMR (CDCl₃, ppm): δ 5.37 (1H, br s), 5.06 (1H, br s), 4.25-4.15 (2H, m), 4.15 (2H, q, J=7.1 Hz), 3.76 (3H, s), 3.72-3.61 (2H, m), 3.28 (3H, s), 2.60 (2H, t, J=6.1 Hz), 2.20 (1H, t, J=2.4 Hz), 1.26 (3H, t, J=7.1 Hz). ESI-MS (m/z): 309 [M+H]⁺.

### 3-[4-(N-Methoxy-N-methylamino)-6-prop-2-ynylamino-[1,3,5]triazin-2-ylamino]-propionic acid ethyl ester hydrochloride (136a)

A 2M HCl/diethyl ether (275 μL, 0.55 mmol) solution was added to a solution of 3-[4-(N-methoxy-N-methyl-amino)-6-prop-2-ynylamino-[1,3,5]triazin-2-ylamino]-propionic acid ethyl ester (135) (170 mg, 0.55 mmol) in diethyl ether (5 mL) at 0° C. The mixture was stirred for 0.5 h at 0° C., after which time the volatiles were removed under reduced pressure to yield 3-[4-(N-methoxy-N-methyl-amino)-6-prop-2-ynylamino-[1,3,5]triazin-2-ylamino]-propionic acid ethyl ester hydrochloride (136a) in quantitative yield. 400 MHz ¹H NMR (D₂O, ppm): δ 4.37-4.07 (4H, m), 3.95-3.64 (5H, m), 3.55-3.26 (3H, m), 2.87-2.58 (3H, m), 1.24 (3H, t, J=6.7 Hz). ESI-MS (m/z): 309 [M+H]⁺.

### 3-[4-(N-Methoxy-N-methyl-amino)-6-prop-2-ynylamino-[1,3,5]triazin-2-ylamino]-propionic acid (137)

6M HCl (6 mL) was added to a solution of 3-[4-(N-methoxy-N-methyl-amino)-6-prop-2-ynylamino-[1,3,5]triazin-2-ylamino]-propionic acid ethyl ester (135) (400 mg, 1.30 mmol) in THF (10 mL), and the reaction mixture was stirred at room temperature for 24 h. After this time, the pH of the solution was adjusted to ca. 5 by addition of NH₄OH (25% solution, ˜5 mL). The resultant precipitate was collected by filtration and dried to yield 3-[4-(N-methoxy-N-methyl-amino)-6-prop-2-ynylamino-[1,3,5]triazin-2-ylamino]-propionic acid (137) (270 mg, 74%). 400 MHz H NMR (DMSO-d₆, ppm): δ 12.12 (1H, br s), 7.42-7.19 (1H, m), 7.15-6.75 (1H, m), 4.08-3.89 (2H, m), 3.79-3.56 (3H, m), 3.50-3.36 (2H, m), 3.23-3.07 (3H, m), 2.99 (1H, s), 2.63-2.38 (2H, m, overlapped with DMSO). ESI-MS (m/7): 281 [M+H]⁺; melting point: 164-166° C.

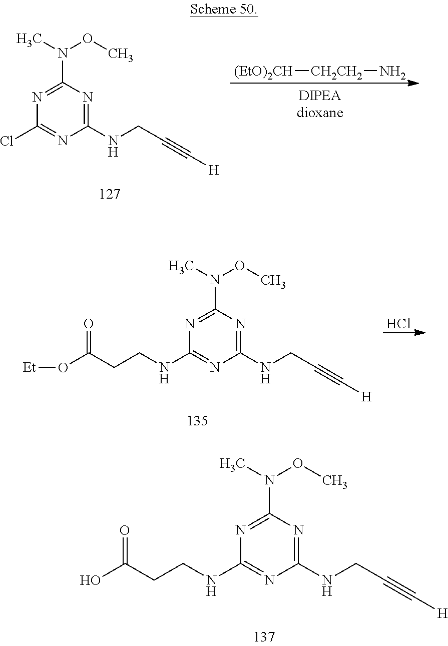

### Example 58

### N-Propyl-N′-prop-2-ynyl-[1,3,5]triazine-2,4,6-triamine (138) (Scheme 51)

A solution of 6-chloro-N-propyl-N′-prop-2-ynyl-[1,3,5]triazine-2,4-diamine (3) (451 mg, 2.00 mmol) and NH₄OH (25% solution, 3.0 mL) in 1,4-dioxane (5 mL) was heated at 80° C. for 16 h in a closed vial. A saturated NaHCO₃ solution (30 mL) was added and the resulting suspension was extracted with CH₂Cl₂ (3×30 mL). The combined organic extracts were washed with water (50 mL) and dried over anhydrous solid Na₂SO₄. The solvent was removed under reduced pressure, and the residue was purified by flash column chromatography using gradient elution from CH₂Cl₂/EtOH (99:1) to CH₂Cl₂/EtOH (95:5) to yield N-propyl-N′-prop-2-ynyl-[1,3,5]triazine-2,4,6-triamine (138) (250 mg, 61%). 400 MHz ¹H NMR (DMSO-d₆, ppm): δ 5.08 (1H, br s), 4.89 (1H, br s), 4.79 (2H, br s), 4.18 (2H, s), 3.40-3.24 (2H, m), 2.21 (1H, t, J=2.4 Hz), 1.57 (2H, sextet, J=7.3 Hz), 0.95 (3H, t, J=7.3 Hz). ESI-MS (m/z): 207 [M+H]⁺.

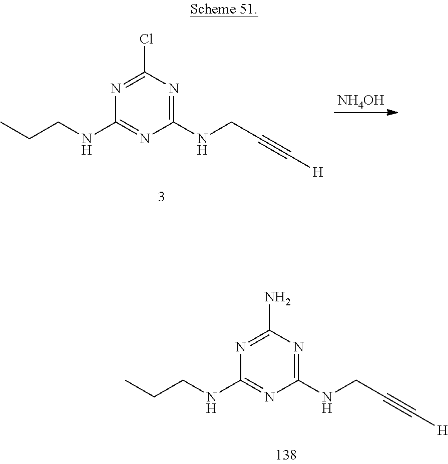

### Examples 59-60

### N-[4-(N′-Methoxy-N′-methyl-amino)-6-prop-2-ynylamino-[1,3,5]triazin-2-yl]-N-propyl acetamide (141)

### N-[4-(N′-Methoxy-N′-methyl-amino)-6-prop-2-ynylamino-[1,3,5]triazin-2-yl]-N-propyl adamantylamide (143) (Scheme 52)

### N-(4-Chloro-6-n-propylamino-[1,3,5]triazin-2-yl)-O,N-dimethyl-hydroxylamine (139)

A solution of n-propylamine (2.23 mL, 27.11 mmol) and N,N-diisopropylethylamine (4.69 mL, 27.11 mmol) in acetonitrile (50 mL) was added gradually to a solution of cyanuric chloride (1) (5.00 g, 27.11 mmol) in acetonitrile (50 mL) at −20° C. The reaction mixture was stirred for 2 h during which time the reaction temperature rose from −20° C. to 0° C.). After this time, O,N-dimethyl-hydroxylamine hydrochloride (2.64 g, 27.11 mmol) was added to the mixture followed by N,N-diisopropylethylamine (9.38 mL, 54.22 mmol). The mixture was heated at 50° C. for 2 h, and then the volatiles were removed by evaporation. A saturated NaHCO₃ solution (100 mL) was added and the resulting suspension was extracted with EtOAc (2×75 mL). The combined organic extracts were washed with water (100 mL), then with a brine solution (100 mL) and lastly dried over solid anhydrous Na₂SO₄. After filtration, the solvent was removed under reduced pressure and the residue was crystallized from EtOAc to afford N-(4-chloro-6-n-propylamino-[1,3,5]triazin-2-yl)-O,N-dimethyl-hydroxylamine (139) (5.69 g, 91%). 400 MHz ¹H NMR (CDCl₃, ppm): δ 5.49-5.35 (1H, m), 3.82-3.75 (3H, m), 3.43-3.32 (5H, m), 1.65-1.51 (2H, m), 0.99-0.92 (3H, m). ESI-MS (m/z): 232, 234 [M+H]⁺.

### N-[4-Chloro-6-(N′-methoxy-N′-methyl-amino)-[1,3,5]triazin-2-yl]-N-propyl-acetamide (140)

Lithium bis(trimethylsilyl)amide (1M in THF, 2.37 mL, 2.37 mmol) was added dropwise to a solution of N-(4-chloro-6-n-propylamino-[1,3,5]triazin-2-yl)-O,N-dimethyl-hydroxylamine (139) (500 mg, 2.16 mmol) in THF (10 mL) at −78° C., and the reaction mixture was stirred for 5 minutes. Acetyl chloride (0.31 mL, 4.32 mmol) was added dropwise to reaction and the mixture was warmed to ambient temperature and stirred for 18 h. After this time, a saturated NaHCO₃ (30 mL) was added and the mixture was extracted with CH₂Cl₂ (3×30 mL). The combined organic extracts were washed with water (100 mL) and dried over solid anhydrous Na₂SO₄. After filtration, the solvent was evaporated under reduced pressure. The resultant residue was purified by flash column chromatography using gradient elution from PE/EtOAc (97/3) to PE/EtOAc (80/20) to yield N-[4-chloro-6-(N-methoxy-N-methyl-amino)-[1,3,5]triazin-2-yl]-N-propyl-acetamide (140) (337 mg, 63%). 400 MHz ¹H NMR (CDCl₃, ppm): δ 3.99-3.94 (2H, m), 3.82 (3H, s), 3.41 (3H, m), 2.60 (3H, s), 1.67-1.55 (2H, m), 0.90 (3H, t, J=7.4 Hz). ESI-MS (m/z): 274, 276 [M+H]+.

### N-[4-(N′-methoxy-N′-methyl-amino)-6-prop-2-ynylamino-[1,3,5]triazin-2-yl]-N-propyl acetamide (141)

A mixture of N-[4-chloro-6-(N-methoxy-N-methyl-amino)-[1,3,5]triazin-2-yl]-N-propyl-acetamide (140) (373 mg, 1.36 mmol) and propargylamine (0.24 mL, 6.40 mmol) in THF (4 mL) was heated at 60° C. for 18 h. A saturated NaHCO₃ solution (30 mL) was added, and the mixture was extracted with CH₂Cl₂ (3×30 mL). The combined organic extracts were washed with water (100 mL) and dried over solid anhydrous Na₂SO₄. After filtration, the solvent was evaporated under reduced pressure and the residue was purified by flash column chromatography using gradient elution from PE/EtOAc (94/6) to PE/EtOAc (60/40) to yield N-[4-(N′-methoxy-N′-methyl-amino)-6-prop-2-ynylamino-[1,3,5]triazin-2-yl]-N-propyl acetamide (141). 400 MHz ¹H NMR (CDCl₃, ppm): δ 5.42 (0.6H, s), 5.24 (0.4H, br s), 4.29-4.13 (2H, m), 4.02-3.87 (2H, m), 3.79 (3H, s), 3.32 (3H, s), 2.63-2.48 (3H, m), 2.22 (1H, s), 1.69-1.55 (2H, m, overlapped with water), 0.90 (3H, t, J=7.1 Hz). ESI-MS (m/z): 293 [M+H]+.

### N-[4-Chloro-6-(N′-methoxy-N′-methyl-amino)-[1,3,5]triazin-2-yl]-N-propyl-adamantyl amide (142)

N-(4-Chloro-6-n-propylamino-[1,3,5]triazin-2-yl)-O,N-dimethyl-hydroxylamine (139) and 1-adamantanecarbonyl chloride were reacted as described using the procedure described for compound (140) to afford N-[4-chloro-6-(N′-methoxy-N′-methyl-amino)-[1,3,5]triazin-2-yl]-N-propyl-adamantyl amide (142) in 50% yield. 400 MHz ¹H NMR (CDCl₃, ppm): δ 3.73 (3H, s), 3.70-3.64 (2H, m), 3.38 (3H, s), 2.05-1.98 (9H, m), 1.72-1.63 (8H, m), 0.94 (3H, t, J=7.4 Hz). ESI-MS (m/z): 394, 396 [M+H]+.

### N-[4-(N′-methoxy-N′-methyl-amino)-6-prop-2-ynylamino-[1,3,5]triazin-2-yl]-N-propyl-adamantylamide (143)

N-[4-Chloro-6-(N′-methoxy-N′-methyl-amino)-[1,3,5]triazin-2-yl]-N-propyl-adamantyl amide (142) and propargylamine were reacted using the procedure described for compound (141) to afford N-[4-(N′-methoxy-N′-methyl-amino)-6-prop-2-ynylamino-[1,3,5]triazin-2-yl]-N-propyl-adamantylamide (143) in 43% yield. 400 MHz ¹H NMR (CDCl₃, ppm): δ 5.29 (0.6H, br s), 5.13 (0.4H, br s), 4.24-4.19 (2H, m), 3.78 (3H, s), 3.68-3.63 (2H, m), 3.32 (3H, s), 2.23-2.19 (1H, m), 2.03-1.93 (9H, m), 1.70-1.58 (8H, m), 0.91 (3H, t, J=7.2 Hz). ESI-MS (m/z): 413 [M+H]+.

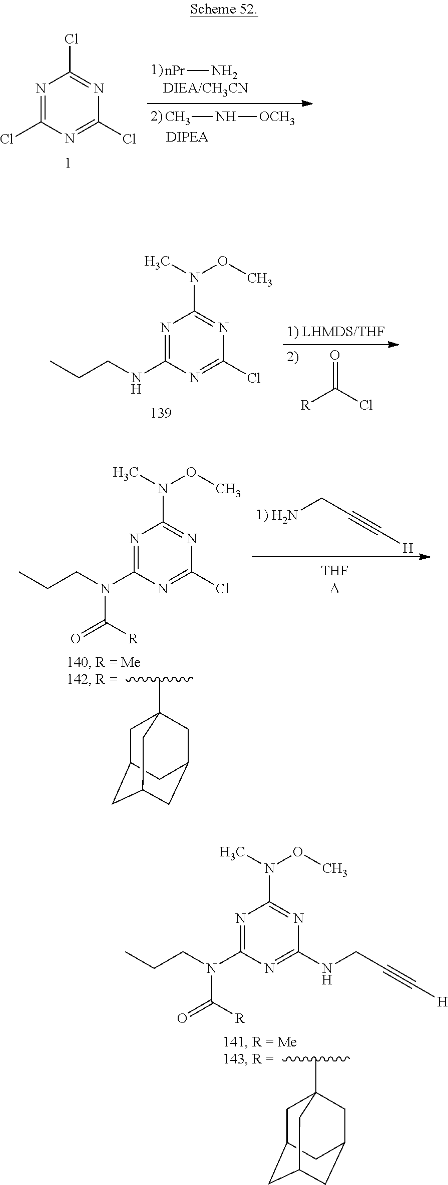

### Example 61

### N-Ethyl-N′-methyl-N″-prop-2-ynyl-[1,3,5]triazine-2,4,6-triamine (145) and corresponding hemisulfate salt (146b) (Scheme 53)

### 6-Chloro-N-methyl-N′-prop-2-ynyl-[1,3,5]triazine-2,4-diamine (144)

To the solution of cyanuric chloride (1) (5.00 g, 27.11 mmol) in THF (50 mL), a mixture of propargylamine (1.74 mL, 27.11 mmol) and N,N-diisopropylethylamine (4.69 mL, 27.11 mmol) in THF (30 mL) was added gradually at −20° C. The reaction mixture was stirred for 2 h (reaction temperature from −20° C. to 0° C.). After this time, a methylamine/THF solution (2 M, 16.3 mL, 32.60 mmol) was added, followed by N,N-diisopropylethylamine (4.69 mL, 27.11 mmol). The mixture was stirred at room temperature for 16 h. The resultant precipitate were filtered, washed with hot water and dried to yield 6-chloro-N-methyl-N′-prop-2-ynyl-[1,3,5]triazine-2,4-diamine (144) (3.70 g, 69%). 400 MHz ¹H NMR (DMSO-d₆, ppm): δ 8.30-7.54 (2H, m), 4.13-3.95 (2H, m), 3.09 (1H, t, J=2.3 Hz), 2.82-2.71 (3H, m). ESI-MS (m/z): 198, 200 [M+H]⁺.

### N-Ethyl-N′-methyl-N″-prop-2-ynyl-[1,3,5]triazine-2,4,6-triamine (145)

A solution of 6-chloro-N-methyl-N′-prop-2-ynyl-[1,3,5]triazine-2,4-diamine (144) (273 mg, 1.38 mmol) and ethylamine (70% water solution, 1.5 mL) in 1,4-dioxane (4 mL) was heated at 70° C. for 2 h in a closed vial. After this time, an aqueous saturated NaHCO₃ solution (20 mL) was added and the resulting suspension was extracted with EtOAc (2×30 mL). The combined organic extracts were washed with water (50 mL), then with a brine solution (50 mL), and lastly dried over solid anhydrous Na₂SO₄. After filtration, the solvent was removed under reduced pressure and the residue was purified by flash column chromatography using gradient elution from petroleum ether/EtOAc (1:1) to petroleum ether/EtOAc (1:9) to yield N-ethyl-N′-methyl-N″-prop-2-ynyl-[1,3,5]triazine-2,4,6-triamine (145) (250 mg, 87%). 400 MHz ¹H NMR (CDCl₃, ppm): δ 5.06 (1H, br s), 4.83 (2H, br s), 4.27-4.09 (2H, m), 3.47-3.30 (2H, m), 2.91 (3H, d, J=4.8 Hz), 2.20 (1H, t, J=2.5 Hz), 1.17 (3H, t, J=7.2 Hz). ESI-MS (m/z): 207 [M+H]⁺.

### N-Ethyl-N′-methyl-N″-prop-2-ynyl-[1,3,5]triazine-2,4,6-triamine hemisulfate (146b)

To a solution of N-ethyl-N′-methyl-N″-prop-2-ynyl-[1,3,5]triazine-2,4,6-triamine (145) (230 mg, 1.12 mmol) in 1,4-dioxane (4 mL) was added 95% H₂SO₄ (31 μL, 0.56 mmol). The mixture was stirred for 0.5 h at room temperature, and then the volatiles were removed under reduced pressure. The residue was triturated with Et₂O/EtOH to yield N-ethyl-N′-methyl-N″-prop-2-ynyl-[1,3,5]triazine-2,4,6-triamine hemisulfate (146b) (200 mg, 70%). 400 MHz ¹H NMR (D₂O, ppm): δ 4.34-4.08 (2H, m), 3.62-3.30 (2H, m), 3.09-2.81 (3H, m), 2.71-2.64 (1H, m), 1.28-1.13 (3H, m). ESI-MS (m/z): 207 [M+H]⁺; melting point: 108-110° C.

### Example 62

### N-Cyclopropyl-N′-methyl-N″-prop-2-ynyl-[1,3,5]triazine-2,4,6-triamine (147) and corresponding hemisulfate salt (148b) (Scheme 53)

### N-Cyclopropyl-N′-methyl-N″-prop-2-ynyl-[1,3,5]triazine-2,4,6-triamine (147)

A solution of 6-chloro-N-methyl-N′-prop-2-ynyl-[1,3,5]triazine-2,4-diamine (144) (273 mg, 1.38 mmol) and cyclopropylamine (574 μL, 8.28 mmol) in 1,4-dioxane (4 mL) was heated at 60° C. for 16 h in a closed vial. An aqueous saturated NaHCO₃ solution (20 mL) was then added and the resulting suspension was extracted with EtOAc (2×30 mL). The combined organic extracts were washed with water (50 mL), then with a brine solution (50 mL), and lastly dried over solid anhydrous Na₂SO₄. After filtration, the solvent was removed under reduced pressure and the residue was purified by flash column chromatography using gradient elution from petroleum ether/EtOAc (1:1) to petroleum ether/EtOAc (1:9) to yield N-cyclopropyl-N′-methyl-N″-prop-2-ynyl-[1,3,5]triazine-2,4,6-triamine (147) (260 mg, 86%). 400 MHz ¹H NMR (CDCl₃, ppm): δ 5.18-4.95 (2H, m), 4.87 (1H br s), 4.29-4.09 (2H, m), 2.92 (3H, d, J=4.8 Hz), 2.81-2.66 (1H, m), 2.20 (1H, t, J=2.5 Hz), 0.77-0.70 (2H, m), 0.56-0.46 (2H, m). ESI-MS (m/z): 219 [M+H]⁺.

### N-Cyclopropyl-N′-methyl-N″-prop-2-ynyl-[1,3,5]triazine-2,4,6-triamine hemisulfate (148b)

N-Cyclopropyl-N′-methyl-N″-prop-2-ynyl-[1,3,5]triazine-2,4,6-triamine (147) and 95% H₂SO₄ were reacted according to the procedure described for compound (146b) to afford N-cyclopropyl-N′-methyl-N″-prop-2-ynyl-[1,3,5]triazine-2,4,6-triamine hemisulfate (148b) (62% yield). 400 MHz ¹H NMR (D₂O, ppm): δ 4.40-4.10 (2H, m), 3.13-2.83 (3H, m), 2.81-2.59 (2H, m), 0.99-0.84 (2H, m), 0.77-0.64 (2H, m). ESI-MS (m/z): 219 [M+H]⁺; melting point: 124-126° C.

### Example 63

### N-Butyl-N′-methyl-N″-prop-2-ynyl-[1,3,5]triazine-2,4,6-triamine (149) and corresponding hemisulfate salt (150b) (Scheme 53)

### N-Butyl-N′-methyl-N″-prop-2-ynyl-[1,3,5]triazine-2,4,6-triamine (149)

6-Chloro-N-methyl-N′-prop-2-ynyl-[1,3,5]triazine-2,4-diamine (144) and butylamine were reacted according to the procedure described for compound (145) to afford N-butyl-N′-methyl-N″-prop-2-ynyl-[1,3,5]triazine-2,4,6-triamine (149) (93% yield). 400 MHz ¹H NMR (CDCl₃, ppm): δ 5.03 (1H, br s), 4.82 (2H, br s), 4.29-4.06 (2H, m), 3.47-3.24 (2H, m), 2.95-2.85 (3H, m), 2.20 (1H, t, J=2.4 Hz), 1.58-1.47 (2H, m), 1.43-1.32 (2H, m), 0.92 (3H, t, J=7.4 Hz). ESI-MS (m/z): 235 [M+H]⁺.

### N-Butyl-N′-methyl-N″-prop-2-ynyl-[1,3,5]triazine-2,4,6-triamine hemisulfate (150b)

N-Butyl-N′-methyl-N″-prop-2-ynyl-[1,3,5]triazine-2,4,6-triamine (149) and 95% H₂SO₄ were reacted according to the procedure described for compound (146b) to afford N-butyl-N′-methyl-N″-prop-2-ynyl-[1,3,5]triazine-2,4,6-triamine hemisulfate (150b) (99% yield). 400 MHz ¹H NMR (D₂O, ppm): δ 4.36-4.09 (2H, m), 3.55-3.26 (2H, m), 3.06-2.83 (3H, m), 2.68 (1H, t, J=2.3 Hz), 1.67-1.50 (2H, m), 1.44-1.29 (2H, m), 0.92 (3H, t, J=7.4 Hz). ESI-MS (m/z): 235 [M+H]⁺; melting point: 145-147° C.

### Example 64

### N-Cyclopropylmethyl-N′-methyl-N″-prop-2-ynyl-[1,3,5]triazine-2,4,6-triamine (151) and corresponding hemisulfate salt (152b) (Scheme 53)

### N-Cyclopropylmethyl-N′-methyl-N″-prop-2-ynyl-[1,3,5]triazine-2,4,6-triamine (151)

6-Chloro-N-methyl-N′-prop-2-ynyl-[1,3,5]triazine-2,4-diamine (144) and cyclopropylmethylamine were reacted according to the procedure described for compound (145) to afford N-cyclopropylmethyl-N′-methyl-N″-prop-2-ynyl-[1,3,5]triazine-2,4,6-triamine (151) (53% yield). 400 MHz ¹H NMR (CDCl₃, ppm): δ 4.94 (2H, br s), 4.77 (1H, br s), 4.28-4.09 (2H, m), 3.31-3.13 (2H, m), 2.92 (3H, d, J=5.0 Hz), 2.20 (1H, t, J=2.5 Hz), 1.09-0.95 (1H, m), 0.54-0.44 (2H, m), 0.26-0.18 (2H, m). ESI-MS (m/z): 233 [M+H]⁺.

### N-Cyclopropylmethyl-N′-methyl-N″-prop-2-ynyl-[1,3,5]triazine-2,4,6-triamine hemisulfate (152b)

N-Cyclopropylmethyl-N′-methyl-N″-prop-2-ynyl-[1,3,5]triazine-2,4,6-triamine (151) and 95% H₂SO₄ were reacted according to the procedure described for compound (146b) to afford N-cyclopropylmethyl-N′-methyl-N″-prop-2-ynyl-[1,3,5]triazine-2,4,6-triamine hemisulfate (152b) (77% yield). 400 MHz ¹H NMR (D₂O, ppm): δ 4.35-4.08 (2H, m), 3.43-3.15 (2H, m), 3.08-2.89 (3H, m), 2.73-2.61 (1H, m), 1.19-1.03 (1H, m), 0.61-0.50 (2H, m), 0.35-0.25 (2H, m). ESI-MS (m/z): 233 [M+H]⁺; melting point: 130-132° C.

### Example 65

### N-Methyl-N′-prop-2-ynyl-N″-(3,3,3-trifluoro-propyl)-[1,3,5]triazine-2,4,6-triamine (153) and corresponding hemisulfate salt (154b) (Scheme 53)

### N-Methyl-N′-prop-2-ynyl-N″-(3,3,3-trifluoro-propyl)-[1,3,5]triazine-2,4,6-triamine (153)

6-Chloro-N-methyl-N′-prop-2-ynyl-[1,3,5]triazine-2,4-diamine (144) and 3,3,3-trifluoro-propylamine were reacted according to the procedure described for compound (145) to afford N-methyl-N′-prop-2-ynyl-N″-(3,3,3,-trifluoro-propyl)-[1,3,5]triazine-2,4,6-triamine (153) (69% yield). 400 MHz ¹H NMR (CDCl₃, ppm): δ 5.40-5.03 (2H, m), 4.95 (1H, br s), 4.25-4.10 (2H, m), 3.67-3.54 (2H, m), 2.98-2.85 (3H, m), 2.51-2.35 (2H, m), 2.20 (1H, t, J=2.5 Hz). ESI-MS (m/z): 275 [M+H]⁺.

### N-Methyl-N′-prop-2-ynyl-N″-(3,3,3-trifluoro-propyl)-[1,3,5]triazine-2,4,6-triamine hemisulfate (154b)

N-Methyl-N′-prop-2-ynyl-N″-(3,3,3-trifluoro-propyl)-[1,3,5]triazine-2,4,6-triamine (153) and 95% H₂SO₄ were reacted according to the procedure described for compound (146b) to afford N-methyl-N′-prop-2-ynyl-N″-(3,3,3-trifluoro-propyl)-[1,3,5]triazine-2,4,6-triamine hemisulfate (154b) (81% yield). 400 MHz ¹H NMR (D₂O, ppm): δ 4.34-4.13 (2H, m), 3.81-3.62 (2H, m), 3.06-2.86 (3H, m), 2.72-2.66 (1H, m), 2.66-2.49 (2H, m). ESI-MS (m/z): 275 [M+H]⁺; melting point: 149-151° C.

### Example 66

### N-Methyl-N′-(2,2,3,3,3-pentafluoro-propyl)-N″-prop-2-ynyl-[1,3,5]triazine-2,4,6-triamine sulfate (155) and corresponding hemisulfate salt (156b) (Scheme 53)

### N-Methyl-N′-(2,2,3,3,3-pentafluoro-propyl)-N″-prop-2-ynyl-[1,3,5]triazine-2,4,6-triamine (155)

6-Chloro-N-methyl-N′-prop-2-ynyl-[1,3,5]triazine-2,4-diamine (144) and 2,2,3,3,3-pentafluoro-propylamine were reacted according to the procedure described for compound (145) to afford N-methyl-N′-(2,2,3,3,3-pentafluoro-propyl)-N″-prop-2-ynyl-[1,3,5]triazine-2,4,6-triamine (155) (58% yield). 400 MHz ¹H NMR (CDCl₃, ppm): δ 5.37-4.80 (3H, m), 4.31-4.03 (4H, m), 2.92 (3H, d, J=4.4 Hz), 2.21 (1H, t, J=2.5 Hz). ESI-MS (m/z): 311 [M+H]⁺.

### N-Methyl-N′-(2,2,3,3,3-pentafluoro-propyl)-N″-prop-2-ynyl-[1,3,5]triazine-2,4,6-triamine hemisulfate (156b)

N-Methyl-N′-(2,2,3,3,3-pentafluoro-propyl)-N″-prop-2-ynyl-[1,3,5]triazine-2,4,6-triamine (155) and 95% H₂SO₄ were reacted according to the procedure described for compound (146b) to afford N-methyl-N′-(2,2,3,3,3-pentafluoro-propyl)-N″-prop-2-ynyl-[1,3,5]triazine-2,4,6-triamine hemisulfate (156b) (84% yield). 400 MHz ¹H NMR (D₂O, ppm): δ 4.44-4.14 (4H, m), 3.05-2.89 (3H, m), 2.72-2.65 (1H, m). ESI-MS (m/z): 311 [M+H]⁺; melting point: 197-199° C.

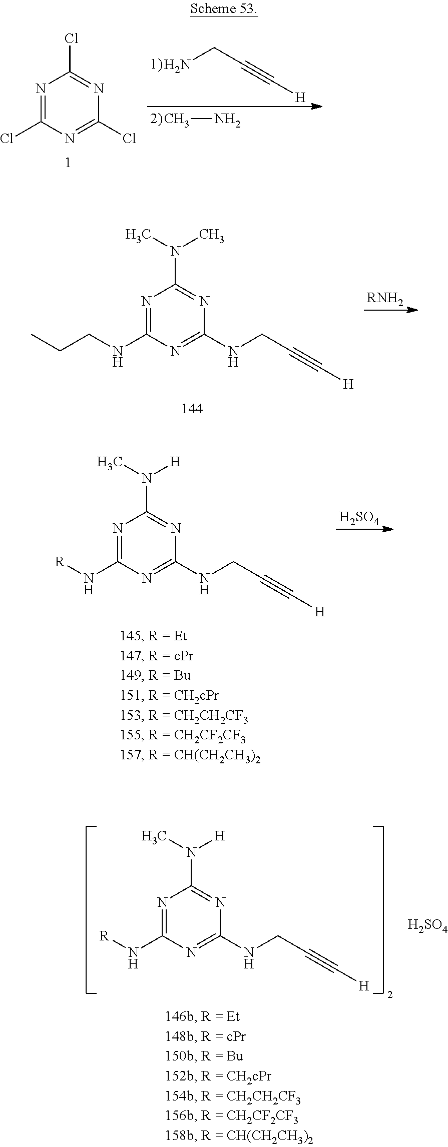

### Example 67

### N-(1-Ethyl-propyl)-N′-methyl-N″-prop-2-ynyl-[1,3,5]triazine-2,4,6-triamine sulfate (157) and corresponding hemisulfate salt (158b) (Scheme 53)

### N-(1-Ethyl-propyl)-N′-methyl-N″-prop-2-ynyl-[1,3,5]triazine-2,4,6-triamine (157)

6-Chloro-N-methyl-N′-prop-2-ynyl-[1,3,5]triazine-2,4-diamine (144) and 1-ethyl-propylamine were reacted according to the procedure described for compound (145) to afford N-(1-ethyl-propyl)-N′-methyl-N″-prop-2-ynyl-[1,3,5]triazine-2,4,6-triamine (157) (89% yield). 400 MHz ¹H NMR (CDCl₃, ppm): δ 4.97 (1H, br s), 4.83 (1H, br s), 4.68 (1H, br s), 4.25-4.10 (2H, m), 3.96-3.78 (1H, m), 2.91 (3H, d, J=4.6 Hz), 2.20 (1H, t, J=2.5 Hz), 1.63-1.51 (2H, m), 1.50-1.36 (2H, m), 0.90 (6H, t, J=7.4 Hz). ESI-MS (m/z): 249 [M+H]⁺.

### N-(1-Ethyl-propyl)-N′-methyl-N″-prop-2-ynyl-[1,3,5]triazine-2,4,6-triamine hemisulfate (157b)

N-(1-Ethyl-propyl)-N′-methyl-N″-prop-2-ynyl-[1,3,5]triazine-2,4,6-triamine (157) and 95% H₂SO₄ were reacted according to the procedure described for compound (146b) to afford N-(1-ethyl-propyl)-N′-methyl-N″-prop-2-ynyl-[1,3,5]triazine-2,4,6-triamine hemisulfate (157b) (73% yield). 400 MHz ¹H NMR (D₂O ppm): δ 4.35-4.11 (2H, m), 4.10-3.93 (1H, m), 3.04-2.88 (3H, m), 2.70-2.63 (1H, m), 1.73-1.58 (2H, m), 1.57-1.41 (2H, m), 0.90 (6H, t, J=7.3 Hz). ESI-MS (m/z): 249 [M+H]⁺; melting point: 161-163° C.

### Example 68

### N,N-Dimethyl-N′-propyl-N″-prop-2-ynyl-[1,3,5]triazine-2,4,6-triamine (159) and corresponding hemisulfate salt (160b)

### N,N-Dimethyl-N′-propyl-N″-prop-2-ynyl-[1,3,5]triazine-2,4,6-triamine (159)

A solution of 6-chloro-N-propyl-N′-prop-2-ynyl-[1,3,5]triazine-2,4-diamine (3) (400 mg, 1.77 mmol) and dimethylamine (2M in THF, 2.66 mL, 5.32 mmol) in 1,4-dioxane (8 mL) was heated at 70° C. for 18 h in a closed vial. An aqueous saturated NaHCO₃ solution (15 mL) was added, and the resulting suspension was extracted with EtOAc (3×10 mL). The combined organic extracts were washed with water (20 mL), then with a brine solution (20 mL), and lastly dried over solid anhydrous Na₂SO₄. The solvent was removed under reduced pressure and the residue was purified by flash column chromatography using CHCl₃ as eluent to yield N,N-dimethyl-N′-propyl-N″-prop-2-ynyl-[1,3,5]triazine-2,4,6-triamine (159) (390 mg, 94%). 400 MHz ¹H NMR (CDCl₃, ppm): δ 5.05-4.71 (2H, m), 4.25-4.13 (2H, m), 3.36-3.28 (2H, m), 3.08 (6H, s), 2.18 (1H, t, J=2.5 Hz), 1.63-1.51 (2H, m), 0.94 (3H, t, J=7.3 Hz). ESI-MS (m/z): 235 [M+H]⁺.

### N,N-Dimethyl-N′-propyl-N″-prop-2-ynyl-[1,3,5]triazine-2,4,6-triamine hemisulfate (160b)

To a solution of N,N-dimethyl-N′-propyl-N″-prop-2-ynyl-[1,3,5]triazine-2,4,6-triamine (159) (390 mg, 1.66 mmol) in 1,4-dioxane (8 mL) was added 95% H₂SO₄ (47 μL, 1.66 mmol). The mixture was stirred for 1 h at room temperature and then volatiles were removed under reduced pressure. The residue was co-evaporated with toluene (2×5 mL) and then triturated with Et₂O to yield N,N-dimethyl-N′-propyl-N″-prop-2-ynyl-[1,3,5]triazine-2,4,6-triamine hemisulfate (160b) (435 mg, 92%). 400 MHz ¹H NMR (D₂O, ppm): δ 4.32-4.14 (2H, m), 3.50-3.28 (2H, m), 3.28-3.04 (6H, m), 2.70-2.63 (1H, m), 1.71-1.54 (2H, m), 0.93 (3H, t, J=7.3 Hz). ESI-MS (m/z): 235 [M+H]⁺; melting point: 157-159° C.

### Example 69

### N,N-Ethyl-methyl-N′-propyl-N″-prop-2-ynyl-[1,3,5]triazine-2,4,6-triamine (161) and corresponding hemisulfate salt (162b)

### N,N-Ethyl-methyl-N′-propyl-N″-prop-2-ynyl-[1,3,5]triazine-2,4,6-triamine (161)

6-Chloro-N-propyl-N′-prop-2-ynyl-[1,3,5]triazine-2,4-diamine (3) and N,N-ethylmethylamine were reacted according to the procedure described for compound (159) to afford N,N-ethyl-methyl-N′-propyl-N″-prop-2-ynyl-[1,3,5]triazine-2,4,6-triamine (161) (89% yield). 400 MHz ¹H NMR (CDCl₃, ppm): δ 4.85 (1H, br s), 4.78 (1H, br s), 4.97-4.67 (2H, m), 4.24-4.09 (2H, m), 3.65-3.51 (2H, m), 3.38-3.26 (2H, m), 3.05 (3H, s), 2.18 (1H, t, J=2.5 Hz), 1.63-1.50 (2H, m), 1.12 (3H, t, J=7.3 Hz), 0.94 (3H, t, J=7.3 Hz). ESI-MS (m/z): 249 [M+H]⁺.

### N,N-Ethyl-methyl-N′-propyl-N″-prop-2-ynyl-[1,3,5]triazine-2,4,6-triamine hemisulfate (162a)

N,N-Ethyl-methyl-N′-propyl-N″-prop-2-ynyl-[1,3,5]triazine-2,4,6-triamine (161) and 95% H₂SO₄ were reacted according to the procedure described for compound (160b) to afford N,N-ethyl-methyl-N′-propyl-N″-prop-2-ynyl-[1,3,5]triazine-2,4,6-triamine hemisulfate (162a) (83% yield). 400 MHz ¹H NMR (CDCl₃ ppm) 13.73 (1H, br s), 8.10-8.01 (1H, m), 7.73-7.63 (1H, m), 4.12 (1H, dd, J=5.6, 2.5 Hz), 4.09 (1H, dd, J=5.6, 2.5 Hz), 3.69-3.56 (2H, m), 3.34-3.24 (2H, m), 3.15 (1.5H, s), 3.13 (1.5H, s), 2.14 (0.5H, t, J=2.5 Hz), 2.13 (0.5H, t, J=2.5 Hz), 1.65-1.53 (2H, m), 1.20-1.11 (3H, m), 0.92-0.86 (3H, m). ESI-MS (m/z): 249 [M+H]⁺; melting point: 132-134° C. Anal. Calcd. For C₂₄H₄₂N₁₂O₄S C, 48.47; H, 7.12; N, 28.26%. Found C, 48.04; H, 7.13; N, 27.99%.

### Example 70

### N-Ethyl-N′-propyl-N″-prop-2-ynyl-[1,3,5]triazine-2,4,6-triamine (163) and corresponding hemisulfate salt (164b)

### N-Ethyl-N′-propyl-N″-prop-2-ynyl-[1,3,5]triazine-2,4,6-triamine (163)

6-Chloro-N-propyl-N′-prop-2-ynyl-[1,3,5]triazine-2,4-diamine (3) and ethylamine (70% in water solution, 2.66 mL, 5.32 mmol) were reacted according to the procedure described for compound (159) to afford N-ethyl-N′-propyl-N″-prop-2-ynyl-[1,3,5]triazine-2,4,6-triamine (163) (88% yield). 400 MHz ¹H NMR (CDCl₃, ppm): δ 5.38-5.14 (1H, m), 5.10-4.70 (2H, m), 4.31-4.07 (2H, m), 3.48-3.19 (4H, m), 2.19 (1H, t, J=2.5 Hz), 1.62-1.48 (2H, m), 1.16 (3H, t, J=7.3 Hz), 0.93 (3H, t, J=7.3 Hz). ESI-MS (m/z): 235 [M+H]⁺.

### N-Ethyl-N′-propyl-N″-prop-2-ynyl-[1,3,5]triazine-2,4,6-triamine hemisulfate (164b)

N-Ethyl-N′-methyl-N″-prop-2-ynyl-[1,3,5]triazine-2,4,6-triamine (163) and 95% H₂SO₄ were reacted according to the procedure described for compound (160b) to afford N-ethyl-N′-propyl-N″-prop-2-ynyl-[1,3,5]triazine-2,4,6-triamine hemisulfate (164b) (59% yield). 400 MHz ¹H NMR (CDCl₃, ppm): δ 5.38-5.14 (1H, m), 5.10-4.70 (2H, m), 4.31-4.07 (2H, m), 3.48-3.19 (4H, m), 2.19 (1H, t, J=2.5 Hz), 1.62-1.48 (2H, m), 1.16 (3H, t, J=7.3 Hz), 0.93 (3H, t, J=7.3 Hz). ESI-MS (m/z): 235 [M+H]⁺; melting point: 121-123° C.

### Example 71

### N-Propyl-N′-propyl-N″-prop-2-ynyl-[1,3,5]triazine-2,4,6-triamine (165) and corresponding hemisulfate salt (166b)

### N-Propyl-N′-propyl-N″-prop-2-ynyl-[1,3,5]triazine-2,4,6-triamine (165)

6-Chloro-N-propyl-N′-prop-2-ynyl-[1,3,5]triazine-2,4-diamine (3) and propylamine were reacted according to the procedure described for compound (159) to afford N-propyl-N′-propyl-N″-prop-2-ynyl-[1,3,5]triazine-2,4,6-triamine (165) (88% yield). 400 MHz ¹H NMR (CDCl₃, ppm): δ 5.09-4.64 (3H, m), 4.25-4.10 (2H, m), 3.39-3.22 (4H, m), 2.19 (1H, t, J=2.5 Hz), 1.63-1.49 (4H, m), 0.94 (6H, t, J=7.3 Hz). ESI-MS (m/z): 249 [M+H]⁺.

### N-Propyl-N′-propyl-N″-prop-2-ynyl-[1,3,5]triazine-2,4,6-triamine hemisulfate (166a)

N-Propyl-N′-propyl-N″-prop-2-ynyl-[1,3,5]triazine-2,4,6-triamine (165) and 95% H₂SO₄ were reacted according to the procedure described for compound (160b) to afford N-propyl-N′-propyl-N″-prop-2-ynyl-[1,3,5]triazine-2,4,6-triamine hemisulfate (166a) (84% yield). 400 MHz ¹H NMR (D₂O ppm): δ 4.32-4.11 (2H, m), 3.53-3.09 (4H, m), 2.58-2.41 (1H, m), 1.71-1.51 (4H, m), 0.93 (6H, t, J=7.3 Hz). ESI-MS (m/z): 249 [M+H]⁺; melting point: 175-177° C. Anal. Calcd. For C₂₄H₄₂N₁₂O₄S C, 48.47; H, 7.12; N, 28.26%. Found C, 48.52; H, 7.20; N, 28.20%.

### Example 72

### N-Cyclopropyl-N′-propyl-N″-prop-2-ynyl-[1,3,5]triazine-2,4,6-triamine (167) and corresponding hemisulfate salt (168b)

### N-Cyclopropyl-N′-propyl-N″-prop-2-ynyl-[1,3,5]triazine-2,4,6-triamine (167)

6-Chloro-N-propyl-N′-prop-2-ynyl-[1,3,5]triazine-2,4-diamine (3) and cyclopropylamine were reacted according to the procedure described for compound (159) to afford N-cyclopropyl-N′-propyl-N″-prop-2-ynyl-[1,3,5]triazine-2,4,6-triamine (167) (94% yield). 400 MHz ¹H NMR (CDCl₃, ppm): δ 5.20-4.80 (3H, m), 4.27-4.10 (2H, m), 3.40-3.23 (2H, m), 2.79-2.66 (1H, m), 2.19 (1H, t, J=2.5 Hz), 1.63-1.50 (2H, m), 0.93 (3H, t, J=7.3 Hz), 0.80-0.67 (2H, m), 0.57-0.44 (2H, m). ESI-MS (m/z): 247 [M+H]⁺.

### N-Cyclopropyl-N′-propyl-N″-prop-2-ynyl-[1,3,5]triazine-2,4,6-triamine hemisulfate (168a)

N-Cyclopropyl-N′-propyl-N″-prop-2-ynyl-[1,3,5]triazine-2,4,6-triamine (167) and 95% H₂SO₄ were reacted according to the procedure described for compound (160b) to afford N-cyclopropyl-N′-propyl-N″-prop-2-ynyl-[1,3,5]triazine-2,4,6-triamine hemisulfate (168a) (79% yield). 400 MHz ¹H NMR (D₂O ppm): δ 4.37-3.06 (2H, m), 3.53-3.25 (2H, m), 2.97-2.57 (2H, m), 1.72-1.50 (2H, m), 1.01-0.79 (2H, m), 0.93 (3H, t, J=7.3 Hz), 0.77-0.59 (2H, m). ESI-MS (m/z): 247 [M+H]⁺; melting point: 137-139° C.

### Example 73

### N-Isopropyl-N′-propyl-N″-prop-2-ynyl-[1,3,5]triazine-2,4,6-triamine (169) and corresponding hemisulfate salt (170b)

### N-Isopropyl-N′-propyl-N″-prop-2-ynyl-[1,3,5]triazine-2,4,6-triamine (169)

6-Chloro-N-propyl-N′-prop-2-ynyl-[1,3,5]triazine-2,4-diamine (3) and isopropylamine were reacted according to the procedure described for compound (159) to afford N-isopropyl-N′-propyl-N″-prop-2-ynyl-[1,3,5]triazine-2,4,6-triamine (169) (91% yield). 400 MHz ¹H NMR (CDCl₃, ppm): δ 5.11 (1H, s), 4.89 (1H, s), 4.72 (1H, s), 4.27-4.00 (3H, m), 3.39-3.17 (2H, m), 2.19 (1H, t, J=2.5 Hz), 1.63-1.49 (2H, m), 1.17 (6H, d, J=6.5 Hz), 0.93 (3H, t, J=7.3 Hz). ESI-MS (m/z): 249 [M+H]⁺.

### N-Isopropyl-N′-propyl-N″-prop-2-ynyl-[1,3,5]triazine-2,4,6-triamine hemisulfate (170b)

N-Isopropyl-N′-propyl-N″-prop-2-ynyl-[1,3,5]triazine-2,4,6-triamine (169) and 95% H₂SO₄ were reacted according to the procedure described for compound (160b) to afford N-isopropyl-N′-propyl-N″-prop-2-ynyl-[1,3,5]triazine-2,4,6-triamine hemisulfate (170b) (76% yield). 400 MHz ¹H NMR (D₂O ppm): δ 4.33-3.93 (3H, m), 3.49-3.22 (2H, m), 2.69-2.64 (1H, m), 1.68-1.53 (2H, m), 1.23 (6H, d, J=6.4 Hz), 0.93 (3H, t, J=7.3 Hz). ESI-MS (m/z): 249 [M+H]⁺; melting point: 173-175° C. Anal. Calcd. For C₂₄H₄₂N₁₂O₄S C, 48.47; H, 7.12; N, 28.26%. Found C, 48.07; H, 7.12; N, 28.07%.

### Example 74

### N-Butyl-N′-propyl-N″-prop-2-ynyl-[1,3,5]triazine-2,4,6-triamine (171) and corresponding hemisulfate salt (172b)

### N-Butyl-N′-propyl-N″-prop-2-ynyl-[1,3,5]triazine-2,4,6-triamine (171)

6-Chloro-N-propyl-N′-prop-2-ynyl-[1,3,5]triazine-2,4-diamine (3) and butylamine were reacted according to the procedure described for compound (159) to afford N-butyl-N′-propyl-N″-prop-2-ynyl-[1,3,5]triazine-2,4,6-triamine (171) (81% yield). 400 MHz ¹H NMR (CDCl₃, ppm): δ 5.06-4.62 (3H, m), 4.27-4.07 (2H, m), 3.44-3.19 (4H, m), 2.19 (1H, t, J=2.5 Hz), 1.82-1.68 (2H, m), 1.62-1.46 (4H, m), 1.41-1.33 (2H, m), 0.94 (3H, t, J=7.3 Hz), 0.93 (3H, t, J=7.3 Hz). ESI-MS (m/z): 263 [M+H]⁺.

### N-Butyl-N′-propyl-N″-prop-2-ynyl-[1,3,5]triazine-2,4,6-triamine hemisulfate (172b)

N-Butyl-N′-propyl-N″-prop-2-ynyl-[1,3,5]triazine-2,4,6-triamine (171) and 95% H₂SO₄ were reacted according to the procedure described for compound (160b) to afford N-butyl-N′-propyl-N″-prop-2-ynyl-[1,3,5]triazine-2,4,6-triamine hemisulfate (172b) (75% yield). 400 MHz ¹H NMR (CDCl₃ ppm): δ 8.17 (0.2H, br s), 8.04-7.79 (1H, m), 7.70-7.48 (0.8H, m), 5.56-5.40 (1H, m), 4.21 (1H, dd, J=5.5, 2.4 Hz), 4.14 (0.5H, dd, J=5.5, 2.4 Hz), 4.07 (0.5H, dd, J=5.5, 2.4 Hz), 2.26 (0.5H, t, J=2.4 Hz), 2.17 (0.5H, t, J=2.4 Hz), 1.68-1.47 (4H, m), 1.45-1.26 (2H, m), 1.00-0.86 (6H, m). ESI-MS (m/z): 263 [M+H]⁺; melting point: 140-142° C.

### Example 75

### N-Cyclopropylmethyl-N′-propyl-N″-prop-2-ynyl-[1,3,5]triazine-2,4,6-triamine (173) and corresponding hemisulfate salt (174b)

### N-Cyclopropylmethyl-N′-propyl-N″-prop-2-ynyl-[1,3,5]triazine-2,4,6-triamine (173)

6-Chloro-N-propyl-N′-prop-2-ynyl-[1,3,5]triazine-2,4-diamine (3) and cyclopropylmethylamine were reacted according to the procedure described for compound (159) to afford N-cyclopropylmethyl-N′-propyl-N″-prop-2-ynyl-[1,3,5]triazine-2,4,6-triamine (173) (83% yield). 400 MHz ¹H NMR (CDCl₃, ppm): δ 5.22-4.76 (3H, m), 4.24-4.08 (2H, m), 3.40-3.08 (4H, m), 2.19 (1H, t, J=2.5 Hz), 1.61-1.51 (2H, m), 1.08-0.89 (1H, m), 0.94 (3H, t, J=7.3 Hz), 0.55-0.42 (2H, m), 0.27-0.14 (2H, m). ESI-MS (m/z): 261 [M+H]⁺.

### N-Cyclopropylmethyl-N′-propyl-N″-prop-2-ynyl-[1,3,5]triazine-2,4,6-triamine hemisulfate (174b)

N-Cyclopropylmethyl-N′-propyl-N″-prop-2-ynyl-[1,3,5]triazine-2,4,6-triamine (173) and 95% H₂SO₄ were reacted according to the procedure described for compound (160b) to afford N-cyclopropylmethyl-N′-propyl-N″-prop-2-ynyl-[1,3,5]triazine-2,4,6-triamine hemisulfate (174b) (68% yield). 400 MHz ¹H NMR (CDCl₃ ppm): δ 8.49-8.41 (0.2H, m), 8.26-8.10 (0.5H, m), 7.96-7.80 (0.8H, m), 7.65-7.46 (0.5H, m), 5.66-5.43 (1H, m), 4.24-4.17 (1H, m), 4.16-4.06 (1H, m), 3.43-3.14 (4H, m), 2.28-2.15 (1H, m), 1.67-1.52 (2H, m), 1.10-0.99 (1H, m), 0.99-0.85 (3H, m), 0.60-0.44 (2H, m), 0.29-0.18 (2H, m). ESI-MS (m/z): 261 [M+H]⁺; melting point: 118-121° C.

### Example 76

### In Vitro Metabolic Stability Assay in Rat Liver Microsomes (RLM) and Human Liver Microsomes (HLM)

### Assay Description:

Liver microsomes were incubated with the test compound for a series of time points. Control compound (verapamil), with a known high clearance rate, was included in every experiment for comparison with the test compound. Analysis was conducted by evaluating the disappearance of parent compound over time. Microsomes from various animal species may be used to conduct a species comparison with human data.

### Materials and Reagent Preparation:

A 0.5 M stock of potassium phosphate buffer was diluted in deionized water to make up 50 mM working buffer. A large scale solution that was sufficient for several experiments was prepared, filtered-sterilized through a 0.2-micron filter using vacuum and stored at room temperature. A 8 mM NADPH solution was prepared by dissolving 100 mg NADPH sodium salt powder in 15 mL potassium phosphate buffer as prepared above to yield a final concentration of 8 mM (aliquoted and stored at −20° C.). Stock solution (5 mM) of test compounds were prepared by dissolving the material in methanol or 50/50 methanol/water. Separately, RLM (male, pooled) and/or HLM (mix-gender, pooled) with stock concentration of 20 mg/mL was obtained.

### Procedure:

A 10 mL (or 20 mL) of the previously prepared 0.05 M potassium phosphate buffer was dispensed into a 50-mL conical tube and 1M MgCl₂ stock (20 L or 40 μL) was added to a final concentration of 1.5 mM Mg²⁺. The solution was then aliquoted into Falcon tubes (for each test compound). The 5 mM compound stock solutions (4 μL) were used to make up a final compound concentration of 5 μM. The final organic content in the assay was 0.1%. From this, 300 μL of the compounds-containing buffer solutions were divided into cluster tubes (duplicate reactions with microsomal incubations, and a singleton reaction for negative control). The 20 mg/mL microsomal stocks were diluted in potassium phosphate buffer to yield a concentration of 4 mg/mL. Then, 50 μL of the 4 mg/mL microsomal working solution were placed into the duplicate reaction wells. For the negative control wells, 50 μL of potassium phosphate buffer was used (without MgCl₂). The cluster tubes were pre-warmed for approximately 10 min in a 37° C. 50-RPM shaking incubator. In the meantime, the appropriate volume (1.5 mL or 3 mL) of the 8 mM NADPH solution was warmed. The enzyme reaction was started by the addition of 50 μL pre-warmed NADPH solution to all cluster tubes using a multi-channel pipette. From this, 50 μL aliquots were removed at pre-determined time points (0, 5, 10, 15, 30, and 60 minutes) into a collection plate containing 150 μL acetonitrile containing propranolol as internal standard. After collecting the last time point, the plate(s) was/were centrifuged for 10 min at 2000g and the supernatant was transferred for LC-MS/MS analysis.

### Data Interpretation:

The percent parent compound remaining was determined relative to 0-minute incubation samples for each replicate, from which the elimination half-life was calculated based on the natural log of % compound remaining vs. time plot. The following parameters were calculated to estimate the compounds in vitro metabolic stability:
Cₘₚ=concentration of microsomal proteins (mg/mL);
t_{1/2}=the half-life (min), where t_{1/2} is equal to 0.693/slope;
CLᵢₙₜ=the intrinsic hepatic clearance (uL/min/mg), where CLᵢₙₜ=0.693/(t_{1/2}×Cₘₚ)
The metabolic stability of a test compound was categorized as follows:
Low clearance: CLᵢₙₜ (μL/min/mg)<10 (RLM) or 5 (HLM)
Moderate clearance: 10≦CLᵢₙₜ (μL/min/mg)≦60 (RLM) or 5≦CLᵢₙₜ (μL/min/mg)≦35 (HLM)
High clearance: CLᵢₙₜ (μL/min/mg)>60 (RLM) or 35 (HLM)

### Example 77

### Pharmacokinetic and Oral Bioavailability of Compound 5a in the Rat

### In-Life Procedures:

Sprague-Dawley rats (200-300 g) were fed a standard laboratory rodent diet and housed in individual cages on a 12-hour light and 12-hour dark cycle with room temperature maintained at 22±3° C. and relative humidity at 50±20%. Rats were fasted overnight before dosing, with food returned after the 6 hour blood samples were obtained. Bedding was removed until after the 6 hour blood samples were obtained. Water was provided ad libitum throughout the study.

### Oral Study:

Dosing solution of test compound was prepared in desired oral formulation for oral administration via gavage needle at 10-30 mg/kg (10 mL/kg) to three or four rats. All blood samples (250 uL per sample) were taken via jugular vein or femoral vein at 0 (predose), 15, and 30 min and 1, 2,4, 6, 8, and 24 h after oral dosing. Fluid replacement (1.5 mL of 0.9% sodium chloride injection, USP) were administered subcutaneously once after the 2 hr blood sampling. Blood samples were collected in BD Microtainer tubes coated with anticoagulant, placed on ice, and within 30 minutes, centrifuged at 15,000g for 3 min to obtain plasma samples. All plasma samples were stored at −70° C. until analysis by LC-MS/MS.

### Intravenous Study:

Dosing solution of test compound was prepared in desired intravenous formulation for intravenous bolus injection via tail vein, jugular vein, or femoral vein at 2-5 mg/kg (2 mL/kg) to three or four rats. All blood samples (250 uL per sample) were taken via jugular vein or femoral vein at 0 (predose), 5, 15, and 30 min and 1, 2, 4, 6, 8, and 24 h following intravenous administration. Fluid replacement (1.5 mL of 0.9% sodium chloride injection, USP) was administered subcutaneously once after the 2 hr blood sampling. Blood samples were collected in BD Microtainer tubes coated with anticoagulant, placed on ice, and within 30 minutes, centrifuged at 15,000g for 3 minutes to obtain plasma samples. All plasma samples were stored at −70° C. until analysis by LC-MS/MS.

### Bioanalytical Assay:

Plasma samples (incurred study samples, calibration standards, quality controls) were normally prepared as follows. Two volumes of acetonitrile containing an internal standard was added to one volume of plasma to precipitate plasma proteins. Samples were centrifuged (3,000 g for 5 min) and supernatant was removed for analysis by LC-MS-MS. Calibration standards were prepared by adding appropriate volumes of stock solution directly into blank plasma and treated identically to collected plasma samples. Calibration standards were typically prepared in the range of 2 ng/ml to 10 μg/mL for quantitation. Quality control samples were prepared in parallel at high, medium and low concentrations in an identical manner and they were used to ensure the quality of the assay results. No more that 2 of the 6 QC standards were allowed to differ by more than 20% of their nominal value. LC-MS-MS analysis was performed utilizing multiple reaction monitoring for detection of characteristic ions for each test compound, additional related analytes and internal standard. All ion source and tandem MS instrument parameters for the analytes were optimized for high sensitivity and selectivity.

### Pharmacokinetic Data Analysis:

All pharmacokinetic parameters were determined based on a non-compartmental approach using WinNonlin software (Pharsight, Version 5.1). The terminal elimination half-life (t_{1/2}) was calculated as ln2/λz using the slope (λz) from linear regression analysis of the terminal phase of the plasma concentration-time curve on a semi-log scale. The area under the plasma concentration-time curve (AUC_{inf}) was determined by non-compartmental analysis using the linear trapezoidal rule and extrapolated to infinity as Cₗₐₛₜ/λz using the last measurable concentration (Cₗₐₛₜ) and terminal slope (λz). The plasma concentration at time zero (C₀) following intravenous administration was estimated by linear extrapolation from the first two time points after dosing. The mean residence time (MRT) was obtained by dividing the area under the first moment curve (AUMC_{inf}) by AUC_{inf}. The systemic plasma clearance (CLₚ) was calculated as intravenous dose divided by AUC_{inf}. The volume of distribution at steady state (Vₛₛ) was determined as the product of CLₚ and MRT. The time to reach maximum plasma concentration (Tₘₐₓ) was based on the time to reach observed maximum concentrations. The maximum plasma concentration (Cₘₐₓ) was the observed maximum concentration occurring at Tₘₐₓ. The absolute oral bioavailability (F) was calculated as the percentage ratio of mean dose-normalized oral AUC_{inf} to dose-normalized intravenous AUC_{inf}.

Summary statistics for bioanalytical data and calculated pharmacokinetic parameters such as means, standard deviations, and coefficients of variation were determined using WinNonlin or Microsoft Excel applications.

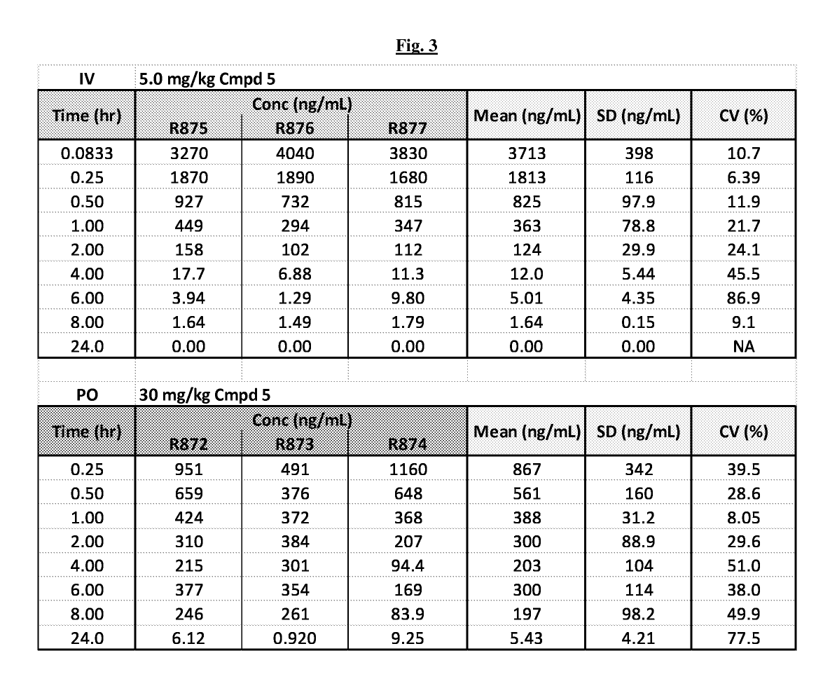

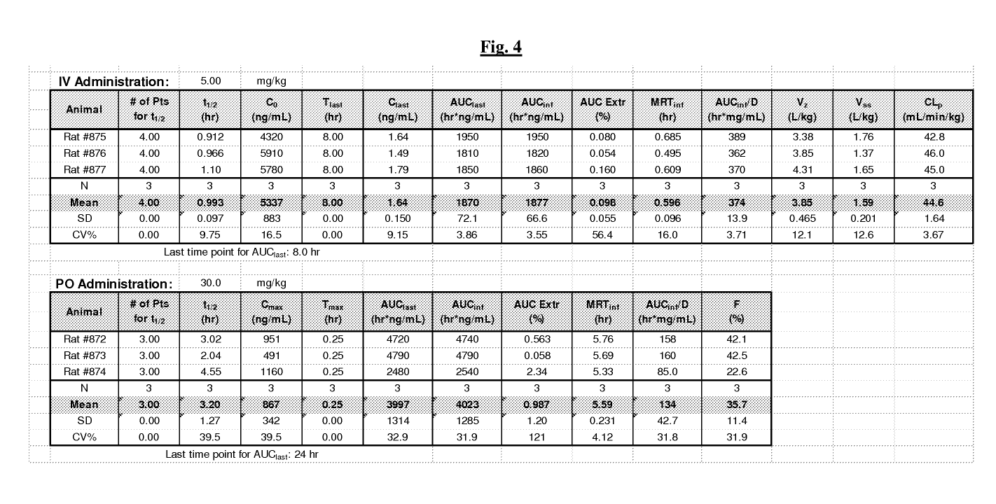

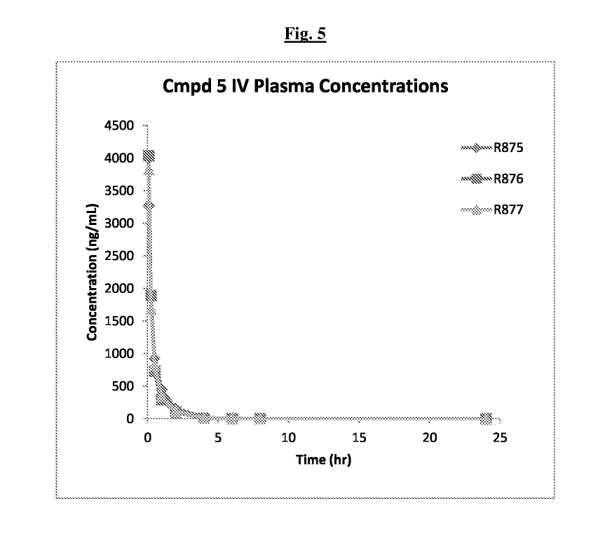

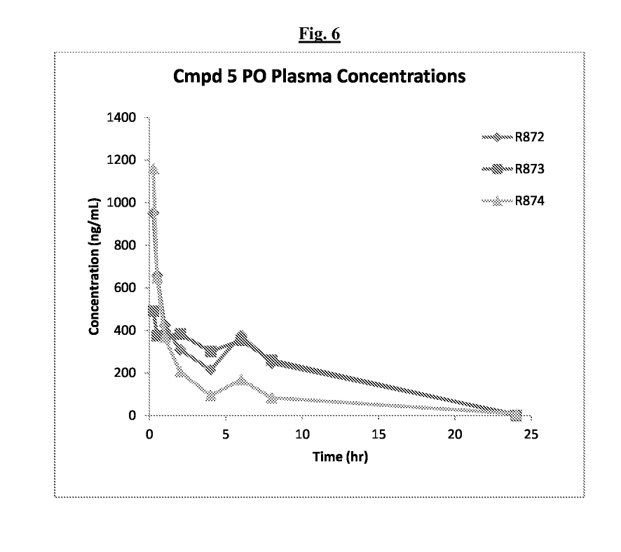

The compounds of the invention unexpectedly displayed enhanced oral bioavailability in comparison to the compounds described in the prior art, as exemplified in the data illustrated herein. Compound 5a was found to have an oral bioavailability of 36%. FIG. 3 is a table illustrating plasma concentrations upon dosing of Compound 5a to the rat. FIG. 4 is a table illustrating pharmacokinetic parameters of Compound 5a in the rat. FIGS. 5-7 illustrate plasma concentrations of Compound 5a dosed IV and PO.

### Example 78

### Effect of Compounds 5a, 7a and 9a on Respiratory Rate (RR), Tidal Volume (V_{T}), and the Product Minute Volume Using an Anesthetized Rat Spirometry Screening Assay

Anesthetized rats provide a quick method of screening compounds for respiratory and cardiovascular activity. In contrast to the conscious rat model, this model offers an experimental set up with less variation in the baseline cardiovascular and respiratory measures. Compounds screened in this assay may be examined in a conscious rat model.

Rats were initially anesthetized with 3% isoflurane (inhaled) and femoral artery and vein cannulas were surgically inserted. Once cannulated, the rats were transitioned to urethane anesthesia (1.5 g/kg; i.v.) and a tracheal cut-down was performed. After placing the tracheal cannula, it was connected to a pneumotach to record respiratory airflow from which respiratory rate (RR), tidal volume (V_{T}), and their product minute volume were derived. After the surgical preparation was complete, animals were allowed to stabilize for 30 minutes while respiratory rate, tidal volume, minute volume, blood pressure and heart rate were recorded continuously. Arterial blood gases (ABG) were obtained from arterial blood collected from the femoral artery. ABG measurements were taken before and 6 minutes after vehicle and each dose of compound administered. Compounds being screened were administered via bolus injections through the venous cannula followed by a saline flush (total time of administration is approximately 30 seconds), and the animal was monitored for at least 6 minutes for changes in cardiovascular efforts. Compounds were prepared in formulations identified to ensure optimal solubility. As such, vehicle controls were matched for the formulation of each compound tested. Dosing of the compound being screened was 0.1 and 0.3 mg/kg. The next dose was not administered until all cardiovascular and respiratory measures had returned to baseline levels. The positive control compounds used were N-[4,6-di-(n-propylamino)-[1,3,5]triazin-2-yl]-O,N-dimethyl-hydroxylamine or N-[2,6-di-(n-propylamino)-[1,3]pyrimidin-4-yl]-O,N-dimethyl-hydroxylamine, both administered at the end of each screening experiment (0.3 mg/kg dose) to validate the experiment and also to serve as a measure with which the compound being screened could be compared.

Data were analyzed by collecting cardiovascular and respiratory data in 30 second averages (BINs). Data were plotted 2 minutes before challenge and then 6 minutes after challenge.

### Results:

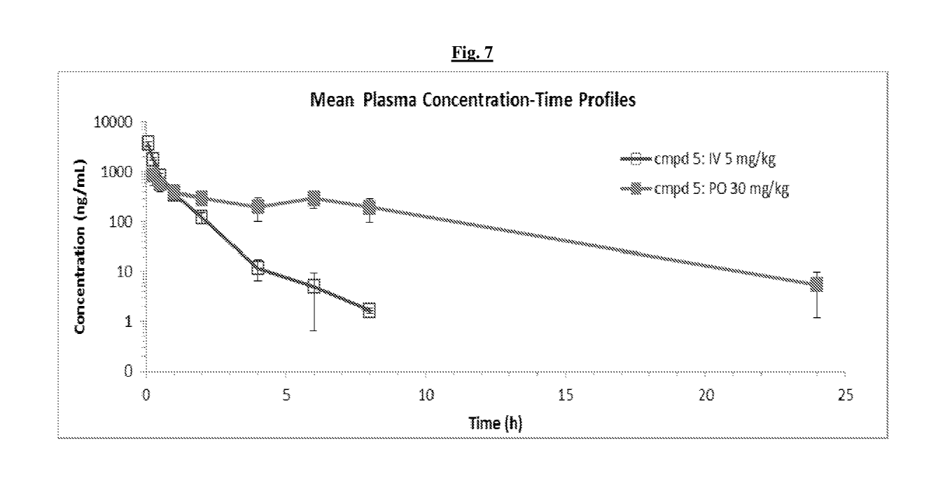

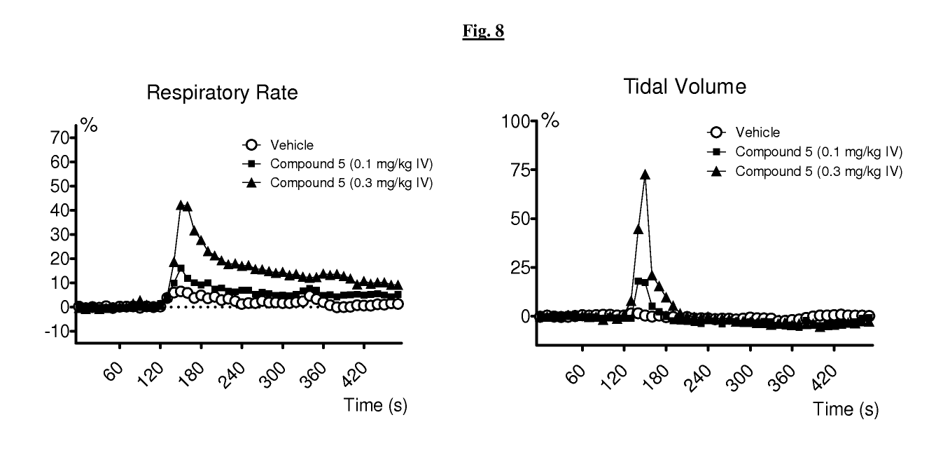

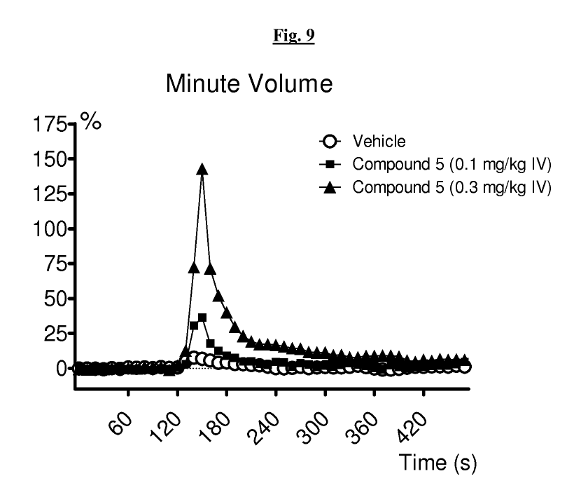

Compound 5a (0.3 mg/kg IV bolus) caused an immediate and short term (approximately 2 minutes duration) increase in minute ventilation by up to 143% above baseline values (FIG. 9). This increase was due to stimulatory effects on both respiratory rate (up to 42% increase) and tidal volume (up to 73% increase) (FIG. 8). The relative effects of Compound 5a on tidal volume were larger than its effects on respiratory rate.

Compound 7a (0.3 mg/kg IV bolus) caused an immediate and short term (approximately 2 minutes duration) increase in minute ventilation by up to 85% above baseline values (FIG. 11). This increase was due to stimulatory effects on both respiratory rate (up to 46% increase) and tidal volume (up to 27% increase) (FIG. 10). The relative effects of Compound 7a on respiratory rate were larger than its effects on tidal volume.

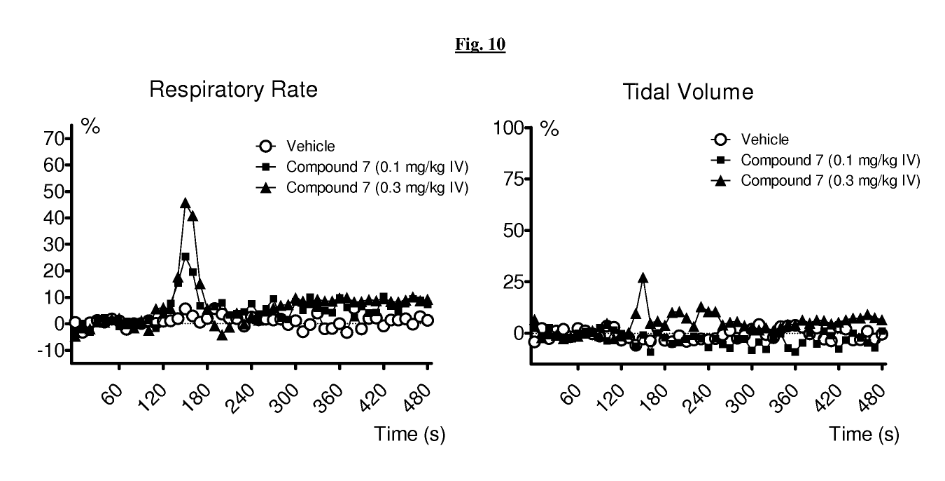

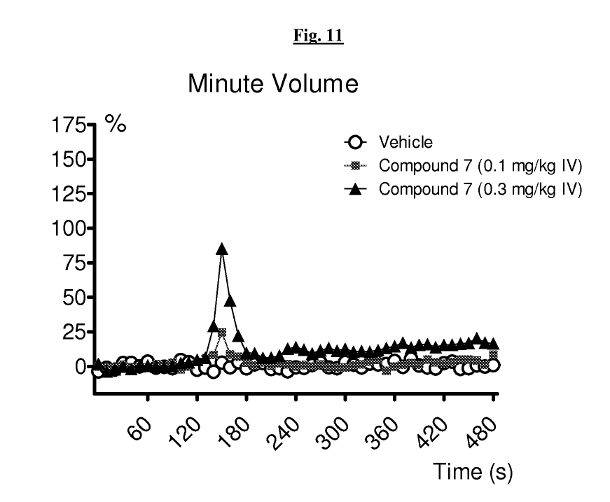

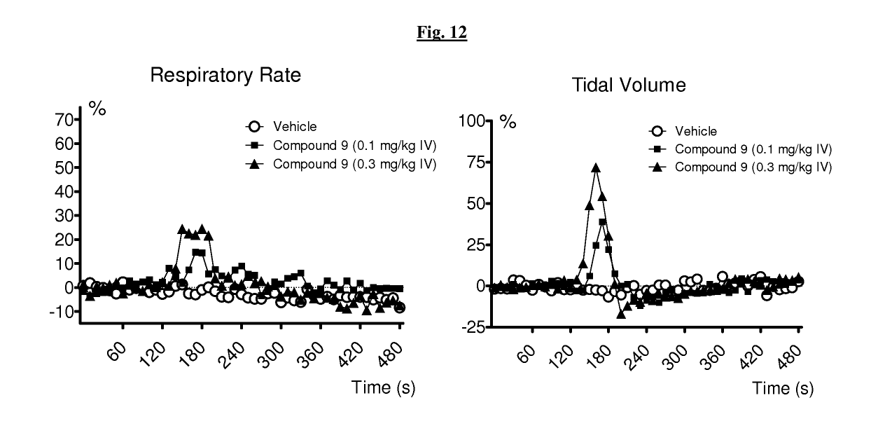

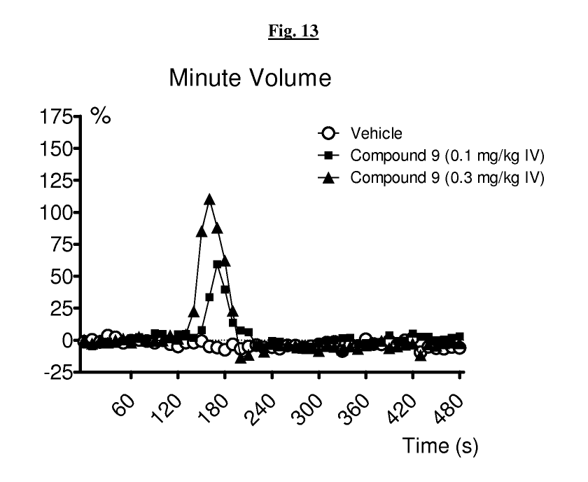

Compound 9a (0.3 mg/kg IV bolus) caused an immediate and short term (approximately 2 minutes duration) increase in minute ventilation by up to 110% above baseline values (FIG. 13). This increase was due to stimulatory effects on both respiratory rate (up to 24% increase) and tidal volume (up to 72% increase) (FIG. 12). The relative effects of Compound 9a on tidal volume were larger than its effects on respiratory rate.

FIG. 8 illustrates the effect of Compound 5a on respiratory rate and tidal volume dosed IV in the rat. FIG. 9 illustrates the effect of Compound 5a on minute volume dosed IV in the rat. FIG. 10 illustrates the effect of Compound 7a on respiratory rate and tidal volume dosed IV in the rat. FIG. 11 illustrates the effect of Compound 7a on minute volume dosed IV in the rat. FIG. 12 illustrates the effect of Compound 9a on respiratory rate and tidal volume dosed IV in the rat. FIG. 13 illustrates the effect of Compound 9a on minute volume dosed IV in the rat.

### Example 79

### Microsomal Stability and Ventilatory Activity

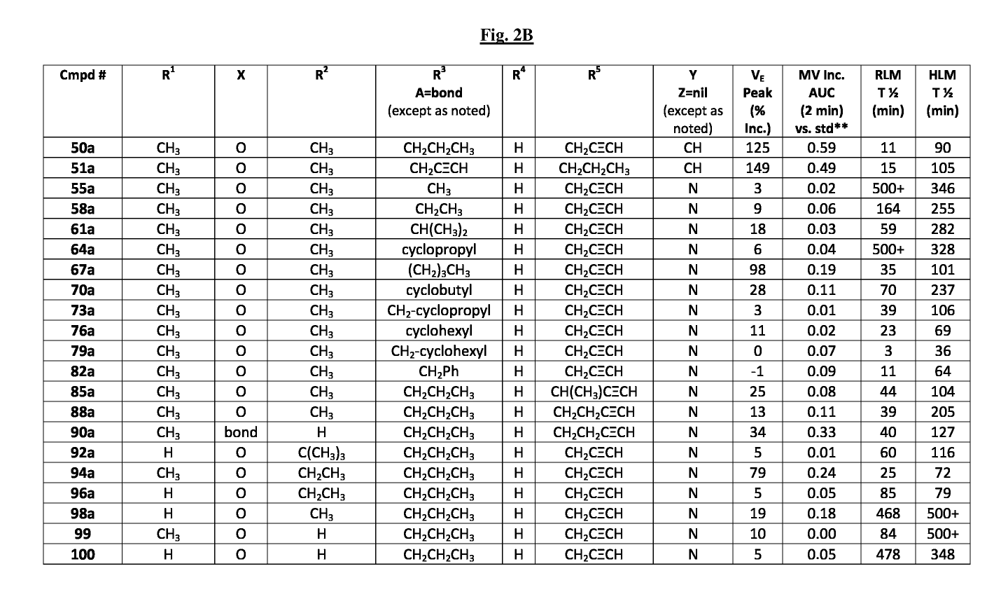

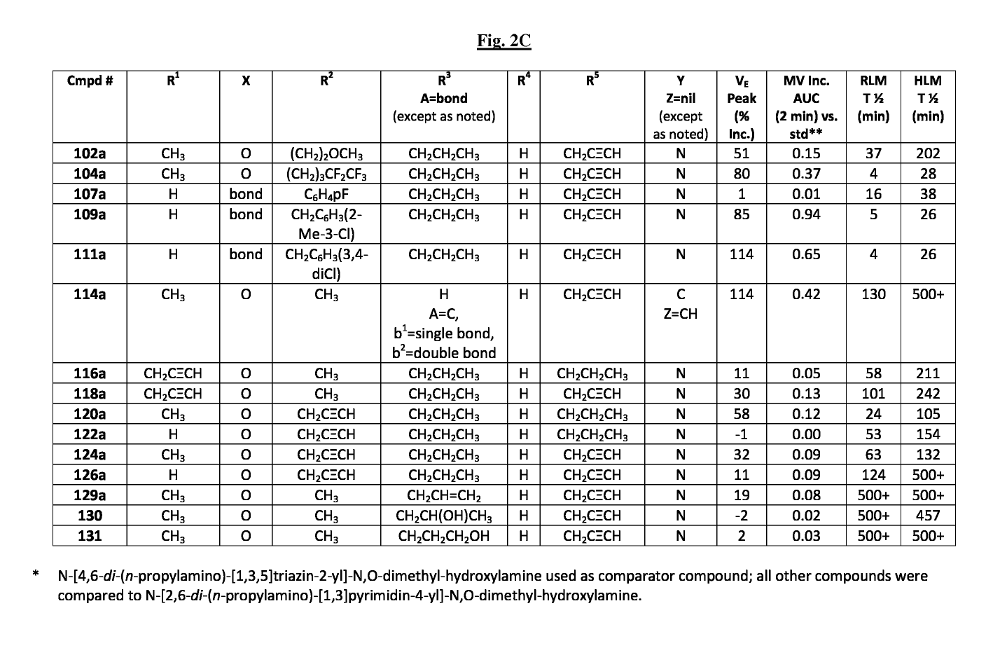

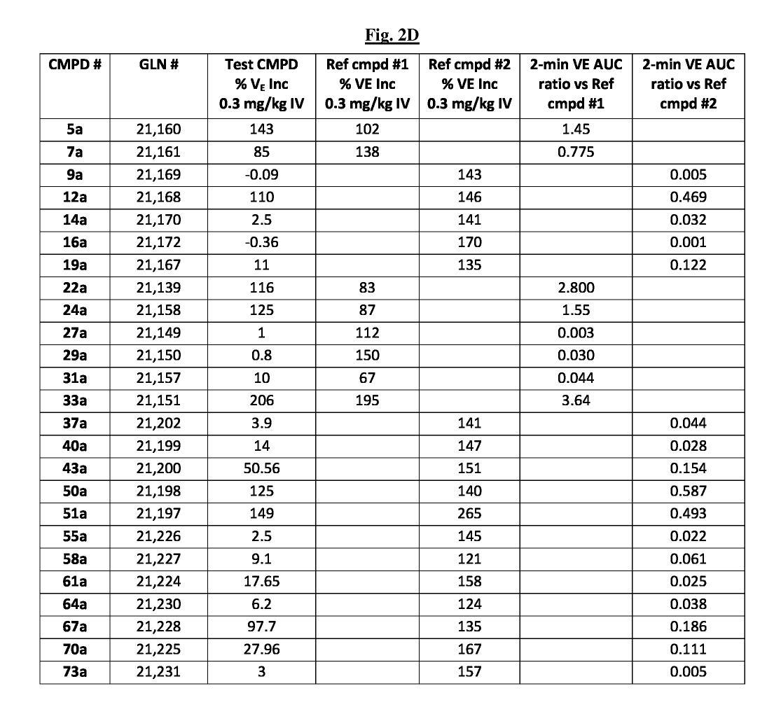

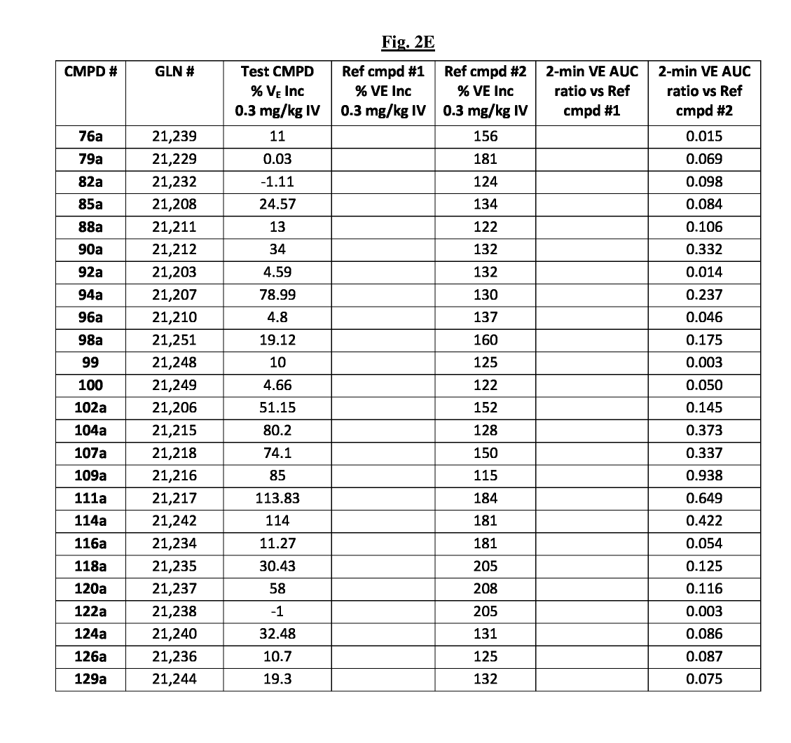

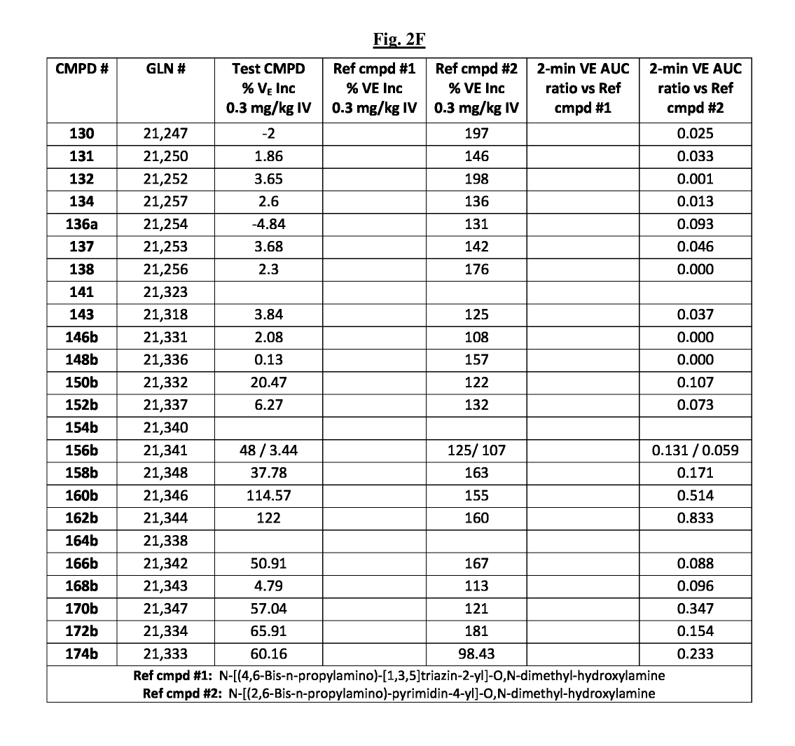

FIGS. 2A-2F summarize the effects of individual compounds on peak minute ventilation (increase in V_{E}), overall increase in 2 minute area under the curve (AUC) which is a cumulative measure of effect, along with compound stability (half-life) in rat and human microsomes.

### Example 80

### Effect of Compound 5b on Minute Volume (V_{E}) and Mean Blood Pressure (MBP) in the Rat Upon Oral Dosing

Compounds were administered orally to rat and evaluated for effects on ventilatory and cardiovascular parameters.

Male Sprague Dawley rats (0.30-0.38 kg, n=4) were surgically implanted with ECG/blood pressure telemeters (Telemetry Research, Inc., Auckland, New Zweland) prior to data collection. The animals were acclimated to plethysmography chambers for a minimum period of 60 minutes. After this time, test compound (20 mg/kg, PO) or vehicle (0.9% saline) was administered by oral gavage. Respiratory and cardiovascular data were collected immediately thereafter for a period of 3 h using plethysmography (Buxco, Inc.) and telemetry (LabChart data acquisition software, AD instruments, Inc.). Each rat received test compound and vehicle during the course of the study and thus each animal served as its own control. Vehicle effects were subtracted from drug response (Drug-Veh) and the difference was reported as a mean percent (%) change over time±SE.

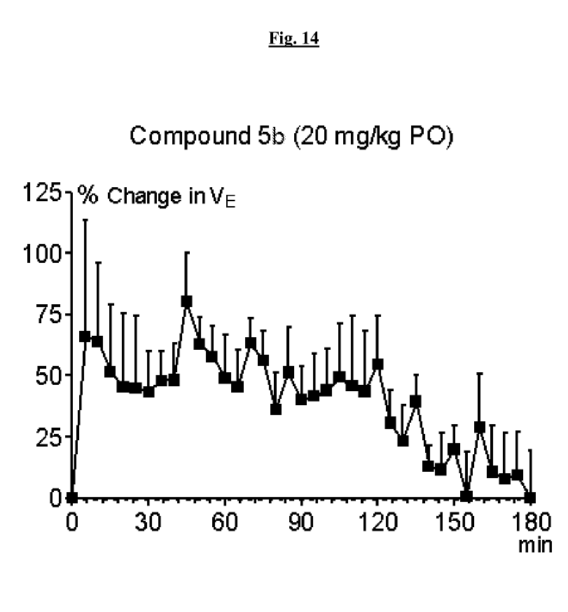

Compound 5b (20 mg/kg PO) produced an increase in minute ventilation of approximately 50% compared to baseline, with a duration of 120 min (FIG. 14). During this period, there was no significant effect on mean blood pressure (FIG. 15).

### Example 81

### Effect of Compound 5b on Apnea, Sleep Architecture and Ventilatory Response

The objective of this study was to quantify the effects of select compounds of this invention on apnea, ventilation and sleep structure in a rat model. The study consisted of two treatment conditions: (1) chronic morphine and test compound, such as Compound 5b administered orally (7 mg/kg, PO), and (2) chronic morphine and oral vehicle. Adult male lean Zucker rats were anesthetized for electroencephalogram (EEG) and electromyogram (EMG) telemeter implantation. At least 1 week was permitted post-surgery before animals were used further. Morphine sulfate was added to the drinking water of singly housed rats beginning with 0.1 mg/ml morphine and increasing the concentration in increments so that a final concentration of 0.6 mg/ml was achieved within 2 weeks. All respiratory measurements were made while animals were unrestrained in whole-body plethysmography chambers to permit animals to sleep and move freely. To avoid eliciting withdrawal, morphine water was continuously available during each plethysmography experiment. Minute volume and the number of central sleep apneas (CSA) were measured during all treatment conditions. A period of at least 1 to 1.5 h was permitted for animals to acclimate to the chamber before data collection began. The biopotential telemeters and their receivers were placed directly under the plethysmographic chambers and were used to continuously to capture EEG, EMG, and body temperature signals. Only data collected between the hours of 10 am to 4:30 pm were used in the final analysis to control for the effects of circadian rhythm on breathing.

Compound 5b (7 mg/kg) or an equivalent volume of vehicle was administered via an oral gavage tube at 12 pm. Data collected between 10 am and 12 pm represented baseline (pre-drug) conditions. Data collected between 12 pm and 4:30 pm represented post-drug conditions. Analyses of the EEG and EMG waveforms for the purpose of staging sleep-wake behavior in rats as awake, NREM sleep, and REM sleep were based on previous sleep studies involving rodents. Central apneas were defined as a respiratory cycle period that was more than or equal to twice the average cycle period during baseline recordings, which is consistent with how others have defined central apneas in rodents. Percent time in each sleep-wake state, minute volume, and central sleep apnea counts were compared between treatment groups using a two-way ANOVA (factors: drug treatment and time). Breathing data during wakefulness was not analyzed because movement prevents measurement of accurate volumes when using whole-body plethysmography. When differences were detected with ANOVA, Student-Neuman-Keuls post hoc tests were run for all main effects and interactions. Differences were considered significant when p<0.05. Values are expressed as means±SE.

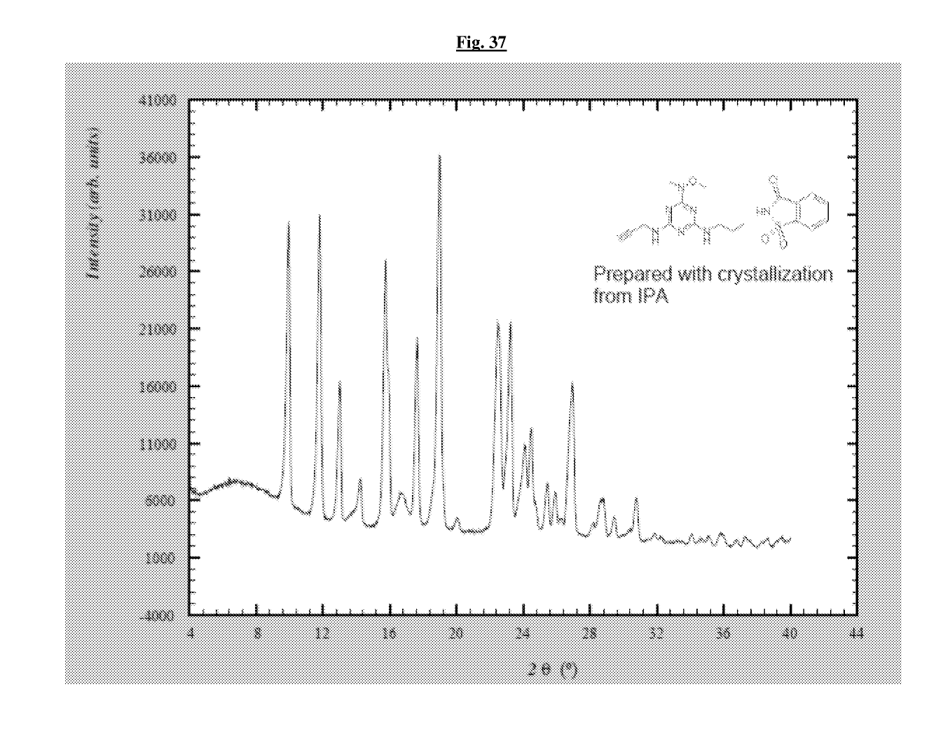

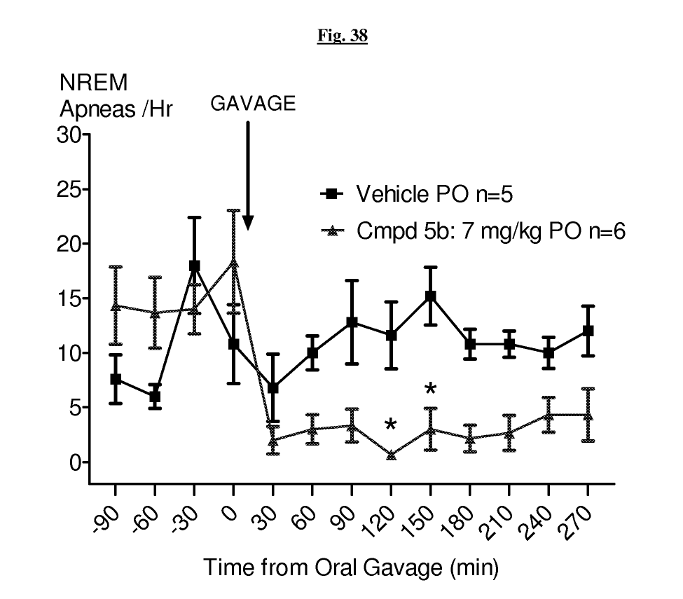

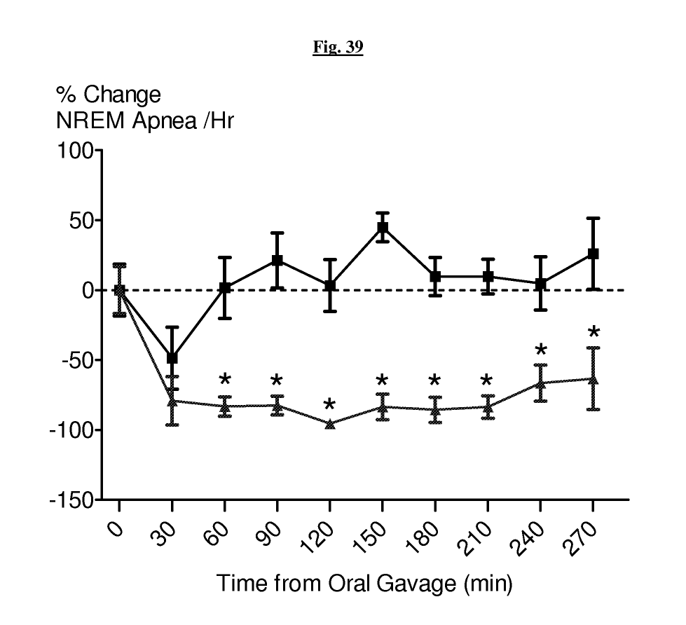

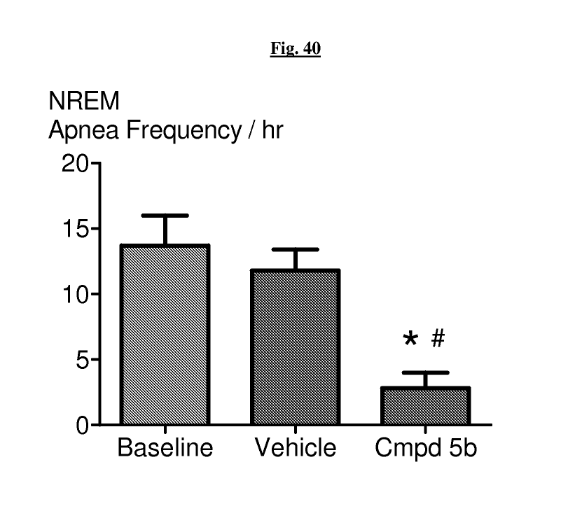

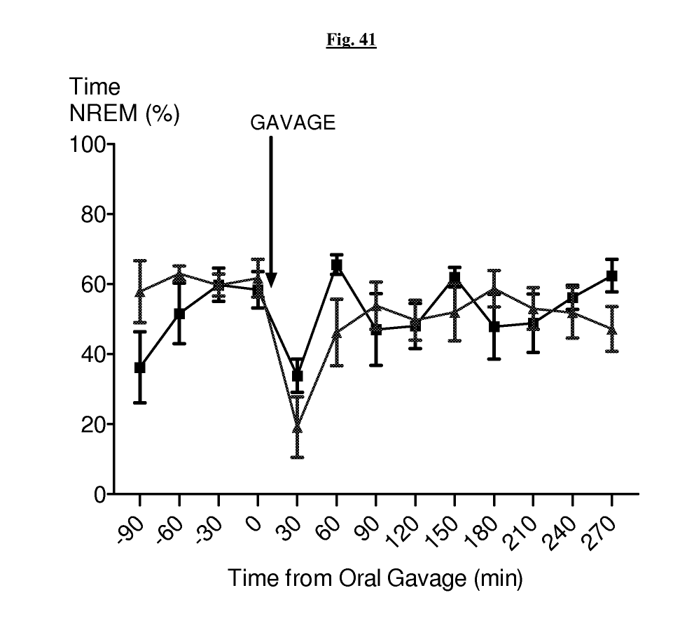

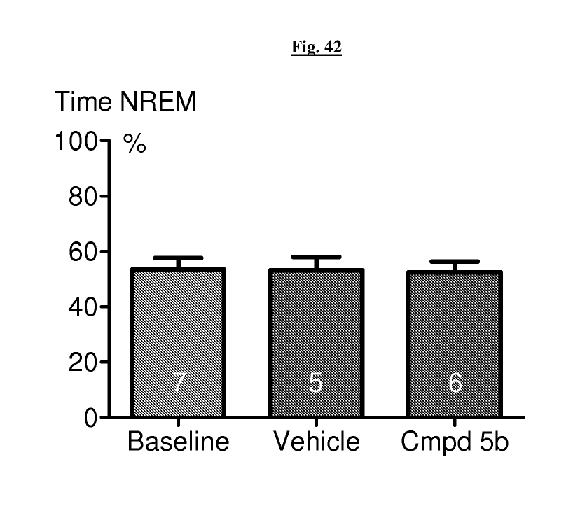

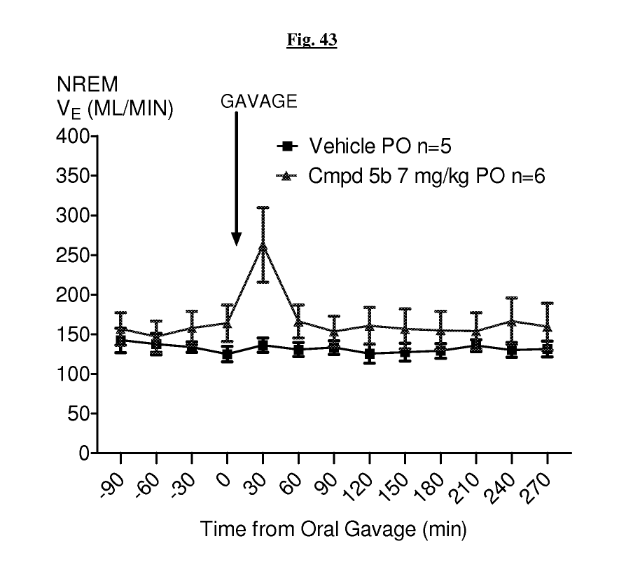

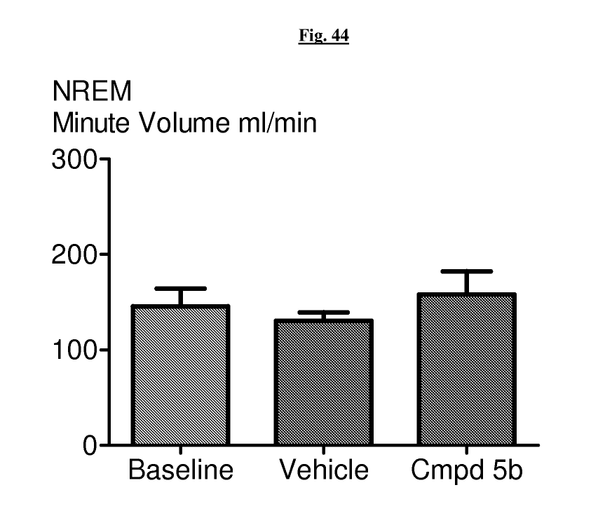

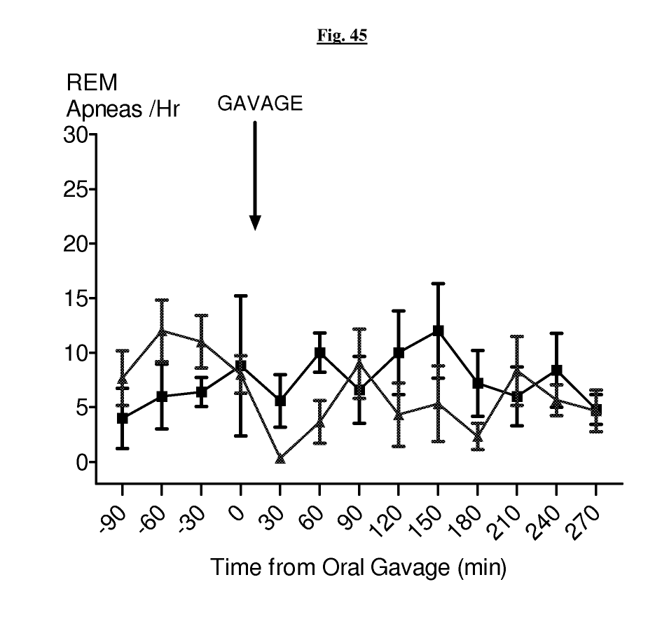

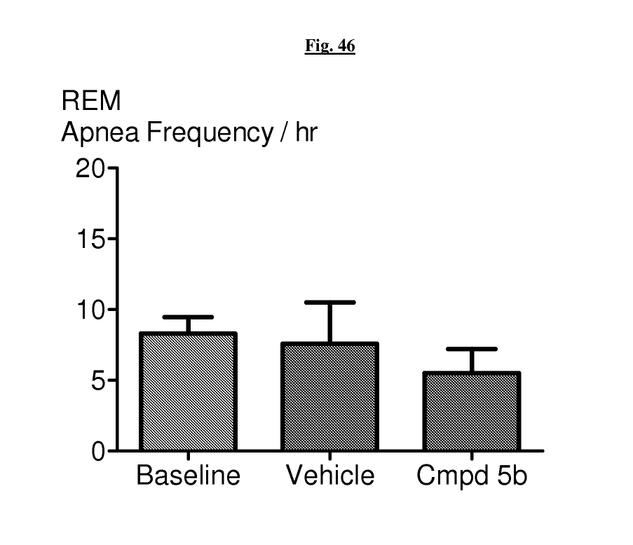

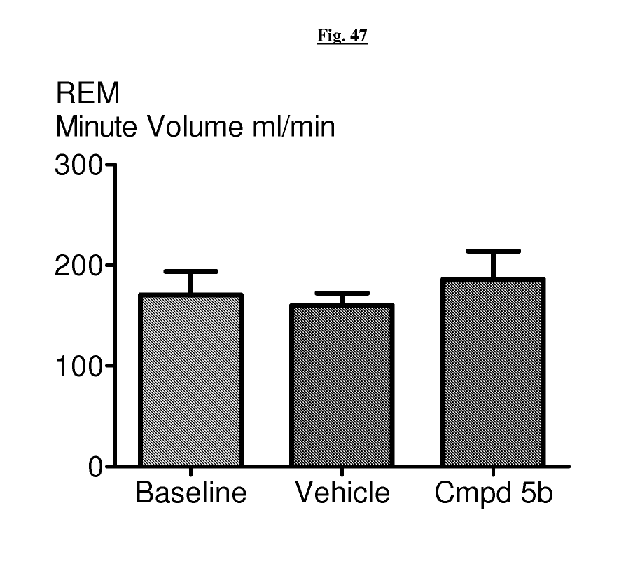

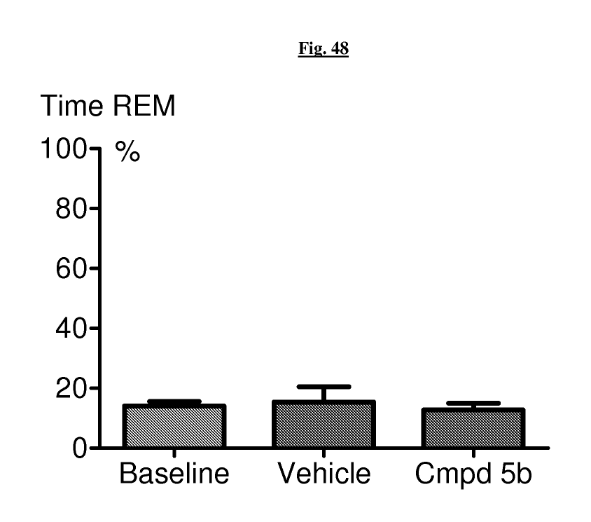

Compound 5b (7 mg/kg PO) significantly reduces the frequency of central apneas during NREM sleep (FIGS. 38-44) but does alter time spent in NREM nor stimulates ventilation during NREM. Compound 5b (7 mg/kg PO) does not reduce the frequency of central apneas during REM sleep (FIGS. 45-48) and does not alter time spent in REM sleep nor stimulate minute volume.

### Example 82

### Carotid sinus nerve transection (CSNTx) in rats receiving saline or Compound 5b

The objective of this study was to assess the role of carotid body sinus nerve activity on ventilation, as measured by minute volume, in rats treated with compounds of this invention, such as Compound 5b, versus a saline-treated group.

Urethane anesthetized adult male Sprague Dawley rats and tracheal spirometry (pneumotachometry) techniques were used (Example 56). Vehicle (saline) and Compound 5b (0.1 and 0.3 mg/kg) were administered as IV boluses to 5 rats. The carotid sinus nerves were isolated in the neck and transected thereby denervating both carotid bodies. Vehicle and Compound 5b were re-administered IV. Animals were exposed to low inspired O₂ (hypoxia) to confirm functionally complete nerve transections. The change in minute volume ΔV_{E} (% of baseline above baseline) in response to saline/Compound 5b before and after carotid body denervation was measured.

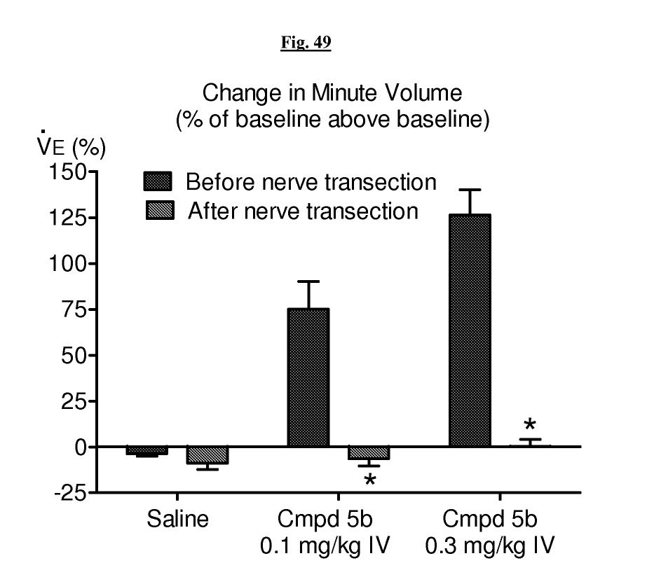

Compound 5b dose-dependently increased minute volume in all rats prior to carotid body denervation (FIG. 49). Carotid sinus nerve transection completely abolished the ventilatory response to Compound 5b given via IV bolus. SHAM surgery had no effect on the ventilatory response to Compound 5b. This data suggest that the carotid body mediates all of the ventilatory effects of Compound 5b (at doses tested) in urethane anesthetized rats.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A compound of formula (I) or a salt thereof: 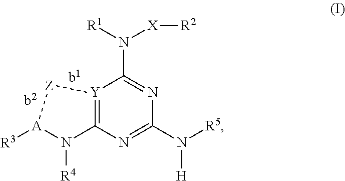
wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl or substituted alkynyl;
R⁴ is H, alkyl, or substituted alkyl;
R⁵ is alkyl, propargylic, substituted propargylic, homopropargylic, or substituted homopropargylic,
wherein at least one substituent selected from the group consisting of R¹, R², R³ and R⁵ is alkynyl or substituted alkynyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and:
(i) Z is H, bond b² is a single bond, and A is CH; or,
(ii) Z is nil, bond b² is nil, and A is a single bond; and,
if Y is C, then bond b¹ is a single bond, and:
(i) Z is CH₂, bond b² is a single bond, and A is CH; or,
(ii) Z is CH, bond b² is a double bond, and A is C.

2. The compound of claim 1, wherein (i) R³ is H, alkyl or substituted alkyl, and R⁵ is propargylic, substituted propargylic, homopropargylic, or substituted homopropargylic, or (ii) R³ is H or alkynyl, and R⁵ is alkyl, propargylic, substituted propargylic, homopropargylic, or substituted homopropargylic.

3. The compound of claim 1, wherein the compound is at least one selected from the group consisting of:
(i) Y is N, bond b¹ is nil, Z is H, bond b² is a single bond, A is CH, and the at least one compound is a compound of formula (II-a): 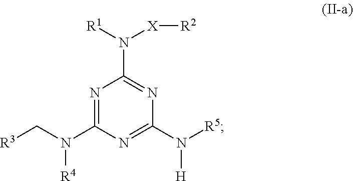
(ii) Y is N, bond b¹ is nil, Z is nil, bond b² is nil, and A is a bond, and the compound of the invention is a 1,3,5-triazine of formula (II-b); 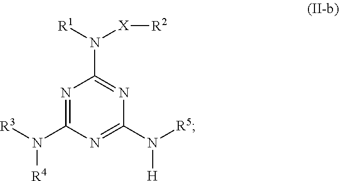
(iii) Y is CR⁶, bond b¹ is nil, Z is H, bond b² is a single bond, A is CH, and the at least one compound is a compound of formula (III-a); 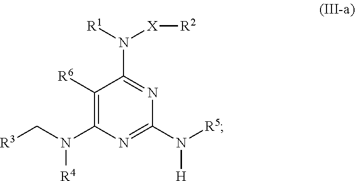
(iv) Y is CR⁶, bond b¹ is nil, Z is nil, bond b² is nil, and A is a bond, and the compound of the invention is a pyrimidine of formula (III-b): 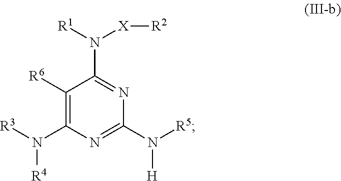
(v) Y is C, bond b¹ is a single bond, Z is CH₂, bond bis a single bond, A is CH, and the at least one compound is a compound of formula (IV): 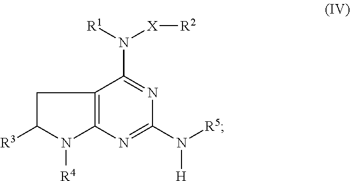
(vi) Y is C, bond b¹ is a single bond, Z is CH, bond b² is a double bond, A is C, and the at least one compound is a compound of formula (V):

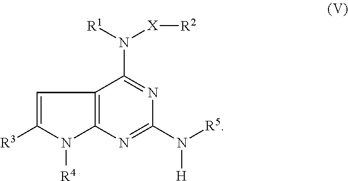

4. The compound of claim 1, wherein the at least one compound is selected from the group consisting of:
O,N-Dimethyl-N-[4(-n-propylamino)-6-(prop-2-ynylamino)-[1,3,5]triazin-2-yl]-hydroxylamine;
N-Methyl-N′-n-propyl-N″-prop-2-ynyl-[1,3,5]triazine-2,4,6-triamine;
N-(4-Fluorobenzyl)-O-methyl-N-[4-(n-propylamino)-6-(prop-2-ynylamino)-[1,3,5]triazin-2-yl]-hydroxylamine;
N-(4-Fluorobenzyl)-N′-n-propyl-N″-prop-2-ynyl-[1,3,5]triazine-2,4,6-triamine;
N-[4-(4-Fluorobenzylamino)-6-(prop-2-ynylamino)-[1,3,5]triazin-2-yl]-O,N-dimethyl-hydroxylamine;
N-(4-Fluoro-benzyl)-N-[4-(4-fluorobenzylamino)-6-(prop-2-ynylamino)-[1,3,5]triazin-2-yl]-O-methyl-hydroxylamine;
N,N′-Bis-(4-fluorobenzyl)-N″-prop-2-ynyl-[1,3,5]triazine-2,4,6-triamine;
N-(4,6-Bis-prop-2-ynylamino-[1,3,5]triazin-2-yl)-O,N-dimethyl-hydroxylamine;
N-Methyl-N′,N″-di-prop-2-ynyl-[1,3,5]triazine-2,4,6-triamine;
N,N′-Bis-(4-fluoro-benzyl)-N″-n-propyl-[1,3,5]triazine-2,4,6-triamine;
O-(4-Fluorophenyl)-N-(4-n-propylamino-6-prop-2-ynylamino-[1,3,5]triazin-2-yl)-hydroxylamine;
N-[4-(1,1-Dimethyl-prop-2-ynylamino)-6-n-propylamino-[1,3,5]triazin-2-yl]-O,N-dimethyl-hydroxylamine;
O,N-Dimethyl-N-(4-n-propylamino-6-but-2-ynylamino-[1,3,5]triazin-2-yl)-hydroxylamine;
O,N-Dimethyl-N-(6-n-propylamino-2-prop-2-ynylamino-pyrimidin-4-yl)-hydroxylamine;
O,N-Dimethyl-N-(2-n-propylamino-6-prop-2-ynylamino-pyrimidin-4-yl)-hydroxylamine;
O,N-Dimethyl-N-(4-methylamino-6-prop-2-ynylamino-[1,3,5]triazin-2-yl)-hydroxylamine;
O,N-Dimethyl-N-(4-ethylamino-6-prop-2-ynylamino-[1,3,5]triazin-2-yl)-hydroxylamine;
O,N-Dimethyl-N-(4-isopropylamino-6-prop-2-ynylamino-[1,3,5]triazin-2-yl)-hydroxylamine;
O,N-Dimethyl-N-(4-cyclopropylamino-6-prop-2-ynylamino-[1,3,5]triazin-2-yl)-ydroxylamine;
O,N-Dimethyl-N-(4-n-butylamino-6-prop-2-ynylamino-[1,3,5]triazin-2-yl)-hydroxylamine;
O,N-Dimethyl-N-(4-cyclobutylamino-6-prop-2-ynylamino-[1,3,5]triazin-2-yl)-hydroxylamine;
O,N-Dimethyl-N-(4-cyclopropylmethylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-benzylamino-6-prop-2-ynylamino-[1,3,5]triazin-2-yl)-hydroxylamine;
O,N-Dimethyl-N-[4-(1-methyl-prop-2-ynylamino)-6-n-propylamino-[1,3,5]triazin-2-yl]-hydroxylamine;
O,N-Dimethyl-N-(4-but-3-ynylamino-6-n-propylamino-[1,3,5]triazin-2-yl)-hydroxylamine;
N-But-3-ynyl-N′-methyl-N″-n-propyl-[1,3,5]triazine-2,4,6-triamine;
O-tert-Butyl-N-(4-n-propylamino-6-prop-2-ynylamino-[1,3,5]triazin-2-yl)-hydroxylamine;
O-Ethyl-N-methyl-N-(4-n-propylamino-6-prop-2-ynylamino-[1,3,5]triazin-2-yl)-hydroxylamine;
O-Ethyl-N-(4-n-propylamino-6-prop-2-ynylamino-[1,3,5]triazin-2-yl)-hydroxylamine;
O-Methyl-N-(4-n-propylamino-6-prop-2-ynylamino-[1,3,5]triazin-2-yl)-hydroxylamine;
N-Methyl-N-(4-n-propylamino-6-prop-2-ynylamino-[1,3,5]triazin-2-yl)-hydroxylamine;
N-(4-n-Propylamino-6-prop-2-ynylamino-[1,3,5]triazin-2-yl)-hydroxylamine;
O-(2-Methoxy-ethyl)-N-methyl-N-(4-n-propylamino-6-prop-2-ynylamino-[1,3,5]triazin-2-yl)-hydroxylamine;
N-Methyl-O-(4,4,5,5,5-pentafluoropentyl)-N-(4-n-propylamino-6-prop-2-ynylamino-[1,3,5]triazin-2-yl)-hydroxylamine;
N-(4-Fluorophenyl)-N′-propyl-N″-prop-2-ynyl-[1,3,5]triazine-2,4,6-triamine;
N-(3-Chloro-2-methyl-benzyl)-N′-n-propyl-N″-prop-2-ynyl-[1,3,5]triazine-2,4,6-triamine;
N-(3,4-Dichlorobenzyl)-N′-n-propyl-N″-prop-2-ynyl-[1,3,5]triazine-2,4,6-triamine;
O,N-Dimethyl-N-(2-prop-2-ynylamino-7H-pyrrolo[2,3-d]pyrimidin-4-yl)-hydroxylamine;
N-(4,6-Bis-n-propylamino-[1,3,5]triazin-2-yl)-O-methyl-N-prop-2-ynyl-hydroxylamine;
O-Methyl-N-(4-n-propylamino-6-prop-2-ynylamino-[1,3,5]triazin-2-yl)-N-prop-2-ynyl-hydroxylamine;
N-(4,6-Bis-n-propylamino-[1,3,5]triazin-2-yl)-N-methyl-O-prop-2-ynyl-hydroxylamine;
N-(4,6-Bis-n-propylamino-[1,3,5]triazin-2-yl)-O-prop-2-ynyl-hydroxylamine;
N-Methyl-N-(4-n-propylamino-6-prop-2-ynylamino-[1,3,5]triazin-2-yl)-O-prop-2-ynyl-hydroxylamine;
N-(4-n-Propylamino-6-prop-2-ynylamino-[1,3,5]triazin-2-yl)-O-prop-2-ynyl-hydroxylamine;
N-(4-Allylamino-6-prop-2-ynylamino-[1,3,5]triazin-2-yl)-O,N-dimethyl-hydroxylamine;
1-[4-(N-Methoxy-N-methyl-amino)-6-prop-2-ynylamino-[1,3,5]triazin-2-ylamino]-propan-2-ol;
3-[4-(N-Methoxy-N-methyl-amino)-6-prop-2-ynylamino-[1,3,5]triazin-2-ylamino]-propan-1-ol;
N-(4-Amino-6-prop-2-ynylamino-[1,3,5]triazin-2-yl)-O,N-dimethyl-hydroxylamine;
3-[4-(N-Methoxy-N-methylamino)-6-prop-2-ynylamino-[1,3,5]triazin-2-ylamino]-propionaldehyde;
3-[4-(N-Methoxy-N-methylamino)-6-prop-2-ynylamino-[1,3,5]triazin-2-ylamino]-propionic acid ethyl ester hydrochloride;
N-Propyl-N′-prop-2-ynyl-[1,3,5]triazine-2,4,6-triamine;
N-[4-(N′-Methoxy-N′-methyl-amino)-6-prop-2-ynylamino-[1,3,5]triazin-2-yl]-N-propyl acetamide;
N-[4-(N′-Methoxy-N′-methyl-amino)-6-prop-2-ynylamino-[1,3,5]triazin-2-yl]-N-propyl adamantylamide;
N-Ethyl-N′-methyl-N″-prop-2-ynyl-[1,3,5]triazine-2,4,6-triamine;
N-Cyclopropyl-N′-methyl-N″-prop-2-ynyl-[1,3,5]triazine-2,4,6-triamine;
N-Butyl-N′-methyl-N″-prop-2-ynyl-[1,3,5]triazine-2,4,6-triamine;
N-Cyclopropylmethyl-N′-methyl-N″-prop-2-ynyl-[1,3,5]triazine-2,4,6-triamine;
N-Methyl-N′-prop-2-ynyl-N″-(3,3,3-trifluoro-propyl)-[1,3,5]triazine-2,4,6-triamine;
N-Methyl-N′-(2,2,3,3,3-pentafluoro-propyl)-N″-prop-2-ynyl-[1,3,5]triazine-2,4,6-triamine;
N-(1-Ethyl-propyl)-N′-methyl-N″-prop-2-ynyl-[1,3,5]triazine-2,4,6-triamine;
N,N-Dimethyl-N′-propyl-N″-prop-2-ynyl-[1,3,5]triazine-2,4,6-triamine;
N,N-Ethyl-methyl-N′-propyl-N″-prop-2-ynyl-[1,3,5]triazine-2,4,6-triamine;
N-Ethyl-N′-propyl-N″-prop-2-ynyl-[1,3,5]triazine-2,4,6-triamine;
N-Propyl-N′-propyl-N″-prop-2-ynyl-[1,3,5]triazine-2,4,6-triamine;
N-Cyclopropyl-N′-propyl-N″-prop-2-ynyl-[1,3,5]triazine-2,4,6-triamine;
N-Isopropyl-N′-propyl-N″-prop-2-ynyl-[1,3,5]triazine-2,4,6-triamine;
N-Butyl-N′-propyl-N″-prop-2-ynyl-[1,3,5]triazine-2,4,6-triamine;
N-Cyclopropylmethyl-N′-propyl-N″-prop-2-ynyl-[1,3,5]triazine-2,4,6-triamine;
a salt thereof, and any combinations thereof.

5. The compound of claim 4, wherein the compound is selected from the group consisting of O,N-dimethyl-N-[4-(n-propylamino)-6-(prop-2-ynylamino-[1,3,5]triazin-2-yl]-hydroxylamine; N-methyl-N′-n-propyl-N″-prop-2-ynyl-[1,3,5]triazine-2,4,6-triamine; a salt thereof; and any combinations thereof.

6. The compound of claim 1, wherein the salt comprises an acid addition salt, and the acid is at least one selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, maleic, glucuronic, fumaric, pyruvic, aspartic, glutamic, benzoic, anthranilic, mandelic, pamoic, 4-hydroxybenzoic, phenylacetic, methanesulfonic, ethanesulfonic, alginic, benzenesulfonic, pantothenic, sulfanilic, stearic, trifluoromethanesulfonic, 2-hydroxyethanesulfonic, p-toluenesulfonic, cyclohexylaminosulfonic, β-hydroxybutyric, salicylic, galactaric and galacturonic, and any combinations thereof.

7. A pharmaceutical composition comprising a compound of claim 1 and at least one pharmaceutically acceptable carrier.

8. The composition of claim 7, further comprising at least one agent selected from the group consisting of doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists and ampakines.

9. The composition of claim 8, wherein the compound and the agent are physically mixed or physically separated in the composition.

10. The composition of claim 7, further comprising at least one additional agent that causes changes in breathing control.

11. The composition in claim 10, wherein the additional agent is at least one selected from the group consisting of opioid narcotics, benzodiazepines, sedatives, sleeping aids, hypnotics, propofol, and any combinations thereof.

12. The composition of claim 10, wherein the compound and the additional agent are physically mixed or physically separated in the composition.

13. The composition of claim 7, wherein the composition allows for modified delivery of the compound following oral administration to a subject.

14. The composition of claim 13, wherein the composition minimizes delivery of the compound to the stomach of the subject and maximizes delivery of the compound to the intestine of the subject.

15. A method of preventing or treating a breathing control disorder or disease in a subject in need thereof, the method comprising administering to the subject an effective amount of a pharmaceutical composition comprising at least one pharmaceutically acceptable carrier and at least one compound of formula (I) or a salt thereof: 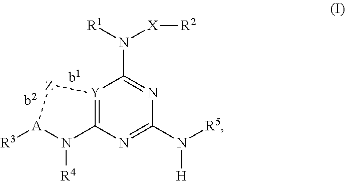
wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl or substituted alkynyl;
R⁴ is H, alkyl, or substituted alkyl;
R⁵ is alkyl, propargylic, substituted propargylic, homopropargylic, or substituted homopropargylic,
wherein at least one substituent selected from the group consisting of R¹, R², R³ and R⁵ is alkynyl or substituted alkynyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and:
(i) Z is H, bond b² is a single bond, and A is CH; or, (ii) Z is nil, bond b² is nil, and A is a single bond; and,
if Y is C, then bond b¹ is a single bond, and:
(i) Z is CH₂, bond b² is a single bond, and A is CH; or, (ii) Z is CH, bond b² is a double bond, and A is C.

16. The method of claim 15, wherein the breathing control disorder or disease is at least one selected from the group consisting of respiratory depression, sleep apnea, apnea of prematurity, obesity-hypoventilation syndrome, primary alveolar hypoventilation syndrome, dyspnea, altitude sickness, hypoxia, hypercapnia, chronic obstructive pulmonary disease (COPD), sudden infant death syndrome (SIDS), congenital central hypoventilation syndrome, Alzheimer's disease, Parkinson's disease, stroke, Duchenne muscular dystrophy, and brain and spinal cord traumatic injury.

17. The method of claim 16, wherein the respiratory depression is caused by an anesthetic, a sedative, a sleeping aid, an anxiolytic agent, a hypnotic agent, alcohol or a narcotic.

18. The method of claim 15, wherein the subject is further administered at least one agent useful for treating the breathing disorder or disease.

19. The method of claim 18, wherein the agent is at least one selected from the group consisting of doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists and ampakines.

20. The method of claim 18, wherein the compound and the agent are separately administered to the subject.

21. The method of claim 18, wherein the compound and the agent are co-administered to the subject, further wherein the compound and the agent are physically mixed or physically separated when administered to the subject.

22. The method of claim 15, wherein the subject is further administered at least one additional therapeutic agent that changes normal breathing control in the subject.

23. The method of claim 22, wherein that at least one additional agent is selected from the group consisting of opioid narcotics, benzodiazepines, sedatives, sleeping aids, hypnotics, propofol, and any combinations thereof.

24. The method of claim 15, wherein the composition is administered in conjunction with the use of a mechanical ventilation device or positive airway pressure device on the subject.

25. The method of claim 15, wherein the subject is a mammal or bird.

26. The method of claim 25, wherein the mammal is a human.

27. The method of claim 15, wherein the composition is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

28. The method of claim 15, wherein the at least one compound is selected from the group consisting of:
O,N-Dimethyl-N-[4(-n-propylamino)-6-(prop-2-ynylamino)-[1,3,5]triazin-2-yl]-hydroxylamine;
N-Methyl-N′-n-propyl-N″-prop-2-ynyl-[1,3,5]triazine-2,4,6-triamine;
N-(4-Fluorobenzyl)-O-methyl-N-[4-(n-propylamino)-6-(prop-2-ynylamino)-[1,3,5]triazin-2-yl]-hydroxylamine;
N-(4-fluorobenzyl)-N′-n-propyl-N″-prop-2-ynyl-[1,3,5]triazine-2,4,6-triamine;
N-[4-(4-Fluorobenzylamino)-6-(prop-2-ynylamino)-[1,3,5]triazin-2-yl]-O,N-dimethyl-hydroxylamine;
N-(4-fluoro-benzyl)-N-[4-(4-fluorobenzylamino)-6-(prop-2-ynylamino)-[1,3,5]triazin-2-yl]-O-methyl-hydroxylamine;
N,N′-Bis-(4-fluorobenzyl)-N″-prop-2-ynyl-[1,3,5]triazine-2,4,6-triamine;
N-(4,6-Bis-prop-2-ynylamino-[1,3,5]triazin-2-yl)-O,N-dimethyl-hydroxylamine;
N-Methyl-N′,N″-di-prop-2-ynyl-[1,3,5]triazine-2,4,6-triamine;
N,N′-Bis-(4-fluoro-benzyl)-N″-n-propyl-[1,3,5]triazine-2,4,6-triamine;
O-(4-fluorophenyl)-N-(4-n-propylamino-6-prop-2-ynylamino-[1,3,5]triazin-2-yl)-hydroxylamine;
N-[4-(1,1-Dimethyl-prop-2-ynylamino)-6-n-propylamino-[1,3,5]triazin-2-yl]-O,N-dimethyl-hydroxylamine;
O,N-Dimethyl-N-(4-n-propylamino-6-but-2-ynylamino-[1,3,5]triazin-2-yl)-hydroxylamine;
O,N-Dimethyl-N-(6-n-propylamino-2-prop-2-ynylamino-pyrimidin-4-yl)-hydroxylamine;
O,N-Dimethyl-N-(2-n-propylamino-6-prop-2-ynylamino-pyrimidin-4-yl)-hydroxylamine;
O,N-Dimethyl-N-(4-methylamino-6-prop-2-ynylamino-[1,3,5]triazin-2-yl)-hydroxylamine;
O,N-Dimethyl-N-(4-ethylamino-6-prop-2-ynylamino-[1,3,5]triazin-2-yl)-hydroxylamine;
O,N-Dimethyl-N-(4-isopropylamino-6-prop-2-ynylamino-[1,3,5]triazin-2-yl)-hydroxylamine;
O,N-Dimethyl-N-(4-cyclopropylamino-6-prop-2-ynylamino-[1,3,5]triazin-2-yl)-hydroxylamine;
O,N-Dimethyl-N-(4-n-buylamino-6-prop-2-ynylamino-[1,3,5]triazin-2-yl)-hydroxylamine;
O,N-Dimethyl-N-(4-cyclobutylamino-6-prop-2-ynylamino-[1,3,5]triazin-2-yl)-hydroxylamine;
O,N-Dimethyl-N-(4-cyclopropylmethylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-benzylamino-6-prop-2-ynylamino-[1,3,5]triazin-2-yl)-hydroxylamine;
O,N-Dimethyl-N-[4-(1-methyl-prop-2-ynylamino)-6-n-propylamino-[1,3,5]triazin-2-yl]-hydroxylamine;
O,N-Dimethyl-N-(4-but-3-ynylamino-6-n-propylamino-[1,3,5]triazin-2-yl)-hydroxylamine;
N-But-3-ynyl-N′-methyl-N″-n-propyl-[1,3,5]triazine-2,4,6-triamine;
O-tert-Butyl-N-(4-n-propylamino-6-prop-2-ynylamino-[1,3,5]triazin-2-yl)-hydroxylamine;
O-Ethyl-N-methyl-N-(4-n-propylamino-6-prop-2-ynylamino-[1,3,5]triazin-2-yl)-hydroxylamine;
O-Ethyl-N-(4-n-propylamino-6-prop-2-ynylamino-[1,3,5]triazin-2-yl)-hydroxylamine;
O-Methyl-N-(4-n-propylamino-6-prop-2-ynylamino-[1,3,5]triazin-2-yl)-hydroxylamine;
N-Methyl-N-(4-n-propylamino-6-prop-2-ynylamino-[1,3,5]triazin-2-yl)-hydroxylamine,
N-(4-n-Propylamino-6-prop-2-ynylamino-[1,3,5]triazin-2-yl)-hydroxylamine;
O-(2-Methoxy-ethyl)-N-methyl-N-(4-n-propylamino-6-prop-2-ynylamino-[1,3,5]triazin-2-yl)-hydroxylamine;
N-Methyl-O-(4,4,5,5,5-pentafluoropentyl)-N-(4-n-propylamino-6-prop-2-ynylamino-[1,3,5]triazin-2-yl)-hydroxylamine;
N-(4-Fluorophenyl)-N′-propyl-N″-prop-2-ynyl-[1,3,5]triazine-2,4,6-triamine;
N-(3-Chloro-2-methyl-benzyl)-N′-n-propyl-N″-prop-2-ynyl-[1,3,5]triazine-2,4,6-triamine;
N-(3,4-Dichlorobenzyl)-N′-n-propyl-N″-prop-2-ynyl-[1,3,5]triazine-2,4,6-triamine;
O,N-Dimethyl-N-(2-prop-2-ynylamino-7H-pyrrolo[2,3-d]pyrimidin-4-yl)-hydroxylamine;
N-(4,6-Bis-n-propylamino-[1,3,5]triazin-2-yl)-O-methyl-N-prop-2-ynyl-hydroxylamine;
O-Methyl-N-(4-n-propylamino-6-prop-2-ynylamino-[1,3,5]triazin-2-yl)-N-prop-2-ynyl-hydroxylamine;
N-(4,6-Bis-n-propylamino-[1,3,5]triazin-2-yl)-N-methyl-O-prop-2-ynyl-hydroxylamine;
N-(4,6-Bis-n-propylamino-[1,3,5]triazin-2-yl)-O-prop-2-ynyl-hydroxylamine;
N-Methyl-N-(4-n-propylamino-6-prop-2-ynylamino-[1,3,5]triazin-2-yl)-O-prop-2-ynyl-hydroxylamine;
N-(4-n-Propylamino-6-prop-2-ynylamino-[1,3,5]triazin-2-yl)-O-prop-2-ynyl-hydroxylamine;
N-(4-Allylamino-6-prop-2-ynylamino-[1,3,5]triazin-2-yl)-O,N-dimethyl-hydroxylamine;
1-[4-(N-Methoxy-N-methyl-amino)-6-prop-2-ynylamino-[1,3,5]triazin-2-ylamino]-propan-2-ol;
3-[4-(N-Methoxy-N-methyl-amino)-6-prop-2-ynylamino-[1,3,5]triazin-2-ylamino]-propan-1-ol;
N-(4-Amino-6-prop-2-ynylamino-[1,3,5]triazin-2-yl)-O,N-dimethyl-hydroxylamine;
3-[4-(N-Methoxy-N-methylamino)-6-prop-2-ynylamino-[1,3,5]triazin-2-ylamino]-propionaldehyde;
3-[4-(N-Methoxy-N-methylamino)-6-prop-2-ynylamino-[1,3,5]triazin-2-ylamino]-propionic acid ethyl ester hydrochloride;
N-Propyl-N′-prop-2-ynyl-[1,3,5]triazine-2,4,6-triamine;
N-[4-(N′-Methoxy-N′-methyl-amino)-6-prop-2-ynylamino-[1,3,5]triazin-2-yl]-N-propyl acetamide;
N-[4-(N′-Methoxy-N′-methyl-amino)-6-prop-2-ynylamino-[1,3,5]triazin-2-yl]-N-propyl adamantylamide;
N-Ethyl-N′-methyl-N″-prop-2-ynyl-[1,3,5]triazine-2,4,6-triamine;
N-Cyclopropyl-N′-methyl-N″-prop-2-ynyl-[1,3,5]triazine-2,4,6-triamine;
N-Butyl-N′-methyl-N″-prop-2-ynyl-[1,3,5]triazine-2,4,6-triamine;
N-Cyclopropylmethyl-N′-methyl-N″-prop-2-ynyl-[1,3,5]triazine-2,4,6-triamine;
N-Methyl-N′-prop-2-ynyl-N″-(3,3,3-trifluoro-propyl)-[1,3,5]triazine-2,4,6-triamine;
N-Methyl-N′-(2,2,3,3,3-pentafluoro-propyl)-N″-prop-2-ynyl-[1,3,5]triazine-2,4,6-triamine;
N-(1-Ethyl-propyl)-N′-methyl-N″-prop-2-ynyl-[1,3,5]triazine-2,4,6-triamine;
N,N-Dimethyl-N′-propyl-N″-prop-2-ynyl-[1,3,5]triazine-2,4,6-triamine;
N,N-Ethyl-methyl-N′-propyl-N″-prop-2-ynyl-[1,3,5]triazine-2,4,6-triamine;
N-Ethyl-N′-propyl-N″-prop-2-ynyl-[1,3,5]triazine-2,4,6-triamine;
N-Propyl-N′-propyl-N″-prop-2-ynyl-[1,3,5]triazine-2,4,6-triamine;
N-Cyclopropyl-N′-propyl-N″-prop-2-ynyl-[1,3,5]triazine-2,4,6-triamine;
N-Isopropyl-N′-propyl-N″-prop-2-ynyl-[1,3,5]triazine-2,4,6-triamine;
N-Butyl-N′-propyl-N″-prop-2-ynyl-[1,3,5]triazine-2,4,6-triamine;
N-Cyclopropylmethyl-N′-propyl-N″-prop-2-ynyl-[1,3,5]triazine-2,4,6-triamine;
a salt thereof, and any combinations thereof.

29. The method of claim 15, wherein the compound of formula (I) is selected from the group consisting of O,N-dimethyl-N-[4-(n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; a salt thereof; and any combinations thereof.

30. The method of claim 15, wherein the salt comprises an acid addition salt, and the acid is at least one selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, pamoic, methanesulfonic, ethanesulfonic, benzenesulfonic, pantothenic, sulfanilic, stearic, alginic, trifluoromethanesulfonic, 2-hydroxyethanesulfonic, p-toluenesulfonic, cyclohexylaminosulfonic, β-hydroxybutyric, salicylic, galactaric and galacturonic, and any combinations thereof.

31. A method of preventing destabilization or stabilizing breathing rhythm in a subject in need thereof, the method comprising administering to the subject an effective amount of a pharmaceutical composition comprising at least one pharmaceutically acceptable carrier and at least one compound of formula (I) or a salt thereof: 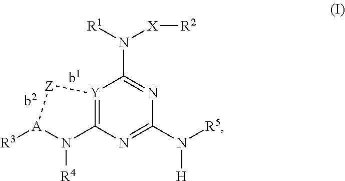
wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl or substituted alkynyl;
R⁴ is H, alkyl, or substituted alkyl;
R⁵ is alkyl, propargylic, substituted propargylic, homopropargylic, or substituted homopropargylic,
wherein at least one substituent selected from the group consisting of R¹, R², R³ and R⁵ is alkynyl or substituted alkynyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and:
(i) Z is H, bond b² is a single bond, and A is CH; or,
(ii) Z is nil, bond b² is nil, and A is a single bond; and,
if Y is C, then bond b¹ is a single bond, and:
(i) Z is CH₂, bond b² is a single bond, and A is CH; or,
(ii) Z is CH, bond b² is a double bond, and A is C.

32. The method of claim 31, wherein the destabilization is associated with a breathing control disorder or disease selected from the group consisting of respiratory depression, sleep apnea, apnea of prematurity, obesity-hypoventilation syndrome, primary alveolar hypoventilation syndrome, dyspnea, altitude sickness, hypoxia, hypercapnia, chronic obstructive pulmonary disease (COPD), sudden infant death syndrome (SIDS), congenital central hypoventilation syndrome, Alzheimer's disease, Parkinson's disease, stroke, Duchenne muscular dystrophy, and brain and spinal cord traumatic injury.

33. The method of claim 32, wherein the respiratory depression is caused by an anesthetic, a sedative, a sleeping aid, an anxiolytic agent, a hypnotic agent, alcohol or a narcotic.

34. The method of claim 31, wherein the subject is further administered at least one agent useful for treating the breathing disorder or disease.

35. The method of claim 34, wherein the agent is selected from the group consisting of doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists and ampakines.

36. The method of claim 34, wherein the compound and the agent are separately administered to the subject.

37. The method of claim 34, wherein the compound and the agent are co-administered to the subject, further wherein the compound and the agent are physically mixed or physically separated when administered to the subject.

38. The method of claim 31, wherein the subject is further administered at least one additional therapeutic agent that changes normal breathing control in the subject.

39. The method of claim 38, wherein the additional agent is at least one selected from the group consisting of opioid narcotics, benzodiazepines, sedatives, sleeping aids, hypnotics, propofol, and any combinations thereof.

40. The method of claim 31, wherein the composition is administered in conjunction with the use of a mechanical ventilation device or positive airway pressure device on the subject.

41. The method of claim 31, wherein the subject is a mammal or bird.

42. The method of claim 31, wherein the composition is administered to the subject by at least one route selected from the group consisting of a nasal, inhalational, topical, oral, buccal, rectal, pleural, peritoneal, vaginal, intramuscular, subcutaneous, transdermal, epidural, intrathecal and intravenous routes.

43. The method of claim 31, wherein the compound is selected from the group consisting of:
O,N-Dimethyl-N-[4(-n-propylamino)-6-(prop-2-ynylamino)-[1,3,5]triazin-2-yl]-hydroxylamine;
N-Methyl-N′-n-propyl-N″-prop-2-ynyl-[1,3,5]triazine-2,4,6-triamine;
N-(4-Fluorobenzyl)-O-methyl-N-[4-(n-propylamino)-6-(prop-2-ynylamino)-[1,3,5]triazin-2-yl]-hydroxylamine;
N-(4-Fluorobenzyl)-N′-n-propyl-N″-prop-2-ynyl-[1,3,5]triazine-2,4,6-triamine;
N-[4-(4-Fluorobenzylamino)-6-(prop-2-ynylamino)-[1,3,5]triazin-2-yl]-O,N-dimethyl-hydroxylamine;
N-(4-Fluoro-benzyl)-N-[4-(4-fluorobenzylamino)-6-(prop-2-ynylamino)-[1,3,5]triazin-2-yl]-O-methyl-hydroxylamine;
N,N′-Bis-(4-fluorobenzyl)-N″-prop-2-ynyl-[1,3,5]triazine-2,4,6-triamine;
N-(4,6-Bis-prop-2-ynylamino-[1,3,5]triazin-2-yl)-O,N-dimethyl-hydroxylamine;
N-Methyl-N′,N″-di-prop-2-ynyl-[1,3,5]triazine-2,4,6-triamine;
N,N′-Bis-(4-fluoro-benzyl)-N″-n-propyl-[1,3,5]triazine-2,4,6-triamine;
O-(4-Fluorophenyl)-N-(4-n-propylamino-6-prop-2-ynylamino-[1,3,5]triazin-2-yl)-hydroxylamine;
N-[4-(1,1-Dimethyl-prop-2-ynylamino)-6-n-propylamino-[1,3,5]triazin-2-yl]-O,N-dimethyl-hydroxylamine;
O,N-Dimethyl-N-(4-n-propylamino-6-but-2-ynylamino-[1,3,5]triazin-2-yl)-hydroxylamine;
O,N-Dimethyl-N-(6-n-propylamino-2-prop-2-ynylamino-pyrimidin-4-yl)-hydroxylamine;
O,N-Dimethyl-N-(2-n-propylamino-6-prop-2-ynylamino-pyrimidin-4-yl)-hydroxylamine;
O,N-Dimethyl-N-(4-methylamino-6-prop-2-ynylamino-[1,3,5]triazin-2-yl)-hydroxylamine;
O,N-Dimethyl-N-(4-ethylamino-6-prop-2-ynylamino-[1,3,5]triazin-2-yl)-hydroxylamine;
O,N-Dimethyl-N-(4-isopropylamino-6-prop-2-ynylamino-[1,3,5]triazin-2-yl)-hydroxylamine;
O,N-Dimethyl-N-(4-cyclopropylamino-6-prop-2-ynylamino-[1,3,5]triazin-2-yl)-hydroxylamine;
O,N-Dimethyl-N-(4-n-butylamino-6-prop-2-ynylamino-[1,3,5]triazin-2-yl)-hydroxylamine;
O,N-Dimethyl-N-(4-cyclobutylamino-6-prop-2-ynylamino-[1,3,5]triazin-2-yl)-hydroxylamine;
O,N-Dimethyl-N-(4-cyclopropylmethylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-benzylamino-6-prop-2-ynylamino-[1,3,5]triazin-2-yl)-hydroxylamine;
O,N-Dimethyl-N-[4-(1-methyl-prop-2-ynylamino)-6-n-propylamino-[1,3,5]triazin-2-yl]-hydroxylamine;
O,N-Dimethyl-N-(4-but-3-ynylamino-6-n-propylamino-[1,3,5]triazin-2-yl)-hydroxylamine;
N-But-3-ynyl-N′-methyl-N″-n-propyl-[1,3,5]triazine-2,4,6-triamine;
O-tert-Butyl-N-(4-n-propylamino-6-prop-2-ynylamino-[1,3,5]triazin-2-yl)-hydroxylamine;
O-Ethyl-N-methyl-N-(4-n-propylamino-6-prop-2-ynylamino-[1,3,5]triazin-2-yl)-hydroxylamine;
O-Ethyl-N-(4-n-propylamino-6-prop-2-ynylamino-[1,3,5]triazin-2-yl)-hydroxylamine;
O-Methyl-N-(4-n-propylamino-6-prop-2-ynylamino-[1,3,5]triazin-2-yl)-hydroxylamine;
N-Methyl-N-(4-n-propylamino-6-prop-2-ynylamino-[1,3,5]triazin-2-yl)-hydroxylamine;
N-(4-n-Propylamino-6-prop-2-ynylamino-[1,3,5]triazin-2-yl)-hydroxylamine;
O-(2-Methoxy-ethyl)-N-methyl-N-(4-n-propylamino-6-prop-2-ynylamino-[1,3,5]triazin-2-yl)-hydroxylamine;
N-Methyl-O-(4,4,5,5,5-pentafluoropentyl)-N-(4-n-propylamino-6-prop-2-ynylamino-[1,3,5]triazin-2-yl)-hydroxylamine;
N-(4-Fluorophenyl)-N′-propyl-N″-prop-2-ynyl-[1,3,5]triazine-2,4,6-triamine;
N-(3-Chloro-2-methyl-benzyl)-N′-n-propyl-N″-prop-2-ynyl-[1,3,5]triazine-2,4,6-triamine;
N-(3,4-Dichlorobenzyl)-N′-n-propyl-N″-prop-2-ynyl-[1,3,5]triazine-2,4,6-triamine;
O,N-Dimethyl-N-(2-prop-2-ynylamino-7H-pyrrolo[2,3-d]pyrimidin-4-yl)-hydroxylamine;
N-(4,6-Bis-n-propylamino-[1,3,5]triazin-2-yl)-O-methyl-N-prop-2-ynyl-hydroxylamine;
O-Methyl-N-(4-n-propylamino-6-prop-2-ynylamino-[1,3,5]triazin-2-yl)-N-prop-2-ynyl-hydroxylamine;
N-(4,6-Bis-n-propylamino-[1,3,5]triazin-2-yl)-N-methyl-O-prop-2-ynyl-hydroxylamine;
N-(4,6-Bis-n-propylamino-[1,3,5]triazin-2-yl)-O-prop-2-ynyl-hydroxylamine;
N-Methyl-N-(4-n-propylamino-6-prop-2-ynylamino-[1,3,5]triazin-2-yl)-O-prop-2-ynyl-hydroxylamine;
N-(4-n-Propylamino-6-prop-2-ynylamino-[1,3,5]triazin-2-yl)-O-prop-2-ynyl-hydroxylamine;
N-(4-Allylamino-6-prop-2-ynylamino-[1,3,5]triazin-2-yl)-O,N-dimethyl-hydroxylamine;
1-[4-(N-Methoxy-N-methyl-amino)-6-prop-2-ynylamino-[1,3,5]triazin-2-ylamino]-propan-2-ol;
3-[4-(N-Methoxy-N-methyl-amino)-6-prop-2-ynylamino-[1,3,5]triazin-2-ylamino]-propan-1-ol;
N-(4-Amino-6-prop-2-ynylamino-[1,3,5]triazin-2-yl)-O,N-dimethyl-hydroxylamine;
3-[4-(N-Methoxy-N-methylamino)-6-prop-2-ynylamino-[1,3,5]triazin-2-ylamino]-propionaldehyde;
3-[4-(N-Methoxy-N-methylamino)-6-prop-2-ynylamino-[1,3,5]triazin-2-ylamino]-propionic acid ethyl ester hydrochloride;
N-Propyl-N′-prop-2-ynyl-[1,3,5]triazine-2,4,6-triamine;
N-[4-(N′-Methoxy-N′-methyl-amino)-6-prop-2-ynylamino-[1,3,5]triazin-2-yl]-N-propyl acetamide;
N-[4-(N′-Methoxy-N′-methyl-amino)-6-prop-2-ynylamino-[1,3,5]triazin-2-yl]-N-propyl adamantylamide;
N-Ethyl-N′-methyl-N″-prop-2-ynyl-[1,3,5]triazine-2,4,6-triamine;
N-Cyclopropyl-N′-methyl-N″-prop-2-ynyl-[1,3,5]triazine-2,4,6-triamine;
N-Butyl-N′-methyl-N″-prop-2-ynyl-[1,3,5]triazine-2,4,6-triamine;
N-Cyclopropylmethyl-N′-methyl-N″-prop-2-ynyl-[1,3,5]triazine-2,4,6-triamine;
N-Methyl-N′-prop-2-ynyl-N″-(3,3,3-trifluoro-propyl)-[1,3,5]triazine-2,4,6-triamine;
N-Methyl-N′-(2,2,3,3,3-pentafluoro-propyl)-N″-prop-2-ynyl-[1,3,5]triazine-2,4,6-triamine;
N-(1-Ethyl-propyl)-N′-methyl-N″-prop-2-ynyl-[1,3,5]triazine-2,4,6-triamine;
N,N-Dimethyl-N′-propyl-N″-prop-2-ynyl-[1,3,5]triazine-2,4,6-triamine;
N,N-Ethyl-methyl-N′-propyl-N″-prop-2-ynyl-[1,3,5]triazine-2,4,6-triamine;
N-Ethyl-N′-propyl-N″-prop-2-ynyl-[1,3,5]triazine-2,4,6-triamine;
N-Propyl-N′-propyl-N″-prop-2-ynyl-[1,3,5]triazine-2,4,6-triamine;
N-Cyclopropyl-N′-propyl-N″-prop-2-ynyl-[1,3,5]triazine-2,4,6-triamine;
N-Isopropyl-N′-propyl-N″-prop-2-ynyl-[1,3,5]triazine-2,4,6-triamine;
N-Butyl-N′-propyl-N″-prop-2-ynyl-[1,3,5]triazine-2,4,6-triamine;
N-Cyclopropylmethyl-N′-propyl-N″-prop-2-ynyl-[1,3,5]triazine-2,4,6-triamine;
any salt thereof, and any combinations thereof.

44. The method of claim 31, wherein the compound of formula (I) is selected from the group consisting of O,N-dimethyl-N-[4-(n-propylamino)-6-(prop-2-ynylamino)-[1,3,5]triazin-2-yl]-hydroxylamine; N-Methyl-N′-n-propyl-N″-prop-2-ynyl-[1,3,5]triazine-2,4,6-triamine; a salt thereof; and any combinations thereof.

45. The method of claim 31, wherein the salt comprises an acid addition salt, and the acid is at least one selected from the group consisting of sulfuric, hydrochloric, hydrobromic, hydroiodic, nitric, carbonic, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, pamoic, methanesulfonic, ethanesulfonic, benzenesulfonic, pantothenic, sulfanilic, stearic, alginic, trifluoromethanesulfonic, 2-hydroxyethanesulfonic, p-toluenesulfonic, cyclohexylaminosulfonic, β-hydroxybutyric, salicylic, galactaric and galacturonic, and any combinations thereof.

46. A method of preparing O,N-dimethyl-N-[4-(n-propylamino)-6-(prop-2-ynylamino)-[1,3,5]triazin-2-yl]-hydroxylamine or a salt thereof, the method comprising the steps of:
(a) contacting cyanuric chloride with n-propyl amine in a solvent in the presence of a base;
(b) adding propargyl amine and a base to the mixture of step (a) and heating the resulting mixture;
(c) isolating from the mixture of step (b) solid 6-chloro-N-propyl-N′-prop-2-ynyl-[1,3,5]triazine-2,4-diamine;
(d) contacting the product of step (c) with O,N-dimethylhydroxylamine in a solvent at a temperature;
(e) isolating from the mixture of step (d) solid O,N-dimethyl-N-[4-(n-propylamino)-6-(prop-2-ynylamino)-[1,3,5]triazin-2-yl]-hydroxylamine; and,
(f) optionally contacting the product of step (e) with an acid, thereby forming an acid addition salt of O,N-dimethyl-N-[4-(n-propylamino)-6-(prop-2-ynylamino)-[1,3,5]triazin-2-yl]-hydroxylamine.

47. The method of claim 46, wherein the acid addition salt formed in step (f) is at least one selected from the group consisting of:
a sulfuric acid addition salt with an XRPD spectrum as illustrated in FIG. 22, 23, 24 or 25;
an L(+)-tartaric acid addition salt with an XRPD spectrum as illustrated in FIG. 27;
a maleic acid addition salt with an XRPD spectrum as illustrated in FIG. 29;
a DL-mandelic acid addition salt with an XRPD spectrum as illustrated in FIG. 31;
a malonic acid addition salt with an XRPD spectrum as illustrated in FIG. 33;
a fumaric acid addition salt with an XRPD spectrum as illustrated in FIG. 35; and,
a saccharin addition salt with an XRPD spectrum as illustrated in FIG. 37.

48. The method of claim 46, wherein the solid O,N-dimethyl-N-[4-(n-propylamino)-6-(prop-2-ynylamino)-[1,3,5]triazin-2-yl]-hydroxylamine has an XRPD spectrum as illustrated in FIG. 18 or 19.

49. The method of claim 46, wherein the product of step (f) is contacted with a base in a solvent, thereby yielding O,N-dimethyl-N-[4-(n-propylamino)-6-(prop-2-ynylamino)-[1,3,5]triazin-2-yl]-hydroxylamine free base.

50. The method of claim 49, wherein the O,N-dimethyl-N-[4-(n-propylamino)-6-(prop-2-ynylamino)-[1,3,5]triazin-2-yl]-hydroxylamine free base is contacted with an additional acid that is distinct from the acid in step (f), thereby yielding the additional acid addition salt of O,N-dimethyl-N-[4-(n-propylamino)-6-(prop-2-ynylamino)-[1,3,5]triazin-2-yl]-hydroxylamine.

51. The method of claim 46, wherein formation of 6-chloro-N,N′-propyl-[1,3,5]triazine-2,4-diamine in step (a) is minimized.

52. The method of claim 46, wherein the propargyl amine used in step (b) comprises less than 0.01 weight % of 2-chloroallyl amine.

53. The method of claim 46, wherein the propargyl amine used in step (b) comprises a 2:1 propargyl amine-sulfuric acid addition salt.

54. The method of claim 46, wherein the isolated compound in step (c) contains less than 0.5% 6-chloro-N,N′-propyl-[1,3,5]triazine-2,4-diamine.

55. The method of claim 46, wherein step (e) comprises the steps of:
cooling the mixture of step (d) below 60° C.;
diluting the resulting mixture with 2 volumes of water with vigorous stirring over about 2-3 h;
seeding the resulting system with a crystal of O,N-dimethyl-N-[4-(n-propylamino)-6-(prop-2-ynylamino)-[1,3,5]triazin-2-yl]-hydroxylamine;
stirring the resulting system for 10-20 h, whereby crystallization of O,N-dimethyl-N-[4-(n-propylamino)-6-(prop-2-ynylamino)-[1,3,5]triazin-2-yl]-hydroxylamine takes place.

56. The method of claim 46, wherein the solid O,N-dimethyl-N-[4-(n-propylamino)-6-(prop-2-ynylamino)-[1,3,5]triazin-2-yl]-hydroxylamine contains less that 0.01 weight % of N,O-dimethyl-N-(4-n-propylamino-6-(2-chloro-prop-2-enylamino)-[1,3,5]triazin-2-yl)-hydroxylamine.

57. A method of preparing the compound O,N-dimethyl-N-[4-(n-propylamino)-6-(prop-2-ynylamino)-[1,3,5]triazin-2-yl]-hydroxylamine or a salt thereof, wherein the compound is substantially free of N,O-Dimethyl-N-[4-n-propylamino-6-(2-chloro-prop-2-enylamino)-[1,3,5]triazin-2-yl]-hydroxylamine, the method comprising the steps of:
(a) contacting cyanuric chloride with n-propyl amine in a solvent in the presence of a base;
(b) adding N,O-dimethylhydroxylamine, optionally along with a base, to the mixture of step (a) and heating the resulting mixture;
(c) isolating from the mixture of step (b) the compound 6-chloro-N-n-propyl-N′-prop-2-ynyl-[1,3,5]triazine-2,4-diamine;
(d) contacting the compound isolated in step (c) with trialkyl amine in a solvent at a temperature, and isolating the compound 4-(N-methoxy-N-methyl-amino)-6-n-propylamino-[1,3,5]triazin-2-yl]-trimethyl-ammonium chloride;
(e) contacting the compound isolated in step (d) with a salt of tetrafluoroboric acid in a solvent at a temperature, and isolating the compound 4-(N-methoxy-N-methyl-amino)-6-n-propylamino-[1,3,5]triazin-2-yl]-trimethyl-ammonium tetrafluoroborate;
(f) contacting the compound isolated in step (e) with propargyl amine at a temperature, and isolating the compound N,O-dimethyl-N-(4-n-propylamino-6-prop-2-ynylamino-[1,3,5]triazin-2-yl)-hydroxylamine;
(g) optionally crystallizing the compound isolated in step (f) thus yielding crystalline N,O-dimethyl-N-(4-n-propylamino-6-prop-2-ynylamino-[1,3,5]triazin-2-yl)-hydroxylamine;
(h) optionally contacting the product isolated in step (f) or (g) with about one molar equivalent of maleic acid, and isolating the hydrogen maleinate salt of N,O-dimethyl-N-(4-n-propylamino-6-prop-2-ynylamino-[1,3,5]triazin-2-yl)-hydroxylamine;
(i) optionally contacting the product of step (h) with a base in a solvent, and isolating N,O-dimethyl-N-(4-n-propylamino-6-prop-2-ynylamino-[1,3,5]triazin-2-yl)-hydroxylamine free base; and,
(j) optionally contacting the compound isolated in step (g) or (i) about one molar equivalent of L(+)-tartartic acid in a solvent, and isolating the L(+)-hydrogen tartrate salt of N,O-dimethyl-N-(4-n-propylamino-6-prop-2-ynylamino-[1,3,5]triazin-2-yl)-hydroxylamine.

58. The method of claim 57, wherein the compound O,N-dimethyl-N-[4-(n-propylamino)-6-(prop-2-ynylamino)-[1,3,5]triazin-2-yl]-hydroxylamine or a salt thereof contains <0.002 weight % N,O-dimethyl-N-(4-n-propylamino-6-(2-chloro-prop-2-enyl)amino-[1,3,5]triazin-2-yl)-hydroxylamine.

59. A composition comprising O,N-dimethyl-N-[4-(n-propylamino)-6-(prop-2-ynylamino)-[1,3,5]triazin-2-yl]-hydroxylamine or a salt thereof selected from the group consisting of:
(a) a crystalline form of O,N-dimethyl-N-(4-n-propylamino-6-prop-2-ynylamino-[1,3,5]triazin-2-yl)-hydroxylamine with a XRPD spectrum as illustrated in FIG. 18 or 19;
(b) a crystalline form of O,N-dimethyl-N-(4-n-propylamino-6-prop-2-ynylamino-[1,3,5]triazin-2-yl)-hydroxylamine/sulfuric acid (1:1) addition salt with a XRPD spectrum as illustrated in FIG. 22;
(c) a crystalline form of O,N-dimethyl-N-(4-n-propylamino-6-prop-2-ynylamino-[1,3,5]triazin-2-yl)-hydroxylamine/sulfuric acid (2:1) addition salt with a XRPD spectrum as illustrated in FIG. 23;
(d) a crystalline form of O,N-dimethyl-N-(4-n-propylamino-6-prop-2-ynylamino-[1,3,5]triazin-2-yl)-hydroxylamine/sulfuric acid (1:2) addition salt with a XRPD spectrum as illustrated in FIG. 24;
(e) an amorphous form of O,N-dimethyl-N-(4-n-propylamino-6-prop-2-ynylamino-[1,3,5]triazin-2-yl)-hydroxylamine/sulfuric acid (4:3) addition salt with a XRPD spectrum as illustrated in FIG. 25;
(f) a crystalline form of O,N-dimethyl-N-(4-n-propylamino-6-prop-2-ynylamino-[1,3,5]triazin-2-yl)-hydroxylamine/L(+)-tartaric acid (1:1) addition salt with a XRPD spectrum as illustrated in FIG. 27;
(g) a crystalline form of O,N-dimethyl-N-(4-n-propylamino-6-prop-2-ynylamino-[1,3,5]triazin-2-yl)-hydroxylamine/maleic acid (1:1) addition salt with a XRPD spectrum as illustrated in FIG. 29;
(h) a crystalline form of O,N-dimethyl-N-(4-n-propylamino-6-prop-2-ynylamino-[1,3,5]triazin-2-yl)-hydroxylamine/DL-mandelic acid (1:1) addition salt with a XRPD spectrum as illustrated in FIG. 31;
(i) a crystalline form of O,N-dimethyl-N-(4-n-propylamino-6-prop-2-ynylamino-[1,3,5]triazin-2-yl)-hydroxylamine/malonic acid (1:1) addition salt with a XRPD spectrum as illustrated in FIG. 33;
(j) a crystalline form of O,N-dimethyl-N-(4-n-propylamino-6-prop-2-ynylamino-[1,3,5]triazin-2-yl)-hydroxylamine/fumaric acid (1:1) addition salt with a XRPD spectrum as illustrated in FIG. 35;
(k) a crystalline form of O,N-dimethyl-N-(4-n-propylamino-6-prop-2-ynylamino-[1,3,5]triazin-2-yl)-hydroxylamine/saccharin (1:1) addition salt with a XRPD spectrum as illustrated in FIG. 37;
and any combinations thereof.

60. A composition comprising [4-(N-methoxy-N-methyl-amino)-6-n-propylamino-[1,3,5]triazin-2-yl]-trimethyl-ammonium tetrafluoroborate.
